(19) United States
(12) Patent Application Publication
(10) Pub. No.: US 2009/0181377 A1
(43) Pub. Date: Jul. 16, 2009

(54) HUMAN RIBOSOMAL DNA (rDNA) AND RIBOSOMAL RNA (rRNA) NUCLEIC ACIDS AND USES THEREOF

(75) Inventors: Denis Drygin, San Diego, CA (US); Emil Michelotti, Foster City, CA (US); Sean O'Brien, Carlsbad, CA (US); William G. Rice, Del Mar, CA (US); Adam Siddiqui-Jain, San Diego, CA (US); Jeffrey P. Whitten, Santee, CA (US)

Correspondence Address: MORRISON & FOERSTER LLP, 12531 HIGH BLUFF DRIVE, SUITE 100, SAN DIEGO, CA, 92130-2040, US

(73) Assignee: Cylene Pharmaceuticals, Inc., San Diego, CA (US)

(21) Appl. No.: 12/025,705

(22) Filed: Feb. 4, 2008

Related U.S. Application Data
(62) Division of application No. 11/506,588, filed on Aug. 18, 2006, now abandoned.
(60) Provisional application No. 60/709,598, filed on Aug. 19, 2005.
(60) Provisional application No. 60/732,460, filed on Nov. 1, 2005.
(60) Provisional application No. 60/751,593, filed on Dec. 19, 2005.
(60) Provisional application No. 60/775,924, filed on Feb. 22, 2006.
(60) Provisional application No. 60/779,327, filed on Mar. 2, 2006.
(60) Provisional application No. 60/783,801, filed on Mar. 16, 2006.
(60) Provisional application No. 60/789,109, filed on Apr. 3, 2006.

(51) Int. Cl. C12Q 1/68; C07H 21/02

(52) U.S. Cl. 435/6; 536/23.1

(57) ABSTRACT

Provided herein are isolated nucleic acids that comprise a human rRNA or rDNA subsequence and related compositions and methods of use.

### RELATED PATENT APPLICATIONS

This application is a divisional of patent application Ser. No. 11/506,588 filed on Aug. 18, 2006 (pending) which claims the benefit of provisional application No. 60/732,460 filed on Nov. 1, 2005, provisional application No. 60/751,593 filed on Dec. 19, 2005, provisional application No. 60/775,924 filed on Feb. 22, 2006, provisional patent application No. 60/779,327 filed on Mar. 2, 2006, provisional patent application No. 60/783,801 filed on Mar. 16, 2006 and provisional patent application No. 60/789,109 filed on Apr. 3, 2006, each entitled HUMAN RIBOSOMAL DNA (rDNA) AND RIBOSOMAL RNA (rRNA) NUCLEIC ACIDS AND USES THEREOF, each naming Denis Drygin et al. as inventors and having attorney docket no. 532233002501, 532233002502, 5322233002503, 532233002504, 532233002505 and 532233002506, respectively. This application also claims the benefit of provisional application No. 60/709,598 filed on Aug. 19, 2005, entitled RIBOSOMAL DNA (rDNA) AND RIBOSOMAL RNA (rRNA) QUADRUPLEX NUCLEIC ACIDS AND METHODS FOR INDUCING APOPTOSIS, naming Denis Drygin et al. and having attorney docket no. 532233002500. Each of these patent applications is incorporated herein by reference in its entirety, including all text, nucleic acid sequences, chemical structures, tables and drawings.

### REFERENCE TO SEQUENCE LISTING SUBMITTED VIA EFS-WEB

This application is being filed electronically via the USPTO EFS-WEB server, as authorized and set forth in MPEP §1730 II.B.2(a)(A), and this electronic filing includes an electronically submitted sequence (SEQ ID) listing. The entire content of this sequence listing is herein incorporated by reference for all purposes. The sequence listing is identified on the electronically filed .txt file as follows:

| File Name | Date of Creation | Size (bytes) |
|---|---|---|
| 532232002510Seqlist.txt | Feb. 1, 2008 | 88,277 bytes |

### FIELD OF THE INVENTION

The invention relates to nucleic acids having selected nucleotide sequences identified in human ribosomal RNA, DNA sequences encoding the foregoing and related uses, including, without limitation, assays and treatments.

### BACKGROUND

Proteins in cells are synthesized in a process referred to as “translation.” Proteins are translated from messenger ribonucleic acids (mRNAs), the latter having been transcribed from deoxyribonucleic acid (DNA) nucleotide sequences. Each protein is synthesized as a chain of amino acids, and in the translation process ribosomes bind to and travel along the mRNA and sequentially add each amino acid in the chain. A ribosome bound to an mRNA selects a tRNA-loaded amino acid according to nucleotide triplets (i.e., codons) sequentially arranged along the mRNA.

A human ribosome is an 80S particle that comprises a 605 large subunit and a 40S small subunit. The “S” designation in “80S,” “60S” and “40S” refers to a “Svedberg unit,” a sedimentation measure of particle size. Each ribosome subunit is an assembly of proteins and functional RNA, which serves as a docking region for tRNA-loaded amino acids. The functional RNA is referred to as “ribosomal RNA (rRNA)” and it is synthesized by polymerase I and III enzymes that utilize a region of genomic DNA, referred to as “ribosomal DNA (rDNA),” as a template. The rDNA sequence is repeated approximately 400 times in the human genome. Ribosomal RNA biogenesis begins with the synthesis of a 47S precursor rRNA, which is iteratively cleaved into smaller, mature 18S, 5.8S and 28S rRNA by the coordinated action of a variety of endonucleases, exonucleases, RNA helicases and other protein factors. The 18S rRNA is assembled into the 40S ribosomal subunit and the 28S and 5.8S rRNA are assembled into the 60S ribosomal subunit. Human ribosome biogenesis occurs mainly in the nucleolus, a specialized compartment in the cell nucleus.

### SUMMARY

It has been discovered that guanine-rich nucleotide sequences having a quadruplex nucleotide sequence motif are present in human genomic rDNA and in the encoded rRNA. These nucleotide sequences were discovered by searching human rDNA for the guanine-rich nucleotide sequence ((G₃₊)N₁₋₇)₃G₃₊ (SEQ ID NO:230), where G is guanine, N is any nucleotide and “G₃₊” is three or more guanines. Nucleotide sequences also were discovered by searching human rDNA for the cytosine-rich nucleotide sequence ((C₃₊)N₁₋₇)₃C₃₊ (SEQ ID NO:231), where C is cytosine, N is any nucleotide and “C₃₊” is three or more cytosines. A representative nucleotide sequence of human genomic rDNA is set forth in SEQ ID NO: 1.

Thus, provided herein is an isolated nucleic acid comprising nucleotide sequence ((G₃₊)N₁₋₇)₃G₃₊ (SEQ ID NO:23) or ((C₃₊)N₁₋₇)₃C₃₊ (SEQ ID NO:231) from a human rRNA or rDNA nucleotide sequence, or complement thereof, wherein G is guanine and N is any nucleotide. In some embodiments, the nucleotide sequence is 100 or fewer nucleotides in length, and sometimes the nucleotide sequence is 50 or fewer nucleotides in length. The isolated nucleic acid may be a plasmid in some embodiments and at times is a linear nucleic acid in other embodiments. The nucleic acid may be 100 or fewer nucleotides in length in some embodiments. The nucleic acid sometimes is DNA and sometimes is RNA, and the nucleotide sequence sometimes is a continuous subsequence of SEQ ID NO: 1. In certain embodiments, the nucleic acid is DNA and contains an rRNA sequence, or complement thereof (i.e., uracil is substituted with thymine). The nucleotide sequence may encode a human 28S rRNA subsequence in certain embodiments, and may be a human 28S rRNA subsequence in some embodiments.

Following are examples of rDNA nucleotide sequences sharing no sequence identity with non-rDNA genomic DNA sequences. DNA sequences are on the coding strand (the non-template strand, the plus (+) strand, or the antisense strand) of rDNA, the nucleotide ranges refer to positions on the 43 kb human ribosomal DNA repeat unit (accession no. U13369), and no exact sequence matches were identified within the NCBI build 35 of the human genome on the coding strand or its reverse complement.

(SEQ ID NO:2)

| | |
|---|---|
| 1197-1221: | GGGTGGACGGGGGGGCCTGGTGCGG; |

(SEQ ID NO:3)

| | |
|---|---|
| 2160-2227: | CCCGGGTGCCCTTGCCCTCGCGGTCCCCGGCCCTCGC |
| | GGCTCTGTGCCCTCTTCCCCGCCCGCCGCCC; |

(SEQ ID NO:4)

| | |
|---|---|
| 2958-2985: | GGGTCGGGGGGTGGGGCCCGGGCCGGGG; |

(SEQ ID NO:5)

| | |
|---|---|
| 3468-3491: | CCCCGCCCCGGCCCCACCGGTCCC; |

(SEQ ID NO:6)

| | |
|---|---|
| 3500-3532: | CCCCCGCGCCCGCTCGCTCCCTCCCGTCCGCCC; |

(SEQ ID NO:7)

| | |
|---|---|
| 6184-6213: | GGGTCGGGGGCGGTGGTGGGCCCGCGGGGG; |

(SEQ ID NO:8)

| | |
|---|---|
| 6915-6944: | CCCGCCCCTTCCCCCTCCCCCCGCGGGCCC; |

(SEQ ID NO:9)

| | |
|---|---|
| 6375-6403: | GGGGGCGGGAACCCCCGGGCGCCTGTGGG; |

(SEQ ID NO:10)

| | |
|---|---|
| 6961-6983: | GGGTGGCGGGGGGAGAGGGGGG; |

(SEQ ID NO:11)

| | |
|---|---|
| 7254-7298: | GGGTCCGGAAGGGGAAGGGTGCCGGCGGGGAGAGAGG |
| | GTCGGGGG; |

(SEQ ID NO:12)

| | |
|---|---|
| 7370-7399: | CCCCGCGCCCCTCCTCCTCCCCGCCGCCCC; |

(SEQ ID NO:13)

| | |
|---|---|
| 7734-7763: | CCCGTCCCGCCCCCGGCCCGTGCCCCTCCC; |

(SEQ ID NO:14)

| | |
|---|---|
| 8440-8494: | CCCGCCCCCCGTTCCTCCCGACCCCTCCACCCGCCCT |
| | CCCTTCCCCCGCCGCCCC; |

(SEQ ID NO:15)

| | |
|---|---|
| 8512-8573: | GGGGGCGGGCTCCGGCGGGTGCGGGGGTGGGCGGGCG |
| | GGGCCGGGGGTGGGGTCGGCGGGGG; |

(SEQ ID NO:16)

| | |
|---|---|
| 8716-8747: | CCCGTCTCCGCCCCCCGGCCCCGCGTCCTCCC; |

(SEQ ID NO:17)

| | |
|---|---|
| 8750-8770: | GGGAGGGCGCGCGGGTCGGGG; |

(SEQ ID NO:18)

| | |
|---|---|
| 8904-8926: | CCCCCCTCCCGGCGCCCACCCCC; |

(SEQ ID NO:19)

| | |
|---|---|
| 9024-9052: | CCCACCCCTCCTCCCCGCGCCCCCGCCCC; |

(SEQ ID NO:20

| | |
|---|---|
| 10137-10179: | CCCCTCCTCCCGCCCACGCCCCGCTCCCCGCCCCCGG |
| | AGCCCC; |

(SEQ ID NO:21)

| | |
|---|---|
| 10817-10839: | GGGCTGGGTCGGTCGGGCTGGGG; |

(SEQ ID NO:22)

| | |
|---|---|
| 10885-10934: | CCCCCCCCACGCCCGGGGCACCCCCCTCGCGGCCCTC |
| | CCCCGCCCCACCC; |

(SEQ ID NO:23)

| | |
|---|---|
| 10951-10969: | CCCTCCCCACCCCGCGCCC; |

(SEQ ID NO:24)

| | |
|---|---|
| 10985-11012: | CCCCCGCTCCCCGTCCTCCCCCCTCCCC; |

(SEQ ID NO:25)

| | |
|---|---|
| 11029-11066: | GGGGCGCGGCGGGGGGAGAAGGGTCGGGGCGGCAGG |
| | GG; |

(SEQ ID NO:26)

| | |
|---|---|
| 11245-11389: | CCCCCCGCCCTACCCCCCCGGCCCCGTCCGCCCCCC |
| | GTTCCCCCC; |

(SEQ ID NO:27)

| | |
|---|---|
| 11888-11912: | CCCCCGGCGCCCCCCCGGTGTCCCC; |

(SEQ ID NO:28)

| | |
|---|---|
| 13174-13194: | GGGCCGGGACGGGGTCCGGGG; |

(SEQ ID NO:29)

| | |
|---|---|
| 13236-13261: | CCCCGTGGCCCGCCGGTCCCCGTCCC; |

(SEQ ID NO:30)

| | |
|---|---|
| 14930-14963: | CCCTCCCTCCCTCCCCCTCCCTCCCTCTCTCCCC; |

(SEQ ID NO:31)

| | |
|---|---|
| 17978-18013: | CCCCCACCCCCCCGTCACCTCCCGCTACCCTCCCCC; |

(SEQ ID NO:32)

| | |
|---|---|
| 20511-20567: | GGGGGTGCGGGAATGAGGGTGTGTGTGGGGAGGGGGT |
| | GCGGGGTGGGGAGGGAGGGG; |

(SEQ ID NO:33)

| | |
|---|---|
| 23408-23434: | GGGGAGAGAGGGGGGAGAGGGGGGGGG; |

(SEQ ID NO:34)

| | |
|---|---|
| 28214-28250: | CCCCAAACCGCCCCCCCCCCCCCGCCTCCCAACACC |
| | C; |

(SEQ ID NO:35)

| | |
|---|---|
| 31239-31275: | CCCCACCCACGCCCCACGCCCCACGTCCCGGGCACCC; |

(SEQ ID NO:36)

| | |
|---|---|
| 31415-31452: | GGGAGGGGTGGGGGTGGGGTGGGTTGGGGGTTGTGGG |
| | G; |

(SEQ ID NO:37)

| | |
|---|---|
| 37405-37431: | CCCGGACCCCCCCTTTCCCCTTCCCCC; |

(SEQ ID NO:38)

| | |
|---|---|
| 39261-39290: | CCCGCCCTCCCTGGTTGCCCAGACAACCCC; |
| and | |

(SEQ ID NO:39)

| | |
|---|---|
| 41667-41709: | CCCTCCCTCCCTCCCTCCCTGCTCCCTTCCCTCCCTC |
| | CTTCCC. |

Following are examples of rDNA nucleotide sequences that share sequence identity with non-rDNA sequences in human genomic DNA. DNA sequences are in the rDNA coding strand, and the nucleotide ranges refer to positions on the 43 kb human rDNA repeat unit (accession no. U13369).

(SEQ ID NO:40)

| | |
|---|---|
| 1310-1333: | CCCCCTCCCTTCCCCAGGCGTCCC; |

(SEQ ID NO:41)

| | |
|---|---|
| 5701-5718: | GGGAGGGAGACGGGGGGG; |

(SEQ ID NO:42)

| | |
|---|---|
| 6535-6553: | GGGCGGGGGGGGCGGGGGG; |

(SEQ ID NO:43)

| | |
|---|---|
| 7499-7517: | CCCGCCCCGCCGCCCGCCC; |

(SEQ ID NO:44)

| | |
|---|---|
| 10111-10127: | CCCCCGCCCCCCCCCCC; |

(SEQ ID NO:45)

| | |
|---|---|
| 13080-13095: | GGGGGTGGGGGGGAGGG; |

(SEQ ID NO:46)

| | |
|---|---|
| 14213-14248: | CCCTCCCTCCCTCCCTCCCTCCCTCCCTCCCTCCCC; |

(SEQ ID NO:47)

| | |
|---|---|
| 16166-16189: | GGGGTGGGGTGGGGTGGGGTGGGG; |

(SEQ ID NO:48)

| | |
|---|---|
| 28148-28177: | CCCCCCGGCTCCCCCCACTACCCACGTCCC; |
| and | |

(SEQ ID NO:49)

| | |
|---|---|
| 41842-41876: | CCCTCCCTCCCTCCCTCCCTCCCTCCCTCCCTCCC. |

A sequence comparison between certain human rDNA sequences and other mammalian species was conducted. The following sequences shared little sequence identity with other mammalian species:

(SEQ ID NO:50)

| | |
|---|---|
| 61843-6213 | GGGTCGGGGGCGGTGGTGGGCCCGCGGGGG; |

(SEQ ID NO:51)

| | |
|---|---|
| 8440-8494 | CCCGCCCCCCGTCCTCCCGACCCCTCCACCCGCCCTCCCTT |
| | CCCCCGCCGCCCC; |
| and | |

(SEQ ID NO:52)

| | |
|---|---|
| 8512-8573 | GGGGGGCGGGTCCGGCGGGTGCGGGGGTGGGCGGGCGGGGC |
| | CGGGGGTGGGGTCGGCGGGGG. |

The following sequences shared significant sequence similarity in another mammalian species (e.g., mouse, rat, chimpanzee):

(SEQ ID NO:53)

| | |
|---|---|
| 8717-8747 | CCCGTCTCCGCCCCCCGGCCCCGCGTCCTCCC; |

(SEQ ID NO:54)

| | |
|---|---|
| 8751-8770 | GGGAGGGCGCGCGGGTCGGGG; |

(SEQ ID NO:55)

| | |
|---|---|
| 10112-10127 | CCCCCGCCCCCCCCCCC; |

(SEQ ID NO:56)

| | |
|---|---|
| 10138-10179 | CCCCTCCTCCCGCCCACGCCCCGCTCCCCGCCCCCGGAG |
| | CCCC; |

(SEQ ID NO:57)

| | |
|---|---|
| 10817-10839 | GGGCTGGGTCGGTCGGGCTGGGG; |
| and | |

(SEQ ID NO:58)

| | |
|---|---|
| 11889-11912 | CCCCCGGCGCCCCCCCGGTGTCCCC. |

The following sequences are G and C-rich sequences in the non-coding strands of rDNA, which in certain embodiments may form a quadruplex structure.

(SEQ ID NO:59)

| | |
|---|---|
| 1222-1197 | CCCCACCAGGCCCCCCCGTCCACCC; |

(SEQ ID NO:60)

| | |
|---|---|
| 1334-1310 | GGGACGCCTGGGGAAGGGAGGGGG; |

(SEQ ID NO:61)

| | |
|---|---|
| 2228-2160 | GGGCGGCGGGCGGGGAAGAGGGCACACACGGGCGAGGGCC |
| | GGGGACCGCGAGGGCAAGGGCACCCGGG; |

(SEQ ID NO:62)

| | |
|---|---|
| 2986-2958 | CCCCGGCCCGGGCCCCACCCCCCGACCC; |

(SEQ ID NO:63)

| | |
|---|---|
| 3492-3468 | GGGACCGGTGGGGCCGGGGCGGGG; |

(SEQ ID NO:64)

| | |
|---|---|
| 3533-3500 | GGGCGGACGGGAGGGAGCGAGCGGGCGCGGGGG; |

(SEQ ID NO:65)

| | |
|---|---|
| 5719-5701 | CCCCCCCGTCTCCCTCCC; |

(SEQ ID NO:66)

| | |
|---|---|
| 6214-6184 | CCCCCGCGGGCCCACCACCGCCCCCGACCC; |

(SEQ ID NO:67)

| | |
|---|---|
| 6945-6915 | GGGCCCGCGGGGGGAGGGGGAAGGGGCGGG; |

(SEQ ID NO:68)

| | |
|---|---|
| 6404-6375 | CCCACAGGCGCCCGGGGGTTCCCGCCCCC; |

(SEQ ID NO:69)

| | |
|---|---|
| 6554-6535 | CCCCCCGCCCCCCCCGCCC; |

(SEQ ID NO:70)

| | |
|---|---|
| 6984-6961 | CCCCCCTCTCCCCCCCGCCACCC; |

(SEQ ID NO:71)

| | |
|---|---|
| 7299-7254 | CCCCCGACCCTCTCTCCCCGCCGGCACCCTTCCCCTTCCG |
| | GACCC; |

(SEQ ID NO:72)

| | |
|---|---|
| 7400-7370 | GGGGCGGCGGGGAGGAGGAGGGGCGCGGGG; |

(SEQ ID NO:73)

| | |
|---|---|
| 7518-7499 | GGGCGGGCGGCGGGGCGGG; |

(SEQ ID NO:74)

| | |
|---|---|
| 7764-7734 | GGGAGGGGCACGGGCCGGGGGCGGGACGGG; |

(SEQ ID NO:75)

| | |
|---|---|
| 8495-8440 | GGGGCGGCGGGGGAAGGGAGGGCGGGTGGAGGGGTCGGGA |
| | GGAACGGGGGGCGGG; |

(SEQ ID NO:76)

| | |
|---|---|
| 8574-8512 | CCCCCGCCGACCCCACCCCCGGCGCCGCCCGCCCACCCCC |
| | GGACCCGCCGGAGCCCGCCCCC; |

(SEQ ID NO:77)

| | |
|---|---|
| 8748-8716 | GGGAGGACGCGGGGCCGGGGGGCGGAGACGGG; |

(SEQ ID NO:78)

| | |
|---|---|
| 8771-8750 | CCCCGACCCGCGCGCCCTCCC; |

(SEQ ID NO:79)

| | |
|---|---|
| 8927-8904 | GGGGGTGGGCGCCGGGAGGGGGG; |

SEQ ID NO:80

| | |
|---|---|
| 9053-9024 | GGGGCGGGGGCGCGGGGAGGAGGGGTGGG; |

(SEQ ID NO:81)

| | |
|---|---|
| 10128-10111 | GGGGGGGGGGGCGGGGG; |

(SEQ ID NO:82)

| | |
|---|---|
| 10180-10137 | GGGGCTCCGGGGGCGGGGAGCGGGGCGTGGGCGGGAGGAG |
| | GGG; |

(SEQ ID NO:83)

| | |
|---|---|
| 10840-10817 | CCCCAGCCCGACCGACCCAGCCC; |

(SEQ ID NO:84)

| | |
|---|---|
| 10935-10885 | GGGTGGGGCGGGGGAGGGCCGCGAGGGGGGTGCCCCGGGC |
| | GTGGGGGGGG; |

(SEQ ID NO:85)

| | |
|---|---|
| 10970-10951 | GGGCGCGGGGTGGGGAGGG; |

(SEQ ID NO:86)

| | |
|---|---|
| 11013-10985 | GGGGAGGGGGGAGGACGGGGAGCGGGGG; |

(SEQ ID NO:87)

| | |
|---|---|
| 11067-11029 | CCCCTGCCGCCCCGACCCTTCTCCCCCCGCCGCGCCCC; |

(SEQ ID NO:88)

| | |
|---|---|
| 11390-11345 | GGGGGGAACGGGGGGCGGACGGGGCCGGGGGGGTAGGGCG |
| | GGGGG; |

(SEQ ID NO:89)

| | |
|---|---|
| 11913-11888 | GGGGACACCGGGGGGGCGCCGGGGG; |

(SEQ ID NO:90)

| | |
|---|---|
| 13096-13080 | CCCTCCCCCCCACCCC; |

(SEQ ID NO:91)

| | |
|---|---|
| 13195-13174 | CCCCGGACCCCGTCCCGGCCC; |

(SEQ ID NO:92)

| | |
|---|---|
| 13262-13236 | GGGACGGGGACCGGCGGGCCACGGGG; |

(SEQ ID NO:93)

| | |
|---|---|
| 14249-14213 | GGGGAGGGAGGGAGGGAGGGAGGGAGGGAGGGAGGG; |

(SEQ ID NO:94)

| | |
|---|---|
| 14964-14930 | GGGGAGAGAGGGAGGGAGGGGGAGGGAGGGAGGG; |

(SEQ ID NO:95)

| | |
|---|---|
| 16190-16166 | CCCCACCCCACCCCACCCCACCCC; |

(SEQ ID NO:96)

| | |
|---|---|
| 18014-17978 | GGGGGAGGGTAGCGGGACGTGACGGGGGGGTGGGGG; |

(SEQ ID NO:97)

| | |
|---|---|
| 20568-20511 | CCCCTCCGTCCCCACCCCGCACCCCCTCCCCACACACACC |
| | CTCATTCCCGCACCCCC; |

(SEQ ID NO:98)

| | |
|---|---|
| 23435-23408 | CCCCCCCCCTCTCCCCCCTCTCTCCCC; |

(SEQ ID NO:99)

| | |
|---|---|
| 28178-28148 | GGGACGTGGGTACTGGGGGGAGCCGGGGGG; |

(SEQ ID NO:100)

| | |
|---|---|
| 28251-28214 | GGGTGTTGGGAGGCGGGGGGGGGGGGGCGGTTTGGGG; |

(SEQ ID NO:101)

| | |
|---|---|
| 31276-31239 | GGGTGCCCGGGACGTGGGGCGTGGGGCGTGGGTGGGG; |

(SEQ ID NO:102)

| | |
|---|---|
| 31453-31415 | CCCCACAACCCCCAACCCACCCCACCCCCACCCCTCCC; |

(SEQ ID NO:103)

| | |
|---|---|
| 37432-37405 | GGGGGAAGGGGAAAGGGGGGGTCCGGG; |

(SEQ ID NO:104)

| | |
|---|---|
| 39291-39261 | GGGGTTGTCTGGGCAACCAGGGAGGGCGGG; |

(SEQ ID NO:105)

| | |
|---|---|
| 41710-41667 | GGGAAGGAGGGAGGGAAGGGAGCAGGGAGGGAGGGAGGG |
| | AGGG; |
| and | |

(SEQ ID NO:106)

| | |
|---|---|
| 41877-41842 | GGGAGGGAGGGAGGGAGGGAGGGAGGGAGGGAGGG. |

In some embodiments, the isolated nucleic acid is RNA, and sometimes includes a nucleotide sequence encoded by a subsequence of SEQ ID NO: 1. In some embodiments, the nucleotide sequence is a human 28S rRNA subsequence.

Following are examples of rRNA and pre-rRNA nucleotide sequences encoded by specified regions in rDNA. The RNA sequences are inferred from rDNA sequence and annotations found within accession number U13369. No matches were identified within genes (as identified by Curwen et al., The Ensembl Automatic Gene Annotation System, Genome Res. 2004 May; 14(5):942-950) along the coding strand (CDS) of the human genome for the DNA sequence transcribed to produce the rRNA and pre-rRNA.

RNA sequence from 5′ external transcribed spacer
region in rDNA

(SEQ ID NO:107)

GGGGUGGACGGGGGGGCCUGGUGGGG;

(SEQ ID NO:108)

GGGUCGGGGGGUGGGGCCCGGGCCGGGG;
RNA sequence from internal transcribed spacer 1
region in rDNA

(SEQ ID NO:109)

GGGAGGGAGACGGGGGGG;

(SEQ ID NO:110)

GGGUCGGGGGCGGUGGUGGGCCCGCGGGGG;

(SEQ ID NO:111)

GGGGGCGGGAACCCCCGGGCGCCUGUGGG;
RNA sequences from internal transcribed spacer 2
region in rDNA

(SEQ ID NO:112)

GGGUGGCGGGGGGGAGAGGGGGG;

(SEQ ID NO:113)

GGGUCCGGAAGGGGAAGGGUGCCGGCGGGGAGAGAGGGUCGGGGG;
RNA sequences within 28S rRATA

(SEQ ID NO:114)

GGGGGCGGGCUCCGGCGGGUGCGGGGGUGGGCGGGCGGGGCCGGGGGUGG
GGUCGGCGGGGG;

(SEQ ID NO:115)

GGGAGGGCGCGCGGGUCGGGG;

(SEQ ID NO:116)

GGGCUGGGUCGGUCGGGCUGGGG;

(SEQ ID NO:117)

GGGGCGCGGCGGGGGGAGAAGGGUCGGGGCGGCAGGGG;
RNA sequences from 3′ external transcribed spacer
region in rDNA

(SEQ ID NO:118)

GGGCCGGGACGGGGUCCGGGG.

Following are rRNA and pre-rRNA sequences exactly matching RNA transcribed from non-rDNA and the rDNA regions from which they are transcribed.

RNA sequence from internal transcribed spacer 1
region in rDNA

(SEQ ID NO:119)

GGGCGGGGGGGGCGGGGGG;
RNA sequence from 3′ external transcribed spacer
region in rDNA

(SEQ ID NO:120)

GGGGUGGGGGGGAGGG;

Following are C-rich rRNA and pre-rRNA sequences in the transcribed region of rDNA, which in certain embodiments may form a quadruplex.

RNA sequence from 5′ external transcribed spacer
region in rDNA

(SEQ ID NO:121)

CCCCCUCCCUUCCCCAGGCGUCCC

(SEQ ID NO:122)

CCCGGGUGCCCUUGCCCUCGCGGUCCCCGGCCCUCGCCCGUGUGUGCCCU
CUUCCCCGCCCGCCGCCC

(SEQ ID NO:123)

CCCCGCCCCGGCCCCACCGGUCCC

(SEQ ID NO:124)

CCCCCGCGCCCGCUCGCUCCCUCCCGUCCGCCC
RATA sequences from internal transcribed spacer 2
region in rDNA

(SEQ ID NO:125)

CCCGCCCCUUCCCCCUCCCCCCGCGGGCCC

(SEQ ID ND:126)

CCCCGCGCCCCUCCUCCUCCCCGCCGCCCC

(SEQ ID NO:127)

CCCGCCCCGCCGCCCGCCC

(SEQ ID NO:128)

CCCGUCCCGCCCCCGGCCCGUGCCCCUCCC
RNA sequences within 28S rRNA

(SEQ ID NO:129)

CCCGCCCCCCGUUCCUCCCGACCCCUCCACCCGCCCUCCCUUCCCCCGCC
GCCCC

(SEQ ID NO:130)

CCCGUCUCCGCCCCCCGGCCCCGCGUCCUCCC

(SEQ ID NO:131)

CCCCCCUCCCGGCGCCCACCCCC

(SEQ ID ND:132)

CCCACCCCUCCUCCCCGCCCCCCCGCCCC

(SEQ ID NO:133)

CCCCCGCCCCCCCCCCC

(SEQ ID NO:134)

CCCCUCCUCCCGCCCACGCCCCGCUCCCCGCCCCCGGAGCCCC

(SEQ ID NO:135)

CCCCCCCCACGCCCGGGGCACCCCCCUCGCGGCCCUCCCCCGCCCCACCC

(SEQ ID NO:136)

CCCUCCCCACCCCGCGCCC

(SEQ ID NO:137)

CCCCCGCUCCCCGUCCUCCCCCCUCCCC

(SEQ ID NO:138)

CCCCCCGCCCUACCCCCCCGGCCCCGUCCGCCCCCCGUUCCCCCC

(SEQ ID NO:139)

CCCCCGGCGCCCCCCCGGUGUCCCC
RNA sequence from 3′ external transcribed spacer
region in rDNA

(SEQ ID NO:40)

CCCCGUGCCCCGCCGCUCCCCGUCCC

In some embodiments, an isolated nucleic acid described herein is in combination with another nucleic acid described herein and/or another component described hereafter (e.g., protein, antibody). Isolated nucleic acids provided herein sometimes comprise, consist essentially of or consist of one of the foregoing nucleotide sequences or subsequence thereof. In certain embodiments, the nucleic acid is a nucleic acid analog, such as a peptide nucleic acid (PNA) analog or other analog described herein. The nucleotide sequence in the nucleic acid may include one or more nucleotide substitutions, which substitution(s) result(s) in a nucleotide sequence that conforms with the sequence motif ((G₃₊)N₁₋₇)₃G₃₊ (SEQ ID NO:230) or ((C₃₊)N₁₋₇)₃C₃₊ (SEQ ID NO:231) in some embodiments, and sometimes a nucleotide is substituted with a nucleotide analog. In certain embodiments, the nucleic acid or a portion thereof forms a quadruplex structure, such as an intramolecular quadruplex structure. In some embodiments, a composition comprising the isolated nucleic acid also comprises one or more components that stabilize a quadruplex structure, such as potassium ions (e.g., 0.5 mM to 100 mM potassium ions), for example.

In certain embodiments, a nucleic acid comprising a human ribosomal nucleotide sequence, or substantially identical nucleotide sequence thereof, forms a quadruplex structure. The nucleic acid often is in a composition that comprises other components that enable quadruplex formation and sometimes stabilize a quadruplex structure. The human ribosomal nucleotide sequence or substantially identical variant thereof sometimes is G-rich and at times is C-rich, and in certain embodiments conforms to the nucleotide sequence ((G₃₊)N₁₋₇)₃G₃+(SEQ ID NO:230) or ((C₃₊)NI-7)₃C₃₊ (SEQ ID NO:231). The nucleic acid sometimes is RNA, and in some embodiments is DNA. Substantially identical nucleotide sequence variants sometimes are 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more identical to a nucleotide subsequence of SEQ ID NO: 1 or a complement thereof. In certain embodiments, the human ribosomal nucleotide sequence is from one of the following regions of a human ribosomal nucleotide sequence or complement thereof: (a) 5′ETS region, ITS1 region, ITS2 region, 28S rRNA region, 3′ETS region, 18S rRNA region or 5.8S rRNA region of rDNA (e.g., SEQ ID NO: 1); (b) complement of (a); encoded RNA of (a); or encoded RNA of (b).

Also provided herein is a method for identifying a quadruplex forming subsequence candidate in a human rRNA-encoding genomic DNA, which comprises identifying subsequence ((G₃₊)N₁₋₇)₃G₃+ (SEQ ID NO:230) or ((C₃,)N₁₋₇)₃C₃₊ (SEQ ID NO:231) in a human rRNA-encoding genomic DNA, where G is guanine, C is cytosine, “3+” is three or more nucleotides and N is any nucleotide. In some embodiments the human rRNA-encoding genomic DNA is SEQ ID NO: 1.

Provided also are methods that utilize one or more of the ribosomal nucleotide sequences described herein. For example, provided is a method for identifying a molecule that binds to a nucleic acid containing a human ribosomal nucleotide sequence, which comprises: (a) contacting a nucleic acid containing a human ribosomal nucleotide sequence described herein, a compound that binds to the nucleic acid and a test molecule, and (b) detecting the amount of the compound bound or not bound to the nucleic acid, whereby the test molecule is identified as a molecule that binds to the nucleic acid when less of the compound binds to the nucleic acid in the presence of the test molecule than in the absence of the test molecule. The compound sometimes is in association with a detectable label, and at times is radiolabeled. In certain embodiments, the compound is a quinolone analog (e.g., a quinolone analog described herein) or a porphyrin. The nucleic acid may be in association with a solid phase in certain embodiments. The nucleic acid may be DNA, RNA or an analog thereof, and may comprise a nucleotide sequence described above in specific embodiments. The nucleic acid may form a quadrupled, such as an intramolecular quadrupled, in certain embodiments.

Also provided herein is a method for identifying a molecule that modulates an interaction between a ribosomal nucleic acid and a protein that interacts with the nucleic acid, which comprises: (a) contacting a nucleic acid containing a human ribosomal nucleotide sequence and the protein with a test molecule, where the nucleic acid is capable of binding to the protein, and (b) detecting the amount of the nucleic acid bound or not bound to the protein, whereby the test molecule is identified as a molecule that modulates the interaction (e.g., a different amount of the nucleic acid binds to the protein in the presence of the test molecule than in the absence of the test molecule). In some embodiments, the protein is selected from the group consisting of Nucleolin, Fibrillarin, RecQ, QPNI and functional fragments of the foregoing. In some embodiments, provided is a method for identifying a molecule that causes nucleolin displacement, which comprises (a) contacting a nucleic acid containing a human ribosomal nucleotide sequence and a nucleolin protein with a test molecule, where the nucleic acid is capable of binding to the nucleolin protein, and (b) detecting the amount of the nucleic acid bound or not bound to the nucleolin protein, whereby the test molecule is identified as a molecule that causes nucleolin displacement when less of the nucleic acid binds to the nucleolin protein in the presence of the test molecule than in the absence of the test molecule. In some embodiments, the nucleolin protein is in association with a detectable label, and the nucleolin protein sometimes is in association with a solid phase. The nucleic acid sometimes is in association with a detectable label, and the nucleic acid may be in association with a solid phase in certain embodiments. The nucleic acid may be DNA, RNA or an analog thereof, and may comprise a nucleotide sequence described above in specific embodiments. In some embodiments the test molecule is a quinolone analog. Provided also is a composition comprising a nucleic acid having a ribosomal nucleotide sequence provided herein, or substantially identical sequence thereof, and a protein that binds to the nucleotide sequence (e.g., Nucleolin, Fibrillarin, RecQ, QPN1 and functional fragments of the foregoing).

Also provided herein is a method for identifying a modulator of nucleic acid synthesis, which comprises contacting a template nucleic acid, a primer oligonucleotide having a nucleotide sequence complementary to a template nucleic acid nucleotide sequence, extension nucleotides, a polymerase and a test molecule under conditions that allow the primer oligonucleotide to hybridize to the template nucleic acid, where the template nucleic acid comprises a human ribosomal nucleotide sequence, and detecting the presence, absence or amount of an elongated primer product synthesized by extension of the primer nucleic acid, whereby the test molecule is identified as a modulator of nucleic acid synthesis when less of the elongated primer product is synthesized in the presence of the test molecule than in the absence of the test molecule. In certain embodiments, the method is directed to identifying a modulator or RNA synthesis, and in certain embodiments, identifying a modulator of nucleolar RNA synthesis. The template nucleic acid sometimes is DNA and at times is RNA, and the template can include any one or more of the ribosomal nucleotide sequences described herein. The polymerase sometimes is a DNA polymerase and at times is a RNA polymerase.

Provided also is a composition comprising a nucleic acid described herein. In some embodiments, a composition comprises a nucleic acid that includes a nucleotide sequence complementary to a human rDNA or rRNA nucleotide sequence described herein. The composition may comprise a pharmaceutically acceptable carrier in some embodiments, and the composition sometimes comprises the nucleic acid and a compound that binds to a human ribosomal nucleotide sequence in the nucleic acid (e.g., specifically binds to the nucleotide sequence). In certain embodiments, the compound is a quinolone analog, such as a compound described herein.

Also provided is a cell or animal comprising an isolated nucleic acid described herein. Any suitable type of cell can be utilized, and sometimes the cell is a cell line maintained or proliferated in tissue culture. The isolated nucleic acid may be incorporated into one or more cells of an animal, such as a research animal (e.g., rodent (e.g., mouse, rat, guinea pig, hamster, rabbit), cat, dog, monkey or ape).

Also provided is a cell comprising a compound that binds to a human ribosomal nucleotide sequence described herein. In certain embodiments, provided is an animal comprising such a cell. In some embodiments, the compound is localized in the nucleolus. In certain embodiments, one or more of H2AX, p53, chk1, p38 MAPK and chk2 proteins are phosphorylated, and sometimes H2AX, p53, chk1 and p38 MAPK proteins are substantially phosphorylated but not the chk2 protein. In some embodiments, JUN protein kinase (JNK) is phosphorylated. In certain embodiments, nucleolin is redistributed from nucleoli into the nucleoplasm.

Provided herein is a method for inhibiting rRNA synthesis in cells, which comprises contacting cells with a compound that interacts with rRNA or rDNA in an amount effective to reduce rRNA synthesis in cells. Such methods may be conducted in vitro, in vivo and/or ex vivo. Accordingly, some in vivo and ex vivo embodiments are directed to a method for inhibiting rRNA synthesis in cells of a subject, which comprises administering a compound that interacts with rRNA or rDNA to a subject need thereof in an amount effective to reduce rRNA synthesis in cells of the subject. In some embodiments, cells can be contacted with one or more compounds, one or more of which interact with rRNA or rDNA (e.g., one drug or drug and co-drug(s) methodologies). In certain embodiments, a compound is a quinolone derivative, such as a quinolone derivative described herein (e.g., compound A-1 or B-1). In related embodiments, cells are contacted with a compound that interacts with rRNA or rDNA and one or more co-molecules (e.g., co-drugs) that exert other effects in cells. For example, a co-drug may be selected that reduces cell proliferation or reduces tissue inflammation. Non-limiting examples of co-drugs are provided hereafter.

Provided also is a method for effecting a cellular response by contacting a cell with a compound that binds to a human ribosomal nucleotide sequence and/or structure described herein. The cellular response sometimes is (a) substantial phosphorylation of H2AX, p53, chk1, JUNK and p38 MAPK proteins; (b) redistribution of nucleolin from nucleoli into the nucleoplasm; (c) release of cathepsin D from lysosomes; (d) induction of apoptosis; (e) induction of chromosomal laddering; (f) induction of apoptosis without arresting cell cycle progression; and (g) induction of apoptosis and inducing cell cycle arrest (e.g., S-phase and/or G1 arrest).

Also provided herein is method for inducing apoptosis without arresting cell cycle progression, which comprises contacting a cell with a compound that binds (e.g., specifically binds) to a human ribosomal nucleotide sequence and/or structure described herein in amount effective for inducing apoptosis. Provided also is method for inducing apoptosis without arresting cell cycle progression, which comprises administering a compound that binds (e.g., specifically binds) to a human ribosomal nucleotide sequence and/or structure described herein to a subject in need thereof in amount effective for inducing apoptosis. The subject may be a rodent (e.g., mouse, rat, hamster, guinea pig, rabbit), cat, dog, ungulate, monkey, ape or human, and compound may be administered to a subject in any suitable and convenient form to induce apoptosis (e.g., oral, parenteral, intravenous, transdermal). An example of such a compound is a quinolone analog of formula 2C or 3A. In certain embodiments, the quinolone analog has structure A-1. Cell cycle progression often is not arrested significantly in any one phase of the cycle.

Provided also is method for inducing apoptosis and arresting cell cycle progression (e.g., S phase cell cycle arrest and/or G1 cell cycle arrest), which comprises contacting a cell with a compound that binds (e.g., specifically binds) to a human ribosomal nucleotide sequence and/or structure described herein in amount effective for inducing apoptosis. Provided also is method for inducing apoptosis and arresting cell cycle progression (e.g., S phase cell cycle arrest or G1 cell cycle arrest), which comprises administering a compound that binds (e.g., specifically binds) to a human ribosomal nucleotide sequence and/or structure described herein to a subject in need thereof in amount effective for inducing apoptosis. The subject may be a rodent (e.g., mouse, rat, hamster, guinea pig, rabbit), cat, dog, ungulate, monkey, ape or human, and compound may be administered to a subject in any suitable and convenient form to induce apoptosis (e.g., oral, parenteral, intravenous, transdermal). An example of such a compound is a quinolone analog of formula 2D. In certain embodiments, the quinolone analog has structure B-1. Cell cycle progression often is arrested significantly at one phase, and sometimes two phases.

In the foregoing methods, chromosomal DNA laddering sometimes is induced by the compound. In specific embodiments, cells are pancreatic cells, colorectal cells, renal cells and Burkitt's lymphoma cells, or the foregoing are targeted in a subject.

Also provided is a method for determining whether a compound is toxic to a cell or a subject, which comprises contacting a cell with the compound and determining the phosphorylation state of a JNK protein, and optionally a p38MAPK protein, whereby the compound is determined as toxic to the cell or subject when a phosphorylation level of the JNK protein, and optionally the p38MAPK protein, is greater in cells contacted with the compound as compared to cells not contacted with the compound. In some embodiments, the toxicity is inflammation. The method sometimes comprises the step of comparing JNK protein, and optionally p38MAPK protein, phosphorylation levels in cells contacted with the compound to cells not contacted with the compound, and sometimes predetermined JNK protein and or p38MAPK protein phosphorylation levels in cells not treated with the compound are compared to phosphorylation levels in cells treated with the compound. In certain embodiments, the JNK protein is a particular isoform of the JNK protein, and the p38MAPK protein is a particular p38MAPK protein isoform. Phosphorylation of the JNK protein or p38MAPK protein can be determined in any convenient manner, examples of which are described hereafter. The methods may be utilized to determine toxicity of a quinolone compound to cells or cells of a subject, which can be a quinolone compound of a formula set forth herein.

### BRIEF DESCRIPTION OF THE DRAWINGS

FIG. 1 and FIG. 2 show quinolone analogs can interfere with a quadruplex nucleic acid/binding protein interaction.

FIG. 3 shows circular dichroism scans of particular ribosomal nucleic acid nucleotide sequences that include mixed conformation (“M”; e.g., nucleic acid 6914T), parallel conformation (“P”; e.g., nucleic acid 10110T), antiparallel conformation (“A”; e.g., nucleic acid 9749NT) and complex conformation (“C”; e.g., nucleic acid 8762NT) quadruplex structures.

FIGS. 4A, 4B and 4C show effects of compound A-1 on synthesis of rRNA and c-MYC RNA.

### DETAILED DESCRIPTION

Ribosomal nucleic acids and related methods described herein are useful in a variety of applications. For example, the nucleotide sequences described herein can serve as targets for screening interacting molecules (e.g., in screening assays). The interacting molecules may be utilized as novel therapeutics or for the discovery of novel therapeutics. Ribosomal nucleic acid interacting molecules can serve as tools for identifying other target nucleotide sequences (e.g., target screening assays) or other interacting molecules (e.g., competition screening assays). The nucleotide sequences or complementary sequences thereof also can be utilized as aptamers or serve as basis for generating aptamers. The aptamers can be utilized as therapeutics or in assays for identifying novel interacting molecules.

Nucleic Acids

Provided herein are isolated ribosomal nucleic acids having rDNA or rRNA nucleotide sequences described herein, or substantially identical variants thereof. In some embodiments, the nucleotide sequence includes or is part of a 28S, 18S or 5.8S rRNA human nucleotide sequence, or a substantially identical variant thereof. The nucleotide sequence sometimes includes or is part of SEQ ID NO: 1, or a substantially identical variant thereof. A “ribosomal nucleic acid” or “ribosomal nucleotide sequence” can include a human rRNA nucleotide sequence, a human rDNA nucleotide sequence, or a human pre-rRNA nucleotide sequence, and sometimes is a substantially identical variant of the foregoing.

A nucleic acid may be single-stranded, double-stranded, triplex, linear or circular. The nucleic acid sometimes is a RNA, at times is DNA, and may comprise one or more nucleotide derivatives or analogs of the foregoing (e.g., 1, 2, 3, 4, 5, 6, 7, 8, 9, 10 or more analog or derivative nucleotides). In some embodiments, the nucleic acid is entirely comprised of one or more analog or derivative nucleotides, and sometimes the nucleic acid is composed of about 50% or fewer, about 25% or fewer, about 10% or fewer or about 5% or fewer analog or derivative nucleotide bases. One or more nucleotides in an analog or derivative nucleic acid may comprise a nucleobase modification or backbone modification, such as a ribose or phosphate modification (e.g., ribosepeptide nucleic acid (PNA) or phosphothioate linkages), as compared to a RNA or DNA nucleotide. Nucleotide analogs and derivatives are known to the person of ordinary skill in the art, and non-limiting examples of such modifications are set forth in U.S. Pat. No. 6,455,308 (Freier et al.); in U.S. Pat. Nos. 4,469,863; 5,536,821; 5,541,306; 5,637,683; 5,637,684; 5,700,922; 5,717,083; 5,719,262; 5,739,308; 5,773,601; 5,886,165; 5,929,226; 5,977,296; 6,140,482; and in WIPO publications WO 00/56746 and WO 01/14398. Methods for synthesizing nucleic acids comprising such analogs or derivatives are disclosed, for example, in the patent publications cited above, and in U.S. Pat. Nos. 6,455,308; 5,614,622; 5,739,314; 5,955,599; 5,962,674; 6,117,992; and in WO 00/75372.

A nucleic acid or ribosomal nucleotide sequence therein sometimes is about 8 to about 80 nucleotides in length, at times about 8 to about 50 nucleotides in length, and sometimes from about 10 to about 30 nucleotides in length. In some embodiments, the nucleic acid or ribosomal nucleotide sequence therein sometimes is about 500 or fewer, about 400 or fewer, about 300 or fewer, about 200 or fewer, about 150 or fewer, about 100 or fewer, about 90 or fewer, about 80 or fewer, about 70 or fewer, about 60 or fewer, or about 50 or fewer nucleotides in length, and sometimes is about 40 or fewer, about 35 or fewer, about 30 or fewer, about 25 or fewer, about 20 or fewer, or about 15 or fewer nucleotides in length. A nucleic acid sometimes is larger than the foregoing lengths, such as in embodiments in which it is in plasmid form, and can be about 600, about 700, about 800, about 900, about 1000, about 1100, about 1200, about 1300, or about 1400 base pairs in length or longer in certain embodiments.

Nucleic acids described herein often are isolated. The term “isolated” as used herein refers to material removed from its original environment (e.g., the natural environment if it is naturally occurring, or a host cell if expressed exogenously), often is purified from other materials in an original environment, and thus is altered “by the hand of man” from its original environment. The term “purified” as used herein with reference to molecules does not refer to absolute purity. Rather, “purified” refers to a substance in a composition that contains fewer substance species in the same class (e.g., nucleic acid or protein species) other than the substance of interest in comparison to the sample from which it originated. The term “purified” refers to a substance in a composition that contains fewer nucleic acid species other than the nucleic acid of interest in comparison to the sample from which it originated. Sometimes, a nucleic acid is “substantially pure,” indicating that the nucleic acid represents at least 50% of nucleic acid on a mass basis of the composition. Often, a substantially pure nucleic acid is at least 75% pure on a mass basis of the composition, and sometimes at least 95% pure on a mass basis of the composition. The nucleic acid may be purified from a biological source and/or may be manufactured. Nucleic acid manufacture processes (e.g., chemical synthesis and recombinant DNA processes) and purification processes are known to the person of ordinary skill in the art. For example, synthetic oligonucleotides can be synthesized using standard methods and equipment, such as by using an ABI™3900 High Throughput DNA Synthesizer, which is available from Applied Biosystems (Foster City, Calif.).

As described above, a nucleic acid may comprise a substantially identical sequence variant of a nucleotide sequence described herein. The term “substantially identical variant” as used herein refers to a nucleotide sequence sharing sequence identity to a ribosomal nucleotide sequence described. Included are nucleotide sequences 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more or 99% or more sequence identity to a ribosomal nucleotide sequence described herein. In certain embodiments, the substantially identical variant is 91% or more identical to a ribosomal nucleotide sequence described herein. One test for determining whether two nucleotide sequences are substantially identical is to determine the percent of identical nucleotide sequences shared.

Calculations of sequence identity can be performed as follows. Sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is sometimes 30% or more, 40% or more, 50% or more, often 60% or more, and more often 70% or more, 80% or more, 90% or more, or 100% of the length of the reference sequence. The nucleotides or amino acids at corresponding nucleotide or polypeptide positions, respectively, are then compared among the two sequences. When a position in the first sequence is occupied by the same nucleotide or amino acid as the corresponding position in the second sequence, the nucleotides or amino acids are deemed to be identical at that position. The percent identity between the two sequences is a function of the number of identical positions shared by the sequences, taking into account the number of gaps, and the length of each gap, introduced for optimal alignment of the two sequences. Comparison of sequences and determination of percent identity between two sequences can be accomplished using a mathematical algorithm. Percent identity between two amino acid or nucleotide sequences can be determined using the algorithm of Meyers & Miller, CABIOS 4: 11-17 (1989), which has been incorporated into the ALIGN program (version 2.0), using a PAM120 weight residue table, a gap length penalty of 12 and a gap penalty of 4. Also, percent identity between two amino acid sequences can be determined using the Needleman & Wunsch, J. Mol. Biol. 48: 444-453 (1970) algorithm which has been incorporated into the GAP program in the GCG software package (available at the world wide web address: gcg.com), using either a Blossum 62 matrix or a PAM250 matrix, and a gap weight of 16, 14, 12, 10, 8, 6, or 4 and a length weight of 1, 2, 3, 4, 5, or 6. Percent identity between two nucleotide sequences can be determined using the GAP program in the GCG software package (available at the world wide web address: gcg.com), using a NWSgapdna.CMP matrix and a gap weight of 40, 50, 60, 70, or 80 and a length weight of 1, 2, 3, 4, 5, or 6. A set of parameters often used is a Blossum 62 scoring matrix with a gap open penalty of 12, a gap extend penalty of 4, and a frameshift gap penalty of 5.

Another manner for determining whether two nucleic acids are substantially identical is to assess whether a polynucleotide homologous to one nucleic acid will hybridize to the other nucleic acid under stringent conditions. As use herein, the term “stringent conditions” refers to conditions for hybridization and washing. Stringent conditions are known to those skilled in the art and can be found in Current Protocols in Molecular Biology, John Wiley & Sons, N.Y., 6.3.1-6.3.6 (1989). Aqueous and non-aqueous methods are described in that reference and either can be used. An example of stringent hybridization conditions is hybridization in 6× sodium chloride/sodium citrate (S SC) at about 45° C., followed by one or more washes in 0.2×SSC, 0.1% SDS at 50° C. Another example of stringent hybridization conditions are hybridization in 6× sodium chloride/sodium citrate (SSC) at about 45° C., followed by one or more washes in 0.2×SSC, 0.1% SDS at 55° C. A further example of stringent hybridization conditions is hybridization in 6× sodium chloride/sodium citrate (S SC) at about 45° C., followed by one or more washes in 0.2×SSC, 0.1% SDS at 60° C. Often, stringent hybridization conditions are hybridization in 6× sodium chloride/sodium citrate (SSC) at about 45° C., followed by one or more washes in 0.2×SSC, 0.1% SDS at 65° C. More often, stringency conditions are 0.5M sodium phosphate, 7% SDS at 65° C., followed by one or more washes at 0.2×SSC, 1% SDS at 65° C.

Specific ribosomal nucleotide sequences described herein can be used as “query sequences” to perform a search against public databases to identify other family members or related sequences, for example. The query sequences can be utilized to search for substantially identical sequences in organisms other than humans (e.g., apes, rodents (e.g., mice, rats, rabbits, guinea pigs), ungulates (e.g., equines, bovines, caprines, porcines), reptiles, amphibians and avians). Such searches can be performed using the NBLAST and XBLAST programs (version 2.0) of Altschul et al., J. Mol. Biol. 215: 403-10 (1990). BLAST nucleotide searches can be performed with the NBLAST program, score=100, wordlength=12 to obtain nucleotide sequences homologous to ribosomal nucleotide sequences described herein. BLAST polypeptide searches can be performed with the XBLAST program, score=50, wordlength=3 to obtain amino acid sequences homologous to those encoded by herein. To obtain gapped alignments for comparison purposes, Gapped BLAST can be utilized as described in Altschul et al., Nucleic Acids Res. 25(17): 3389-3402 (1997). When utilizing BLAST and Gapped BLAST programs, default parameters of the respective programs (e.g., XBLAST and NB LAST) can be used (see the world wide web address: ncbi.nlm.nih.gov).

In specific embodiments, a ribosomal nucleotide sequence does not include one or more of the following sequences:

| | |
|---|---|
| (SEQ ID NO:141) | AUUCAUAAGGAGUACUCGAUCACGCGAAGU, |
| (SEQ ID NO:142) | ACAUUCGAACCGACACCUGUGCCUUACCGCGU; |
| (SEQ ID NO:143) | AUUGUCAGAGACUCGAGCGUACCAACUGGU; |
| (SEQ ID NO:144) | ACAUUAUCAAUCUAGCUAGGGUGUACACAAGU; |
| (SEQ ID NO:145) | ACAUUCGAACCAACCUGACACCCUAUCCCAGU; |
| (SEQ ID NO:146) | AUUGCGACCGGUUCUGCCAAUACUCGAGGUUG; |
| (SEQ ID NO:147) | AUUAGGGUGUGAAUGUGCUGAUCAACGCGU; |
| (SEQ ID NO:148) | ACAUUCGAAUGUCAAUGCGCAAGUAGACCGGU; |
| (SEQ ID NO:149) | AUUGAUCAAUAUUCGACCACCCUGCAGCGU; |
| (SEQ ID NO:150) | AUUGCGCAUGUCACGCUUCGAAGCCGCUGU; |
| (SEQ ID NO:151) | AUUCGACCG; |
| (SEQ ID NO:152) | GAUCGAUGUGGG; |
| or | |
| (SEQ ID NO:153) | GAUCGAUCUGG. |

In certain embodiments, a ribosomal nucleotide sequence does not include one or more of the following sequences:

(SEQ ID NO:154)

TCTCTCGGTGGCCGGGGCTCGTCGGGGTTTTGGGTCCGTCC;

(SEQ ID NO:155)

ACTGTCGTACTTGATATTTTGGGGTTTTGGGG;

(SEQ ID NO:156)

TGGACCAGACCTAGCAGCTATGGGGGAGCTGGGGAAGGTGGGATGTGA;
or

(SEQ ID NO:157)

AGACCTAGCAGCTATGGGGGAGCTGGGGTATA.

In some embodiments, an isolated nucleic acid can include a nucleotide sequence that encodes a nucleotide sequence described herein. In other embodiments, the nucleic acid includes a nucleotide sequence that encodes the complement of a nucleotide sequence described herein. For example, a ribosomal sequence described herein, or a sequence complementary to a ribosomal nucleotide sequence described herein, may be included within a longer nucleotide sequence in the nucleic acid. The encoded nucleotide sequence sometimes is referred to herein as an “aptamer” and can be utilized in screening methods or as a therapeutic. In certain embodiments, the aptamer is complementary to a nucleotide sequence herein and can hybridize to a target nucleotide sequence. The hybridized aptamer may form a duplex or triplex with the target complementary nucleotide sequence, for example. The aptamer can be synthesized by the encoding sequence in an in vitro or in vivo system. When synthesized in vitro, an aptamer sometimes contains analog or derivative nucleotides. When synthesized in vivo, the encoding sequence may integrate into genomic DNA in the system or replicate autonomously from the genome (e.g., within a plasmid nucleic acid). An aptamer sometimes is selected by a measure of binding or hybridization affinity to a particular protein or nucleic acid target. In certain embodiments the aptamer may bind to one or more protein molecules within a cell or in plasma and induce a therapeutic response or be used as a method to detect the presence of the protein(s).

In certain embodiments, a human ribosomal nucleotide sequence in an isolated nucleic acid is from one of the following regions of a human ribosomal nucleotide sequence or complement thereof: (a) 5′ETS region, ITS1 region, ITS2 region, 28S rRNA encoding region, 3′ETS region, 18S rRNA encoding region or 5.8S rRNA encoding region of rDNA (e.g., SEQ ID NO: 1); (b) complement of (a); (c) encoded RNA of (a); or (d) encoded RNA of (b). In SEQ ID NO: 1, the 5′ETS region spans from about position 1 to about position 3656; the ITS1 region spans from about position 5528 to about position 6622; the ITS2 region spans from about position 6780 to about position 7934, the 28S rRNA encoding region spans from about position 7935 to about position 12969, the 3′ETS region spans from about position 12970 to about position 13350, the 18S rRNA encoding region spans from about position 3657 to about position 5527; and the 5.8S rRNA encoding region spans from about position 6623 to about position 6779. In certain embodiments, a ribosomal nucleotide sequence in an isolated nucleic acid is from (a) 5′ETS region, ITS I region, ITS2 region, 28S rRNA encoding region or 3′ETS region of rDNA (e.g., SEQ ID NO: 1); (b) complement of (a); (c) encoded RNA of (a); or (d) encoded RNA of (b).

The isolated nucleic acid may be provided or contacted with other molecules under conditions that allow formation of a quadruplex structure, and sometimes stabilize the structure. The term “quadruplex structure,” as used herein refers to a structure within a nucleic acid that includes one or more guanine-tetrad (G-tetrad) structures or cytosine-tetrad structures (C-tetrad or “i-motif”). G-tetrads can form in quadruplex structures via Hoogsteen hydrogen bonds. A quadruplex structure may be intermolecular (i.e., formed between two, three, four or more separate nucleic acids) or intramolecular (i.e., formed within a single nucleic acid). In some embodiments, a quadruplex-forming nucleic acid is capable of forming a parallel quadruplex structure having four parallel strands (e.g., propeller structure), antiparallel quadruplex structure having two stands that are antiparallel to the two parallel strands (e.g., chair or basket quadruplex structure) or a partially parallel, also referred to as a “mixed parallel,” quadruplex structure having one strand that is antiparallel to three parallel strands (e.g., a chair-eller or basket-eller quadruplex structure). Such structures are described in U.S. Patent Application Publication Nos. 2004/0005601 and PCT Application PCT/US2004/037789, for example. One or more quadruplex structures may form within a nucleic acid, and may form at one or more regions in the nucleic acid. Depending upon the length of the nucleic acid, the entire nucleic acid may form the quadruplex structure, and often a portion of the nucleic forms a particular quadruplex structure. A variety of methods for determining the particular quadruplex conformation (e.g., parallel, antiparallel, mixed parallel) adopted by a nucleic acid sequence or subsequence are known, and described herein (e.g., circular dichroism).

Conditions that allow quadruplex formation and stabilization are known to the person of ordinary skill in the art, and optimal quadruplex-forming conditions can be tested. Ion type, ion concentration, counteranion type and incubation time can be varied, and the artisan of ordinary skill can routinely determine whether a quadruplex conformation forms and is stabilized for a given set of conditions by utilizing the methods described herein. For example, cations (e.g., monovalent cations such as potassium) can stablize quadruplex structures. The nucleic acid may be contacted in a solution containing ions for a particular time period, such as about 5 minutes, about 10 minutes, about 20 minutes, about 30 minutes, about 40 minutes, about 50 minutes or about 60 minutes or more, for example. A quadruplex structure is stabilized if it can form a functional quadruplex in solution, or if it can be detected in solution.

One nucleic acid sequence can give rise to different quadruplex orientations, where the different conformations depend in part upon the nucleotide sequence of the nucleic acid and conditions under which they form, such as the concentration of potassium ions present in the system and the time within which the quadruplex is allowed to form. Multiple conformations can be in equilibrium with one another, and can be in equilibrium with duplex nucleic acid if a complementary strand exists in the system. The equilibrium may be shifted to favor one conformation over another such that the favored conformation is present in a higher concentration or fraction over the other conformation or other conformations. The term “favor” or “stabilize” as used herein refers to one conformation being at a higher concentration or fraction relative to other conformations. The term “hinder” or “destabilize” as used herein refers to one conformation being at a lower concentration. One conformation may be favored over another conformation if it is present in the system at a fraction of 50% or greater, 75% or greater, or 80% or greater or 90% or greater with respect to another conformation (e.g., another quadruplex conformation, another paranemic conformation, or a duplex conformation). Conversely, one conformation may be hindered if it is present in the system at a fraction of 50% or less, 25% or less, or 20% or less and 10% or less, with respect to another conformation.

Equilibrium may be shifted to favor one quadruplex form over another form by methods described herein. A quadruplex forming region in a nucleic acid may be altered in a variety of manners. Alternations may result from an insertion, deletion, or substitution of one or more nucleotides. Substitutions can include a single nucleotide replacement of a nucleotide, such as a guanine that participates in a G-tetrad, where one, two, three, or four of more of such guanines in the quadruplex nucleic acid may be substituted. Also, one or more nucleotides near a guanine that participates in a G-tetrad may be deleted or substituted or one or more nucleotides may be inserted (e.g., within one, two, three or four nucleotides of a guanine that participates in a G-tetrad. A nucleotide may be substituted with a nucleotide analog or with another DNA or RNA nucleotide (e.g., replacement of a guanine with adenine, cytosine or thymine), for example. Ion concentrations and the time with which quadruplex DNA is contacted with certain ions can favor one conformation over another. Ion type, counterion type, ion concentration and incubation times can be varied to select for a particular quadruplex conformation. In addition, compounds that interact with quadruplex DNA may favor one form over the other and thereby stabilize a particular form.

Standard procedures for determining whether a quadruplex structure forms in a nucleic acid are known to the person of ordinary skill in the art. Also, different quadruplex conformations can be identified separately from one another using standard known procedures known to the person of ordinary skill in the art. Examples of such methods, such as characterizing quadruplex formation by polymerase arrest and circular dichroism, for example, are described in the Examples section hereafter.

Identification of Ribosomal Nucleotide Sequence Interacting Molecules

Provided are methods for identifying agents that interact with a ribosomal nucleic acid described herein. Assay components, such as one or more ribosomal nucleic acids and one or more test molecules, are contacted and the presence or absence of an interaction is observed. Assay components may be contacted in any convenient format and system by the artisan. As used herein, the term “system” refers to an environment that receives the assay components, including but not limited to microtiter plates (e.g., 96-well or 384-well plates), silicon chips having molecules immobilized thereon and optionally oriented in an array (see, e.g., U.S. Pat. No. 6,261,776 and Fodor, Nature 364: 555-556 (1993)), microfluidic devices (see, e.g., U.S. Pat. Nos. 6,440,722; 6,429,025; 6,379,974; and 6,316,781) and cell culture vessels. The system can include attendant equipment, such as signal detectors, robotic platforms, pipette dispensers and microscopes. A system sometimes is cell free, sometimes includes one or more cells, sometimes includes or is a cell sample from an animal (e.g., a biopsy, organ, appendage), and sometimes is a non-human animal. Cells may be extracted from any appropriate subject, such as a mouse, rat, hamster, rabbit, guinea pig, ungulate (e.g., equine, bovine, porcine), monkey, ape or human subject, for example.

The artisan can select test molecules and test conditions based upon the system utilized and the interaction and/or biological activity parameters monitored. Any type of test molecule can be utilized, including any reagent described herein, and can be selected from chemical compounds, antibodies and antibody fragments, binding partners and fragments, and nucleic acid molecules, for example. Specific embodiments of each class of such molecules are described hereafter. One or more test molecules may be added to a system in assays for identifying ribosomal nucleic acid interacting molecules. Test molecules and other components can be added to the system in any suitable order. A sample exposed to a particular condition or test molecule often is compared to a sample not exposed to the condition or test molecule so that any changes in interactions or biological activities can be observed and/or quantified.

Assay systems sometimes are heterogeneous or homogeneous. In heterogeneous assays, one or more reagents and/or assay components are immobilized on a solid phase, and complexes are detected on the solid phase at the end of the reaction. In homogeneous assays, the entire reaction is carried out in a liquid phase. In either approach, the order of addition of reactants can be varied to obtain different information about the molecules being tested. For example, test compounds that agonize target molecule/binding partner interactions can be identified by conducting the reaction in the presence of the test molecule in a competition format. Alternatively, test molecules that agonize preformed complexes, e.g., molecules with higher binding constants that displace one of the components from the complex, can be tested by adding a test compound to the reaction mixture after complexes have been formed.

In a heterogeneous assay embodiment, one or more assay components are anchored to a solid surface (e.g., a microtiter plate), and a non-anchored component often is labeled, directly or indirectly. One or more assay components may be immobilized to a solid support in heterogeneous assay embodiments. The attachment between a component and the solid support may be covalent or non-covalent (see, e.g., U.S. Pat. No. 6,022,688 for non-covalent attachments). The term “solid support” or “solid phase” as used herein refers to a wide variety of materials including solids, semi-solids, gels, films, membranes, meshes, felts, composites, particles, and the like. Suitable solid phases include those developed and/or used as solid phases in solid phase binding assays (e.g., U.S. Pat. Nos. 6,261,776; 5,900,481; 6,133,436; and 6,022,688; WIPO publication WO 01/18234; chapter 9 of Immunoassay, E. P. Diamandis and T. K. Christopoulos eds., Academic Press: New York, 1996; Leon et al., Bioorg. Med. Chem. Lett. 8: 2997 (1998); Kessler et al., Agnew. Chem. Int. Ed. 40: 165 (2001); Smith et al., J. Comb. Med. 1: 326 (1999); Orain et al., Tetrahedron Lett. 42: 515 (2001); Papanikos et al., J. Am. Chem. Soc. 123: 2176 (2001); Gottschling et al., Bioorg. And Medicinal Chem. Lett. 11: 2997 (2001)). Examples of suitable solid phases include membrane filters, cellulose-based papers, beads (including polymeric, latex and paramagnetic particles), glass (e.g., glass slide), polyvinylidene fluoride (PVDF), nylon, silicon wafers, microchips, microparticles, nanoparticles, chromatography supports, TentaGels, AgroGels, PEGA gels, SPOCC gels, multiple-well plates (e.g., microtiter plate), nanotubes and the like that can be used by those of skill in the art to sequester molecules. The solid phase can be non-porous or porous. Assay components may be oriented on a solid phase in an array. Thus provided are arrays comprising one or more, two or more, three or more, etc., of assay components described herein (e.g., ribosomal nucleic acids) immobilized at discrete sites on a solid support in an ordered array. Such arrays sometimes are high-density arrays, such as arrays in which each spot comprises at least 100 molecules per square centimeter.

A partner of the immobilized species sometimes is exposed to the coated surface with or without a test molecule in certain heterogeneous assay embodiments. After the reaction is complete, unreacted components are removed (e.g., by washing) such that a significant portion of any complexes formed remain immobilized on the solid surface. Where the non-immobilized species is pre-labeled, the detection of label immobilized on the surface is indicative of complex formation. Where the non-immobilized species is not pre-labeled, an indirect label can be used to detect complexes anchored to the surface (e.g., by using a labeled antibody specific for the initially non-immobilized species). Depending upon the order of addition of reaction components, test compounds that inhibit complex formation or disrupt preformed complexes can be detected.

In certain embodiments, a protein or peptide test molecule or assay component is linked to a phage via a phage coat protein. Molecules capable of interacting with the protein or peptide linked to the phage are immobilized to a solid phase, and phages displaying proteins or peptides that interact with the immobilized components adhere to the solid support. Nucleic acids from the adhered phages then are isolated and sequenced to determine the sequence of the protein or peptide that interacted with the components immobilized on the solid phase. Methods for displaying a wide variety of peptides or proteins as fusions with bacteriophage coat proteins are well known (Scott and Smith, Science 249: 386-390 (1990); Devlin, Science 249: 404-406 (1990); Cwirla et al., Proc. Natl. Acad. Sci. 87: 6378-6382 (1990); Felici, J. Mol. Biol. 222: 301-310 (1991)). Methods are also available for linking the test polypeptide to the N-terminus or the C-terminus of the phage coat protein. The original phage display system was disclosed, for example, in U.S. Pat. Nos. 5,096,815 and 5,198,346. This system used the filamentous phage M13, which required that the cloned protein be generated in E. coli and required translocation of the cloned protein across the E. coli inner membrane. Lytic bacteriophage vectors, such as lambda, T4 and T7 are more practical since they are independent of E. coli secretion. T7 is commercially available and described in U.S. Pat. Nos. 5,223,409; 5,403,484; 5,571,698; and 5,766,905.

In heterogeneous assay embodiments, the reaction can be conducted in a liquid phase in the presence or absence of test molecule, where the reaction products are separated from unreacted components, and the complexes are detected (e.g., using an immobilized antibody specific for one of the binding components to anchor any complexes formed in solution, and a labeled antibody specific for the other partner to detect anchored complexes). Again, depending upon the order of addition of reactants to the liquid phase, test compounds that inhibit complex or that disrupt preformed complexes can be identified.

In some homogeneous assay embodiments, a preformed complex comprising a reagent and/or other component is prepared. One or more components in the complex (e.g., ribosomal nucleic acid, nucleolin protein, or nucleic acid binding compound) is labeled. In some embodiments, a signal generated by a label is quenched upon complex formation (e.g., U.S. Pat. No. 4,109,496 that utilizes this approach for immunoassays). Addition of a test molecule that competes with and displaces one of the species from the preformed complex can result in the generation of a signal above background or reduction in a signal. In this way, test substances that disrupt ribosomal nucleic acid/test molecule complexes can be identified.

In an embodiment for identifying test molecules that antagonize or agonize formation of a complex comprising a ribosomal nucleic acid, a reaction mixture containing components of the complex is prepared under conditions and for a time sufficient to allow complex formation. The reaction mixture often is provided in the presence or absence of the test molecule. The test molecule can be included initially in the reaction mixture, or can be added at a time subsequent to the addition of the target molecule and its binding partner. Control reaction mixtures are incubated without the test molecule or with a placebo. Formation of any complex is detected. Decreased formation of a complex in the reaction mixture containing test molecule as compared to in a control reaction mixture indicates that the molecule antagonizes complex formation. Alternatively, increased formation of a complex in the reaction mixture containing test molecule as compared to in a control reaction mixture indicates that the molecule agonizes target molecule/binding partner complex formation. In certain embodiments, complex formation ribosomal nucleic acid/interacting molecule can be compared to complex formation of a modified ribosomal nucleic acid/interacting molecule (e.g., nucleotide replacement in the ribosomal nucleic acid). Such a comparison can be useful in cases where it is desirable to identify test molecules that modulate interactions of modified nucleic acid but not non-modified nucleic acid.

In some embodiments, the artisan detects the presence or absence of an interaction between assay components (e.g., a ribosomal nucleic acid and a test molecule). As used herein, the term “interaction” typically refers to reversible binding of particular system components to one another, and such interactions can be quantified. A molecule may “specifically bind” to a target when it binds to the target with a degree of specificity compared to other molecules in the system (e.g., about 75% to about 95% or more of the molecule is bound to the target in the system). Often, binding affinity is quantified by plotting signal intensity as a function of a range of concentrations or amounts of a reagent, reactant or other system component. Quantified interactions can be expressed in terms of a concentration or amount of a reagent required for emission of a signal that is 50% of the maximum signal (IC50). Also, quantified interactions can be expressed as a dissociation constant (K_{d} or Kᵢ) using kinetic methods known in the art. Kinetic parameters descriptive of interaction characteristics in the system can be assessed, including for example, assessing Kₘ, k_{cat}, kₒₙ, and/or k_{off} parameters.

A variety of signals can be detected to identify the presence, absence or amount of an interaction. One or more signals detected sometimes are emitted from one or more detectable labels linked to one or more assay components. In some embodiments, one or more assay components are linked to a detectable label. A detectable label can be covalently linked to an assay component, or may be in association with a component in a non-covalent linkage. Non-covalent linkages can be effected by a binding pair, where one binding pair member is in association with the assay component and the other binding pair member is in association with the detectable label. Any suitable binding pair can be utilized to effect a non-covalent linkage, including, but not limited to, antibody/antigen, antibody/antibody, antibody/antibody fragment, antibody/antibody receptor, antibody/protein A or protein G, hapten/anti-hapten, biotin/avidin, biotin/streptavidin, folic acid/folate binding protein, vitamin B12/intrinsic factor, nucleic acid/complementary nucleic acid (e.g., DNA, RNA, PNA). Covalent linkages also can be effected by a binding pair, such as a chemical reactive group/complementary chemical reactive group (e.g., sulfhydryl/maleimide, sulfhydryl/haloacetyl derivative, amine/isotriocyanate, amine/succinimidyl ester, and amine/sulfonyl halides). Methods for attaching such binding pairs to reagents and effecting binding are known to the artisan.

Any detectable label suitable for detection of an interaction can be appropriately selected and utilized by the artisan. Examples of detectable labels are fluorescent labels such as fluorescein, rhodamine, and others (e.g., Anantha, et al., Biochemistry (1998) 37:2709 2714; and Qu & Chaires, Methods Enzymol. (2000) 321:353 369); radioactive isotopes (e.g., ¹²⁵I, ¹³¹I, ³⁵S, ³¹P, ³²P, ¹⁴C, ³H, ⁷Be, ²⁸Mg, ⁵⁷Co, ⁶⁵Zn, ⁶⁷Cu, ⁶⁸Ge, ⁸²Sr, ⁸³Rb, ⁹⁵Tc, ⁹⁶Tc, ¹⁰³Pd, ¹⁰⁹Cd, and ¹²⁷Xe); light scattering labels (e.g., U.S. Pat. No. 6,214,560, and commercially available from Genicon Sciences Corporation, CA); chemiluminescent labels and enzyme substrates (e.g., dioxetanes and acridinium esters), enzymic or protein labels (e.g., green fluorescence protein (GFP) or color variant thereof, luciferase, peroxidase); other chromogenic labels or dyes (e.g., cyanine), and labels described previously.

A fluorescence signal is generally monitored in assays by exciting a fluorophore at a specific excitation wavelength and then detecting fluorescence emitted by the fluorophore at a different emission wavelength. Many nucleic acid interacting fluorophores and their attendant excitation and emission wavelengths are known (e.g., those described above). Standard methods for detecting fluorescent signals also are known, such as by using a fluorescence detector. Background fluorescence may be reduced in the system with the addition of photon reducing agents (see, e.g., U.S. Pat. No. 6,221,612), which can enhance the signal to noise ratio.

Another signal that can be detected is a change in refractive index at a solid optical surface, where the change is caused by the binding or release of a refractive index enhancing molecule near or at the optical surface. These methods for determining refractive index changes of an optical surface are based upon surface plasmon resonance (SPR). SPR is observed as a dip in light intensity reflected at a specific angle from the interface between an optically transparent material (e.g., glass) and a thin metal film (e.g., silver or gold). SPR depends upon the refractive index of the medium (e.g., a sample solution) close to the metal surface. A change of refractive index at the metal surface, such as by the adsorption or binding of material near the surface, will cause a corresponding shift in the angle at which SPR occurs. SPR signals and uses thereof are further exemplified in U.S. Pat. Nos. 5,641,640; 5,955,729; 6,127,183; 6,143,574; and 6,207,381, and WIPO publication WO 90/05295 and apparatuses for measuring SPR signals are commercially available (Biacore, Inc., Piscataway, N.J.). In certain embodiments, an assay component can be linked via a linker to a chip having an optically transparent material and a thin metal film, and interactions between and/or with the reagents can be detected by changes in refractive index. An assay component linked to a chip for SPR analysis, in certain embodiments, can be (1) a rDNA or rRNA subsequence, sometimes containing a quadruplex-forming sequence, (2) a rDNA or rRNA binding protein (e.g., nucleolin), or (3) a rDNA or rRNA binding molecule (e.g., compound A-1), for example.

Other signals representative of structure may also be detected, such as NMR spectral shifts (see, e.g., Arthanari & Bolton, Anti-Cancer Drug Design 14: 317-326 (1999)), mass spectrometric signals and fluorescence resonance energy transfer (FRET) signals (e.g., Lakowicz et al., U.S. Pat. No. 5,631,169; Stavrianopoulos et al. U.S. Pat. No. 4,868,103). in FRET approaches, a fluorophore label on a first, “donor” molecule is selected such that its emitted fluorescent energy will be absorbed by a fluorescent label on a second, “acceptor” molecule, which in turn is able to fluoresce due to the absorbed energy. Alternately, the “donor” polypeptide molecule may simply utilize the natural fluorescent energy of tryptophan residues. Labels are chosen that emit different wavelengths of light, such that the “acceptor” molecule label may be differentiated from that of the “donor”. Since the efficiency of energy transfer between the labels is related to the distance separating the molecules, the spatial relationship between the molecules can be assessed. In a situation in which binding occurs between the molecules, the fluorescent emission of the “acceptor” molecule label in the assay should be maximal. A FRET binding event can be conveniently measured using standard fluorometric detection means well known (e.g., using a fluorimeter). Molecules useful for FRET are known (e.g., fluorescein and terbium). FRET can be utilized to detect interactions in vitro or in vivo.

Interaction assays sometimes are performed in a heterogeneous format in which interactions are detected by monitoring detectable label in association with or not in association with a target linked to a solid phase. An example of such a format is an immunoprecipitation assay. Multiple separation processes are available, such as gel electrophoresis, chromatography, sedimentation (e.g., gradient sedimentation) and flow cytometry processes, for example. Flow cytometry processes include, for example, flow microfluorimetry (FMF) and fluorescence activated cell sorting (FACS); U.S. Pat. Nos. 6,090,919 (Cormack, et al.); 6,461,813 (Lorens); and 6,455,263 (Payan)). In some embodiments, cells also may be washed of unassociated detectable label, and detectable label associated with cellular components may be visualized (e.g., by microscopy).

In specific assay embodiments, provided is a method for identifying a molecule that binds to a nucleic acid containing a human ribosomal nucleotide sequence, which comprises: (a) contacting a nucleic acid containing a human ribosomal nucleotide sequence described herein, a compound that binds to the nucleic acid and a test molecule, and (b) detecting the amount of the compound bound or not bound to the nucleic acid, whereby the test molecule is identified as a molecule that binds to the nucleic acid containing the human ribosomal nucleotide sequence when less of the compound binds to the nucleic acid in the presence of the test molecule than in the absence of the test molecule. The compound sometimes is in association with a detectable label, and at times is radiolabeled. In certain embodiments, the compound is a quinolone analog (e.g., a quinolone analog described herein). In specific embodiments, the compound is a radiolabeled compound of formula A, and in specific embodiments, the compound is radiolabeled compound A-1. Methods for radiolabeling compounds are known (e.g., U.S. patent application 60/718,021, filed Sep. 16, 2005, entitled METHODS FOR PREPARING RADIOACTIVE QUINOLONE ANALOGS). In some embodiments, the compound is a porphyrin (e.g., TMPyP4 or an expanded porphyrin described in U.S. patent application publication no. 20040110820 (e.g., Se₂SAP)). In the latter embodiments, fluorescence of the porphyrin sometimes is detected as the signal. The nucleic acid and/or another assay component sometimes is in association with a solid phase in certain embodiments. The nucleic acid may be DNA, RNA or an analog thereof, and may comprise a nucleotide sequence described above in specific embodiments. The nucleic acid may form a quadruplex, such as an intramolecular quadruplex.

In other specific assay embodiments, provided is a method for identifying a molecule that causes nucleolin displacement, which comprises (a) contacting a nucleic acid containing a human ribosomal nucleotide sequence and a nucleolin protein with a test molecule, wherein the nucleic acid is capable of binding to the nucleolin protein, and (b) detecting the amount of the nucleic acid bound or not bound to the nucleolin protein, whereby the test molecule is identified as a molecule that causes nucleolin displacement when less of the nucleic acid binds to the nucleolin protein in the presence of the test molecule than in the absence of the test molecule. In some embodiments, the nucleolin protein is in association with a detectable label, and the nucleolin protein may be in association with a solid phase. The nucleic acid sometimes is in association with a detectable label, and the nucleic acid may be in association with a solid phase in certain embodiments. Any convenient combination of the foregoing may be utilized. The nucleic acid may be DNA, RNA or an analog thereof, and may comprise a nucleotide sequence described above in specific embodiments. The nucleic acid may comprise G-quadruplex sequences and/or hairpin structures, sometimes composed of a five base pair stem and seven to ten nucleotide loop (e.g., U/GCCCGA motif) Any nucleolin protein may be utilized, such as a nucleolin having a sequence of accession no. NM_{—}005381, or a fragment or substantially identical sequence variant of the foregoing capable of binding a nucleic acid. Examples of nucleolin domains are RRM domains (e.g., amino acids 278-640) and RGG domains (e.g., amino acids 640-709). In some embodiments the test molecule is a quinolone analog. Nucleolin distribution can be detected by immunofluorescence microscopy in cells.

In a certain assay embodiments, provided are methods for identifying a molecule that modulates ribosomal RNA (rRNA) synthesis, which comprise: contacting cells with a test molecule, contacting the rRNA with one or more primers that amplify a portion thereof and a labeled probe that hybridizes to the amplification product, detecting the amount of the amplification product by hybridization of the labeled probe, whereby a test molecule that reduces or increases the amount of amplification product is identified as a molecule that modulates rRNA synthesis. In some embodiments, the methods comprise contacting cells with a test molecule, contacting the mixture with one or more primers that amplify a portion of rRNA and a labeled probe that hybridizes to the amplification product, detecting the amount of the amplification product by hybridization of the labeled probe, whereby a test molecule that reduces or increases the amount of amplification product is identified as a molecule that modulates rRNA synthesis. The labeled probe in some embodiments is added after the primers are added and the rRNA is amplified, and in certain embodiments, the labeled probe and the primers are added at the same time. The portion of rRNA amplified sometimes is at the 5′ end of the rRNA. In certain embodiments, the test molecule is a quinolone analog, such as a quinolone analog of formula 3 or 3A or of formula 2 or 2A-2D. In certain multiplex embodiments, the above-described method is carried out using multiple probes in a single reaction (e.g., two or more probes), each of which hybridize to distinct amplification products (e.g., rDNA product and a comparison product (e.g., c-Myc product)) and contains a unique detectable tag. In such multiplex embodiments, multiple distinct probes, and optionally, multiple distinct primer pairs for amplifying a target sequence region, can be provided.

Some embodiments are directed to 53. A composition comprising a probe oligonucleotide that specifically hybridizes to a target sequence in a nucleotide sequence comprising ((G3+)N1-7)3G3+ (SEQ ID NO:230) or ((C3+)N1-7)3C3+ (SEQ ID NO:231) in a human ribosomal DNA or RNA, or complement thereof, where: G is guanine, C is cytosine, 3+ is three or more nucleotides and N is any nucleotide, and the probe oligonucleotide comprises a detectable label. In some embodiments, the target region comprises a nucleotide sequence at the 5′ end of rDNA or rRNA, and sometimes is a (a) 5′ETS region, ITS1 region, ITS2 region, 28S rRNA region, 3′ETS region, 18S rRNA region or 5.8S rRNA region of rDNA (e.g., SEQ ID NO: 1); (b) complement of (a); encoded RNA of (a); or encoded RNA of (b). The template sometimes is DNA, and the target sequence sometimes comprises a human ribosomal nucleotide sequence from SEQ ID NO: 1. In certain embodiments, the template is RNA, and sometimes the target sequence is encoded by a nucleotide sequence in SEQ ID NO: 1. The composition sometimes further comprises a template-dependent nucleic acid polymerase having a 5′ to 3′ nuclease activity. The probe oligonucleotide can be labeled at the 5′ terminus and the probe can comprises a tail of non-nucleic acids or a sequence of nucleotides which is non-complementary to the target nucleic acid sequence. In certain embodiments, the probe oligonucleotide comprises a first and second label. The first and second labels can be interactive signal generating labels effectively positioned on the probe oligonucleotide to quench the generation of detectable signal. The first label sometimes is a fluorophore and the second label sometimes is a quenching agent, and the first label can be at the 5 ′terminus and the second label may be at the 3′ terminus. The 3′ terminus of the probe oligonucleotide is blocked in some embodiments, and the probe oligonucleotide sometimes is detectable by fluorescence. The probe oligonucleotide sometimes comprises a ligand having a specific binding partner, where the ligand sometimes is biotin, avidin or streptavidin. The composition in certain embodiments further comprises one or more primer oligonucleotides that specifically hybridize to a human ribosomal template DNA or RNA adjacent to the target sequence or complement thereof, and the composition sometimes further comprises one or more extension nucleotides.

Certain embodiments are directed to a reaction mixture for use in a process for the amplification and detection of a target nucleic acid sequence in a sample which reaction mixture, prior to amplification, comprises a pair of oligonucleotide primers and a labeled oligonucleotide, where: the pair of oligonucleotide primers comprises a first a primer complementary to the target nucleic acid and which primes the synthesis of a first extension product that is complementary to the target nucleic acid, and a second primer complementary to the first extension product and which primes the synthesis of a second extension product; and the labeled oligonucleotide hybridizes to a region of the target nucleic acid or the complement of the target nucleic acid, where the region is between one member of the primer pair and the complement of the other member of the primer pair, and the region is a region of rDNA or rRNA. In certain embodiments, the region is at the 5′ end of rDNA or rRNA, and sometimes is from (a) 5′ETS region, ITS1 region, ITS2 region, 28S rRNA region, 3′ETS region, 18S rRNA region or 5.8S rRNA region of rDNA (e.g., SEQ ID NO: 1); (b) complement of (a); encoded RNA of (a); or encoded RNA of (b). Sometimes, the reaction mixture further comprises a template-dependent nucleic acid polymerase having a 5′ to 3′ nuclease activity. In certain embodiments, the labeled oligonucleotide is labeled at the 5′ terminus, and sometimes the labeled oligonucleotide further comprises a tail of non-nucleic acids or a sequence of nucleotides which is non-complementary to the target nucleic acid sequence. The labeled oligonucleotide may comprise a first and second label, and sometimes the first and second labels are interactive signal generating labels effectively positioned on the labeled oligonucleotide to quench the generation of detectable signal. The 3′ terminus of the labeled oligonucleotide can be blocked, and sometimes the labeled oligonucleotide is detectable by fluorescence. In certain embodiments, the first label is a fluorophore and the second label is a quenching agent. Sometimes the first label is at the 5′ terminus and the second label is at the 3′ terminus. In certain embodiments, the labeled oligonucleotide comprises a ligand having a specific binding partner, and sometimes the ligand is biotin. PCR methods, components and reaction mixtures are described in U.S. Pat. Nos. 4,683,202; 4,683,195; 4,965,188; 6,214,979; 5,804,375; 5,210,015; 5,487,972 and 5,538,848, for example, and primers and probes that hybridize to rDNA or rRNA sequences described herein can be applied to embodiments described in these patents.

Also provided are kits for detecting a target nucleic acid sequence in a sample comprising: (a) at least one labeled oligonucleotide containing a sequence complementary to a region of the target nucleic acid, where the labeled oligonucleotide anneals within the target nucleic acid sequence bounded by the oligonucleotide primers of part (b) and where the labeled oligonucleotide is complementary to an rDNA or rRNA sequence and where the labeled oligonucleotide is blocked at the 3′ terminus to prohibit incorporation of the labeled oligonucleotide into a primer extension product, where the blocking is achieved by adding a chemical moiety to the 3′ hydroxyl of the last nucleotide, which moiety does not also serve as a label for subsequent detection or by removing the 3′-hydroxyl; and (b) a set of oligonucleotide primers, where a first primer contains a sequence complementary to a region in one strand of the target nucleic acid sequence and primes the synthesis of a first extension product, and a second primer contains a sequence complementary to a region in the first extension product and primes the synthesis of a nucleic acid strand complementary to the first extension product, and where each oligonucleotide primer is selected to anneal to its complementary template upstream of any labeled oligonucleotide annealed to the same nucleic acid strand. In some embodiments the blocking is achieved by adding a chemical moiety to the 3′ hydroxyl of the last nucleotide of the labeled oligonucleotide, which chemical moiety is a phosphate group. In certain embodiments the blocking is achieved by removing the 3′-hydroxyl from the labeled oligonucleotide. Certain kits further comprise a nucleic acid polymerase having a 5′ to 3′ nuclease activity, such as a thermostable enzyme (e.g., from a Thermus species). The labeled oligonucleotide may be detectable by fluorescence, and can be labeled at the 5′ terminus. The labeled oligonucleotide sometimes comprises first and second labels where the first label is separated from the second label by a nuclease susceptible cleavage site. In certain embodiments the first label is at the 5′ terminus and the second label is at the 3′ terminus. The labeled oligonucleotide sometimes comprises a pair of interactive signal-generating labels positioned on the labeled oligonucleotide to quench the generation of detectable signal, and sometimes the first label is a fluorophore and the second label is a quencher which interacts therewith.

Also provided is a detectably labeled oligonucleotide probe, which probe is blocked at the 3′ terminus to prohibit polymerase catalyzed extension of the probe, where the blocking is achieved either by adding a chemical moiety to the 3′ hydroxyl of the terminal nucleotide, which chemical moiety does not also serve as a label for subsequent detection, or by removing the 3′ hydroxyl; and where the labeled oligonucleotide probe comprises a pair of non-radioactive interactive labels consisting of a first label and a second label, the first label and second label attached to the oligonucleotide directly or indirectly, and where the first label is separated from the second label by a nuclease susceptible cleavage site; and where the probe hybridizes to a rDNA or rRNA nucleotide sequence. In certain embodiments, the probe specifically hybridizes to the 5′ end of rDNA or rRNA, and sometimes is from (a) 5′ETS region, ITS1 region, ITS2 region, 28S rRNA region, 3′ETS region, 18S rRNA region or 5.8S rRNA region of rDNA (e.g., SEQ ID NO: 1); (b) complement of (a); encoded RNA of (a); or encoded RNA of (b). In some embodiments, the first label is at the 5′ terminus and the second label is at the 3′ terminus of the probe, and sometimes the first and second labels comprise a pair of interactive signal-generating labels positioned on the labeled oligonucleotide to quench the generation of detectable signal. In certain embodiments, the first label is a fluorophore and the second label is a quencher which interacts therewith.

Test molecules identified as having an effect in an assay described herein can be analyzed and compared to one another (e.g., ranked). Molecules identified as having an interaction or effect in a methods described herein are referred to as “candidate molecules.” Provided herein are candidate molecules identified by screening methods described herein, information descriptive of such candidate molecules, and methods of using candidate molecules (e.g., for therapeutic treatment of a condition).

Accordingly, provided is structural information descriptive of a candidate molecule identified by a method described herein. In certain embodiments, information descriptive of molecular structure (e.g., chemical formula or sequence information) sometimes is stored and/or renditioned as an image or as three-dimensional coordinates. The information often is stored and/or renditioned in computer readable form and sometimes is stored and organized in a database. In certain embodiments, the information may be transferred from one location to another using a physical medium (e.g., paper) or a computer readable medium (e.g., optical and/or magnetic storage or transmission medium, floppy disk, hard disk, random access memory, computer processing unit, facsimile signal, satellite signal, transmission over an internet or transmission over the world-wide web).

Ribosomal Nucleotide Sequence Interacting Molecules

Multiple types of ribosomal nucleotide sequence interacting molecules can be constructed, identified and utilized by the person of ordinary skill in the art. Examples of such interacting molecules are compounds, nucleic acids and antibodies. Any of these types of molecules may be utilized as test molecules in assays described herein.

Compounds can be obtained using any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive (see, e.g., Zuckermann et al., J. Med. Chem. 37: 2678-85 (1994)); spatially addressable parallel solid phase or solution phase libraries; synthetic library methods requiring deconvolution; “one-bead one-compound” library methods; and synthetic library methods using affinity chromatography selection. Biological library and peptoid library approaches are typically limited to peptide libraries, while the other approaches are applicable to peptide, non-peptide oligomer or small molecule libraries of compounds (Lam, Anticancer Drug Des. 12: 145, (1997)). Examples of methods for synthesizing molecular libraries are described, for example, in DeWitt et al., Proc. Natl. Acad. Sci. U.S.A. 90: 6909 (1993); Erb et al., Proc. Natl. Acad. Sci. USA 91: 11422 (1994); Zuckermann et al., J. Med. Chem. 37: 2678 (1994); Cho et al., Science 261: 1303 (1993); Carrell et al., Angew. Chem. Int. Ed. Engl. 33: 2059 (1994); Carell et al., Angew. Chem. Int. Ed. Engl. 33: 2061 (1994); and in Gallop et al., J. Med. Chem. 37: 1233 (1994). Libraries of compounds may be presented in solution (e.g., Houghten, Biotechniques 13: 412-421 (1992)), or on beads (Lam, Nature 354: 82-84 (1991)), chips (Fodor, Nature 364: 555-556 (1993)), bacteria or spores (Ladner, U.S. Pat. No. 5,223,409), plasmids (Cull et al., Proc. Natl. Acad. Sci. USA 89: 1865-1869 (1992)) or on phage (Scott and Smith, Science 249: 386-390 (1990); Devlin, Science 249: 404-406 (1990); Cwirla et al., Proc. Natl. Acad. Sci. 87: 6378-6382 (1990); Felici, J. Mol. Biol. 222: 301-310 (1991); Ladner supra.).

A compound sometimes is a small molecule. Small molecules include, but are not limited to, peptides, peptidomimetics (e.g., peptoids), amino acids, amino acid analogs, polynucleotides, polynucleotide analogs, nucleotides, nucleotide analogs, organic or inorganic compounds (I.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

A ribosomal nucleotide sequence interacting compound sometimes is a quinolone analog or derivative. In certain embodiments, the compound is of formula 1:

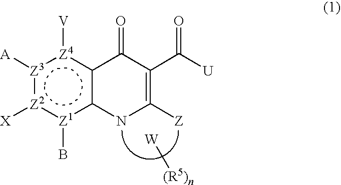

and pharmaceutically acceptable salts, esters and prodrugs thereof;

wherein B, X, A, or V is absent if Z¹, Z², Z³, or Z⁴, respectively, is N , and independently H, halo, azido, R², CH₂R², SR², OR² or NR¹R² if Z¹, Z², Z³, or Z⁴, respectively, is C; or

A and V, A and X, or X and B may form a carbocyclic ring, heterocyclic ring, aryl or heteroaryl, each of which may be optionally substituted and/or fused with a cyclic ring;

Z is O, S, NR¹, CH₂, or C═O;

Z¹, Z², Z³ and Z⁴ are C or N, provided any two N are non-adjacent;

W together with N and Z forms an optionally substituted 5- or 6-membered ring that is fused to an optionally substituted saturated or unsaturated ring; said saturated or unsaturated ring may contain a heteroatom and is monocyclic or fused with a single or multiple carbocyclic or heterocyclic rings;

U is R², OR², NR¹R², NR¹—(CR¹₂)ₙ—NR³R⁴, or N═CR¹R², wherein in N═CR¹R²R¹ and R² together with C may form a ring;

in each NR¹R², R¹ and R² together with N may form an optionally substituted ring;

in NR³R⁴, R³ and R⁴ together with N may form an optionally substituted ring;

R¹ and R³ are independently H or C₁₋₆ alkyl;

each R² is H, or a C₁₋₁₀ alkyl or C₂₋₁₀ alkenyl each optionally substituted with a halogen, one or more non-adjacent heteroatoms, a carbocyclic ring, a heterocyclic ring, an aryl or heteroaryl, wherein each ring is optionally substituted; or R² is an optionally substituted carbocyclic ring, heterocyclic ring, aryl or heteroaryl;

R⁴ is H, a C₁₋₁₀ alkyl or C₂₋₁₀ alkenyl optionally containing one or more non-adjacent heteroatoms selected from N, O and S, and optionally substituted with a carbocyclic or heterocyclic ring; or R³ and R⁴ together with N may form an optionally substituted ring;

each R⁵ is a substituent at any position on ring W; and is H, OR², amino, alkoxy, amido, halogen, cyano or an inorganic substituent; or R⁵ is C₁₋₆ alkyl, C₂₋₆ alkenyl, C₂₋₆ alkynyl, —CONHR¹, each optionally substituted by halo, carbonyl or one or more non-adjacent heteroatoms; or two adjacent R⁵ are linked to obtain a 5-6 membered optionally substituted carbocyclic or heterocyclic ring that may be fused to an additional optionally substituted carbocyclic or heterocyclic ring; and

n is 1-6.

In the above formula (1), B may be absent when Z¹ is N, or is H or a halogen when Z¹ is C. In certain embodiments, U sometimes is not H. In some embodiments, at least one of Z¹-Z⁴ is N when U is OH, OR² or NH₂.

In some embodiments, the compound has the general formula (2A) or (2B):

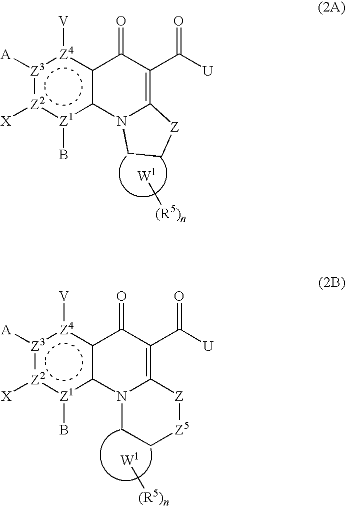

wherein A, B, V, X, U, Z, Z¹, Z², Z³, Z⁴, R⁵ and n are as defined in formula (1);

Z¹ is O, NR¹, CR⁶, or C═O;

R⁶ is H, C₁₋₆ alkyl, hydroxyl, alkoxy, halo, amino or amido; and

Z and Z⁵ may optionally form a double bond.

In some embodiments, compounds of the following formula (2C), or a pharmaceutically acceptable salt, ester or prodrug thereof, are utilized:

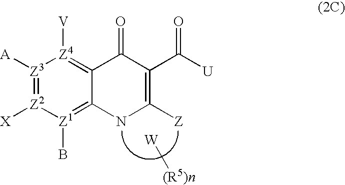

wherein substituents are set forth above.

In some embodiments, compounds of the following formula (2D), or a pharmaceutically acceptable salt, ester or prodrug thereof, are utilized:

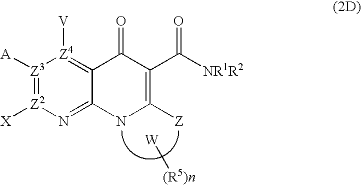

wherein substituents are set forth above. In certain embodiments, compounds of formula (2D) substantially arrest cell cycle, such as G1 phase arrest and/or S phase arrest, for example.

In certain aspects, the compound has the general formula (3):

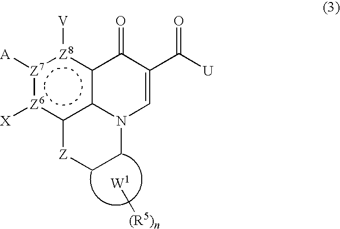

wherein A, U, V, X, R⁵, Z and n are as described above in formula (1);

W¹ is an optionally substituted aryl or heteroaryl, which may be monocyclic, or fused with a single or multiple ring and optionally containing a heteroatom; and

Z⁶, Z⁷, and Z⁸ are independently C or N, provided any two N are non-adjacent.

In the above formula (3), each of Z⁶, Z⁷, and Z⁸ may be C. In some embodiments, one or two of Z⁶, Z⁷, and Z⁸ is N, provided any two N are non-adjacent.

In the above formula, W together with N and Z in formula (1), or W¹ in formula (2A), (2B) or (3) forms an optionally substituted 5- or 6-membered ring that is fused to an optionally substituted aryl or heteroaryl selected from the group consisting of:

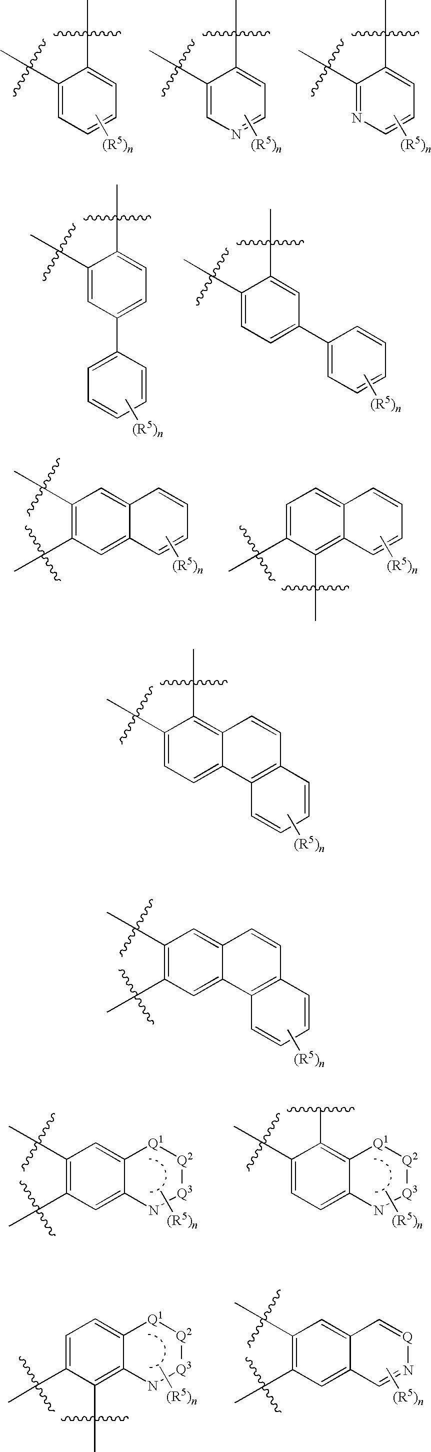

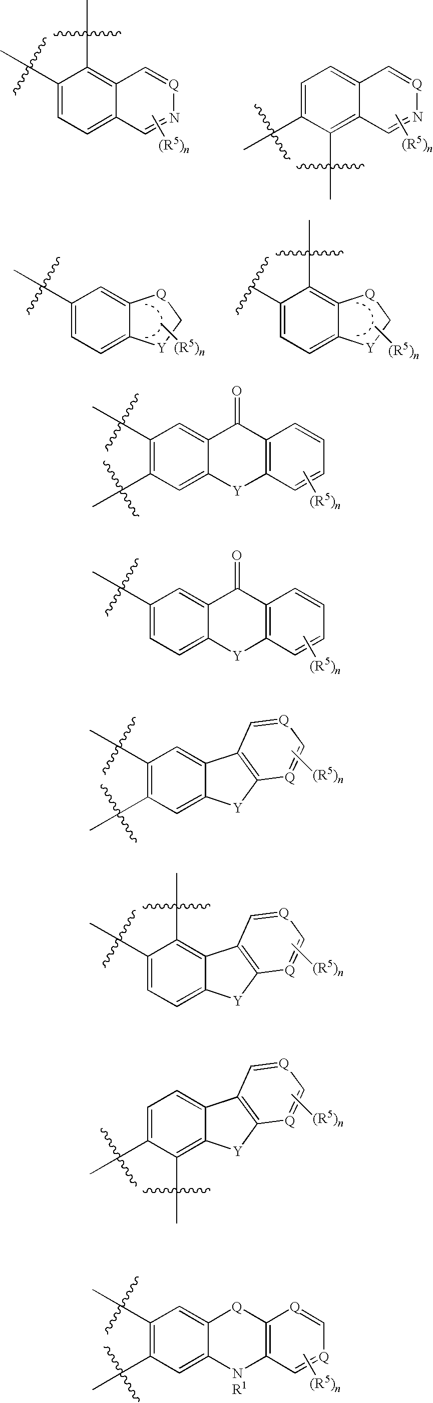

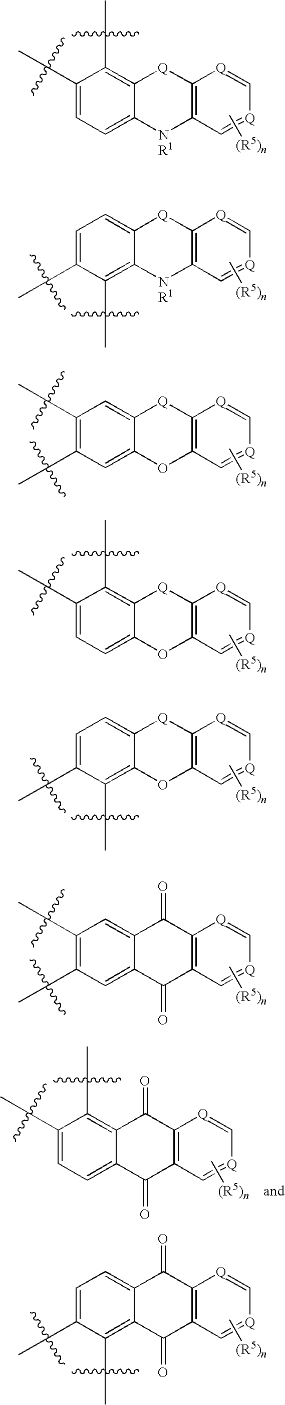

wherein each Q, Q¹, Q², and Q³ is independently CH or N;

Y is independently O, CH, C═O or NR^{X};

n and R⁵ is as defined above.

In certain embodiments, W together with N and Z in formula (1) form a group having the formula selected from the group consisting of

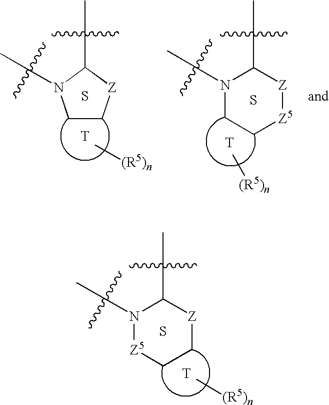

wherein Z is O, S, CR¹, NR¹, or C═O;

each Z⁵ is CR⁶, NR¹, or C═O, provided Z and Z¹ if adjacent are not both NR¹;

each R¹ is H, C₁₋₆ alkyl, COR² or S(O)ₚR² wherein p is 1-2;

R⁶ is H, or a substituent known in the art, including but not limited to hydroxyl, alkyl, alkoxy, halo, amino, or amido; and

ring S and ring T may be saturated or unsaturated.

In some embodiments, W together with N and Z in formula (1) forms a 5- or 6-membered ring that is fused to a phenyl. In other embodiments, W together with N and Z forms a 5- or 6-membered ring that is optionally fused to another ring, when U is NR¹R², provided U is not NH₂. In certain embodiments, W together with N and Z forms a 5- or 6-membered ring that is not fused to another ring, when U is NR¹R² (e.g., NH₂).

In the above formula (1), (2A), (2B) or (3), U may be NR¹R², wherein R¹ is H, and R² is a C₁₋₁₀ alkyl optionally substituted with a heteroatom, a C₃₋₆ cycloalkyl, aryl or a 5-14 membered heterocyclic ring containing one or more N, O or S. For example, R² may be a C₁₋₁₀ alkyl substituted with an optionally substituted morpholine, thiomorpholine, imidazole, aminodithiadazole, pyrrolidine, piperazine, pyridine or piperidine. In other examples, R¹ and R² together with N form an optionally substituted piperidine, pyrrolidine, piperazine, morpholine, thiomorpholine, imidazole, or aminodithiazole.

In some embodiments, U is NR¹—(CR¹₂)ₙ—NR³R⁴; n is 1-4; and R³ and R⁴ in NR³R⁴ together form an optionally substituted piperidine, pyrrolidine, piperazine, morpholine, thiomorpholine, imidazole, or aminodithiazole. In some examples, U is NH—(CH₂)ₙ—NR³R⁴ wherein R³ and R⁴ together with N form an optionally substituted pyrrolidine, which may be linked to (CH₂)ₙ at any position in the pyrrolidine ring. In one embodiment, R³ and R⁴ together with N form an N-methyl substituted pyrrolidine. In some embodiments, U is 2-(1-methylpyrrolidin-2-yl)ethylamino or (2-pyrrolidin-1-yl)ethanamino.

In the above formula (1), (2A) or (2B) or (3), Z may be S or NR¹.

In some embodiments, at least one of B, X, or A in formula (1), (2A) or (2B) is halo and Z¹, Z², and Z³ are C. In other embodiments, X and A are not each H when Z² and Z³ are C. In the above formula (1), (2A) and (2B), V may be H. In particular embodiments, U is not OH.

In an embodiment, each of Z¹, Z², Z³ and Z⁴ in formula (1), (2A) or (2B) are C. In another embodiment, three of Z¹, Z², Z³ and Z⁴ is C, and the other is N. For example, Z¹, Z² and Z³ are C, and Z⁴ is N. Alternatively, Z¹, Z² and Z⁴ are C, and Z³ is N. In other examples, Z¹, Z³ and Z⁴ are C and Z² is N. In yet other examples, Z², Z³ and Z⁴ are C, and Z¹ is N.

In certain embodiments, two of Z¹, Z², Z³ and Z⁴ in formula (1), (2A) or (2B) are C, and the other two are non-adjacent nitrogens. For example, Z¹ and Z³ may be C, and Z² and Z⁴ are N. Alternatively, Z¹ and Z³ may be N, and Z² and Z⁴ may be C. In other examples, Z¹ and Z⁴ are N, and Z² and Z³ are C. In particular examples, W together with N and Z forms a 5- or 6-membered ring that is fused to a phenyl.

In some embodiments, each of B, X, A, and V in formula (1), (2A) or (2B) is H and Z¹-Z⁴ are C. In many embodiments, at least one of B, X, A, and V is H and the corresponding adjacent Z¹-Z⁴ atom is C. For example, any two of B, X, A, and V may be H. In one example, V and B may both be H. In other examples, any three of B, X, A, and V are H and the corresponding adjacent Z¹-Z⁴ atom is C.

In certain embodiments, one of B, X, A, and V is a halogen (e.g., fluorine) and the corresponding adjacent Z¹-Z⁴ is C. In other embodiments, two of X, A, and V are halogen or SR², wherein R² is a C₀₋₁₀ alkyl or C₂₋₁₀ alkenyl optionally substituted with a heteroatom, a carbocyclic ring, a heterocyclic ring, an aryl or a heteroaryl; and the corresponding adjacent Z²-Z⁴ is C. For example, each X and A may be a halogen. In other examples, each X and A if present may be SR², wherein R² is a C₀₋₁₀ alkyl substituted with phenyl or pyrazine. In yet other examples, V, A and X may be alkynyls, fluorinated alkyls such as CF₃, CH₂CF₃, perfluorinated alkyls, etc.; cyano, nitro, amides, sulfonyl amides, or carbonyl compounds such as COR².

In each of the above formulas, U, and X, V, and A if present may independently be NR¹R², wherein R¹ is H, and R² is a C₁₋₁₀ alkyl optionally substituted with a heteroatom, a C₃₋₆ cycloalkyl, aryl or a 5-14 membered heterocyclic ring containing one or more N, O or S. If more than one NR¹R² moiety is present in a compound within the invention, as when both A and U are NR¹R² in a compound according to any one of the above formula, each R¹ and each R² is independently selected. In one example, R² is a C₁₋₁₀ alkyl substituted with an optionally substituted 5-14 membered heterocyclic ring. For example, R² may be a C₁₋₁₀ alkyl substituted with morpholine, thiomorpholine, imidazole, aminodithiadazole, pyrrolidine, piperazine, pyridine or piperidine. Alternatively, R¹ and R² together with N may form an optionally substituted heterocyclic ring containing one or more N, O or S. For example, R¹ and R² together with N may form piperidine, pyrrolidine, piperazine, morpholine, thiomorpholine, imidazole, or aminodithiazole.

Illustrative examples of optionally substituted heterocyclic rings include but are not limited to tetrahydrofuran, 1,3-dioxolane, 2,3-dihydrofuran, tetrahydropyran, benzofuran, isobenzofuran, 1,3-dihydro-isobenzofuran, isoxazole, 4,5-dihydroisoxazole, piperidine, pyrrolidine, pyrrolidin-2-one, pyrrole, pyridine, pyrimidine, octahydro-pyrrolo[3,4-b]pyridine, piperazine, pyrazine, morpholine, thiomorpholine, imidazole, aminodithiadazole, imidazolidine-2,4-dione, benzimidazole, 1,3-dihydrobenzimidazol-2-one, indole, thiazole, benzothiazole, thiadiazole, thiophene, tetrahydro-thiophene 1,1-dioxide, diazepine, triazole, diazabicyclo[2.2.1]heptane, 2,5-diazabicyclo[2.2.1]heptane, and 2,3,4,4a,9,9a-hexahydro-1H-β-carboline.

In some embodiments, the compound has general formula (1), (2A), (2B) or (3), wherein:

each of A, V and B if present is independently H or halogen (e.g. chloro or fluoro);

X is —(R⁵)R¹R², wherein R⁵ is C or N and wherein in each —(R⁵)R¹R², R¹ and R² together may form an optionally substituted aryl or heteroaryl ring;

Z is NH or N-alkyl (e.g., N—CH₃);

W together with N and Z in formula (1), or W¹ in formula (2A), (2B) or (3) forms an optionally substituted 5- or 6-membered ring that is fused with an optionally substituted aryl or heteroaryl ring; and

U is —R⁵R⁶—(CH₂)ₙ—CHR²—NR³R⁴, wherein R⁶ is H or C₁₋₁₀ alkyl and wherein in the —CHR²—NR³R⁴ moiety each R³ or R⁴ together with the C may form an optionally substituted heterocyclic or heteroaryl ring, or wherein in the —CHR²—NR³R⁴ moiety each R³ or R⁴ together with the N may form an optionally substituted carbocyclic, heterocyclic, aryl or heteroaryl ring.

In certain embodiments, the compound has formula (1), (2A), (2B) or (3), wherein:

A if present is H or halogen (e.g., chloro or fluoro);

X if present is —(R⁵)R¹R², wherein R⁵ is C or N and wherein in each —(R⁵)R¹R², R¹ and R² together may form an optionally substituted aryl or heteroaryl ring;

Z is NH or N-alkyl (e.g., N—CH₃);

W together with N and Z in formula (1), or W¹ in formula (2A), (2B) or (3) forms an optionally substituted 5- or 6-membered ring that is fused with an optionally substituted aryl or heteroaryl ring; and

U is —R⁵R⁶—(CH₂)ₙ—CHR²—NR³R⁴, wherein R⁶ is H or alkyl and wherein in the —CHR²—NR³R⁴ moiety each R³ or R⁴ together with the C may form an optionally substituted heterocyclic or heteroaryl ring, or wherein in the —CHR²—NR³R⁴ moiety each R³ or R⁴ together with the N may form an optionally substituted carbocyclic, heterocyclic, aryl or heteroaryl ring.

In each of the above formula, each optionally substituted moiety may be substituted with one or more halo, OR², NR¹R², carbamate, C₁₋₁₀ alkyl, C₂₋₁₀ alkenyl, each optionally substituted by halo, C═O, aryl or one or more heteroatoms; inorganic substituents, aryl, carbocyclic or a heterocyclic ring. Other substituents include but are not limited to alkynyl, cycloalkyl, fluorinated alkyls such as CF₃, CH₂CF₃, perfluorinated alkyls, etc.; oxygenated fluorinated alkyls such as OCF₃ or CH₂CF₃, etc.; cyano, nitro, COR², NR²COR², sulfonyl amides; NR²SOOR²; SR², SOR², COOR², CONR²₂, OCOR², OCOOR², OCONR²₂, NRCOOR², NRCONR²₂, NRC(NR)(NR²₂), NR(CO)NR²₂, and SOONR²₂, wherein each R² is as defined in formula 1.

As used herein, the term “alkyl” refers to a carbon-containing compound, and encompasses compounds containing one or more heteroatoms. The term “alkyl” also encompasses alkyls substituted with one or more substituents including but not limited to OR¹, amino, amido, halo, ═O, aryl, heterocyclic groups, or inorganic substituents.

As used herein, the term “carbocycle” refers to a cyclic compound containing only carbon atoms in the ring, whereas a “heterocycle” refers to a cyclic compound comprising a heteroatom. The carbocyclic and heterocyclic structures encompass compounds having monocyclic, bicyclic or multiple ring systems.

As used herein, the term “aryl” refers to a polyunsaturated, typically aromatic hydrocarbon substituent, whereas a “heteroaryl” or “heteroaromatic” refer to an aromatic ring containing a heteroatom. The aryl and heteroaryl structures encompass compounds having monocyclic, bicyclic or multiple ring systems.

As used herein, the term “heteroatom” refers to any atom that is not carbon or hydrogen, such as nitrogen, oxygen or sulfur.

Illustrative examples of heterocycles include but are not limited to tetrahydrofuran, 1,3-dioxolane, 2,3-dihydrofuran, pyran, tetrahydropyran, benzofuran, isobenzofuran, 1,3-dihydro-isobenzofuran, isoxazole, 4,5-dihydroisoxazole, piperidine, pyrrolidine, pyrrolidin-2-one, pyrrole, pyridine, pyrimidine, octahydro-pyrrolo[3,4-b]pyridine, piperazine, pyrazine, morpholine, thiomorpholine, imidazole, imidazolidine-2,4-dione, 1,3-dihydrobenzimidazol-2-one, indole, thiazole, benzothiazole, thiadiazole, thiophene, tetrahydro-thiophene 1,1-dioxide, diazepine, triazole, guanidine, diazabicyclo[2.2.1]heptane, 2,5-diazabicyclo[2.2.1]heptane, 2,3,4,4a,9,9a-hexahydro-1H-β-carboline, oxirane, oxetane, tetrahydropyran, dioxane, lactones, aziridine, azetidine, piperidine, lactams, and may also encompass heteroaryls. Other illustrative examples of heteroaryls include but are not limited to furan, pyrrole, pyridine, pyrimidine, imidazole, benzimidazole and triazole.

As used herein, the term “inorganic substituent” refers to substituents that do not contain carbon or contain carbon bound to elements other than hydrogen (e.g., elemental carbon, carbon monoxide, carbon dioxide, and carbonate). Examples of inorganic substituents include but are not limited to nitro, halogen, sulfonyls, sulfinyls, phosphates, etc.

Synthetic procedures for preparing the compounds of the present invention have been described in PCT/US05/011108 and PCT/US2005/26977, each of which is incorporated herein by reference in its entirety. Other variations in the synthetic procedures known to those with ordinary skill in the art may also be used to prepare the compounds of the present invention.

The compounds of the present invention may be chiral. As used herein, a chiral compound is a compound that is different from its mirror image, and has an enantiomer. Furthermore, the compounds may be racemic, or an isolated enantiomer or stereoisomer. Methods of synthesizing chiral compounds and resolving a racemic mixture of enantiomers are well known to those skilled in the art. See, e.g., March, “Advanced Organic Chemistry,” John Wiley and Sons, Inc., New York, (1985), which is incorporated herein by reference.

Illustrative examples of compounds having the above formula are shown in Table I (A-C), and in the Examples. The present invention also encompasses other compounds having any one formula (1), (2A), (2B) and (3), comprising substituents U, A, X, V, and B independently selected from the substituents exemplified in Table 1 (A-C). For example, the isopropyl substituent in the last two compounds shown in Table IA may be replaced with an acetyl substituent, or the N—CH₃ in the fused ring may be replaced with an NH group. Furthermore, the fluoro group may be replaced with H. Thus, the present invention is not limited to the specific combination of substituents described in various embodiments below.

In some embodiments, compounds of the following formula (3A), or a pharmaceutically acceptable salt, ester or prodrug thereof, are utilized:

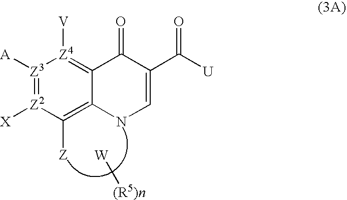

wherein substituents are set forth above.

In some embodiments, a compound has the following formula A-1,

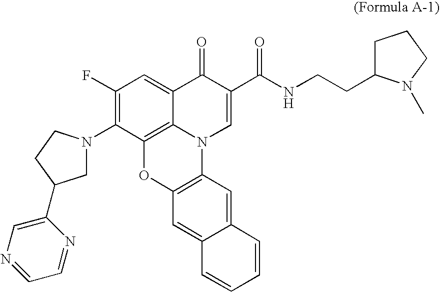

or a pharmaceutically acceptable salt, ester or prodrug thereof, and may be utilized in a method or composition described herein.

In some embodiments, a compound having the following formula B-1:

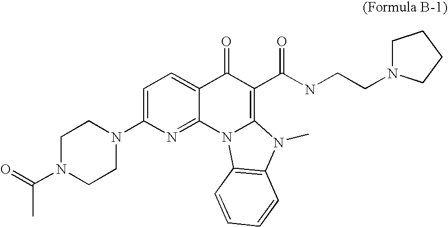

or a pharmaceutically acceptable salt, prodrug or ester thereof, may be utilized in a method or composition described herein.

In certain aspects, the compound is of formula 4, or a pharmaceutically acceptable salt, prodrug or ester thereof:

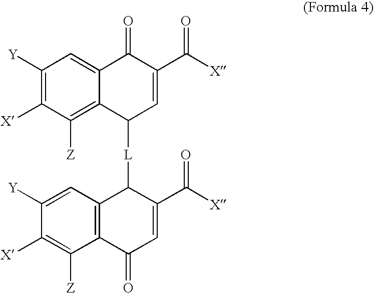

where X′ is hydroxy, alkoxy, carboxyl, halogen, CF₃, amino, amido, sulfide, 3-7 membered carbocycle or heterocycle, 5- or 6-membered aryl or heteroaryl, fused carbocycle or heterocycle, bicyclic compound, NR¹R², NCOR³, N(CH₂)N NR¹R², or N(CH₂)ₙR³, where the N in N(CH₂), NR¹R² and N(CH₂)ₙR³ is optionally linked to a C₁₋₁₀ alkyl, and each X′ is optionally linked to one or more substituents;

X″ is hydroxy, alkoxy, amino, amido, sulfide, 3-7 membered carbocycle or heterocycle, 5- or 6-membered aryl or heteroaryl, fused carbocycle or heterocycle, bicyclic compound, NR¹R², NCOR³, N(CH₂)ₙNR¹R², or N(CH₂)ₙR³, where the N in N(CH₂)ₙ NR¹R² and N(CH₂)ₙR³ is optionally linked to a C1-10 alkyl, and X″ is optionally linked to one or more substituents;

Y is H, halogen, or CF₃;

R¹, R² and R³ are independently H, C1-C6 alkyl, C₁-C₆ substituted alkyl, C3-C6 cycloalkyl, C1-C6 alkoxyl, carboxyl, imine, guanidine, 3-7 membered carbocycle or heterocycle, 5- or 6-membered aryl or heteroaryl, fused carbocycle or heterocycle, or bicyclic compound, where each R¹, R² and R³ are optionally linked to one or more substituents;

Z is a halogen;

and L is a linker having the formula Ar¹-L1-Ar², where Ar1 and Ar2 are aryl or heteroaryl.

In the above formula (4), L1 may be (CH₂)ₘ where m is 1-6, or a heteroatom optionally linked to another heteroatom such as a disulfide. Each of Ar1 and Ar2 may independently be aryl or heteroaryl, optionally substituted with one or more substituents. In one example, L is a [phenyl —S—S-phenyl] linker linking two quinolinone. In a particular embodiment, L is a [phenyl —S—S-phenyl] linker linking two identical quinoline species.

In the above formula (4), X″ may be hydroxy, alkoxy, amino, amido, sulfide, 3-7 membered carbocycle or heterocycle, 5- or 6-membered aryl or heteroaryl, fused carbocycle or heterocycle, bicyclic compound, NR¹R², NCOR³, N(CH₂)ₙNR¹R², or N(CH₂)ₙR³, where the N in N(CH₂)ₙ NR¹R² and N(CH₂)ₙR³ is optionally linked to a C₁₀ alkyl, and X″ is optionally linked to one or more substituents.

Illustrative examples of compounds of the foregoing formulae are set forth in Tables 1A-1C, Table 2, Table 3 and Table 4 in U.S. provisional application No. 60/775,924 filed on Feb. 22, 2006, which is incorporated herein by reference.

Quinolone analogs also can include compounds described, and hereby incorporated by reference, in U.S. Pat. No. 5,817,669, and the following compound described in US 2006/0025437 A1:

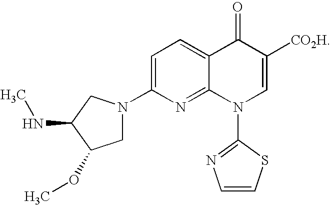

The person of ordinary skill in the art can select and prepare a ribosomal nucleotide sequence interacting nucleic acid molecule. In certain embodiments, the interacting nucleic acid molecule contains a sequence complementary to a ribosomal nucleotide sequence described herein, and is termed an “antisense” nucleic acid. Antisense nucleic acids may comprise or consist of analog or derivative nucleic acids, such as polyamide nucleic acids (PNA), locked nucleic acids (LNA) and other 2′ modified nucleic acids, and others exemplified in U.S. Pat. Nos. 4,469,863; 5,536,821; 5,541,306; 5,637,683; 5,637,684; 5,700,922; 5,717,083; 5,719,262; 5,739,308; 5,773,601; 5,886,165; 5,929,226; 5,977,296; 6,140,482; 5,614,622; 5,739,314; 5,955,599; 5,962,674; 6,117,992; WIPO publications WO 00/56746, WO 00/75372 and WO 01/14398, and related publications. An antisense nucleic acid sometimes is designed, prepared and/or utilized by the artisan to inhibit a ribosomal nucleic acid. The antisense nucleic acid can be complementary to an entire coding strand, or to a portion thereof or a substantially identical sequence thereof. In another embodiment, the antisense nucleic acid molecule is antisense to a “noncoding region” of the coding strand of a nucleotide sequence. An antisense nucleic acid can be complementary to the entire coding region of a ribosomal nucleotide sequence, and often the antisense nucleic acid is an oligonucleotide antisense to only a portion of a coding or noncoding region of the ribosomal nucleotide sequence. For example, the antisense oligonucleotide can be complementary to the region surrounding the translation start site of the mRNA, e.g., between the −10 and +10 regions of the target gene nucleotide sequence of interest. An antisense oligonucleotide can be, for example, about 7, 10, 15, 20, 25, 30, 35, 40, 45, 50, 55, 60, 65, 70, 75, 80, or more nucleotides in length.

An antisense nucleic acid can be constructed using standard chemical synthesis or enzymic ligation reactions. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used). Antisense nucleic acid also can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

When utilized in animals, antisense nucleic acids typically are administered to a subject (e.g., by direct injection at a tissue site or intravenous administration) or generated in situ such that they hybridize with or bind to cellular mRNA and/or genomic DNA encoding a polypeptide and thereby inhibit expression of the polypeptide, for example, by inhibiting transcription and/or translation. Alternatively, antisense nucleic acid molecules can be modified to target selected cells and then are administered systemically. For systemic administration, antisense molecules can be modified such that they specifically bind to receptors or antigens expressed on a selected cell surface, for example, by linking antisense nucleic acid molecules to peptides or antibodies which bind to cell surface receptors or antigens. Antisense nucleic acid molecules can also be delivered to cells using the vectors described herein. Sufficient intracellular concentrations of antisense molecules are achieved by incorporating a strong promoter, such as a CMV promoter, pol II promoter or pol III promoter, in the vector construct.

Antisense nucleic acid molecules sometimes are alpha-anomeric nucleic acid molecules. An alpha-anomeric nucleic acid molecule forms specific double-stranded hybrids with complementary RNA in which, contrary to the usual beta-units, the strands run parallel to each other (Gaultier et al., Nucleic Acids. Res. 15: 6625-6641 (1987)). Antisense nucleic acid molecules also can comprise a 2′-o-methylribonucleotide (Inoue et al., Nucleic Acids Res. 15: 6131-6148 (1987)) or a chimeric RNA-DNA analogue (Inoue et al., FEBS Lett. 215: 327-330 (1987)). Antisense nucleic acids sometimes are composed of DNA or PNA or any other nucleic acid derivatives described previously.

An antisense nucleic acid is a ribozyme in some embodiments. A ribozyme having specificity for a ribosomal nucleotide sequence can include one or more sequences complementary to such a nucleotide sequence, and a sequence having a known catalytic region responsible for mRNA cleavage (e.g., U.S. Pat. No. 5,093,246 or Haselhoff and Gerlach, Nature 334: 585-591 (1988)). For example, a derivative of a Tetrahymena L-19 IVS RNA is sometimes utilized in which the nucleotide sequence of the active site is complementary to the nucleotide sequence to be cleaved in a mRNA (e.g., Cech et al. U.S. Pat. No. 4,987,071; and Cech et al. U.S. Pat. No. 5,116,742). Ribosomal nucleotide sequences also may be utilized to select a catalytic RNA having a specific ribonuclease activity from a pool of RNA molecules (e.g., Bartel & Szostak, Science 261: 1411-1418 (1993)).

Specific binding reagents sometimes are nucleic acids that can form triple helix structures with a ribosomal nucleotide sequence. Triple helix formation can be enhanced by generating a “switchback” nucleic acid molecule. Switchback molecules are synthesized in an alternating 5′-3′,3′-5′ manner, such that they base pair with first one strand of a duplex and then the other, eliminating the necessity for a sizeable stretch of purines or pyrimidines being present on one strand of a duplex.

An artisan may select an interfering RNA (RNAi) or siRNA ribosomal nucleotide sequence interacting agent for use. The nucleic acid selected sometimes is the RNAi or siRNA or a nucleic acid that encodes such products. The term “RNAi” as used herein refers to double-stranded RNA (dsRNA) which mediates degradation of specific mRNAs, and can also be used to lower or eliminate gene expression. The term “short interfering nucleic acid”, “siNA”, “short interfering RNA”, “siRNA”, “short interfering nucleic acid molecule”, “short interfering oligonucleotide molecule”, or “chemically-modified short interfering nucleic acid molecule” as used herein refers to any nucleic acid molecule directed against a gene. For example, a siRNA is capable of inhibiting or down regulating gene expression or viral replication, for example by mediating RNA interference “RNAi” or gene silencing in a sequence-specific manner; see for example Zamore et al., 2000, Cell, 101, 25-33; Bass, 2001, Nature, 411, 428-429; Elbashir et al., 2001, Nature, 411, 494-498; and Kreutzer et al., International PCT Publication No. WO 00/44895; Zernicka-Goetz et al., International PCT Publication No. WO 01/36646; Fire, International PCT Publication No. WO 99/32619; Plaetinck et al., International PCT Publication No. WO 00/01846; Mello and Fire, International PCT Publication No. WO 01/29058; Deschamps-Depaillette, International PCT Publication No. WO 99/07409; and Li et al., International PCT Publication No. WO 00/44914; Allshire, 2002, Science, 297, 1818-1819; Volpe et al., 2002, Science, 297, 1833-1837; Jenuwein, 2002, Science, 297, 2215-2218; and Hall et al., 2002, Science, 297, 2232-2237; Hutvagner and Zamore, 2002, Science, 297, 2056-60; McManus et al., 2002, RNA, 8, 842-850; Reinhart et al., 2002, Gene & Dev., 16, 1616-1626; and Reinhart & Bartel, 2002, Science, 297, 1831). There is no particular limitation in the length of siRNA as long as it does not show toxicity. Examples of modified RNAi and siRNA include STEALTH™ forms (Invitrogen Corp., Carlsbad, Calif.), forms described in U.S. Patent Publication No. 2004/0014956 (application Ser. No. 10/357,529) and U.S. patent application Ser. No. 11/049,636, filed Feb. 2, 2005), shRNA, MIRs and other forms described hereafter.

A siNA can be a double-stranded polynucleotide molecule comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof. The siNA can be assembled from two separate oligonucleotides, where one strand is the sense strand and the other is the antisense strand, wherein the antisense and sense strands are self-complementary (i.e. each strand comprises nucleotide sequence that is complementary to nucleotide sequence in the other strand; such as where the antisense strand and sense strand form a duplex or double stranded structure, for example wherein the double stranded region is about 19 base pairs); the antisense strand comprises nucleotide sequence that is complementary to nucleotide sequence in a target nucleic acid molecule or a portion thereof and the sense strand comprises nucleotide sequence corresponding to the target nucleic acid sequence or a portion thereof. Alternatively, the siNA is assembled from a single oligonucleotide, where the self-complementary sense and antisense regions of the siNA are linked by means of a nucleic acid based or non-nucleic acid-based linker(s). The siNA can be a polynucleotide with a duplex, asymmetric duplex, hairpin or asymmetric hairpin secondary structure, having self-complementary sense and antisense regions, wherein the antisense region comprises nucleotide sequence that is complementary to nucleotide sequence in a separate target nucleic acid molecule or a portion thereof and the sense region having nucleotide sequence corresponding to the target nucleic acid sequence or a portion thereof. The siNA can be a circular single-stranded polynucleotide having two or more loop structures and a stem comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof, and wherein the circular polynucleotide can be processed either in vivo or in vitro to generate an active siNA molecule capable of mediating RNAi. The siNA can also comprise a single stranded polynucleotide having nucleotide sequence complementary to nucleotide sequence in a target nucleic acid molecule or a portion thereof (for example, where such siNA molecule does not require the presence within the siNA molecule of nucleotide sequence corresponding to the target nucleic acid sequence or a portion thereof), wherein the single stranded polynucleotide can further comprise a terminal phosphate group, such as a 5′-phosphate (see for example Martinez et al., 2002, Cell., 110, 563-574 and Schwarz et al., 2002, Molecular Cell, 10, 537-568), or 5′,3′-diphosphate. In certain embodiments, the siNA molecule of the invention comprises separate sense and antisense sequences or regions, wherein the sense and antisense regions are covalently linked by nucleotide or non-nucleotide linkers molecules as is known in the art, or are alternately non-covalently linked by ionic interactions, hydrogen bonding, van der waals interactions, hydrophobic interactions, and/or stacking interactions. In certain embodiments, the siNA molecules of the invention comprise nucleotide sequence that is complementary to nucleotide sequence of a target gene. In another embodiment, the siNA molecule of the invention interacts with nucleotide sequence of a target gene in a manner that causes inhibition of expression of the target gene.

The double-stranded RNA portions of siRNAs in which two RNA strands pair are not limited to the completely paired forms, and may contain non-pairing portions due to mismatch (the corresponding nucleotides are not complementary), bulge (lacking in the corresponding complementary nucleotide on one strand), and the like. Non-pairing portions can be contained to the extent that they do not interfere with siRNA formation. The “bulge” used herein preferably comprise 1 to 2 non-pairing nucleotides, and the double-stranded RNA region of siRNAs in which two RNA strands pair up contains preferably 1 to 7, more preferably 1 to 5 bulges. In addition, the “mismatch” used herein is contained in the double-stranded RNA region of siRNAs in which two RNA strands pair up, preferably 1 to 7, more preferably 1 to 5, in number. In a preferable mismatch, one of the nucleotides is guanine, and the other is uracil. Such a mismatch is due to a mutation from C to T, G to A, or mixtures thereof in DNA coding for sense RNA, but not particularly limited to them. Furthermore, in the present invention, the double-stranded RNA region of siRNAs in which two RNA strands pair up may contain both bulge and mismatched, which sum up to, preferably 1 to 7, more preferably 1 to 5 in number. The terminal structure of siRNA may be either blunt or cohesive (overhanging) as long as siRNA enables to silence the target gene expression due to its RNAi effect.

As used herein, siRNA molecules need not be limited to those molecules containing only RNA, but further encompasses chemically-modified nucleotides and non-nucleotides. In addition, as used herein, the term RNAi is meant to be equivalent to other terms used to describe sequence specific RNA interference, such as post transcriptional gene silencing, translational inhibition, or epigenetics. For example, siRNA molecules of the invention can be used to epigenetically silence genes at both the post-transcriptional level or the pre-transcriptional level. In a non-limiting example, epigenetic regulation of gene expression by siRNA molecules of the invention can result from siRNA mediated modification of chromatin structure to alter gene expression (see, for example, Verdel et al., 2004, Science, 303, 672-676; Pal-Bhadra et al., 2004, Science, 303, 669-672; Allshire, 2002, Science, 297, 1818-1819; Volpe et al., 2002, Science, 297, 1833-1837; Jenuwein, 2002, Science, 297, 2215-2218; and Hall et al., 2002, Science, 297, 2232-2237).

RNAi may be designed by those methods known to those of ordinary skill in the art. In one example, siRNA may be designed by classifying RNAi sequences, for example 1000 sequences, based on functionality, with a functional group being classified as having greater than 85% knockdown activity and a non-functional group with less than 85% knockdown activity. The distribution of base composition was calculated for entire the entire RNAi target sequence for both the functional group and the non-functional group. The ratio of base distribution of functional and non-functional group may then be used to build a score matrix for each position of RNAi sequence. For a given target sequence, the base for each position is scored, and then the log ratio of the multiplication of all the positions is taken as a final score. Using this score system, a very strong correlation may be found of the functional knockdown activity and the log ratio score. Once the target sequence is selected, it may be filtered through both fast NCBI blast and slow Smith Waterman algorithm search against the Unigene database to identify the gene-specific RNAi or siRNA. Sequences with at least one mismatch in the last 12 bases may be selected.

Nucleic acid reagents include those which are engineered, for example, to produce dsRNAs. Examples of such nucleic acid molecules include those with a sequence that, when transcribed, folds back upon itself to generate a hairpin molecule containing a double-stranded portion. One strand of the double-stranded portion may correspond to all or a portion of the sense strand of the mRNA transcribed from the gene to be silenced while the other strand of the double-stranded portion may correspond to all or a portion of the antisense strand. Other methods of producing dsRNAs may be used, for example, nucleic acid molecules may be engineered to have a first sequence that, when transcribed, corresponds to all or a portion of the sense strand of the mRNA transcribed from the gene to be silenced and a second sequence that, when transcribed, corresponds to all or portion of an antisense strand (i.e., the reverse complement) of the mRNA transcribed from the gene to be silenced.

Nucleic acid molecules which mediate RNAi may also be produced ex vivo, for example, by oligonucleotide synthesis. Oligonucleotide synthesis may be used for example, to design dsRNA molecules, as well as other nucleic acid molecules (e.g., other nucleic acid molecules which mediate RNAi) with one or more chemical modification (e.g., chemical modifications not commonly found in nucleic acid molecules such as the inclusion of 2′-O-methyl, 2′-O-ethyl, 2′-methoxyethoxy, 2′-O-propyl, 2′-fluoro, etc. groups).

In some embodiments, a dsRNA to be used to silence a gene may have one or more (e.g., one, two, three, four, five, six, etc.) regions of sequence homology or identity to a gene to be silenced. Regions of homology or identity may be from about 20 bp (base pairs) to about 5 kbp (kilo base pairs) in length, 20 bp to about 4 kbp in length, 20 bp to about 3 kbp in length, 20 bp to about 2.5 kbp in length, from about 20 bp to about 2 kbp in length, 20 bp to about 1.5 kbp in length, from about 20 bp to about 1 kbp in length, 20 bp to about 750 bp in length, from about 20 bp to about 500 bp in length, 20 bp to about 400 bp in length, 20 bp to about 300 bp in length, 20 bp to about 250 bp in length, from about 20 bp to about 200 bp in length, from about 20 bp to about 150 bp in length, from about 20 bp to about 100 bp in length, from about 20 bp to about 90 bp in length, from about 20 bp to about 80 bp in length, from about 20 bp to about 70 bp in length, from about 20 bp to about 60 bp in length, from about 20 bp to about 50 bp in length, from about 20 bp to about 40 bp in length, from about 20 bp to about 30 bp in length, from about 20 bp to about 25 bp in length, from about 15 bp to about 25 bp in length, from about 17 bp to about 25 bp in length, from about 19 bp to about 25 bp in length, from about 19 bp to about 23 bp in length, or from about 19 bp to about 21 bp in length.

A hairpin containing molecule having a double-stranded region may be used as RNAi. The length of the double stranded region may be from about 20 bp (base pairs) to about 2.5 kbp (kilo base pairs) in length, from about 20 bp to about 2 kbp in length, 20 bp to about 1.5 kbp in length, from about 20 bp to about 1 kbp in length, 20 bp to about 750 bp in length, from about 20 bp to about 500 bp in length, 20 bp to about 400 bp in length, 20 bp to about 300 bp in length, 20 bp to about 250 bp in length, from about 20 bp to about 200 hp in length, from about 20 bp to about 150 bp in length, from about 20 bp to about 100 bp in length, 20 bp to about 90 bp in length, 20 bp to about 80 bp in length, 20 bp to about 70 bp in length, 20 bp to about 60 bp in length, 20 bp to about 50 bp in length, 20 bp to about 40 bp in length, 20 bp to about 30 bp in length, or from about 20 bp to about 25 bp in length. The non-base-paired portion of the hairpin (i.e., loop) can be of any length that permits the two regions of homology that make up the double-stranded portion of the hairpin to fold back upon one another.

Any suitable promoter may be used to control the production of RNA from the nucleic acid reagent, such as a promoter described above. Promoters may be those recognized by any polymerase enzyme. For example, promoters may be promoters for RNA polymerase II or RNA polymerase III (e.g., a U6 promoter, an HI promoter, etc.). Other suitable promoters include, but are not limited to, T7 promoter, cytomegalovirus (CMV) promoter, mouse mammary tumor virus (MMTV) promoter, metalothionine, RSV (Rous sarcoma virus) long terminal repeat, SV40 promoter, human growth hormone (hGH) promoter. Other suitable promoters are known to those skilled in the art and are within the scope of the present invention.

Double-stranded RNAs used in the practice of the invention may vary greatly in size. Further the size of the dsRNAs used will often depend on the cell type contacted with the dsRNA. As an example, animal cells such as those of C. elegans and Drosophila melanogaster do not generally undergo apoptosis when contacted with dsRNAs greater than about 30 nucleotides in length (i.e., 30 nucleotides of double stranded region) while mammalian cells typically do undergo apoptosis when exposed to such dsRNAs. Thus, the design of the particular experiment will often determine the size of dsRNAs employed.

In many instances, the double stranded region of dsRNAs contained within or encoded by nucleic acid molecules used in the practice of the invention will be within the following ranges: from about 20 to about 30 nucleotides, from about 20 to about 40 nucleotides, from about 20 to about 50 nucleotides, from about 20 to about 100 nucleotides, from about 22 to about 30 nucleotides, from about 22 to about 40 nucleotides, from about 20 to about 28 nucleotides, from about 22 to about 28 nucleotides, from about 25 to about 30 nucleotides, from about 25 to about 28 nucleotides, from about 30 to about 100 nucleotides, from about 30 to about 200 nucleotides, from about 30 to about 1,000 nucleotides, from about 30 to about 2,000 nucleotides, from about 50 to about 100 nucleotides, from about 50 to about 1,000 nucleotides, or from about 50 to about 2,000 nucleotides. The ranges above refer to the number of nucleotides present in double stranded regions. Thus, these ranges do not reflect the total length of the dsRNAs themselves. As an example, a blunt ended dsRNA formed from a single transcript of 50 nucleotides in total length with a 6 nucleotide loop, will have a double stranded region of 23 nucleotides.

As suggested above, dsRNAs used in the practice of the invention may be blunt ended, may have one blunt end, or may have overhangs on both ends. Further, when one or more overhang is present, the overhang(s) may be on the 3′ and/or 5′ strands at one or both ends. Additionally, these overhangs may independently be of any length (e.g., one, two, three, four, five, etc. nucleotides). As an example, STEALTH™ RNAi is blunt at both ends.

Also included are sets of RNAi and those which generate RNAi. Such sets include those which either (1) are designed to produce or (2) contain more than one dsRNA directed against the same target gene. As an example, the invention includes sets of STEALTH™ RNAi wherein more than one STEALTH™ RNAi shares sequence homology or identity to different regions of the same target gene.

An antibody or antibody fragment can be generated by and used by the artisan as a ribosomal nucleotide sequence interacting agent. Antibodies sometimes are IgG, IgM, IgA, IgE, or an isotype thereof (e.g., IgG1, IgG2a, IgG2b or IgG3), sometimes are polyclonal or monoclonal, and sometimes are chimeric, humanized or bispecific versions of such antibodies. Polyclonal and monoclonal antibodies that bind specific antigens are commercially available, and methods for generating such antibodies are known. In general, polyclonal antibodies are produced by injecting an isolated antigen (e.g., rDNA or rRNA subsequence described herein) into a suitable animal (e.g., a goat or rabbit); collecting blood and/or other tissues from the animal containing antibodies specific for the antigen and purifying the antibody. Methods for generating monoclonal antibodies, in general, include injecting an animal with an isolated antigen (e.g., often a mouse or a rat); isolating splenocytes from the animal; fusing the splenocytes with myeloma cells to form hybridomas; isolating the hybridomas and selecting hybridomas that produce monoclonal antibodies which specifically bind the antigen (e.g., Kohler & Milstein, Nature 256:495 497 (1975) and StGroth & Scheidegger, J Immunol Methods 5:1 21 (1980)).

Methods for generating chimeric and humanized antibodies also are known (see, e.g., U.S. Pat. No. 5,530,101 (Queen, et al.), U.S. Pat. No. 5,707,622 (Fung, et al.) and U.S. Pat. Nos. 5,994,524 and 6,245,894 (Matsushima, et al.)), which generally involve transplanting an antibody variable region from one species (e.g., mouse) into an antibody constant domain of another species (e.g., human). Antigen-binding regions of antibodies (e.g., Fab regions) include a light chain and a heavy chain, and the variable region is composed of regions from the light chain and the heavy chain. Given that the variable region of an antibody is formed from six complementarity-determining regions (CDRs) in the heavy and light chain variable regions, one or more CDRs from one antibody can be substituted (i.e., grafted) with a CDR of another antibody to generate chimeric antibodies. Also, humanized antibodies are generated by introducing amino acid substitutions that render the resulting antibody less immunogenic when administered to humans.

A specific binding reagent sometimes is an antibody fragment, such as a Fab, Fab′, F(ab)′2, Dab, Fv or single-chain Fv (ScFv) fragment, and methods for generating antibody fragments are known (see, e.g., U.S. Pat. Nos. 6,099,842 and 5,990,296 and PCT/GB00/04317). In some embodiments, a binding partner in one or more hybrids is a single-chain antibody fragment, which sometimes are constructed by joining a heavy chain variable region with a light chain variable region by a polypeptide linker (e.g., the linker is attached at the C-terminus or N-terminus of each chain) by recombinant molecular biology processes. Such fragments often exhibit specificities and affinities for an antigen similar to the original monoclonal antibodies. Bifunctional antibodies sometimes are constructed by engineering two different binding specificities into a single antibody chain and sometimes are constructed by joining two Fab′ regions together, where each Fab′ region is from a different antibody (e.g., U.S. Pat. No. 6,342,221). Antibody fragments often comprise engineered regions such as CDR-grafted or humanized fragments. In certain embodiments the binding partner is an intact immunoglobulin, and in other embodiments the binding partner is a Fab monomer or a Fab dimer.

Compositions, Cells and Animals Comprising Nucleic Acids and/or Interacting Molecules

Provided herein is a composition comprising a nucleic acid described herein. In certain embodiments, a composition comprises a nucleic acid that includes a nucleotide sequence complementary to a human ribosomal DNA or RNA nucleotide sequence described herein. A composition may comprise a pharmaceutically acceptable carrier in some embodiments, and a composition sometimes comprises a nucleic acid and a compound that binds to a human ribosomal nucleotide sequence in the nucleic acid (e.g., specifically binds to the nucleotide sequence). In certain embodiments, the compound is a quinolone analog, such as a compound described herein.

Other compositions provided comprise a compound in association with a component of a complex that synthesizes ribosomal RNA in a cell or a fragment of the component, wherein the compound is a quinolone analog. The quinolone analog sometimes is of formula 3 or 3A, and at times is of formula 2 or 2A-2D. The component sometimes is selected from the group consisting of UBF, TBP, TAF 48, TAF 63, TAF 110 and a RNA polymerase I subunit. Sequences of such components are known, and examples of sequences, as indicated by accession number (HUGO Gene Nomenclature Committee), are shown in the table hereafter.

| Component | Sequence Accession Number |
|---|---|
| Nucleolin | NM_005381 |
| Fibrillarin | AC005393 |
| RecQ | |
| BLM | U39817 |
| Bloom Syndrome | |
| WRN | NM_000553 |
| Werner Syndrome | |
| RecQL | NM_002907 |
| RecQ4 | AB006532 |
| TBP | M55654 |
| RNA Polymerase I | |
| POLR1A | AK025568 |
| POLR1B | AK001678 |
| POLR1C | AF008442 |
| POLR1D | AF077044 |

Also provided is a composition which comprises a compound in association with a protein kinase or fragment thereof, wherein the compound is a quinolone analog. The protein kinase sometimes is a member of a MAP kinase, mTOR or PI3 kinase pathway. A member of a particular pathway includes (a) a protein kinase that is phosphorylated, directly or indirectly, by the named protein kinase, (b) phosphorylates, directly or indirectly, the named protein kinase, or (c) is the named protein kinase or an isoform thereof. An indirect phosphorylation event can be exemplified by the following: a protein that is indirectly phosphorylated by a particular protein kinase can be phosphorylated by a first protein kinase that is initially phosphorylated by a second protein kinase, and any number of intervening protein kinases can exist in the pathway. In certain embodiments, the protein kinase is a cell cycle regulating protein kinase (e.g., cyclin dependent protein kinase such as cdk2 or cdk4), or a RSK protein kinase (e.g., RSK 1 alpha, RSK 1 beta or RSK 2), or is a casein protein kinase, or is an AKT protein kinase (e.g., AKT 1, 2 or 3). The protein kinase sometimes is selected from the group consisting of ABL, S6K, Tic, TrkA, ZIPK, Pim-1, SAPK, Flt3 and DRK3 protein kinases. Sequences of multiple protein kinases are known, and examples of sequences, as indicated by accession number (HUGO Gene Nomenclature Committee), are shown in the table hereafter.

| Protein Kinase | Sequence Accession Number |
|---|---|
| ABL | M14752 |
| P70S6K | AB019245 |
| TIE2 | L06139 |
| TRKA | Y09028 |
| ZIPK | AB007144 |
| Pim-1 | NM_002648 |
| SAPK3 | U66243 |
| FLT3 | U02687 |
| DRAK1 | AB011420 |

Provided also is a composition comprising a nucleic acid and a quinolone compound bound to it, wherein the nucleic acid comprises a human ribosomal nucleic acid nucleotide sequence. In some embodiments, the human ribosomal nucleic acid nucleotide sequence comprises a polynucleotide sequence that forms a nucleic acid structure, and sometimes the compound binds to the nucleic acid structure. Any nucleic acid structure can be utilized, and may be selected from the group consisting of a quadruplex, hairpin, helix, coaxial helix, tetraloop-receptor, A-minor motif, kissing hairpin loops, tRNA D-loop:T-loop, pseudoknot, deoxyribose zipper and ribose zipper. In certain embodiments, the nucleic acid structure is an intramolecular quadruplex, such as a G-quadruplex. The compound in such compositions sometimes is of formula 3 or 3A, and at times is of formula 2 or 2A-2D. In certain embodiments, the ribosomal nucleic acid is rRNA, and sometimes it is rDNA.

Also provided is a cell or animal comprising an isolated nucleic acid described herein. Any type of cell can be utilized, and sometimes the cell is a cell line maintained or proliferated in tissue culture. The isolated nucleic acid may be incorporated into one or more cells of an animal, such as a research animal (e.g., rodent (e.g., mouse, rat, guinea pig, hamster, rabbit), ungulate (e.g., bovine, porcine, equine, caprine), cat, dog, monkey or ape). Methods for inserting compounds and other molecules into cells are known to the person of ordinary skill in the art, such as in methods described hereafter.

A cell may over-express or under-express a ribosomal nucleotide sequence described herein. A cell can be processed in a variety of manners. For example, an artisan may prepare a lysate from a cell reagent and optionally isolate or purify components of the cell, may transfect the cell with a nucleic acid reagent, may fix a cell reagent to a slide for analysis (e.g., microscopic analysis) and can immobilize a cell to a solid phase. A cell that “over-expresses” a ribosomal nucleotide sequence produces at least two, three, four or five times or more of the product as compared to a native cell from an organism that has not been genetically modified and/or exhibits no apparent symptom of a cell-proliferative disorder. Over-expressing cells may be stably transfected or transiently transfected with a nucleic acid that encodes the ribosomal nucleotide sequence. A cell that “under-expresses” a ribosomal nucleotide sequence produces at least five times less of the product as compared to a native cell from an organism that has not been genetically modified and/or exhibits no apparent symptom of a cell-proliferative disorder. In some embodiments, a cell that under-expresses a ribosomal nucleotide sequence contains no nucleic acid that can encode such a product (e.g., the cell is from a knock-out mouse) and no detectable amount of the product is produced. Methods for generating knock-out animals and using cells extracted therefrom are known (e.g., Miller et al., J. Cell. Biol. 165: 407-419 (2004)). A cell that under-expresses a ribosomal nucleotide sequence, for example, sometimes is in contact with a nucleic acid inhibitor that blocks or reduces the amount of the product produced by the cell in the absence of the inhibitor. An over-expressing or under-expressing cell may be within an organism (in vivo) or from an organism (ex vivo or in vitro).

The artisan may select any cell for generating cell compositions of the invention (e.g., cells that over-express or under-express a ribosomal nucleotide sequence). Cells include, but are not limited to, bacterial cells (e.g., Escherichia spp. cells (e.g., Expressway™ HTP Cell-Free E. coli Expression Kit, Invitrogen, California) such as DH10B, Stb12, DH5-alpha, DB3, DB3.1 for example), DB4, DB5, JDP682 and ccdA-over (e.g., U.S. application Ser. No. 09/518,188), Bacillus spp. cells (e.g., B. subtilis and B. megaterium cells), Streptomyces spp. cells, Erwinia spp. cells, Klebsiella spp. cells, Serratia spp. cells (particularly S. marcessans cells), Pseudomonas spp. cells (particularly P. aeruginosa cells), and Salmonella spp. cells (particularly S. typhimurium and S. typhi cells); photosynthetic bacteria (e.g., green non-sulfur bacteria (e.g., Choroflexus spp. (e.g., C. aurantiacus), Chloronema spp. (e.g., C. gigateum)), green sulfur bacteria (e.g., Chlorobium spp. (e.g., C. limicola), Pelodictyon spp. (e.g., P. luteolum), purple sulfur bacteria (e.g., Chromatium spp. (e.g., C. okenii)), and purple non-sulfur bacteria (e.g., Rhodospirillum spp. (e.g., R. rubrum), Rhodobacter spp. (e.g., R. sphaeroides, R. capsulatus), Rhodomicrobium spp. (e.g., R. vanellii)); yeast cells (e.g., Saccharomyces cerevisiae cells and Pichia pastoris cells); insect cells (e.g., Drosophila (e.g., Drosophila melanogaster), Spodoptera (e.g., Spodoptera frugiperda Sf9 and Sf21 cells) and Trichoplusa (e.g., High-Five cells); nematode cells (e.g., C. elegans cells); avian cells; amphibian cells (e.g., Xenopus laevis cells); reptilian cells; and mammalian cells (e.g., NIH3T3, 293, CHO, COS, VERO, C127, BHK, Per-C6, Bowes melanoma and HeLa cells). In specific embodiments, cells are pancreatic cells, colorectal cells, renal cells or Burkitt's lymphoma cells. In some embodiments, pancreatic cell lines such as PC3, HCT116, HT29, MIA Paca-2, HPAC, Hs700T, Panc10.05, Panc 02.13, PL45, SW 190, Hs 766T, CFPAC-1 and PANC-1 are utilized. These and other suitable cells are available commercially, for example, from Invitrogen Corporation, (Carlsbad, Calif.), American Type Culture Collection (Manassas, Va.), and Agricultural Research Culture Collection (NRRL; Peoria, Ill.).

Use of Ribosomal Nucleotide Sequences and Interacting Molecules

Ribosomal nucleotide sequence interacting molecules sometimes are utilized to effect a cellular response, and are utilized to effect a therapeutic response in some embodiments. Accordingly, provided herein is a method for inhibiting rRNA synthesis in cells, which comprises contacting cells with a compound that interacts with rRNA or rDNA in an amount effective to reduce rRNA synthesis in cells. Such methods may be conducted in vitro, in vivo and/or ex vivo. Accordingly, some in vivo and ex vivo embodiments are directed to a method for inhibiting rRNA synthesis in cells of a subject, which comprises administering a compound that interacts with rRNA or rDNA to a subject in need thereof in an amount effective to reduce rRNA synthesis in cells of the subject. In some embodiments, cells can be contacted with one or more compounds, one or more of which interact with rRNA or rDNA (e.g., one drug or drug and co-drug(s) methodologies). In certain embodiments, a compound is a quinolone derivative, such as a quinolone derivative described herein (e.g., a compound of formula A-1 or B-1). The cells often are cancer cells, such as cells undergoing higher than normal proliferation and tumor cells, for example.

In some embodiments, cells are contacted with a compound that interacts with rRNA or rDNA in combination with one or more other therapies (e.g., tumor removal surgery and/or radiation therapy) and/or other molecules (e.g., co-drugs) that exert other effects in cells. For example, a co-drug may be selected that reduces cell proliferation or reduces tissue inflammation. The person of ordinary skill in the art may select and administer a wide variety of co-drugs in a combination approach. Non-limiting examples of co-drugs include avastin, dacarbazine (e.g., multiple myeloma), 5-fluorouracil (e.g., pancreatic cancer), gemcitabine (e.g., pancreatic cancer), and gleevac (e.g., CML).

The term “inhibiting rRNA synthesis” as used herein refers to reducing the amount of rRNA produced by a cell after a cell is contacted with the compound or after a compound is administered to a subject. In certain embodiments, rRNA levels are reduced by about 10%, about 15%, about 20%, about 25%, about 30%, about 40%, about 45%, about 50%, about 55%, about 60%, about 70%, about 75%, about 80%, about 90%, or about 95% or more in a specific time frame, such as about 1 hour, about 2 hours, about 3 hours, about 4 hours, about 5 hours, about 6 hours, about 7 hours, about 8 hours, about 9 hours, about 10 hours, about 12 hours, about 16 hours, about 20 hours, or about 24 hours in particular cells after cells are contacted with the compound or the compound is administered to a subject. Particular cells in which rRNA levels are reduced sometimes are cancer cells or cells undergoing proliferation at greater rates than other cells in a system. Levels of rRNA in a cell can be determined in vitro and in vivo (e.g., see Examples section). In certain embodiments, rRNA synthesis is inhibited without substantially inhibiting DNA replication or protein translation. In the latter embodiments, DNA replication and/or protein translation may be non-substantially reduced when they are reduced by up to 10% in particular cells.

The term “interacting with rRNA” as used herein refers to a direct interaction or indirect interaction of a compound with rRNA. In some embodiments, a compound may directly bind to rRNA, such as a nucleotide sequence region described herein. A compound may directly bind to a rDNA nucleotide sequence that encodes a particular rRNA (e.g., a rDNA sequence described herein) in certain embodiments. In certain embodiments, a compound may bind to and/or stabilize a quadruplex structure in rRNA or rDNA. In some embodiments, a compound may directly bind to a protein that binds to or interacts with a rRNA or rDNA nucleotide sequence, such as a protein involved in rRNA synthesis, a protein involved in rRNA elongation (e.g., a polymerase such as Pol I or Pol III, or a nucleolin protein), a protein involved in pre-rRNA processing (e.g., an endonuclease, exonuclease, RNA helicase), or a protein involved with ribosomal biogenesis (e.g., a ribosomal subunit protein or a protein the facilitates loading of rRNA into a ribosomal subunit), for example.

In certain embodiments, provided also is a method for effecting a cellular response by contacting a cell with a compound that binds to a human ribosomal nucleotide sequence and/or structure described herein. The cellular response sometimes is (a) substantial phosphorylation of H2AX, p53, chk1 and p38 MAPK proteins; (b) redistribution of nucleolin from nucleoli into the nucleoplasm; (c) release of cathepsin D from lysosomes; (d) induction of apoptosis; (e) induction of chromosomal laddering; (f) induction of apoptosis without substantially arresting cell cycle progression; and/or (g) induction of apoptosis and inducing cell cycle arrest (e.g., S-phase and/or G1 arrest).

The term “substantial phosphorylation” as used herein, refers to one or more sites of a particular type of protein or fragment linked to a phosphate moiety. In certain embodiments, phosphorylation is substantial when it is detectable, and in some embodiments, phosphorylation is substantial when about 55% to 99% of the particular type of protein or fragment is phosphorylated or phosphorylated at a particular site. Particular proteins sometimes are H2AX, DNA-PK, p53, chk1, JNK and p38 MAPK proteins or fragments thereof that contain one or more phosphorylation sites. Methods for detecting phosphorylation of such proteins are described herein.

The term “apoptosis” as used herein refers to an intrinsic cell self-destruction or suicide program. In response to a triggering stimulus, cells undergo a cascade of events including cell shrinkage, blebbing of cell membranes and chromatic condensation and fragmentation. These events culminate in cell conversion to clusters of membrane-bound particles (apoptotic bodies), which are thereafter engulfed by macrophages. Chromosomal DNA often is cleaved in cells undergoing apoptosis such that a ladder is visualized when cellular DNA is analyzed by gel electrophoresis. Apoptosis sometimes is monitored by detecting caspase activity, such as caspase S activity, and by monitoring phosphatidyl serine translocation. Methods described herein are designed to preferentially induce apoptosis of cancer cells, such as cancer cells in tumors, over non-cancerous cells.

The term “cell cycle progression” as used herein refers to the process in which a cell divides and proliferates. Particular phases of cell cycle progression are recognized, such as the mitosis and interphase. There are sub-phases within interphase, such as G1, S and G2 phases, and sub-phases within mitosis, such as prophase, metaphase, anaphase, telophase and cytokinesis. Cell cycle progression sometimes is substantially arrested in a particular phase of the cell cycle (e.g., about 90% of cells in a population are arrested at a particular phase, such as G1 or S phase). In some embodiments, cell cycle progression sometimes is not arrested significantly in any one phase of the cycle. For example, a subpopulation of cells can be substantially arrested in the S-phase of the cell cycle and another subpopulation of cells can be substantially arrested at the G1 phase of the cell cycle. In certain embodiments, the cell cycle is not arrested substantially at any particular phase of the cell cycle. Arrest determinations often are performed at one or more specific time points, such as about 4 hours, about 8 hours, about 12 hours, about 16 hours, about 20 hours, about 24 hours, about 36 hours or about 48 hours, and apoptosis may have occurred or may be occurring during or by these time points.

The term “redistribution of nucleolin” refers to migration of the protein nucleolin or a fragment thereof from the nucleolus to another portion of a cell, such as the nucleoplasm. Different types of nucleolin exist and are described herein. Nucleolin sometimes is distributed from the nucleolus when detectable levels of nucleolin are present in another cell compartment (e.g., the nucleolus). Methods for detecting nucleolin are known and described herein. A nucleolus of cells in which nucleolin is redistributed may include about 55% to about 95% of the nucleolin in untreated cells in some embodiments. A nucleolus of cells in which nucleolin is substantially redistributed may include about 5% to about 50% of the nucleolin in untreated cells.

In certain embodiments, specific nucleotide sequences in ribosomal nucleic acids that interact with cellular components are determined by known techniques in the art, such as chromosome immunoprecipitation (ChIP). ChIP assays also can be useful for determining which cellular components are complexed with a specific nucleotide sequence in chromosomal DNA. Generally in ChIP assays chromosomal DNA is cross-linked to molecules in complex with it and the cross-linked product is fragmented. During or after these steps, the chromatin is contacted with one or more antigen binding agents, and before or after this step, fragments are separated. Using such techniques, nucleotide sequences complexed with certain molecules that interact with antigen binding agents can be determined. ChIP assay protocols are known (e.g., world wide web address: protocol-online.org/prot/Molecular_Biology/Protein/Immunoprecipitation/Chromatin_Immunoprecipitation_ChIP_Assay_/).

Molecules are cross-linked using an appropriate chemical linker that yields a reversible or non-reversible linkage (see e.g., Orlando, et al., Methods 11:205-214 (1997)). In an embodiment, formaldehyde is utilized as a reversible cross-linking agent (see e.g., Johnson & Bresnick, Methods 26:27-36 (2002)). The cross-linking agent often is contacted with an organism or a cell (e.g., a non-disrupted cell) and sometimes is contacted with a cell lysate. In an embodiment, a cell is contacted with a cross linking agent and the cell then is lysed. Cells often are exposed to certain molecules or conditions described previously (e.g., a small organic or inorganic molecule or ionizing radiation), before being exposed to a cross-linking agent. Cross-linking agents frequently link adjacent molecules to one another in a cell or sample, such that molecular antigens in a sample sometimes are directly cross-linked with one another, and sometimes are indirectly cross-linked to one another where one or more non-antigen molecules intervene.

After a cross-linked sample is prepared, cross-linked chromatin DNA often is fragmented using an appropriate process, such as sonication or shearing through a needle and syringe, for example. Using sonication, chromatin fragments of about 500 to about 1000 base pairs in length often are obtained. In some embodiments, cross-linked chromatin is separated from other sample components before fragmentation, and sometimes fragmented chromatin is separated from other assay components before the chromatin fragments are contacted with antigen binding agents. Cross-linked chromatin or chromatin fragments are separated from other sample components by an appropriate process, such as density centrifugation, gel electrophoresis or chromatography, for example.

Chromatin (e.g., cross-linked chromatin, fragmented chromatin, or cross-linked and fragmented chromatin) is contacted with one or more antigen binding agents. The antigen binding agents specifically bind to an antigen in a cellular component cross-linked to the chromosomal DNA (e.g., a protein (e.g., transcription factor, polymerase, histone)) or to a component of the chromosomal DNA itself (e.g., BrdU incorporated in the chromosomal DNA). Antigen binding agents often are useful for detecting molecular antigens in association with the chromatin and/or are useful for separating the cross-linked chromatin from other non-cross-linked components in the system (e.g., separating cross-linked chromosome fragments of different sizes from one another). This step may be performed before fragmentation or after, and may be performed before separation of fragments or after. The antigen binding agent sometimes is an antibody or antibody fragment, such as an antibody that specifically binds to a component of a complex that synthesizes ribosomal RNA in the cell. Such antibodies may specifically bind to UBF, TBP, TAF 48, TAF 63, TAF 110 or a RNA polymerase I subunit, for example, or may specifically bind to nucleolin, fibrallarin or RecQ, or a portion of the foregoing proteins. The antigen binding agent can be detected using any convenient method known, such as by detection using a labeled secondary antibody or by detecting a label linked to the antigen binding agent itself, for example. Examples of suitable labels, such as enzyme labels (e.g., peroxidase), fluorescent labels and light scattering labels, are known and available to the person of ordinary skill in the art.

Determining whether a specific cellular component is in complex with a specific chromosomal nucleotide sequence can be assessed by ChIP. In such embodiments, a chromosomal DNA can be contacted with (1) a nucleotide sequence binding agent that specifically detects the nucleotide sequence of interest (e.g., a hybridization probe linked to a detectable label), and (2) a detectable antigen binding agent that specifically binds to a cellular component of interest. In the latter embodiments, detecting co-localized sequence binding agent and the antigen binding agent determines the specific nucleotide sequence is complexed with the cell component of interest (e.g., co-detection of these agents on a single separated and cross-linked chromosomal DNA fragment). Whether two molecular antigens are in proximity to one another in a cross-linked chromosomal DNA can be determined. This analysis can be effected by contacting the chromosomal DNA with two antigen binding agents that generate a detectable signal when bound to complexed antigens in proximity to one another. Examples of such antigen binding agents is a pair of distinct antibodies each linked to a member of a binding pair, such as, for example, a first antibody linked to a first oligonucleotide and a second antibody linked to a second oligonucleotide that can hybridize to the first oligonucleotide. In the latter example, the hybridization product of the first and second oligonucleotide can be detected by PCR (e.g., WO 2005/074417). The antigen binding agent may be linked to a molecule that facilitates separation, such as linkage to a bead or other solid phase, that allows separation of the antigen binding agent and the DNA and other molecules complexed with it. The antigen binding agent may be directly linked (e.g., covalent or non-covalent direct linkage) or indirectly linked (e.g., via a secondary antibody directly linked to a bead or via a biotin-streptavidin linkage) to the agent that facilitates separation.

For embodiments in which the antigen binding agent/chromatin DNA complex is separated, subsequent steps often are performed. For example, the immobilized extension product sometimes is treated with an agent that digests proteins in association with the extension product (e.g. a protease such as pronase that digests antigen proteins and binding partner proteins such as antibodies). In some embodiments, cross-linking is reversed using standard techniques (e.g., heating the sample) and extension product components are separated from one another as described previously. In certain embodiments, one or more chromatin DNA fragments in association with an antigen binding agent are sequenced using standard techniques (e.g., using a TOPO® cloning plasmid).

Thus, provided in certain embodiments is a composition comprising chromosomal DNA cross-linked to one or more cellular components and an antigen binding agent that specifically binds to nucleolin, fibrillarin and/or RecQ. Also provided in specific embodiments is a composition comprising chromosomal DNA cross-linked to one or more cellular components, and an antigen binding agent that specifically binds to UBF, TBP, TAF 48, TAF 63, TAF 110 or a RNA polymerase I subunit. Such compositions sometimes comprise a quinolone analog, such as an analog of formula 3 or 3A or of formula 2 or 2A-2D. In certain embodiments, the chromosomal DNA is a chromosomal fragment. The chromosomal DNA or DNA fragment sometimes comprises a ribosomal nucleic acid nucleotide sequence, such as a ribosomal nucleotide sequence described herein. The ribosomal nucleotide sequence may form, or be capable of forming, a quadruplex structure (e.g., an intramolecular parallel or mixed parallel structure).

In some embodiments, provided is a method for determining a ribosomal nucleic acid nucleotide sequence complexed with a particular cellular component that is complexed with the ribosomal nucleic acid nucleotide sequence, which comprises: contacting chromosomal DNA, or a fragment thereof, cross-linked to one or more cellular components with an antigen binding agent that specifically binds to the particular cellular component; and sequencing the chromosomal DNA, or fragment thereof, cross-linked to the particular cellular component, whereby the ribosomal nucleic acid nucleotide sequence is determined. The particular cellular component sometimes is nucleolin, fibrillarin or RecQ, and can be UBF, TBP, TAF 48, TAF 63, TAF 110 or a RNA polymerase I subunit in some embodiments. Such methods sometimes comprise contacting chromosomal DNA with a quinolone analog, such as an analog of formula 3 or 3A or of formula 2 or 2A-2D. In some embodiments, the chromosomal DNA is a chromosomal fragment. In certain embodiments, a fragment in association with the specific binding agent is separated from other fragments, and at times a fragment in association with the specific binding agent is detected by a detectable label linked to the binding agent. The antigen binding agent sometimes is linked to a solid phase, such as a bead, and sometimes is linked to a detectable label.

Provided also herein is a method for inducing cell apoptosis, which comprises contacting a cell with an amount of a compound effective to induce cell apoptosis, wherein the compound interacts with a protein kinase and interacts with a component of a complex that synthesizes ribosomal RNA in the cell. In certain embodiments, the compound binds to the protein kinase and to the component. The protein kinase sometimes is a member of a mitogen activated protein (MAP) kinase, mTOR or PI3 kinase pathway. In certain embodiments, the protein kinase is a cell cycle regulating protein kinase, is an RSK protein kinase, is a casein kinase, is an AKT protein kinase, or is selected from the group consisting of ABL, S6K, Tie, TrkA, ZIPK, Pim-1, SAPK, Flt3 and DRAK protein kinases. The component sometimes is selected from the group consisting of UBF, TBP, TAF 48, TAF 63, TAF 110 and a RNA polymerase I subunit. In some embodiments, the compound induces apoptosis of proliferating cells preferentially over quiescent cells. The compound sometimes is a quinolone analog, such as a compound of formula 3 or 3A (e.g., formula A-1).

Also provided is a method for inducing cell apoptosis, which comprises contacting a cell with an amount of compound effective to induce cell apoptosis, wherein the compound interacts with a nucleic acid structure of ribosomal DNA. The nucleic acid structure is an intramolecular quadruplex structure in some embodiments, which may interact with nucleolin, fibrallarin or RecQ. The compound sometimes is a quinolone analog, such as a compound of formula 3 or 3A, or formula 2 or 2A-2D.

Provided also is a method for inducing cell apoptosis, which comprises contacting a cell with an amount of a compound effective to induce cell apoptosis, wherein the compound interacts with a region of ribosomal nucleic acid that interacts with nucleolin, fibrillarin and/or RecQ. The region of the ribosomal nucleic acid that interacts with nucleolin, fibrillarin and/or RecQ may comprise a quadruplex structure. The compound sometimes is a quinolone analog, such as a compound of formula 3 or 3A, or formula 2 or 2A-2D. The ribosomal nucleic acid sometimes is rRNA, or may be rDNA.

Cellular signally pathways set forth in FIGS. 6A and 6B have made it possible to select combination therapies that inhibit rRNA biogenesis and thereby inhibit cell proliferation. In an embodiment, a combination therapeutic is a composition which comprises two or more molecules from two or more classes selected from the group consisting of a protein kinase inhibitor, cyclin activator, tumor suppressor activator and ribosomal biogenesis inhibitor. Such a combination therapeutic can be advantageous over single-molecule therapeutics as molecules from two or more of the classes, having lower efficacy and toxicity than single-molecule therapeutics from each of the classes, can be selected and in combination have the same or better efficacy as each single-molecule therapeutic but with lower toxicity. The term “inhibitor” in such combination therapeutic embodiments refers to a molecule that reduces a catalytic activity of the target (e.g., phosphoryl transfer or polymerization of nucleotides) or reduces the likelihood the target interacts with a cellular binding partner. In certain embodiments, the ribosomal biogenesis inhibitor inhibits an interaction between two or more components of a polymerase I complex. In some embodiments, one or more of the components of the polymerase I complex are selected from the group consisting of UBF, SL1, RRN3, TIF1A, TBP, TAF48, TAF 63, TAF 110 and a RNA polymerase I subunit. In an embodiment, the ribosomal biogenesis inhibitor inhibits an interaction between a component of a polymerase I complex and rDNA. In certain embodiments, the ribosomal biogenesis inhibitor inhibits processing of the rRNA transcript into mature rRNA. In some embodiments, the protein kinase inhibitor inhibits the catalytic activity of the protein kinase, and/or may inhibit an interaction between a protein kinase and a protein that interacts with it in a pathway leading to ribosomal biogenesis. In certain embodiments, the protein kinase inhibitor inhibits a protein kinase in a pathway that regulates polymerase I activity, and sometimes the protein kinase is selected from the group consisting of mTOR, S6K, ERK-MAPK, PI3K, AKT, CDK2/4, CK2, CDK7, 8, 9, and UCK2. In certain embodiments, the cyclin activator activates a cyclin in a pathway that regulates a polymerase I complex, and sometimes the cyclin activator is a Cdk1/cyclin B interaction activator. In some embodiments, the tumor suppressor activator activates a tumor suppressor involved with polymerase I regulation, and sometimes is selected from the group consisting of p53, PTEN and Rb. In certain embodiments, the composition comprises a ribosomal biogenesis inhibitor. Sometimes, the composition comprises or consists essentially of a ribosomal biogenesis inhibitor and a protein kinase inhibitor. Examples of the inhibitors and activators discussed above are known. For example, inhibitors of ribosomal biogenesis (e.g., compound A-1); cyclin dependent protein kinases (e.g., Flavopiridol, BSM-387032, Roscovitine and UCN-01); MEK (e.g., PD-0325901, CI-1040 and AZD6244); CK2 (e.g., CIGB-300); mTOR (e.g., AP23573; CCI-779; rapamycin/sirolimus; and SL0101) and PI3K (e.g., SF1126), are known.

A candidate molecule or nucleic acid may be prepared as a formulation or medicament and may be used as a therapeutic. In some embodiments, provided is a method for treating a disorder, comprising administering a molecule identified by a method described herein to a subject in an amount effective to treat the disorder, whereby administration of the molecule treats the disorder. The terms “treating,” “treatment” and “therapeutic effect” as used herein refer to ameliorating, alleviating, lessening, and removing symptoms of a disease or condition. A candidate molecule or nucleic acid may be in a therapeutically effective amount in the formulation or medicament, which is an amount that can lead to a biological effect, such as a reduction in ribosomal biogenesis in certain cells or tissues (e.g., cancer cells and tumors), apoptosis of certain cells (e.g., cancer cells), reduction of proliferation of certain cells, or lead to ameliorating, alleviating, lessening, or removing symptoms of a disease or condition, for example. In some embodiments involving a nucleic acid candidate molecule, such as in gene therapies, antisense therapies, and siRNA or RNAi therapies, the nucleic acid may integrate with a host genome or not integrate. Any suitable formulation of a candidate molecule can be prepared for administration. Any suitable route of administration may be used, including but not limited to oral, parenteral, intravenous, intramuscular, transdermal, topical and subcutaneous routes. The subject may be a rodent (e.g., mouse, rat, hamster, guinea pig, rabbit), ungulate (e.g., bovine, porcine, equine, caprine), fish, avian, reptile, cat, dog, ungulate, monkey, ape or human.

In cases where a candidate molecule is sufficiently basic or acidic to form stable nontoxic acid or base salts, administration of the candidate molecule as a salt may be appropriate. Examples of pharmaceutically acceptable salts are organic acid addition salts formed with acids that form a physiological acceptable anion, for example, tosylate, methanesulfonate, acetate, citrate, malonate, tartarate, succinate, benzoate, ascorbate, a-ketoglutarate, and a-glycerophosphate. Suitable inorganic salts may also be formed, including hydrochloride, sulfate, nitrate, bicarbonate, and carbonate salts. Pharmaceutically acceptable salts are obtained using standard procedures well known in the art, for example by reacting a sufficiently basic candidate molecule such as an amine with a suitable acid affording a physiologically acceptable anion. Alkali metal (e.g., sodium, potassium or lithium) or alkaline earth metal (e.g., calcium) salts of carboxylic acids also are made.

In some embodiments, a candidate molecule is administered systemically (e.g., orally) in combination with a pharmaceutically acceptable vehicle such as an inert diluent or an assimilable edible carrier. A candidate molecule may be enclosed in hard or soft shell gelatin capsules, compressed into tablets, or incorporated directly with the food of the patient's diet. For oral therapeutic administration, the active candidate molecule may be combined with one or more excipients and used in the form of ingestible tablets, buccal tablets, troches, capsules, elixirs, suspensions, syrups, wafers, and the like. Such compositions and preparations should contain at least 0.1% of active candidate molecule. The percentage of the compositions and preparations may be varied and may conveniently be between about 2 to about 60% of the weight of a given unit dosage form. The amount of active candidate molecule in such therapeutically useful compositions is such that an effective dosage level will be obtained.

Tablets, troches, pills, capsules, and the like also may contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active candidate molecule, sucrose or fructose as a sweetening agent, methyl and propylparabens as preservatives, a dye and flavoring such as cherry or orange flavor. Any material used in preparing any unit dosage form is pharmaceutically acceptable and substantially non-toxic in the amounts employed. In addition, the active candidate molecule may be incorporated into sustained-release preparations and devices.

The active candidate molecule also may be administered intravenously or intraperitoneally by infusion or injection. Solutions of the active candidate molecule or its salts may be prepared in a buffered solution, often phosphate buffered saline, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms. The candidate molecule is sometimes prepared as a polymatrix-containing formulation for such administration (e.g., a liposome or microsome). Liposomes are described for example in U.S. Pat. No. 5,703,055 (Felgner, et al.) and Gregoriadis, Liposome Technology vols. I to III (2nd ed. 1993).

Pharmaceutical dosage forms suitable for injection or infusion can include sterile aqueous solutions or dispersions or sterile powders comprising the active ingredient that are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active candidate molecule in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present candidate molecules may be applied in liquid form. Candidate molecules often are administered as compositions or formulations, in combination with a dermatologically acceptable carrier, which may be a solid or a liquid. Examples of useful dermatological compositions used to deliver candidate molecules to the skin are known (see, e.g., Jacquet, et al. (U.S. Pat. No. 4,608,392), Geria (U.S. Pat. No. 4,992,478), Smith, et al. (U.S. Pat. No. 4,559,157) and Wortzman (U.S. Pat. No. 4,820,508).

Candidate molecules may be formulated with a solid carrier, which include finely divided solids such as talc, clay, microcrystalline cellulose, silica, alumina and the like. Useful liquid carriers include water, alcohols or glycols or water-alcohol/glycol blends, in which the present candidate molecules can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers. Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Nucleic acids having ribosomal nucleotide sequences, or complements thereof, can be isolated and prepared in a composition for use and administration. A nucleic acid composition can include pharmaceutically acceptable salts, esters, or salts of such esters of one or more nucleic acids. Naked nucleic acids may be administered to a system, or nucleic acids may be formulated with one or more other molecules.

Compositions comprising nucleic acids can be prepared as a solution, emulsion, or polymatrix-containing formulation (e.g., liposome and microsphere). Examples of such compositions are set forth in U.S. Pat. Nos. 6,455,308 (Freier), 6,455,307 (McKay et al.), 6,451,602 (Popoff et al.), and 6,451,538 (Cowsert), and examples of liposomes also are described in U.S. Pat. No. 5,703,055 (Felgner et al.,) and Gregoriadis, Liposome Technology vols. I to III (2nd ed. 1993). The compositions can be prepared for any mode of administration, including topical, oral, pulmonary, parenteral, intrathecal, and intranutrical administration. Examples of compositions for particular modes of administration are set forth in U.S. Pat. Nos. 6,455,308 (Freier), 6,455,307 (McKay et al.), 6,451,602 (Popoff et al.), and 6,451,538 (Cowsert). Nucleic acid compositions may include one or more pharmaceutically acceptable carriers, excipients, penetration enhancers, and/or adjuncts. Choosing the combination of pharmaceutically acceptable salts, carriers, excipients, penetration enhancers, and/or adjuncts in the composition depends in part upon the mode of administration. Guidelines for choosing the combination of components for an nucleic acid oligonucleotide composition are known, and examples are set forth in U.S. Pat. Nos. 6,455,308 (Freier), 6,455,307 (McKay et al.), 6,451,602 (Popoff et al.), and 6,451,538 (Cowsert).

A nucleic acid may be modified by chemical linkages, moieties, or conjugates that reduce toxicity, enhance activity, cellular distribution, or cellular uptake of the nucleic acid. Examples of such modifications are set forth in U.S. Pat. Nos. 6,455,308 (Freier), 6,455,307 (McKay et al.), 6,451,602 (Popoff et al.), and 6,451,538 (Cowsert).

In another embodiment, a composition may comprise a plasmid that encodes a nucleic acid described herein. Many of the composition components described above for oligonucleotide compositions, such as carrier, excipient, penetration enhancer, and adjunct components, can be utilized in compositions containing expression plasmids. Also, the nucleic acid expressed by the plasmid may include some of the modifications described above that can be incorporated with or in an nucleic acid after expression by a plasmid. Recombinant plasmids are sometimes designed for nucleic acid expression in microbial cells (e.g., bacteria (e.g., E. coli.), yeast (e.g., S. cerviseae), or fungi), and more often the plasmids are designed for nucleic acid expression in eukaryotic cells (e.g., human cells). Suitable host cells are discussed further in Goeddel, Gene Expression Technology: Methods in Enzymology 185, Academic Press, San Diego, Calif. (1990). The plasmid may be delivered to the system or a portion of the plasmid that contains the nucleic acid encoding nucleotide sequence may be delivered.

When nucleic acids are expressed from plasmids in mammalian cells, expression plasmid regulatory elements sometimes are derived from viral regulatory elements. For example, commonly utilized promoters are derived from polyoma, Adenovirus 2, Rous Sarcoma virus, cytomegalovirus, and Simian Virus 40. A plasmid may include an inducible promoter operably linked to the nucleic acid-encoding nucleotide sequence. In addition, a plasmid sometimes is capable of directing nucleic acid expression in a particular cell type by use of a tissue-specific promoter operably linked to the nucleic acid-encoding sequence, examples of which are albumin promoters (liver-specific; Pinkert et al., Genes Dev. 1: 268-277 (1987)), lymphoid-specific promoters (Calame & Eaton, Adv. Immunol. 43: 235-275 (1988)), T-cell receptor promoters (Winoto & Baltimore, EMBO J. 8: 729-733 (1989)), immunoglobulin promoters (Banerji et al., Cell 33: 729-740 (1983) and Queen & Baltimore, Cell 33: 741-748 (1983)), neuron-specific promoters (e.g., the neurofilament promoter; Byrne & Ruddle, Proc. Natl. Acad. Sci. USA 86: 5473-5477 (1989)), pancreas-specific promoters (Edlund et al., Science 230: 912-916 (1985)), and mammary gland-specific promoters (e.g., milk whey promoter; U.S. Pat. No. 4,873,316 and European Application Publication No. 264,166). Developmentally-regulated promoters also may be utilized, which include, for example, murine hox promoters (Kessel & Gruss, Science 249: 374-379 (1990)) and α-fetopolypeptide promoters (Campes & Tilghman, Genes Dev. 3. 537-546 (1989)).

Nucleic acid compositions may be presented conveniently in unit dosage form, which are prepared according to conventional techniques known in the pharmaceutical industry. In general terms, such techniques include bringing the nucleic acid into association with pharmaceutical carrier(s) and/or excipient(s) in liquid form or finely divided solid form, or both, and then shaping the product if required. The nucleic acid compositions may be formulated into any dosage form, such as tablets, capsules, gel capsules, liquid syrups, soft gels, suppositories, and enemas. The compositions also may be formulated as suspensions in aqueous, non-aqueous, or mixed media. Aqueous suspensions may further contain substances which increase viscosity, including for example, sodium carboxymethylcellulose, sorbitol, and/or dextran. The suspension may also contain one or more stabilizers.

Nucleic acids can be translocated into cells via conventional transformation or transfection techniques. As used herein, the terms “transformation” and “transfection” refer to a variety of standard techniques for introducing an nucleic acid into a host cell, which include calcium phosphate or calcium chloride co-precipitation, transduction/infection, DEAE-dextran-mediated transfection, lipofection, electroporation, and iontophoresis. Also, liposome compositions described herein can be utilized to facilitate nucleic acid administration. An nucleic acid composition may be administered to an organism in a number of manners, including topical administration (including ophthalmic and mucous membrane (e.g., vaginal and rectal) delivery), pulmonary administration (e.g., inhalation or insufflation of powders or aerosols, including by nebulizer; intratracheal, intranasal, epidermal and transdermal), oral administration, and parenteral administration (e.g., intravenous, intraarterial, subcutaneous, intraperitoneal injection or infusion, intramuscular injection or infusion; and intracranial (e.g., intrathecal or intraventricular)).

Generally, the concentration of the candidate molecule or nucleic acid in a liquid composition often is from about 0.1 wt % to about 25 wt %, sometimes from about 0.5 wt % to about 10 wt %. The concentration in a semi-solid or solid composition such as a gel or a powder often is about 0.1 wt % to about 5 wt %, sometimes about 0.5 wt % to about 2.5 wt %. A candidate molecule or nucleic acid composition may be prepared as a unit dosage form, which is prepared according to conventional techniques known in the pharmaceutical industry. In general terms, such techniques include bringing a candidate molecule or nucleic acid into association with pharmaceutical carrier(s) and/or excipient(s) in liquid form or finely divided solid form, or both, and then shaping the product if required. The candidate molecule or nucleic acid composition may be formulated into any dosage form, such as tablets, capsules, gel capsules, liquid syrups, soft gels, suppositories, and enemas. The compositions also may be formulated as suspensions in aqueous, non-aqueous, or mixed media. Aqueous suspensions may further contain substances which increase viscosity, including for example, sodium carboxymethylcellulose, sorbitol, and/or dextran. The suspension may also contain one or more stabilizers.

The amount of the candidate molecule or nucleic acid, or an active salt or derivative thereof, required for use in treatment will vary not only with the particular salt selected but also with the route of administration, the nature of the condition being treated and the age and condition of the patient and will be ultimately at the discretion of the attendant physician or clinician. Candidate molecules or nucleic acids generally are used in amounts effective to achieve the intended purpose of reducing the number of targeted cells; detectably eradicating targeted cells; treating, ameliorating, alleviating, lessening, and removing symptoms of a disease or condition; and preventing or lessening the probability of the disease or condition or reoccurrence of the disease or condition. A therapeutically effective amount sometimes is determined in part by analyzing samples from a subject, cells maintained in vitro and experimental animals. For example, a dose can be formulated and tested in assays and experimental animals to determine an IC50 value for killing cells. Such information can be used to more accurately determine useful doses.

A useful candidate molecule or nucleic acid dosage often is determined by assessing its in vitro activity in a cell or tissue system and/or in vivo activity in an animal system. For example, methods for extrapolating an effective dosage in mice and other animals to humans are known to the art (see, e.g., U.S. Pat. No. 4,938,949). Such systems can be used for determining the LD50 (the dose lethal to 50% of the population) and the ED50 (the dose therapeutically effective in 50% of the population) of a candidate molecule or nucleic acid. The dose ratio between a toxic and therapeutic effect is the therapeutic index and it can be expressed as the ratio ED50/LD50. The candidate molecule or nucleic acid dosage often lies within a range of circulating concentrations for which the ED50 is associated with little or no toxicity. The dosage may vary within this range depending upon the dosage form employed and the route of administration utilized. For any candidate molecules or nucleic acids used in the methods described herein, the therapeutically effective dose can be estimated initially from cell culture assays. A dose sometimes is formulated to achieve a circulating plasma concentration range covering the IC50 (i.e., the concentration of the test candidate molecule which achieves a half-maximal inhibition of symptoms) as determined in in vitro assays, as such information often is used to more accurately determine useful doses in humans. Levels in plasma may be measured, for example, by high performance liquid chromatography.

Another example of effective dose determination for a subject is the ability to directly assay levels of “free” and “bound” candidate molecule or nucleic acid in the serum of the test subject. Such assays may utilize antibody mimics and/or “biosensors” generated by molecular imprinting techniques. The candidate molecule or nucleic acid is used as a template, or “imprinting molecule”, to spatially organize polymerizable monomers prior to their polymerization with catalytic reagents. Subsequent removal of the imprinted molecule leaves a polymer matrix which contains a repeated “negative image” of the candidate molecule and is able to selectively rebind the molecule under biological assay conditions (see, e.g., Ansell, et al., Current Opinion in Biotechnology 7: 89-94 (1996) and in Shea, Trends in Polymer Science 2: 166-173 (1994)). Such “imprinted” affinity matrixes are amenable to ligand-binding assays, whereby the immobilized monoclonal antibody component is replaced by an appropriately imprinted matrix (see, e.g., Vlatakis, et al., Nature 361: 645-647 (1993)). Through the use of isotope-labeling, “free” concentration of candidate molecule can be readily monitored and used in calculations of IC50. Such “imprinted” affinity matrixes can also be designed to include fluorescent groups whose photon-emitting properties measurably change upon local and selective binding of candidate molecule or nucleic acid. These changes can be readily assayed in real time using appropriate fiber optic devices, in turn allowing the dose in a test subject to be quickly optimized based on its individual IC50. An example of such a “biosensor” is discussed in Kriz, et al., Analytical Chemistry 67: 2142-2144 (1995).

Exemplary doses include milligram or microgram amounts of the candidate molecule or nucleic acid per kilogram of subject or sample weight, for example, about 1 microgram per kilogram to about 500 milligrams per kilogram, about 100 micrograms per kilogram to about 5 milligrams per kilogram, or about 1 microgram per kilogram to about 50 micrograms per kilogram. It is understood that appropriate doses of a small molecule depend upon the potency of the small molecule with respect to the expression or activity to be modulated. When one or more of these small molecules is to be administered to an animal (e.g., a human) in order to modulate expression or activity of a polypeptide or nucleic acid described herein, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific candidate molecule employed, the age, body weight, general health, gender, and diet of the subject, the time of administration, the route of administration, the rate of excretion, any drug combination, and the degree of expression or activity to be modulated.

In some embodiments, a candidate molecule or nucleic acid is utilized to treat a cell proliferative condition. In such treatments, the terms “treating,” “treatment” and “therapeutic effecf” can refer to reducing or stopping a cell proliferation rate (e.g., slowing or halting tumor growth), reducing the number of proliferating cancer cells (e.g., ablating part or all of a tumor) and alleviating, completely or in part, a cell proliferation condition. Cell proliferative conditions include, but are not limited to, cancers of the colorectum, breast, lung, liver, pancreas, lymph node, colon, prostate, brain, head and neck, skin, liver, kidney, and heart. Examples of cancers include hematopoietic neoplastic disorders, which are diseases involving hyperplastic/neoplastic cells of hematopoietic origin (e.g., arising from myeloid, lymphoid or erythroid lineages, or precursor cells thereof). The diseases can arise from poorly differentiated acute leukemias, e.g., erythroblastic leukemia and acute megakaryoblastic leukemia. Additional myeloid disorders include, but are not limited to, acute promyeloid leukemia (APML), acute myelogenous leukemia (AML) and chronic myelogenous leukemia (CML) (reviewed in Vaickus, Crit. Rev. in Oncol./Hemotol. 11:267-297 (1991)); lymphoid malignancies include, but are not limited to acute lymphoblastic leukemia (ALL), which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease. In a particular embodiment, the cell proliferative disorder is pancreatic cancer, including non-endocrine and endocrine tumors. Illustrative examples of non-endocrine tumors include but are not limited to adenocarcinomas, acinar cell carcinomas, adenosquamous carcinomas, giant cell tumors, intraductal papillary mucinous neoplasms, mucinous cystadenocarcinomas, pancreatoblastomas, serous cystadenomas, solid and pseudopapillary tumors. An endocrine tumor may be an islet cell tumor.

Cell proliferative conditions also include inflammatory conditions, such as inflammation conditions of the skin, including, for example, eczema, discoid lupus erythematosus, lichen planus, lichen sclerosus, mycosis fungoides, photodermatoses, pityriasis rosea, psoriasis. Also included are cell proliferative conditions related to obesity, such as proliferation of adipocytes, for example.

Cell proliferative conditions also include viral diseases, including for example, Acquired Immunodeficiency Syndrome, Adenoviridae Infections, Alphavirus Infections, Arbovirus Infections, Borna Disease, Bunyaviridae Infections, Caliciviridae Infections, Chickenpox, Coronaviridae Infections, Coxsackievirus Infections, Cytomegalovirus Infections, Dengue, DNA Virus Infections, Eethyma, Contagious, Encephalitis, Arbovirus, Epstein-Barr Virus Infections, Erythema Infectiosum, Hantavirus Infections, Hemorrhagic Fevers, Viral, Hepatitis, Viral, Human, Herpes Simplex, Herpes Zoster, Herpes Zoster Oticus, Herpesviridae Infections, Infectious Mononucleosis, Influenza in Birds, Influenza, Human, Lassa Fever, Measles, Molluscum Contagiosum, Mumps, Paramyxoviridae Infections, Phlebotomus Fever, Polyomavirus Infections, Rabies, Respiratory Syncytial Virus Infections, Rift Valley Fever, RNA Virus Infections, Rubella, Slow Virus Diseases, Smallpox, Subacute Sclerosing Panencephalitis, Tumor Virus Infections, Warts, West Nile Fever, Virus Diseases and Yellow Fever. For example, Large T antigen of the SV40 transforming virus acts on UBF, activates it and recruits other viral proteins to Pol I complex, and thereby stimulates cell proliferation to ensure virus propagation. Cell proliferative conditions also include conditions related to angiogenesis (e.g., cancers) and obesity caused by proliferation of adipocytes and other fat cells.

Cell proliferative conditions also include cardiac conditions resulting from cardiac stress, such as hypertension, baloon angioplasty, valvular disease and myocardial infarction. For example, cardiomyocytes are differentiated muscle cells in the heart that constitute the bulk of the ventricle wall, and vascular smooth muscle cells line blood vessels. Although both are muscle cell types, cardiomyocytes and vascular smooth muscle cells vary in their mechanisms of contraction, growth and differentiation. Cardiomyocytes become terminally differentiated shortly after heart formation and thus loose the capacity to divide, whereas vascular smooth muscle cells are continually undergoing modulation from the contractile to proliferative phenotype. Under various pathophysiological stresses such as hypertension, baloon angioplasty, valvular disease and myocardial infarction, for example, the heart and vessels undergo morphologic growth-related alterations that can reduce cardiac function and eventually manifest in heart failure. Thus, provided herein are methods for treating cardiac cell proliferative conditions by administering a compound or nucleic acid described herein in an effective amount to treat the cardiac condition. The compound or nucleic acid may be administered before or after a cardiac stress has occurred or has been detected, and the compound or nucleic acid may be administered after occurrence or detection of hypertension, baloon angioplasty, valvular disease or myocardial infarction, for example. Administration of such a compound or nucleic acid may decrease proliferation of vascular muscle cells and/or smooth muscle cells.

Certain embodiments also are directed to treating symptoms of aging and/or treating conditions pertaining to cell senescence by administration of a candidate molecule or nucleic acid described herein. For example, the premature aging disease of Werner Syndrome results from alterations in the Werner gene, which codes for the WRN DNA helicase. Without being limited by theory, this protein is known to localize to the nucleolus and specifically bind to G-quadruplexes, and mutations in the WRN DNA helicase result in senescence.

Toxicity Assessment Procedures

Provided herein are assays for predicting toxicity of a molecule to cells or a subject. In certain embodiments, phosphorylation of JNK and optionally MAPK is assessed, and the risk of toxicity is assessed based upon the phosphorylation state of these proteins. Full length JNK and MAPK proteins may be utilized, and a fragment of a JNK and/or MAPK protein capable of being phosphorylated may be utilized in certain embodiments. Mutated JNK or MAPK amino acid sequence may be utilized, such as a mutant protein in which one or more phosphorylation sites has been removed (e.g., reduction of phosphorylation sites can reduce background levels). Prediction of toxicity can be expressed in any convenient and informative format, such as a percentage or likelihood of toxicity, and/or gradations (e.g., high, medium, low risk of toxicity). Toxicity sometimes is inflammation or irritation.

Presence or absence of a phosphate moiety on a JNK or MAPK protein or fragment can be detected in a variety of systems selected by the artisan. In some embodiments, the gamma phosphoryl moiety of adenosine triphosphate (ATP), which is transferred to a protein substrate by protein kinases, or a derivative thereof, is detectably labeled. In such embodiments, the detectably labeled gamma phosphoryl moiety transferred to a substrate is detected. In some embodiments, an ATP having a ³²P or ³³P gamma phosphoryl moiety is utilized in an assay. In certain embodiments, The gamma phosphate of ATP can be detectably labeled by a method known to the skilled artisan. In certain embodiments, the gamma moiety includes a sulfur radioisotope (e.g., ³⁵S atom).

In certain embodiments, the JNK and/or MAPK protein is immobilized to a solid phase (e.g., a substrate array) and phosphorylation activity is monitored. A reaction buffer may be utilized in such a system that includes components conducive to phosphorylation reactions. These conditions include, for example, pH, salt concentration, concentration of Mg²⁺, and detergent concentration. After incubation in the reaction buffer, the microarray is washed to remove any labeled ATP and the product is quantified via the detectably labeled phosphate that has been transferred during the kinase reaction from ATP to the substrate. Signal intensity is proportional to the amount of labeled phosphate on the substrate and corresponds to phosphorylation activity. In some embodiments, a substrate is labeled with a detectable phosphoryl moiety and dephosphorylation of the substrate is detected.

Without being bound by theory, some kinases and phosphatases act on a substrate only in a particular molecular context. Such a molecular context may, for example, consist of certain scaffold proteins. In certain embodiments, such scaffold proteins are provided in the assay conditions (e.g., with the reaction buffer). In some embodiments, the scaffold proteins are also immobilized on the surface of a solid support.

In certain embodiments, JNK and/or MAPK phosphorylation is visualized and optionally quantified using antibodies that bind specifically to phosphorylated proteins or peptides. Such antibodies include, but are not limited to antibodies that bind to phospho-serine, antibodies that bind to phosphor-threonine or antibodies that bind to phospho-tyrosine. The antibody sometimes is specific for the phosphoryl amino acid regardless of the amino acid sequence surrounding the phosphoryl amino acid, and in some embodiments, the antibody specifically binds to an epitope comprising the phosphoryl amino acid and one or more surrounding amino acids. The antibody that binds to the phosphorylated protein or peptide may include a detectable label or can be associated with a detectable label during the assay. In some embodiments, a secondary antibody is used to detect the antibody bound to the phosphorylated protein or peptide. The amount of phosphorylated substrate can be detected, and such assays are useful for detecting phosphorylation and/or dephosphorylation activity. In some assay embodiments, phosphorylation is detected by fluorescence polarization after contacting a sample with a peptide substrate linked to a fluorophore and an antibody that specifically binds to the phosphorylated peptide (e.g., PolarScreen™ kinase assay; world wide web address: invitrogen.com/content.cfm?pageid=10568).

In certain assay embodiments, phosphorylation is detected by FRET. In an embodiment a sample is contacted with a peptide substrate linked to two fluorophores capable of FRET (e.g., one fluorophore at the N-terminus and one at the C-terminus) and a protease that specifically cleaves the peptide substrate differentially based upon its phosphorylation state (e.g., Z′-LYTE™ protein kinase and phosphatase assays (world wide web address: invitrogen.com/content.cfm?pageid=9866)). In some embodiments, a sample is contacted with (1) a peptide substrate containing a first fluorophore and (2) a detection molecule linked to a second fluorophore capable of FRET with the first fluorophore linked to the peptide (e.g., LanthaScreen™ TR-FRET Assay (world wide web address: invitrogen.com/content.cfm?pageid=10513)). In the latter embodiments, the detection molecule sometimes is an antibody that specifically binds to phosphorylated peptide and not specifically to non-phosphorylated peptide (e.g., terbium-labeled phospho-tyrosine specific antibody). The detection molecule sometimes is a molecule that is part of a binding pair (e.g., biotin), the peptide is linked to the other binding pair member (e.g., streptavidin or avidin) and the assay system is contacted with a protease that differentially cleaves phosphorylated and non-phosphorylated peptide. These assays can be utilized in homogenous or heterogeneous formats.

In certain embodiments, phosphorylation can be detected using a molecule that binds to phosphate and is linked to a detectable label. A dye can be utilized as a detectable label, such as a dye comprising a metal-chelating moiety. In a specific embodiment, a phosphorylated protein or peptide is detected using a metal-chelating dye. Metal-chelating dyes include, without limitation, BAPTA, IDA, DTPA, phenanthrolines and derivatives thereof (e.g., U.S. Pat. Nos. 4,603,209; 4,849,362; 5,049,673; 5,453,517; 5,459,276; 5,516,911; 5,501,980; and 5,773,227). In specific embodiments, a dye in Pro-Q Diamond stain (Molecular Probes, Oregon) is utilized (e.g., gel or microarray stain).

Other phosphorylation detection systems that may be utilized include commercially available kits such as the PhosphoELISA (Biosource International) and fluorescence-based assays. Suitable fluorescence-based assay systems utilize reagents with novel metal binding amino acid residues exhibiting chelation-enhanced fluorescence (CHEF) upon binding to Mg²⁺ (e.g., US 2005/0080242A2 and US 2005/0080243A1).

Kits

Kits comprise one or more containers, which contain one or more of the compositions and/or components described herein. A kit may comprise one or more of the components in any number of separate containers, packets, tubes, vials, microtiter plates and the like, and in some embodiments, the components may be combined in various combinations in such containers. A kit in some embodiments includes one reagent described herein and provides instructions that direct the user to another reagent described herein that is not included in the kit.

A kit can include reagents described herein in any combination. A kit may comprise one, two, three, four, five or more reagents described herein. For example, a kit can include (1) an isolated nucleic acid that contains a ribosomal nucleotide sequence described herein; (2) a nucleolin protein or fragment thereof and a nucleic acid that binds to it; or (3) an isolated nucleic acid that contains a ribosomal nucleotide sequence described herein and a compound that binds to it linked to a detectable label.

A kit sometimes is utilized in conjunction with a method described herein, and sometimes includes instructions for performing one or more methods described herein and/or a description of one or more compositions or reagents described herein. Instructions and/or descriptions may be in printed form and may be included in a kit insert. A kit also may include a written description of an internet location that provides such instructions or descriptions.

Representative Human rDNA Sequence

Provided hereafter is a representative human rDNA sequence (SEQ ID NO: 1).

```
        1 gctgacacgc tgtcctctgg cgacctgtcg tcggagaggt tgggcctccg gatgcgcgcg
      61 gggctctggc ctcacggtga ccggctagcc ggccgcgctc ctgccttgag ccgcctgccg
    121 cggcccgcgg gcctgctgtt ctctcgcgcg tccgagcgtc ccgactcccg gtgccggccc
    181 gggtccgggt ctctgaccca cccgggggcg gcggggaagg cggcgagggc caccgtgccc
    241 cgtgcgctct ccgctgcggg cgcccggggc gccgcacaac cccacccgct ggctccgtgc
    301 cgtgcgtgtc aggcgttctc gtctccgcgg ggttgtccgc cgccccttcc ccggagtggg
    361 gggtggccgg agccgatcgg ctcgctggcc ggccggcctc cgctcccggg gggctcttcg
    421 atcgatgtgg tgacgtcgtg ctctcccggg ccgggtccga gccgcgacgg gcgaggggcg
    481 gacgttcgtg gcgaacggga ccgtccttct cgctccgccc gcgcggtccc ctcgtctgct
    541 cctctccccg cccgccggcc ggcgtgtggg aaggcgtggg gtgcggaccc cggcccgacc
    601 tcgccgtccc gcccgccgcc ttcgcttcgc gggtgcgggc cggcggggtc ctctgacgcg
    661 gcagacagcc ctgoctgtcg cctccagtgg ttgtcgactt gcgggcggcc cccctccgcg
    721 gcggtggggg tgccgtcccg ccggcccgtc gtgctgccct ctcggggggg gtttgcgcga
    781 gcgtcggctc cgcctgggcc cttgcggtgc tcctggagcg ctccgggttg tccctcaggt
    841 gcccgaggca gaacggtggt gtgtcgttcc cgcccccggc gccccctcct ccggtcgccg
    901 ccgcggtgtc cgcgcgtggg tcctgaggga gctcgtcggt gtggggttcg aggcggtttg
    961 agtgagacga gacgagacgc gcccctccca cgcggggaag ggcgcccgcc tgctctcggt
  1021 gagcgcacgt cccgtgctcc cctctggcgg gtgcgcgcgg gccgtgtgag cgatcgcggt
  1081 gggttcgggc cggtgtgacg cgtgcgccgg ccggccgccg aggggctgcc gttctgccc
  1141 cgaccggtcg tgtgtgggtt gacttcggag gcgctctgcc tcggaaggaa ggaggtgggt
  1201 ggacgggggg gcctggtggg gttgcgcgca cgcgcgcacc ggccgggccc ccgccctgaa
  1261 cgcgaacgct cgaggtggcc gcgcgcaggt gtttcctcgt accgcagggc cccctccctt
  1321 ccccaggcgt ccctcggcgc ctctgcgggc ccgaggagga gcggctggcg ggtgggggga
  1381 gtgtgaccca ccctcggtga gaaaagcctt ctctagcgat ctgagaggcg tgccttgggg
  1441 gtaccggatc ccccgggccg ccgcctctgt ctctgcctcc gttatggtag cgctgccgta
  1501 gcgacccgct cgcagaggac cctcctccgc ttccccctcg acggggttgg gggggagaag
  1561 cgagggttcc gccggccacc gcggtggtgg ccgagtgcgg atcgtcgcct actgtggccc
  1621 gcgcctcccc cttccgagtc gggggaggat cccgccgggc cgggcccggc gctcccaccc
  1681 agcgggttgg gacgcggcgg ccggcgggcg gtgggtgtgc gcgcccggcg ctctgtccgg
  1741 cgcgtgaccc cctccgtccg cgagtcggct ctccgcccgc tcccgtgccg agtcgtgacc
  1801 ggtgccgacg accgcgtttg cgtggcacgg ggtcgggccc gcctggccct gggaaagcgt
  1861 cccacggtgg gggcgcgccg gtctcccgga gcgggaccgg gtcggaggat ggacgagaat
  1921 cacgagcgac ggtggtggtg gcgtgtcggg ttcgtggctg cggtcgctcc ggggcccccg
  1981 gtggcggggc cccggggctc gcgaggcggt tctcggtggg ggccgagggc cgtccggcgt
  2041 cccaggcggg gcgccgcggg accgccctcg tgtctgtggc ggtgggatcc cgcggccgtg
  2101 ttttcctggt ggcccggccg tgcctgaggt ttctccccga gccgccgcct ctgcgggctc
  2161 ccgggtgccc ttgccctcgc ggtccccggc cctcgcccgt ctgtgccctc ttccccgccc
  2221 gccgcccgcc gatcctcttc ttccccccga gcggctcacc ggcttcacgt acgttggtgg
  2281 ccccgcctgg gaccgaaccc ggcaccgcct cgtggggcgc cgccgccggc cactgatcgg
  2341 cccggcgtcc gcgtcccccg gcgcgcgcct tggggaccgg gtcggtggcg cgccgcgtgg
  2401 ggcccggtgg gcttcccgga gggttccggg ggtcggcctg cggcgcgrgc gggggaggag
  2461 acggttccgg gggaccggcc gcggctgcgg cggcggcggt ggtgggggga gccgcgggga
  2521 tcgccgaggg ccggtcggcc gccccgggtg ccccgcggtg ccgccggcgg cggtgaggcc
  2581 ccgcgcgtgt gtcccggctg cggtcggccg cgctcgaggg gtccccgtgg cgtccccttc
  2641 cccgccggcc gccttcctcg cgccttcccc gtcgccccgg cctcgcccgt ggtctctcgt
  2701 cttctcccgg cccgctcttc cgaaccgggt cggcgcgtcc cccgggtgcg cctcgcttcc
  2761 cgggcctgcc gcggcccttc cccgaggcgt ccgtcccggg cgtcggcgtc ggggagagcc
  2821 cgtcctcccc gcgtggcgtc gccccgttcg gcgcgcgcgt gcgcccgagc gcggcccggt
  2881 ggtccctccc ggacaggcgt tcgtgcgacg tgtggcgtgg gtcgacctcc gccttgccgg
  2941 tcgctcgccc tctccccggg tcggggggtg gggcccgggc cggggcctcg gccccggtcg
  3001 ctgcctcccg tcccgggcgg gggcgggcgc gccggccggc ctcggtcgcc ctcccttggc
  3061 cgtcgtgtgg cgtgtgccac ccctgcgccg gcgcccgccg gcggggctcg gagccgggct
  3121 tcggccgggc cccgggccct cgaccggacc ggctgcgcgg gcgctgcggc cgcacggcgc
  3181 gactgtcccc gggccgggca ccgcggtccg cctctcgctc gccgcccgga cgtcggggcc
  3241 gccccgcggg gcgggcggag cgccgtcccc gcctcgccgc cgcccgcggg cgccggccgc
  3301 gcgcgcgcgc gcgtggccgc cggtccctcc cggccgccgg gcgcgggtcg ggccgtccgc
  3361 ctcctcgcgg gcgggcgcga cgaagaagcg tcgcgggtct gtggcgoggg gcccccggtg
  3421 gtcgtgtcgc gtggggggcg ggtggttggg gcgtccggtt cgccgcgccc cgccccggcc
  3481 ccaccggtcc cggccgccgc ccccgcgccc gctcgctccc tcccgtccgc ccgtccgcgg
  3541 cccgtccgtc cgtccgtccg tcgtcctcct cgcttgcggg gcgccgggcc cgtcctcgcg
  3601 aggccccccg gccggccgcc cggccgcgtc gggggctcgc cgcgctctac cttacctacc
  3661 tggttgatcc tgccagtagc atatgcttgt ctcaaagatt aagccatgca tgtctaagta
  3721 cgcacggccg gtacagtgaa actgcgaatg gctcattaaa tcagttatgg ttcctttggt
  3781 cgctcgctcc tctcctactt ggataactgt ggtaattcta gagctaatac atgccgacgg
  3841 gcgctgaccc ccttcgcggg ggggatgcgt gcatttatca gatcaaaacc aacccggtca
  3901 gcccctctcc ggccccggcc ggggggcggg cgccggcggc tttggtgact ctagataacc
  3961 tcgggccgat cgcacgcccc ccgtggcggc gacgacccat tcgaacgtct gccctatcaa
  4021 ctttcgatgg tagtcgccgt gcctaccatg gtgaccacgg gtgacgggga atcagggttc
  4081 gattccggag agggagcctg agaaacggct accacatcca aggaaggcag caggcgcgca
  4141 aattacccac tcccgacccg gggaggtagt gacgaaaaat aacaatacag gactctttcg
  4201 aggccctgta attggaatga gtccacttta aatcctttaa cgaggatcca ttggagggca
  4261 agtctggtgc cagcagccgc ggtaattcca gctccaatag cgratattaa agttgctgca
  4321 gttaaaaagc tcgtagttgg atcttgggag cgggcgggcg gtccgccgcg aggcgagcca
  4381 ccgcccgtcc ccgccccttg cctctcggcg cccccccgat gctcttagct gagtgtcccg
  4441 cggggcccga agcgtttact ttgaaaaaat tagagtgttc aaagcaggcc cgagccgcct
  4501 ggataccgca gctaggaata atggaatagg accgcggttc tattttgttg gttttcggaa
  4561 ctgaggccat gattaagagg gacggccggg ggcattcgta ttgcgccgct agaggtgaaa
  4621 ttcttggacc ggcgcaagac ggaccagagc gaaagcattt gccaagaatg ttttcattaa
  4681 tcaagaacga aagtcggagg ttcgaagacg atcagatacc gtcgtagttc cgaccataaa
  4741 cgatgccgac cggcgatgcg gcggcgttat tcccatgacc cgccgggcag cttccgggaa
  4801 accaaagtct ttgggttccg gggggagtat ggttgcaaag ctgaaactta aaggaattga
  4861 cggaagggca ccaccaggag tggagcctgc ggcttaattt gactcaacac gggaaacctc
  4921 acccggcccg gacacggaca ggattgacag attgatagct ctttctcgat tccgtgggtg
  4981 gtggtgcatg gccgttctta gttggtggag cgatttgtct ggttaattcc gataacgaac
  5041 gagactctgg catgctaact agttacgcga cccccgagcg gtcggcgtcc cccaacttct
  5101 tagagggaca agtggcgttc agccacccga gattgagcaa taacaggtct gtgatgccct
  5161 tagatgtccg gggctgcacg cgcgctacac tgactggctc agcgtgtgcc taccctacgc
  5221 cggcaggcgc gggtaacccg ttgaacccca ttcgtgatgg ggatcgggga ttgcaattat
  5281 tccccatgaa cgagggaatt cccgagtaag tgcgggtcat aagcttgcgt tgattaagtc
  5341 cctgcccttt gtacacaccg cccgtcgcta ctaccgattg gatggtttag tgaggccctc
  5401 ggatcggccc cgccggggtc ggcccacggc cctggcggag cgctgagaag acggtcgaac
  5461 ttgactatct agaggaagta aaagtcgtaa caaggtttcc gtaggtgaac ctgcggaagg
  5521 atcattaacg gagcccggag ggcgaggccc gcggcggcgc cgccgccgcc gcgcgcttcc
  5581 ctccgcacac ccaccccccc accgcgacgc ggcgcgtgcg cgggcggggc ccgcgtgccc
  5641 gttcgttcgc tcgctcgttc gttcgccgcc cggccccgcc gccgcgagag ccgagaactc
  5701 gggagggaga cgggggggag agagagagag agagagagag agagagagag agagagagaa
  5761 agaagggcgt gtcgttggtg tgcgcgtgtc gtggggccgg cgggcggcgg ggagcggtcc
  5821 ccggccgcgg ccccgacgac gtgggtgtcg gcgggcgcgg gggcggttct cggcggcgtc
  5881 gcggcgggtc tgggggggtc tcggtgccct cctccccgcc ggggcccgtc gtccggcccc
  5941 gccgcgccgg ctccccgtct tcggggccgg ccggattccc gtcgcctccg ccgcgccgct
  6001 ccgcgccgcc gggcacggcc ccgctcgctc tecceggect tcccgctagg gcgtctcgag
  6061 ggtcgggggc cggacgccgg tcccctcccc cgcctcctcg tccgcccccc cgccgtccag
  6121 gtacctagcg cgttccggcg cggaggttta aagacccctt ggggggatcg cccgtccgcc
  6181 cgtgggtcgg gggcggtggt gggcccgcgg gggagtcccg tcgggagggg cccggcccct
  6241 cccgcgcctc caccgcggac tccgctcccc ggccggggcc gcgccgccgc cgccgccgcg
  6301 gcggccgtcg ggtgggggct ttacccggcg gccgtcgcgc gcctgccgcg cgtgtggcgt
  6361 gcgccccgcg ccgtgggggc gggaaccccc gggcgcctgt ggggtggtgt ccgcgctcgc
  6421 ccccgcgtgg gcggcgcgcg cctccccgtg gtgtgaaacc ttccgacccc tctccggagt
  6481 ccggtcccgt ttgctgtctc gtctggccgg cctgaggcaa ccccctctcc tcttgggcgg
  6541 ggggggcggg gggacgtgcc gcgccaggaa gggcctcctc ccggtgcgtc gtcgggagcg
  6601 ccctcgccaa atcgacctcg tacgactctt agcggtggat aactcggctc gtgcgtcgat
  6661 gaagaacgca gctagctgcg agaattaatg cgaattgcag gacacattga tcatcgacac
  6721 ttcgaacgca cttgcggccc cgggttcctc ccggggctac gcctgtctga gcgtcgcttg
  6781 ccgatcaatc gccccggggg tgcctccggg ctcctcgggg tgcgcggctg ggggttccct
  6841 cgcagggccc gccgggggcc ctccgtcccc ctaagcgcag acccggcggc gtccgccctc
  6901 ctcttgccgc cgcgcccgcc ccttccccct ccccccgcgg gccctgcgtg gtcacgcgtc
  6961 gggtggcggg ggggagaggg gggcgcgccc ggctgagaga gacggggagg gcggcgccgc
  7021 cgccggaaga cggagaggga aagagagagc cggctcgggc cgagttcccg tggccgccgc
  7081 ctgcggtccg ggttcctccc tcggggggct ccctcgcgcc gcgcgcggct cggggttcgg
  7141 ggttcgtcgg ccccggccgg gtggaaggtc ccgtgcccgt cgtcgtcgtc gtcgcgcgtc
  7201 gtcggcggtg ggggcgtgtt gcgtgcggtg tggtggtggg ggaggaggaa ggcgggtccg
  7261 gaaggggaag ggtgccggcg gggagagagg gtcgggggag cgcgtcccgg tcgccgcggt
  7321 tccgccgccc gcccccggtg gcggcccggc gtccggccga ccggccgctc cccgcgcccc
  7381 tcctcctccc cgccgcccct cctccgaggc cccgcccgtc ctcctcgccc tccccgcgcg
  7441 tacgcgcgcg cgcccgcccg cccggctcgc ctcgcggcgc gtcggccggg gccgggagcc
  7501 cgccccgccg cccgcccgtg gccgcggcgc cggggttcgc gtgtccccgg cggcgacccg
  7561 cgggacgccg cggtgtcgtc cgccgtcgcg cgcccgcctc cggctcgcgg ccgcgccgcg
  7621 ccgcgccggg gccccgtccc gagcttccgc gtcggggcgg cgcggctccg ccgccgcgtc
  7681 ctcggacccg tccccccgac ctccgcgggg gagacgcgcc ggggcgtgcg gcgcccgtcc
  7741 cgcccccggc ccgtgcccct ccctccggtc gtcccgctcc ggcggggcgg cgcgggggcg
  7801 ccgtcggccg cgcgctctct ctcccgtcgc ctctccccct cgccgggccc gtctcccgac
  7861 ggagcgtcgg gcgggcggtc gggccggcgc gattccgtcc gtccgtccgc cgagcggccc
  7921 gtccccctcc gagacgcgac ctcagatcag acgtggcgac ccgctgaatt taagcatatt
  7981 agtcagcgga ggaaaagaaa ctaaccagga ttccctcagt aacggcgagt gaacagggaa
  8041 gagcccagcg ccgaatcccc gccccgcggg gcgcgggaca tgtggcgtac ggaagacccg
  8101 ctccccggcg ccgctcgtgg ggggcccaag tccttctgat cgaggcccag cccgtggacg
  8161 gtgtgaggcc ggtagcggcc ggcgcgcgcc cgggtcttcc cggagtcggg ttgcttggga
  8221 atgcagccca aagcgggtgg taaactccat ctaaggctaa ataccggcac gagaccgata
  8281 gtcaacaagt accgtaaggg aaagttgaaa agaactttga agagagagtt caagagggcg
  8341 tgaaaccgtt aagaggtaaa cgggtggggt ccgcgcagtc cgcccggagg attcaacccg
  8401 gcggcgggtc cggccgtgtc ggcggcccgg cggatctttc ccgccccccg ttcctcccga
  8461 cccctccacc cgccctccct tcccccgccg cccctcctcc tcctccccgg agggggcggg
  8521 ctccggcggg tgcgggggtg ggcgggcggg gccgggggtg gggtcggcgg gggaccgtcc
  8581 cccgaccggc gaccggccgc cgccgggcgc atttccaccg cggcggtgcg ccgcgaccgg
  8641 ctccgggacg gctgggaagg cccggcgggg aaggtggctc ggggggcccc gtccgtccgt
  8701 ccgtcctcct cctcccccgt ctccgccccc cggccccgcg tcctccctcg ggagggcgcg
  8761 cgggicgggg cggcggcggc ggcggcggtg gcggcggcgg cgggggcggc gggaccgaaa
  8821 ccccccccga gtgttacagc ccccccggca gcagcactcg ccgaatcccg gggccgaggg
  8881 agcgagaccc gtcgccgcgc tctcccccct cccggcgccc acccccgcgg ggaatccccc
  8941 gcgagggggg tctcccccgc gggggcgcgc cggcgtctcc tcgtgggggg gccgggccac
  9001 ccctcccacg gcgcgaccgc tctcccaccc ctcctccccg cgcccccgcc ccggcgacgg
  9061 ggggggtgcc gcgcgcgggt cggggggcgg ggcggactgt ccccagtgcg ccccgggcgg
  9121 gtcgcgccgt cgggcccggg ggaggttctc tcggggccac gcgcgcgtcc cccgaagagg
  9181 gggacggcgg agcgagcgca cggggtcggc ggcgacgtcg gctacccacc cgacccgtct
  9241 tgaaacacgg accaaggagt ctaacacgtg cgcgagtcgg gggctcgcac gaaagccgcc
  9301 gtggcgcaat gaaggtgaag gccggcgcgc tcgccggccg aggtgggatc ccgaggcctc
  9361 tccagtccgc cgagggcgca ccaccggccc gtctcgcccg ccgcgccggg gaggtggagc
  9421 acgagcgcac gtgttaggac ccgaaagatg gtgaactatg cctgggcagg gcgaagccag
  9481 aggaaactct ggtggaggtc cgtagcggtc ctgacgtgca aatcggtcgt ccgacctggg
  9541 tataggggcg aaagactaat cgaaccatct agtagctggt tccctccgaa gtttccctca
  9601 ggatagctgg cgctctcgca gacccgacgc acccccgcca cgcagtttta tccggtaaag
  9661 cgaatgatta gaggtcttgg ggccgaaacg atctcaacct attctcaaac tttaaatggg
  9721 taagaagccc ggctcgctgg cgtggagccg ggcgtggaat gcgagtgcct agtgggccac
  9781 ttttggtaag cagaactggc gctgcgggat gaaccgaacg ccgggttaag gcgcccgatg
  9841 ccgacgctca tcagacccca gaaaaggtgt tggttgatat agacagcagg acggtggcca
  9901 tggaagtcgg aatccgctaa ggagtgtgta acaactcacc tgccgaatca actagccctg
  9961 aaaatggatg gcgctggagc gtcgggccca tacccggccg tcgccggcag tcgagagtgg
10021 acgggagcgg cgggggcggc gcgcgcgcgc gcgcgtgtgg tgtgcgtcgg agggcggcgg
10081 cggcggcggc ggcgggggtg tggggtcctt cccccgcccc cccccccacg cctcctcccc
10141 tcctcccgcc cacgccccgc tccccgcccc cggagccccg cggacgctac gccgcgacga
10201 gtaggagggc cgctgcggtg agccttgaag cctagggcgc gggcccgggt ggagccgccg
10261 caggtgcaga tcttggtggt agtagcaaat attcaaacga gaactttgaa ggccgaagtg
10321 gagaagggtt ccatgtgaac agcagttgaa catgggtcag tcggtcctga gagatgggcg
10381 agcgccgttc cgaagggacg ggcgatggcc tccgttgccc tcggccgatc gaaagggagt
10441 cgggttcaga tccccgaatc cggagtggcg gagatgggcg ccgcgaggcg tccagtgcgg
10501 taacgcgacc gatcccggag aagccggcgg gagccccggg gagagttctc ttttctttgt
10561 gaagggcagg gcgccctgga atgggttcgc cccgagagag gggcccgtgc cttggaaagc
10621 gtcgcggttc cggcggcgtc cggtgagctc tcgctggccc ttgaaaatcc gggggagagg
10681 gtgtaaatct cgcgccgggc cgtacccata tccgcagcag gtctccaagg tgaacagcct
10741 ctggcatgtt ggaacaatgt aggtaaggga agtcggcaag ccggatccgt aacttcggga
10801 taaggattgg ctctaagggc tgggtcggtc gggctggggc gcgaagcggg gctgggcgcg
10861 cgccgcggct ggacgaggcg cgcgcccccc ccacgcccgg ggcacccccc tcgcggccct
10921 cccccgcccc acccgcgcgc gccgctcgct ccctccccac cccgcgccct ctctctctct
10981 ctctcccccg ctccccgtcc tcccccctcc ccgggggagc gccgcgtggg ggcgcggcgg
11041 ggggagaagg gtcggggcgg caggggccgc gcggcggccg ccggggcggc cggcgggggc
11101 aggtccccgc gaggggggcc ccggggaccc ggggggccgg cggcggcgcg gactctggac
11161 gcgagccggg cccttcccgt ggatcgccac agctgcggcg ggcgtcgcgg ccgcccccgg
11221 ggagcccggc ggcggcgcgg cgcgcccccc acccccaccc cacgtctcgg tcgcgcgcgc
11281 gtccgctggg ggcgggagcg gtcgggcggc ggcggtcggc gggcggcggg gcggggcggt
11341 tcgtcccccc gccctacccc cccggccccg tccgcccccc gttcccccct cctcctcggc
11401 gcgcggcggc ggcggcggca ggcggcggag gggccgcggg ccggtccccc ccgccgggtc
11461 cgcccccggg gccgcggttc cgcgcgcgcc tcgcctcggc cggcgcctag cagccgactt
11521 agaactggtg cggaccaggg gaatccgact gtttaattaa aacaaagcat cgcgaaggcc
11581 cgcggcgggt gttgacgcga tgtgatttct gcccagtgct ctgaatgtca aagtgaagaa
11641 attcaatgaa gcgcgggtaa acggcgggag taactatgac tctcttaagg tagccaaatg
11701 cctcgtcatc taattagtga cgcgcatgaa tggatgaacg agattcccac tgtccctacc
11761 tactatccag cgaaaccaca gccaagggaa cgggcttggc ggaatcagcg gggaaagaag
11821 accctgttga gcttgactct agtctggcac ggtgaagaga catgagaggt gtagaataag
11881 tgggaggccc ccggcgcccc cccggtgtcc ccgcgagggg cccggggcgg ggtccgcggc
11941 cctgcgggcc gccggtgaaa taccactact ctgatcgttc tttcactgac ccggtgaggc
12001 gggggggcga gcccgagggg ctctcgcttc tggcgccaag cgcccgcccg gccgggcgcg
12061 acccgctccg gggacagtgc caggtgggga gtttgactgg ggcggtacac ctgtcaaacg
12121 gtaacgcagg tgtcctaagg cgagctcagg gaggacagaa acctcccgtg gagcagaagg
12181 gcaaaagctc gcttgatctt gattctcagt acgaatacag accgtgaaag cggggcctca
12241 cgatccttct gaccttttgg gttttaagca ggaggtgtca gaaaagttac cacagggata
12301 actggcttgt ggcggccaag cgttcatagc gacgtcgctt tttgatcctt cgatgtcggc
12361 tcttcctatc attgtgaagc agaactcgcc aagcgttgga ttgttcaccc actaataggg
12421 aacgtgagct gggtttagac cgtcgtgaga caggttagtt ttaccctact gatgatgtgt
12481 tgttgccatg gtaatcctgc tcagcacgag aggaaccgca ggttcagaca tttggtgtat
12541 gtgcttggct gaggagccaa tggggcgaag ctaccatctg tgggattatg actgaacgcc
12601 tctaagtcag aatcccgccc aggcgaacga tacggcagcg ccgcggagcc tcggttggcc
12661 tcggatagcc ggtcccccgc ctgtccccgc cggcgggccg cccccccctc cacgcgcccc
12721 gccgcgggag ggcgcgtgcc ccgccgcgcg ccgggaccgg ggtccggtgc ggagtgccct
12781 tcgtcctggg aaacggggcg cggccggaaa ggcggccgcc ccctcgcccg tcacgcaccg
12841 cacgttcgtg gggaacctgg cgctaaacca tccgtagacg acctgcttct gggtcggggt
12901 ttcgtacgta gcagagcagc tccctcgccg cgatccattg aaagtcagcc ctcgacacaa
12961 gggtttgtcc gcgcgcgcgt gcgtgcgggg ggcccggcgg gcgtgcgcgt tcggcgccgt
13021 ccgtcctccc gttcgtcttc ctccctcccg gcctcccccg ccgaccgcgg cgtggtggtg
13081 gggcgggggg gagggcgcgc gaccccggcc ggccgccccg cttcttcggt tcccgcctcc
13141 tccccgttca cgccggggcg gctcgtccgc tccgggccgg gacggggtcc ggggagcgtg
13201 gtttgggagc cgcggaggcg ccgcgccgag ccgggccccg tggcccgccg gtccccgtcc
13261 cgggggttgg ccgcgcggcg cggtgggggg ccacccgggg tcccggccct cgcgcgccct
13321 tccccctcgc tcctccgcac gggtcgaccg acgaaccgcg ggtggcgggc ggcgggcggc
13381 gagccccacg ggcgtccccg cacccggccg acctccgctc gcgacctctc ctcggtcggg
13441 cctccggggt cgaccgcctg cgcccgcggg cgtgagactc agcggcgtct cgccgtgtcc
13501 cgggtcgacc gcggccttct ccaccgagcg gcggtgtagg agtgcccgtc gggacgaacc
13561 gcaaccggag cgtccccgtc tcggtcggca cctccggggt cgaccagccg ccgcccgcga
13621 gctccggact tagccggcgt ctgcacgtgt cccgggtcga ccagcaggcg gccgccggac
13681 gcagcggcgc acgcacgcga gggcgtcgat tccccttcgc gcgcccgcgc ctccaccggc
13741 ctcggcccgc ggtggagctg ggaccacgcg gaactccctc tcccacattt ttttcagccc
13801 caccgcgagt ttgcgtccgc gggaccttta agagggagtc actgctgccg tcagccagta
13861 ctgcctcctc ctttttcgct tttaggtttt gcttgccttt tttttttttt tttttttttt
13921 ttttttcttt ctttctttct ttctttcttt ctttctttct ttctttcttt cgcttgtctt
13981 cttcttgtgt tctcttcttg ctcttcctct gtctgtctct ctctctctct ctctctctgt
14041 ctctcgctct cgccctctct ctcttctctc tctctctctc tctctctctg tctctcgctc
14101 tcgccctctc tctctctctt ctctctgtct ctctctctct ctctctctct ctctctctct
14161 gtcgctctcg ccctctcgct ctctctctgt ctctgtctgt gtctctctct ctccctccct
14221 ccctccctcc ctccctccct ccctcccctt ccttggcgcc ttctcggctc ttgagactta
14281 gccgctgtct cgccgtaccc cgggtcgacc ggcgggcctt ctccaccgag cggcgtgcca
14341 cagtgcccgt cgggacgagc cggacccgcc gcgtccccgt ctcggtcggc acctccgggg
14401 tcgaccagct gccgcccgcg agctccggac ttagccggcg tctgcacgtg tcccgggtcg
14461 accagcaggc ggccgccgga cgcagcggcg caccgacgga gggcgctgat tcccgttcac
14521 gcgcccgcgc ctccaccggc ctcggcccgc cgtggagctg ggaccacgcg gaactccctc
14581 tcctacattt ttttcagccc caccgcgagt ctgcgtccgc gggaccttta agagggagtc
14641 actgctgccg tcagccagta ctgcctcctc ctttttcgct tttaggtttt gcttgccttt
14761 ttctttcttt ctttcgctct cgctctctcg ctctctccct cgctcgtttc tttctttctc
14821 tttctctctc tctctctctc tctctctctc tctgtctctc gctctcgccc tctctctctc
14881 tttctctctc tctctgtctc tctctctctc tctctctctc tctctctctc cctccctccc
14941 tccccctccc tccctctctc cccttccttg gcgccttctc ggctcttgag acttagccgc
15001 tgtctcgccg tgtcccgggt cgaccggcgg gccttctcca ccgagcggcg tgccacagtg
15061 cccgtcggga cgagccggac ccgccgcgtc cccgtctcgg tcggcacctc cggggtcgac
15121 cagctgccgc ccgcgagctc cggacttagc cggcgtctgc acgtgtcccg ggtcgaccag
15181 caggcggccg ccggacgctg cggcgcaccg acgcgagggc gtcgattccg gttcacgcgc
15241 cggcgacctc caccggcctc ggcccgcggt ggagctggga ccacgcggaa ctccctctcc
15301 cacatttttt tcagccccac cgcgagtttg cgtccgcggg acttttaaga gggagtcact
15361 gctgccgtca gccagtaatg cttcctcctt ttttgctttt tggttttgcc ttgcgttttc
15481 tctctgtctc tctctctctg tctctctctc ctccctccct ccttggtgcc ttctcggctc
15541 gctgctgctg ctgcctctgc ctccacggtt caagcaaaca gcaagttttc tatttcgagt
15601 aaagacgtaa tttcaccatt ttggccgggc tggtctcgaa ctcccgacct agtgatccgc
15661 ccgcctcggc ctcccaaaga ctgctgggag tacagatgtg agccaccatg cccggccgat
15721 tccttccttt tttcaatctt attttctgaa cgctgccgtg tatgaacata catctacaca
15781 cacacacaca cacacacaca cacacacaca cacacacaca cacacacccc gtagtgataa
15841 aactatgtaa atgatatttc cataattaat acgtttatat tatgttactt ttaatggatg
15901 aacatgcatc gaagccccat ttcatttaca cacacgtgta tgtatatcct tcctcccttc
15961 ctccatccat tatttattaa taattttcgt ttatttatct tcttttcttt tggggccggc
16021 ccgcctggtc ttctgtctct gcgctctggt gacctcagcc tcccaaatag ctgggactac
16081 agggatctct taagcccggg aggagaggtt aacgtgggct gtgatcgcac acttccactc
16141 cagcttacgt gggctgcggt gcggtggggt ggggtggggt ggggtggggt gcagagaaaa
16201 cgattgattg cgatctcaat tgccttttag cttcattcat accctgttat ttgctcgttt
16261 attctcatgg gttcttctgt gtcattgtca cgttcatcgt ttgcttgcct gcttgcctgt
16321 ttatttcctt ccttccttcc ttccttcctt ccttccttcc ttccttcctt ccctccctta
16381 ctggcagggt cttcctctgt ctctgccgcc caggatcacc ccaacctcaa cgctttggac
16441 cgaccaaacg gtcgttctgc ctctgatccc tcccatcccc attacctgag actacaggcg
16501 cgcaccacca caccggctga cttttatgtt gtttctcacg ttttccgtag gtaggtatgt
16561 gtgtgtgtgt gtgtgtgtgt gtgtgtgtgt gtgtgtgtgt gtgtgtgtgt gtgtgtatct
16621 atgtatgtac gtatgtatgt atgtatgtga gtgagatggg tttcggggtt ctatcatgtt
16681 gcccacgctg gtctcgaact cctgtcctca agcaatccgc ctgcctgcct cggccgccca
16741 cactgccgct attacaggcg tgagacgctg cgcctggctc cttctacatt tgcctgcctg
16801 cctgcctgcc tgcctgccta tcaatcgtct cctttttagt acggatgtcg tctcgcttta
16861 ttgtccatgc tctgggcaca cgtggtctct tttcaaacct ctatgattat tattattgta
16921 ggcgtcatct cacgtgtcga ggtgatctcg aacttttagg ctccagagat cctcccgcat
16981 cggcctcccg gagtgctgtg atgacacgcg tgggcacggt acgctctggt cgtgtttgtc
17041 gtgggtcggt tctttccgtt tttaatacgg ggaccgcgaa cgaagaaaat tttcagacgc
17101 atctcaccga tccgcctttt cgttcttcct ctttattccc tttagacgga gtttcactct
17161 tgccgcccag ggtggagtac gatggcggct ctcggctcac cgcaccctcc gcctcccagg
17221 ttcaagtgat tctcctgcct cagccttccc gagtagctgg aatgacagag acgagccatc
17281 gtgcccggct aatttttcta tttttagcac agatggggtt tctccatctt ggtcaggctg
17341 gtcttcaact tccgaccgtt ggagaatctt aactttcttg gtggtggttg ttttcctttc
17401 tctttttttt tcttttcttt tctttccttc tcctcccccc cccacccccc ttgtcgtcgt
17461 cctcctcctc ctcctcctcc tcctcctcct cctcctcctc ctcctcctcc tctttcattt
17521 ctttcagctg ggctctccta cttgtgttgc tctgttgctc acgctggtct caaactcctg
17581 gccttgactc ttctcccgtc acatccgccg tctggttgtt gaaatgagca tctctcgtaa
17641 aatggaaaag atgaaagaaa taaacacgaa gacggaaagc aeggtgtgaa cgtttctctt
17701 gccgtctccc ggggtgtacc ttggacccgg aaacacggag ggagcttggc tgagtgggtt
17761 ttcggtgccg aaacctcccg agggcctcct tccctctccc ccttgtcccc gcttctccgc
17821 cagccgaggc tcccaccgcc gcccctggca ttttccatag gagaggtatg ggagaggact
17881 gacacgcctt ccagatctat atcctgccgg acgtatctgg ctcggcgtgc cccaccggct
17941 acctgccacc ttccagggag ctctgaggcg gatgcgaccc caaccccccc gtcacgtccc
18001 gctaccctcc cccggctggc ctttgccggg cgaccccagg ggaaccgcgt tgatgctgct
18061 tcggatcctc cggcgaagac ttccaccgga tgccccgggt gggccggttg ggatcagact
18121 ggaccacccc ggaccgtgct gttcttgggg gtgggttgac gtacagggtg gactggcagc
18181 cccagcattg taaagggtgc gtgggtatgg aaatgtcacc taggatgccc tccttccctt
18241 cggtctgcct tcagctgcct caggcgtgaa gacaacttcc catcggaacc tcttctcttc
18301 cctttctcca gcacacagat gagacgcacg agagggagaa acagctcaat agataccgct
18361 gaccttcatt tgtggaatcc tcagtcatcg acacacaaga caggtgacta ggcagggaca
18421 cagatcaaac actatttccg ggtcctcgtg gtgggattgg tctctctctc tctctctctc
18481 tctctctctc tctctctctc tctcgcacgc gcacgcgcgc acacacacac acaatttcca
18541 tatctagttc acagagcaca ctcacttccc cttttcacag tacgcaggct gagtaaaacg
18601 cgccccaccc tccacccgtt ggctgacgaa accccttctc tacaattgat gaaaaagatg
18661 atctgggccg ggcacgctag ctcacgcctg tcactccggc actttgggag gccgaggcgg
18721 gtggatcgct tggggccggg agtccgagac caggctggcc gacgtggcga aaccccgtct
18781 ctctgaaaaa tagaacgatt agccgggcct ggtggcgtgg gcttggaacc acgaccgctc
18841 gggagactgg ggcgggcgac ttgctccaac cggggaggcc gaggccgcga tgagctgaga
18901 tcgtgccgtg gcgatgcggc ctggatgacg gagcgagacc ccgtctcgag agaatcatga
18961 tgttattata agatgagttg tgcgcggcga tggccgcctg tagtcgcggc tactcgggag
19021 gctgagacga ggagaagatc actcgaggcc ccacaggtcg aggcttcggt cggccgtgac
19081 ccactgtatc ctgggcagtc accggtcaag gagatatgcc ccttccccgt tcgcttttct
19141 ttccttccct tctcttttct tctttttgct tctcttttct ttctttcttt ccttctttct
19201 ttctttcttt ctttctttct ttttcttttt ctctcttccc ctctttcttt cctgccttcc
19261 tgcctttctt cttttcttct ttcctccctt cctcccttcc ttctttcctc ccgcctcagc
19321 ctcccaaagt gctgggatga ctggcgggag gcaccatgcc tgcttggccc aaagagaccc
19381 tcctggaaag tgagacgcag agagcgcctt ccagtgatct cattgactga tttagagacg
19441 gcatctcgct ccgtcacccc ggcagtggtg ccgtcgtaac tcactccctg cagcgcggac
19501 gctcctggac tcgagcgatc cttccacctc agcctccaga gtacagagcc tgggaccgcg
19561 ggcacgcgcc actgtgccca caccgttttt aattgttttt ttttcccccg agacagagtt
19621 tcactctcgt ggcctagact gcagtgcggt ggcgcgatct tggctcaccg caacctctgc
19681 ctcccggttt caagcgattc tcctgcatcg gcctcctgag tagccgggat tgcgggcatg
19741 cgctgccacg tctggctgat ttcgtatctt cagtggagac ggggcttctc catgtcgatc
19801 gggctggttt cgaactcccg acctcaggtg atccgccctc cccggcctcc ggaagtgctg
19861 ggatgacagg cgtgagccac cgcgcccggc cttcattttt aaatgttttc ccacagacgg
19921 ggtctcatca tttctttgca accctcccgc ccggcgtccc aaagtgctgg cgtgacgggc
19981 gtgagccact gcgcctggac tccggggaat gactcacgac caccatcgct ctactgatcc
20041 tttcttcctt tctttctttc tttctttctt tctttctttc tttctttctt tctttcttga
20101 tgaattatct tatgatttat ttgtgtactt atttccagac ggagtctcgc tctgggcggg
20161 gcgaggcgag gcgaggcaca gcgcatcgct ttggaagccg cggcaacgcc tttcaaagcc
20221 ccattcgtat gcacagagcc ttattccctt cctggagttg gagctgatgc cttccgtagc
20281 cttgggcttc tctccattcg gaagcttgac aggcgcaggg ccacccagag gctggctgcg
20341 gctgaggatt agggggtgtg ttggggctga aaaccgggtc ccctattttt gatacctcag
20401 ccgacacatc ccccgaccgc catcgctcgc tcgccctctg agatcccccg cctccaccgc
20461 cttgcaggct cacctcttac tttcattcct tcctctcttg cgtttgagga gggggtgcgg
20521 gaatgagggt gtgtgtgggg agggggtgcg gggtggggac ggaggggagc gtcctaaggg
20581 tcgattcagt gtcatgcctc tttcaccacc accaccacca ccgaagatga cagcaaggat
20641 cggctaaata ccgcgtgttc tcatctagaa gtgggaactt acagatgaca gttcttgcat
20701 gggcagaacg agggggaccg gggacgcgga agtctgctcg agggaggagg ggtggaagga
20761 gagacagctt caggaagaaa acaaaacacg aatactgtcg gacacagcac tgactacccg
20821 ggtgatgaaa tcatctgcac actgaacacc cccgtcacaa gtttacctat gtcacaatct
20881 tgcacacgta tcgcttgaac gacaaataaa agttaggggg gagaagagag gagagagaga
20941 gagagagaga gacagagaga gacagagaga gagagagagg agggagagag gaaaacgaaa
21001 caccacctcc ttgacctgag tcagggggtt tctggccttt tgggagaacg ttcagcgaca
21061 atgcagcatt tgggcccgtt cttttttttt cttcttcttt tctttctttt tttttggact
21121 gagtctctct cgctctgtca cccaggccgc ggtcgcggcg gcgctctctc ggctcactga
21181 aacctccgct tcccgggttc cagtgatcct tcttcggtag ctgggattac aggcgcacac
21241 cacgacggcg ggctcatatt cctatttcca gtagagacgg ggtttctcca cgttggccac
21301 gccggtctcg aactcctgac ctcaaatgat ccgccttcct gggcctccca aagtgctgga
21361 aacgacaggc ctgagccgcc gggatttcag cctttaaaag cgcggccctg ccacctttcg
21421 ctgtggccct tacgctcaga atgacgtgtc ctctctgccg taggttgact ccttgagtcc
21481 cctaggccat tgcactgtag cctgggcagc aagagccaaa ctccgnnccc ceacetcoto
21541 gcgcacataa taactaacta acaaactaac taactaacta aactaactaa ctaactaaaa
21601 tctctacacg tcacccataa gtgtgtgttc ccgtgagagt gatttctaag aaatggtact
21661 gtacactgaa cgcagtggct cacgtctgtc atcccgaggt caggagttcg agacoagooc
21721 ggccaacgtg gtgaaacccc gtctctactg aaaatacgaa atggagtcag gcgccgtggg
21781 gcaggcacct gtaaccccag ctactcggga ggctggggtg gaagaattgc ttgaacctgg
21841 caggcggagg ctgcagtgac ccaagatcgc accactgcac tacagcctgg gcgacagagt
21901 gagacccggt ctccagataa atacgtacat aaataaatac acacatacat acatacatac
21961 atacatacat acatacatac atccatgcat acagatatac aagaaagaaa aaaagaaaag
22021 aaaagaaaga gaaaatgaaa gaaaaggcac tgtattgcta ctgggctagg gccttctctc
22081 tgtctgcttc tctctgttcg tctctgtctt tctctctgcg tctctttctc tgtctgtctg
22141 tctcttcctt tctctctgtc tctgtctctg tcttcgtccc tctctctccc tctctgcctg
22201 tctcactgtg tctgtcttct gtcttactct ctttctctcc ccgtctgtct ctctctctcc
22261 ctctccctcc ctgcttgttt ctctctctcc ctccctgtct gtttctctct ctctctttct
22321 gtctgtttct gtccctctct gtctgtctat gtctttctct gtctgtctct ttctctgtct
22381 gtctgcctct ctccttcttt ttctgtgtct ctctgtcggt ctctctctct ctgtctgtct
22441 gtctgtctct ctctctctct ctctgtgcct atcttctgtc ttactctctt tctctgcctg
22501 tctgtctgtc tctccctccc tttctgtttc tctctctctc tctctctctc tccccctctc
22561 cctgtccgtt tctctccgtc tctctctctt tctgtctgct tctcactgtc tctctctgtc
22621 catctctctc tctctctgtc tgtctctttc gttctctctg tctgtctgtc tctctctctc
22681 tctctctctc tctctctctc tccctgtctg tctgtttctc tctatctctc gctgtccatc
22741 tctgtccttc tatgtctgtc tctttctctg tcagtctgtc agacaccccc gtgccgggta
22801 gggccccgcc cctcccacga aagtgagaag cgcgtgcttc ggtgcttaga gaggccgaga
22861 ggaatctaga caggcgggcc ttgctgggct tccccactcg gtgtatgatt tcgggaggtc
22921 gaggccgggt ccccgcttgg atgcgagggg cattttcaga cttttctctc ggtcacgtgc
22981 ggcgtccgta cttctcctat ctccccgata agctcctcga cttcaacata aacggcgtcc
23041 taagggtcga tttagtgtca tgcctctttc accgccacca ccgaagatga aagcaaagat
23101 cggctaaata ccgcgtgttc tcatctagaa gtgggaactt acagatgaca gttcttgcat
23161 gggcagaacg agggggaccg ggnacgcgga agcctgcttg agggrggagg ggyggaagga
23221 gagacagctt caggaagaaa acaaaacacg aatactgtcg gacacagcac tgactacccg
23281 ggtgatgaaa tcatctgcac actgaacacc cccgtcacaa gcttacctat gtcacagtct
23341 tgctcatgta tgcttgaacg acaaataaaa gttcgggggg gagaagagag gagagagaga
23401 gagagacggg gagagagggg ggagaggggg ggggagagag agagagagag agagagagag
23461 agagagagag agaaagagaa gtaaaaccaa ccaccacctc cttgacctga gtcagggggt
23521 ttctggcctt ttgggagaac gttcagcgac aatgcagtat ttgggcccgt tctttttttc
23581 ttcttcttct tttctttctt cttttttgga ctgagtctct ctcgctctgt cacccaggct
23641 gcggtgcggt ggcgctctct cggcccactg aaacctctgc ttcccgggtt ccagtgattc
23701 ttcttcggta gctgggatta caggtgcgca ccatgacggc cggctcatcg ttctattttt
23761 agtagagacg gggtttctcc acgttggcca cgctggtctc gaactcctga ccacaaatga
23821 tccaccttcc tgggcctccc aaagtgctgg aaacgacagg cctgagccgc cgggatttca
23881 gcctttaaaa gcgcgcggcc ctgccacctt tcgctgcggc ccttacgctc agaatgacgt
23941 gtcctctctg ccataggttg actccttgag tcccctaggc cattgcactg tagcctgggc
24001 agcaagagcc aaactccgtc cccccacctc cccgcgcaca taataactaa ctaactaact
24061 aactaactaa aatctctaca cgtcacccat aagtgtgtgt tcccgtgagg agcgatttct
24121 aagaaatggt actgtacact gaacgcaggc ttcacgtctg tcatcccgag gtcaggagtt
24181 cgagaccagc ccggcccacg tggtgaaacc cccgtctcta ctgaaaatac gaaatggagt
24241 caggcgccgt ggggcaggca cctgtaaccc cagctactcg ggaggctggg gtggaagaat
24301 tgcctgaacc tggcaggcgg aggctgcagt gacccaagat cgcaccactg cactacagcc
24361 tgggcgacag agtgagaccc ggtctccaga taaatacgta cataaataaa tacacacata
24421 catacataca tacatacaac atacatacat acagacatac aagaaagaaa aaaagaaaag
24481 aaaagaaaga gaaaatgaaa gaaaaggcac tgtatcgcta ctgggctagg gccttctctc
24541 tgtctgtttc tctctgttcg tctccgtctt tctctctgtg tctctttctc tgtctgtctg
24601 tctgtctgtc tgtctgtctc tttctttctt tctgtctctg tctttgtccc tctctctccc
24661 tctctgccct gtctcactgt gtctgtcttc tatctcactc tctttctctc cccgtctgtc
24721 tctctctcac tccctccctg tctgcttctc tctctctctc tttctgtctg tttctgtctc
24781 tctctgtctg cctctctctt tctctatctg tctctctctc tgtctgtctg cccctctctt
24841 tctttttctg tgtctctctg tctgcctctc tctctctctg tgcctatctt ctgtcttact
24901 ctctttctct gcctgtctgt ctgtctctct ctgtccctcc ctccctttct gcttctctct
24961 ctctctctct ctctnnnccc tccctgtctg tttctctctg tctccctctc tttctgtctg
25321 tttctcactg tctctctctg tctgcctgtt tcattctctc tgtctctgtc tctgtctctc
25081 tctctctctg tctctccctc tctgtgtgta tcttttgtct tactctcctt ctctgcctgt
25141 ccgtctgtct gtctgtctct ctctctccct gtccctctct ctttctgtct gtttctctct
25201 ctctctctct ctctctctct ctgtctctgt ctttctctgt ctgtcccttt ctctgtctgt
25261 ctgcctctct ctttctcttt ctgtgtctct ctgtccctct ctctgtgcct atcttctgtc
25321 ttactctctt tctctgcctg tctatctgtc tgtctctctc tgtctctctc cctgcctttc
25381 tgtttctctc tctctccctc tctcgctctc tctgtctttc tctctttctc tctgtttctc
25501 gtctgtctct gtctctgtct ctgtctctct ctctctctct ctccttgtct ccctcactgt
25561 gtctgtcttc tgtcttactc tccttctctg cctgtccatc tgtctgtctg tctctctctc
25621 tctctcccta cctttctgtt tctctctcgc tagctctctc tctctctgcc tgtttctctc
25741 tctctgtctc tgtctctctc tctctctctc tctctctctc tgcctctctc actgtgtctg
25801 tcttctgtct tattctcttt ctctctctgt ctctctctct ctctccttta ctgtctgttt
25861 ctctctctct ctctctcttt ctgcctgttt ctctctgtct gtctctgtct ttctctgtct
25921 gtctgcctct ctctttcttt ttctgcgtct ctctgtctct ctctctctct ctctgttcct
25981 atcttctgtc ttactctgtt tccttgcctg cctgcctgtc tgtgtgtctg tctctctctc
26041 tctctctctc tctctctccc tccctttctc tttctctgtc tctctatctc tttctgggtg
26101 tttctctctg tctctctgtc catctctgtc tttctatgtc tgtctctctc tttctctctg
26161 tctctgtctc tgcctctctc tctctctctc tctctctctc tctgtctgtc tctctcactg
26221 tgtgtgtctg tcttctgtct tactctcctt ctctgcctgt ccgtctgtct gtctgtctct
26341 ctctctctgt ctgtctcttt ctctgtctgt ctgtctctct ctttcttttt ctctgtctct
26401 ctgtctctct ctgtgtctgt ctctctgtct gtgcctatct tctgtcttac tctctttctc
26461 tggctgtctg cctgtctctc tctctctctc tgtctgtctc cgtccctctc tccctgtctg
26521 tctgtttctc tctctgcctc tctctctctc tgtctgtctc tttctctgtc tgtctgtctc
26641 tcttctgtct tactctcttt ctctggctgt ctgcctgtct ctctctctct gcctgtctcc
26701 gtccctccct ccctgtctgt ctgtttctct ctctgtctct gtctctctgt ccatctctgt
26761 ctgtctcttt ctctttctct ctctctgtct ctgtctctct ctctctctgc ctgtctctct
26821 cactgtgtct gtcttctgtc ttactctctt tctcttgcct gcctctctgt ctgtctgtct
26881 ctctccctcc atgtctctct ctctctctca ctcactctct ctccgtctct ctctctttct
26941 gtctgtttct ctctctgtct gtctctctcc ctccatgtct ctctctctct ctctcactca
27001 ctctctctcc gtctctctct ctctttctgt ctgtttctct ctctgtctgt cictctccct
27061 ccatgtctct ctctctccct ctcactcact ctctctccgt ctctctctct ctttctgtct
27121 gtttctttgt ctgtctgtct gtctgtctgt ctgtctctct ctctctctct ctctctctct
27181 ctctctgttt gtctttctcc ctccctgtct gtctgtctgt ctctctctct ctgtctctgt
27241 ctctgtctct ctctctttct ctttctgtct gtttctctct atctctcgct gtccatctct
27301 gtctttctat gtctgtctct ttctctgtca gtctgtcaga cacacccgtg ccggtagggc
27361 cctgcccttc cacgagagtg agaagcgcgt gcttcggtgc ttagagaggc cgagaggaat
27421 ctagacaggc gggccttgct gggcttcccc actcggtgta cgatttcggg aggtcgaggc
27481 cgggtccccg cttggatgcg aggggcattt tcagactttt ctctcggtca cgtgtggcgt
27541 ccgtacttct cctatttccc cgataagtct cctcgacttc aacataaact gttaaggccg
27601 gacgccaaca cggcgaaacc ccgtctctac taaaaataca aagctgagtc gggagcggtg
27661 gggcaggccc tgtaatgcca gctcctcggg aggctgaggc gggagaatcg cttgaaccag
27721 ggaagcggag gctgcaggga gccgagatcg cgccactgca ctacggccca ggctgtagag
27781 tgagtgagac tcggtctcta aataaatacg gaaattaatt aattcattaa ttcttttccc
27841 tgctgacgga catttgcagg caggcatcgg ttgtcttcgg gcatcaccta gcggccactg
27901 ttattgaaag tcgacgttga cacggaggga ggtctcgccg acttcaccga gcctggggca
27961 acgggtttct ctctctccct tctggaggcc cctccctatc tccctcgttg cctagggaac
28021 ctcgcctagg gaacctccgc cctgggggcc ctattgttct ttgatcggcg ctttactttt
28081 ctttgtgttt tggcgcctag actcttctac ttgggctttg ggaagggtca gtttaatttt
28141 caagttgccc cccggctccc cccactaccc acgtcccttc accttaattt agtgagncgg
28201 ttaggtgggt ttcccccaaa ccgccccccc ccccccgcct cccaacaccc tgcttggaaa
28261 ccttccagag ccaccccggt gtgcctccgt cttctctccc cttcccccac cccttgccgg
28321 cgatctcatt cttgccaggc tgacatttgc atcggtgggc gtcaggcctc actcgggggc
28381 caccgttttt gaagatgggg gcggcacggt cccacttccc cggaggcagc ttgggccgat
28441 ggcatagccc cttgacccgc gtgggcaagc gggcgggtct gcagttgtga ggcttttccc
28501 cccgctgctt cccgctcagg cctccctccc taggaaagct tcaccctggc tgggtctcgg
28561 tcacctttta tcacgatgtt ttagtttctc cgccctccgg ccagcagagt ttcacaatgc
28621 gaagggcgcc acggctctag tctgggcctt ctcagtactt gcccaaaata gaaacgcttt
28681 ctgaaaacta ataactttnc tcacttaaga tttccaggga cggcgccttg gcccgtgttt
28801 tttcttttca ggtgaagtag aaatccccag ttttcaggaa gacgtctatt ttccccaaga
28861 cacgttagct gccgtttttt cctgttgtga actagcgctt ttgtgactct ctcaacgctg
28921 cagtgagagc cggttgatgt ttacnatcct tcatcatgac atcttatttt ctagaaatcc
28981 gtaggcgaat gctgctgctg ctcttgttgc tgttgttgtt gttgttgttg tcgtcgttgc
29041 tgttgtcgtt gtcgttgttg ttgtcgttgt cgttgttttc aaagtatacc ccggccaccg
29101 tttatgggat caaaagcatt ataaaatatg tgtgattatt tcttgagcac gcccttcctc
29161 cccctatctc tgtctctctg tctgtctctg tctctctctt tctctgtctg tcttctctct
29221 ctctctctct ctgtgcctct ctctctctgc ctgtctgttt ctctctctct gcctctctct
29281 ctctctctct ctctgcctgt ctctctcact gtgtctgtct tctgtctcac tccctttctc
29341 tgtctgtctg tcggtctctc tctctctctc tccctgtctg tatgtttctc tctgtctctg
29401 tctctctctc tetttctgtt tctctccctc cgtctctgtc tttctctgac tgtctctctc
29461 tttccttctc tctgtctctc tctgcctgtc tctctcactc tgtcttctgt cttatctctc
29521 tctctgcctg cctgtctctc tcactctctc tctctgtgtg tctctctctc tctttctgtt
29581 tctctctgtc tctctgtccg tctctgtctt tctctgtctg tctctttgtc tgtctgtctt
29641 tgtctttcct tctctctgtc tctgtctctc tcactgtgtc tgtcttctgt cttagtctct
29701 ctctctctct ctccctgtct gtctgtctct ctctctctct ccccctgtct gtttctctct
29761 ctctctctct ctctctctct ctctgtcttt gtctttcttt ctgtctctgt ctctctctct
29821 ctctctgtgt gtctgtcttc tgtcttactg tctttctctg cctgtctgtc tgtctgtctc
29881 tctctgtctg tctctctctc tctctccccc tgtcggctgt ttctctgtct ctgtctgtgt
29941 ctctctttct gtctgtttct ctctgtctgt ctttctctct ctgtctcttt ctctctgtct
30001 ctctgtctgt ctctgtctct ctctctgtct ctctctctct gtgggggtgt gtgtgtgtgt
30061 gtgtatgtgt gtgtgtgtgt gtgtgtgtgt ctgccttctg tcttactcta tttctctgcc
30121 tgtctgtctg cctgtctgtt tgtctctctc tctctgcctg tctctctccc ttcctgtctg
30181 tttctctctc tttctgtttc tctctgtctc tgtccatctc tgtctttctc cgtctgtctc
30241 tttatctgtc tctctccgtc tgtctcttta tctgtctctc tctctctttc tgtctttctc
30301 tccctgtgta tcgttgtctc tctctgtctg tctctgtctc tgtctctctg tctctctctc
30361 tctctctctc tctctgtctg tctgtccgtc tgtctgtctc ggtctctgcg tctcgctatc
30421 tcccgccctc tctttttttg caaaagaagc ccaagtacat ctaatctaat cccttaccaa
30481 ggcctgaatt cttcacttct gacatcccag atttgatctc cctacagaat gctgtacaga
30541 actggcgagt tgatttctgg acttggacac ctcatagaaa ctacatatga ataaagatcc
30601 aatcctaaaa tctggggtgg cttctccctc gactgtctcg aaaaatcgta cctctgttcc
30661 ccuaggatgc cggaagagtt ttctcaacgt gcatctgccc gtgtcctaag tgatctgtga
30721 ccgagccctg tccgtcctgt ctcaaatatg tacgtgcaaa cacttctctc catttccaca
30781 actacccacg gccccttgtg gaaccactgg ctctttgaaa aaaatcccag aagtggtttt
30841 ggctttctgg ctaggaggcc taagcctgct gagaactttc ctgcccagga tcctcgggac
30901 catgcttgct agcgctggat gagtctctgg aaggacgcac gggactccgc aaagctgacc
30961 tgtcccaccg aggtcaaatg gatacctctg cattggcccg aggcctccga agtacatcac
31021 cgtcaccaac cgtcaccgtc agcatccttg tgagcctgcc caaggccccg cctccgggga
31081 gactcttggg agcccggcct tcgtcggcta aagtccaaag ggatggtgac ttccacccac
31141 aaggtcccac tgaacggcga agatgtggag cgtaggtcag agaggggacc aggaggggag
31201 acgtcccgac aggcgacgag ttcccaaggc tctggccacc ccacccacgc cccacgcccc
31261 acgtcccggg cacccgcggg acaccgccgc tttatcccct cctctgtcca cagccggccc
31321 caceccacca cgcaacccac gcacacacgc tggaggttcc aaaaccacac ggtgtgacta
31381 gagcctgacg gagcgagagc ccatttcacg aggtgggagg ggtgggggtg gggtgggttg
31441 ggggttgtgg ggtctgtggc gagcccgatt ctccctcttg ggtggctaca ggctagaaac
31501 gaatatcgct tcttgggggg aggggcttcc ttaggccatc accgcttgcg ggactacctc
31561 tcaaaccctc ccttgaggcc acaaaataga ttccacccca cccatcgacg tttcccccgg
31621 gtgctggatg tatcctgtca agagacctga gcctgacacc gtcgaattaa acaccttgac
31681 tggctttgtg tgtttgtttg tttctgagat ggagtcttgc tctgtccccc aggctggagt
31741 gcagtggcgt gatctcagct cactggaacc cctgcctcct gggttcaagt gattctcctg
31801 tctcagcgcc accatggccg gctcattttt tttttttttt tttttggtag acacggggtt
31861 tcaccccctt tcattggttt tcactggaga ttctagattc gagccacacc tcattccgtg
31921 coacagagag acttcttttt tttttttttt tttttaagcg caacgcaaca tgtctgcctt
31981 atttgagtgg cttcctatat cattataatt gtgttataga tgaagaaacg gtattaaaca
32041 ctgtgctaat gatagtgaaa gtgaagacaa aagaaaggct atctattttg tggttagaat
32101 aaagttgctc agtatttaga agctacctaa atacgtcagc atttacactc ttcctagtaa
32161 aagctggccg atctgaataa tcctccttta aacaaacaca atttttgata gggttaagat
32221 ttttttaaga atgcgactcc tgcaaaatag ctgaacagac gatacacatt taaaaaaata
32281 acaacacaag gatcaaccag acttgggaaa aaatcgaaaa ccacacaagt cttatgaaga
32341 actgagttct taaaatagga cggagaacgt agctatcgga agagaaggca gtattggcaa
32401 gttgattgtt acgttggtca gcagtagctg gcaccatctt tttggccatc tttcgggcaa
32461 tgtaactact acagcaaaat gagatatgat ccattaaaca acatattcgc aaatcaaaaa
32521 gtgtttcagt aatataatgc ttcagattta gaagcaaatc aaatgataga actccactgc
32581 tgtaataagt caccccaaag atcaccgtat ctgacaaaat aactaccaca gggttatgac
32641 ttcagaatca tactttcttc ttgatatcta cttatgtatt tatttttttt aatttatttc
32701 tcttgagacg cgtctcgctc tgtcgcccag gctggagtgc gatggtgtga tctcggctca
32761 ctgcaaccgc cacctccctg ggttcaagcg attctcctgc ctcagcctcc cgagtagctg
32821 ggactacagg tgcccgccac cacgcccagc taatctttat acttttaata gagacggggt
32881 ttcaccgtgt cggcccggat ggtctcgatc tcttgacctc gtgacccgcc cgcctcggcc
32941 tcccaaagtg ctgggatgac aggcgtgagc cactgagccc ggccttctct tgacgcttaa
33001 actatgaagt cagtccagag aaacgcaata aatgtcaacg gtgaggatgg tgttgaggca
33061 gaagtaggac cacacttttt cctatcttat tcagttgata acaatatgac ctaggcagta
33121 atttcctatg tgcctactta tacacgagta caaaagagta aaacagagag actgctaaat
33181 taaagggtac gtgaagttct tcacagtaac cccgtaaact ggaacactgt caaaaagcag
33241 cagctagtga attgtttcca tgtatttttc tattatccaa taagtgaact atgctattcc
33301 tttccagtct cccaagcact tctcgtcccc atcaccactt cggtgctcga agaaaaagta
33361 agcaaatcaa ggaacacaag ctaaagaaac acacacacaa accaaagaca actacagcgt
33421 ctgcaaaagt ttgctagaag actgaaactg ttgagtataa ggatctggta ttctacgatc
33481 atgagttcac ttcagagttt gttcaagaca tacgtttcgt aaggaaacat cttagttaga
33541 agttattcag cagtaggtac catccctaag tatttttcac caaatccgtg acaataaaga
33601 gctatctaac cagaaaaatt agcgagtacg ggcaccatcc atagggcttt gtctttacgc
33661 ttcattagca cttaccatgc cttacaatgt ctaggattga ccctgatagc atttcgaaaa
33721 caagctaatg ctttgtccag ttcttcagtg aagacaactc acgccctaat gcgctatagg
33781 cataagcatc atttggatcc acttcgagag ttctctggaa gaattgaatc gcaatatcgt
33841 gttcccgttt gcagaccgaa acagtttccc tgcagcacac caggcctctg gctggcgaat
33901 ttttatccat gtctgtgaag tctttggaca gaactgaaag agcaacctct ttcggaggat
33961 gccaaagtgt tgtagagtag atctccatgc cttcgactct gtaattctca atcctcctaa
34021 cctctgagaa ttgtctttca gcttgcgtgg actctgaaag tttacaatag gccntttccg
34081 atttggcaca gtacccaacc ggtattgcag tggtgagaag ctagatggct caagatgctg
34141 atagcttctt tgccgtggta agaacacaaa gctaaataac ctttccccct ttcacgaaga
34201 aggctcatca agccttccgc tgctgctttt tgtagattaa aagcctgaat ctgaggcgcg
34261 attgcggcta ttttcccttc tgaaatgacg gaagagtcca attttgtcac ttccaggcta
34381 gggattttag gtggaaactt catttttaat tttctcctaa ttctcctcgg ttgtggagct
34441 gtcactagtc aagagtcgtg aatttcttcg aggncggtgc atttggggga gatgccatag
34501 tggggctcaa tacctgaggt gttgcccttg tcggcggacc agaactttgt gtttttgcaa
34561 ggactggagt tacctttcgg ctctttcccc tctgcgagaa gacagacggt gttccggttt
34621 ggccgattct ggcaacaggc ttttctgaag gggctccggt ggatggcacg tcagtgacag
34681 acggtgtctc ataccagtgc agttttgtca atagggtccg cctccgggac ttggggtttc
34741 taatggcaaa atgccaacac ttggggttaa tggactaaca gctgctggtc ctcctaataa
34801 acttcgacca gtttttggtt tatgttgaac ctgtttagat catatggaag ttcctgttcc
34861 cagtgggaca gtatcaggtg aaaggacagc tgaatcgata gaagacactg gggagtctgt
34921 attcaaggag tactttgaat tggaagattc taaattccat ccgtttcatt cgacggtgtc
34981 ctggggtgtt tccgtaagaa cggtctcggg ctgtctgtga cataaactag gacgaggtcc
35041 aagtgttgtg gcgcaacact tggacaggca gttgctaaag ctctctagag aggtgaatca
35101 aaatgtttgg tcaggatctg gcttttcccc cctatttcac atcatgattc aaagggacac
35161 cagaggaaag gatttcaacg aaggctcttt tggtcacatt ctgatccttt ggtaagccga
35221 tctgtcttgc aatatacatg tcccgacgat ggaaggggaa agcgagctga atcaccaaac
35281 tcaggaacga taatatcatc gtggcttttc tgcttatgaa acactccacc cgataagatt
35341 tgatcccctt ctgcaagctt gctgagatca acacaacatt tcgcaagcag gcatttgcat
35401 tgcggggtag tacaactgtg tcctttcaag agtctatatg ttttataggc ctttcctgag
35461 cggtaagaac aggtcgccag taagaacaag gcttcttctg agtgtacttc tgcataaagg
35521 cgttctgcgg gggaaaccgc atctcggtag gcatagtggt ttagtgcttg ccatatagca
35581 gcctggacgg gtccctgcag caccgacatc ctcgaggctc aggcccactt tctgcagtgc
35641 cacaggcacc cccccccccc catagcggct ccggcccggc cagccccggc tcatttaaag
35701 gcaccagccg ccgttaccgg gggatggggg agtccgagac agaatgactt ctttatcctg
35761 ctgactctgg aaagcccggc gccctgtgat ccattgcaaa ccgagagtca cctcgtgttt
35821 agaacacgga tccactccca agttcagtgg ggggatgtga ggggtgtggc aggtaggacg
35881 aaggactctc ttccttctga ttcggtccgc acagtggggc ctagggctgg agctctctcc
35941 gtgcggaccg ctgactccct ctaccttggg ttccctcggc cccaccctgg aacgccgggc
36001 cttggcagat tctggccctt tctggccctt cagtcgctgt cagaaacccc atctcatgct
36061 cggatgcccc gagtgactgt ggctcgcacc tctccggaaa cattggaaat ctctcctcta
36121 cgcgcggcca cctgaaacca caggagctcg ggacacacgt gctttcggga gagaatgctg
36181 agagtctctc gccgactctc tcttgacttg agttcttcgt gggtgcgtgg ttaagacgta
36241 gtgagaccag atgtattaac tcaggccggg tgctggtggc tcacgcctgt aaccccaaca
36301 ctttgggagg ccgaggccgt aggatccctc gaggaatcgc ctaaccctgg ggaggttgag
36361 gttgcagtga gtgagccata gttgtgtcac tgtgctccag tctgggcgaa agacagaatg
36421 aggccctgcc acaggcaggc aggcaggcag gcaggcagaa agacaacagc tgtattatgt
36481 tcttctcagg gtaggaagca aaaataacag aatacagcac ttaattaact tttttttttt
36541 ccttcggacg gagtttcact cttggtgccc acgctggagt gcagtggcac catctcggct
36601 caccgcaacc tccacctccc gcgttcaagc gattctcctg cctcagcctc ctgagtagct
36661 gggattacag ggaggagcca ccacacccag ctgattttgt attgttagca gagacggcat
36721 ttctccatgt gggtcaggct ggtctcgaac tggcgacccc agtggatctg cccgccccgg
36781 cctcccaaag tgctggggtg acaggcgtga gccatcgtga ctggccggct acgtttattt
36901 tacatttatt tatttattta tttatttact tatttattta ttttcgagac agactctcgc
36961 tctgctgccc aggctggagt gcagcggcgt gatctcggct cactgcaacg tccgcctccc
37021 gggttcacgc cattctcctg cctcagcctc ccaagtagct gggactacag gcgcccgcca
37081 ccgtgcccgg ctaacttttt gtattttgag tagagatggg gtttcactgt ggtagccagg
37141 atggtctcga tctcctgacc ccgtgatccg tccacctcgg cctcccaaag tgctgggatg
37201 acaggcgtga gccaccggcc ccggcctatt tatctattta ttaactttga gtccaggtta
37261 tgaaaccagt tagtttttgt aatttttttt tttttttttt ttttttgaga cgaggtttca
37321 ccgtgttgcc aaggcttgga ccgagggatc caccggccct cggcctccca aaagtgcggg
37381 gatgacaggc gcgagcctac cgcgcccgga cccccccttt ccccttcccc cgcttgtctt
37441 cccgacagac agtttcacgg cagagcgttt ggctggcgtg cttaaactca ttctaaatag
37501 aaatttggga cgtcagcttc tggcctcacg gactctgagc cgaggagtcc cctggtctgt
37561 ctatcacagg accgtacacg taaggaggag aaaaatcgta acgttcaaag tcagtcattt
37621 tgtgatacag aaatacacgg attcacccaa aacacagaaa ccagtctttt agaaatggcc
37681 ttagccctgg tgtccgtgcc agtgattctt ttcggtttgg accttgactg agaggattcc
37741 cagtcggtct ctcgtctctg gacggaagtt ccagatgatc cgatgggtgg gggacttagg
37801 ctgcgtcccc ccaggagccc tggtcgatta gttgtgggga ccgccttgga gggcgcggtg
37861 acccactgtg ctgtgggagc ctccatcctt ccccccaccc cctccccagg gggatcccaa
37921 ttcattccgg gctgacacgc tcactggcag gcgtcgggca tcacctagcg gtcactgtta
37981 ctctgaaaac ggaggcctca cagaggaagg gagcaccagg ccgcctgcgc acagcctggg
38041 gcaactgtgt cttctccacc gcccccgccc ccacctccaa gttcctccct cccttgttgc
38101 ctaggaaatc gccactttga cgaccgggtc tgattgacct ttgatcaggc aaaaacgaac
38161 aaacagataa ataaataaaa taacacaaaa gtaactaact aaataaaata agtcaataca
38221 acccattaca atacaataag atacgatacg ataggatgcg ataggatacg ataggataca
38281 atacaatagg atacgataca ataceataca atacaataca atacaataca atacaataca
38341 atacaataca atacaatacg ccgggcgcgg tggctcatgc ctgtcatccc gtcactttgg
38401 gatgccgagg tggacgcatc acctgaagtc gggagttgga gacaagcccg accaacatgg
38461 agaaatcccg tctcaactga aaatacaaaa ctagccgggc gcggtggcac atgcctataa
38521 tcccagctgc taggaaggct gaggcaggag aatcgcttga acctgggaag cggaggttgc
38581 agtgagccga gattgcgcca tcgcactcca gtctgagcaa caagagcgaa actccgtctc
38641 aaaaataaat acataaataa atacatacat acatacatac atacatacat acatacatac
38701 ataaattaaa ataaataaat aaaataaaat aaataaatgg gccctgcgcg gtggctcaag
38761 cctgtcatcc cctcaccttg ggaggccaag gccggtggat caagaggcgg tcagaccaac
38821 agggccagta tggtgaaacc ccgtctctac tcacaataca caacattagc cgggcgctgt
38881 gctgtgctgc accgtctgta atcccagcta ctcgggaggc cgagctgagg caggagaatc
38941 gcttgaacct gggaggcgga ggtcgcagtg agccgagatc gcgccactgc aacccagcct
39001 gggcgacaga gcgagactcc gtctccaaaa aatgaaaatg aaaacgaaac gcaacaaaat
39061 aattaaaaag tgagtttctg gggaaaaaga agaaaagaaa aaagaaaaaa acaacaaaac
39121 agaacaaccc caccgtgaca tacacgtacg cttctcgcct ttcgaggcct caaacacgtt
39181 aggaattatg cgtgatttct tttcttaact tcattttatg ttatcatcat gattgatgtt
39241 tcgagacgga gtctcggagg cccgccctcc ctggttgccc agacaacccc gggagacaga
39301 ccctggctgg gcccgattgt tcttctcctt ggtcaggggt ttccctgtct ttcttcgtgt
39361 ctttaacccg cgcggactct tccgcctcgg gtttgacaga tggcagctcc actttaggcc
39421 ttgttgttgt tggggacttt cctgattctc cccagatgta gtgaaagcag gtagattgcc
39481 ttgcctggcc ttgcctggcc ttgccttttc tttctttctt tcttccttca ttactttctc
39541 tttttcttct tcctcttctt cttttttttg agacagagtt tcactcttgt tgcccaggct
39601 agagggcaat ggcgcgatct cggctcaccg caccctccgc ctcccaggtt caagcgattc
39661 tcctgcctca gcctcctgat tagctgggat tacaggcatg ggccaccgtg ctggctgatg
39721 tttgtacttt tagtagagac ggtgtttttc catgttggtc aggctggtct cccactccca
39781 acctcaggtg gtccgcctgc cttagcctcc caaagtgctg ggatgacagg cgtgcaaccg
39841 cgcccagcct ctctctctct ctctctctct ctcgctcgct tgctcgcttg ctttcgtgct
39901 ttcttgcttt cccgttttct tgctttcttt ctttctttcg tttctttcat gcttgctttc
39961 ttgcttgctt gcttgctttc gtgctttctt gctttcctgt tttccttctt tctttctttc
40021 tttctttctt ttgtttcttt cttgcttgct ttcttgcttg cttgcttgct ttcgtgcttt
40141 gcttgcttgc tttcgtgctt tcttgttttc tcgatttctt tcttcctttt gtttccttcc
40201 tgcttgcttt ctcgcttgct tgctttcgtg cttcttgctt tcctgttttc tttctttctt
40321 tctcgatttc tttctttctt ttgtttcttt cctgcttgct ttcttgcttg attgccttcg
40381 tgctttcttg ctctcttgtt ttctttcttt cttttgtttc tttctttctt gcttccttgt
40441 tttcttgctt tcttgcttgc ttgctttcgt gctttcttgt tttcttgctt tctttctttt
40501 gtttctttct tgcttgcttt cttgcttcct tgttttcttg ctttcttgct tgcttgcttt
40561 cgtgctttct ttcttgcttt cttttctttc tttcttttct ttttctttct ttcttgcttt
40621 cttttctttc atcatcatct ttctttcttt cctttctttc tttccttctt tctatctttc
40681 tttctttctt tccttctttc tttctttctt tctttctgtt tcgtcctttt gagacagagt
40741 ttcactcttg tttccacggc tagagtgcaa tggcgcgatc ttggctcacc gcaccctccg
40801 cctcccgggt tcgagcgctt ctcctgcctc cagcctcccg attagcgggg attgacaggg
40861 aggcaccccc acgcctggct tggctgatgt ttgtgttttt agtaggcacg ccgtgtctct
40921 ccatgttgct caggctggtc tccaactccc gacctcctgt gatgcgccca cctcggcctc
40981 tcgaagtgct gggatgacgg gcgtgacgac cgtgcccggc ctgttgactc atttcgcttt
41041 tttatttctt tcgtttccac gcgtttactt atatgtatta atgtaaacgt ttctgtacgc
41101 ttatatgcaa acaacgacaa cgtgtatctc tgcattgaat actcctgcgt acggtaaata
41161 cgtatcggtt gtatggaaat agacttctgt atgatagatg taggtgtctg tgttatacaa
41221 ataaatacac atcgctctat aaagaaggga tcgtcgataa agacgtttat tctacgtatg
41281 aaaagcgtcg tacttacgtg tgtaaatgaa ccgagcgtac gtagctatct ccgttttctt
41341 tcttcctctc ctccgtgttt ttcttccttc ctttcttcct ttctctcctt ctttaggttt
41401 ttcttcctct cttcctctcc ttccttctct ctttctgtcc ctttctcctt cgtgctttat
41581 gtcttttaaa aaattggagt gtttcagaag tttactttgt gtatctacgt tttctaaatt
41641 gtctctcttt tctccatttt cttcctccct ccctccctcc ctccctgctc ccttccctcc
41701 ctccttccct ttcgccatct gtctcttttc cccactcccc tccccccgtc tgtctctgcg
41761 tggattccgg aagagcctac cgattctgcc tctccgtgtg tctgcagcga ccccgcgacc
41821 gagtccttgt gtgttctttc tccctccctc cctccctccc tccctccctc cctccctgct
41881 tccgagaggc atctccagag accgcgccgt gggttgtctt ctgactctgt cgcggtcgag
41941 gcagagacgc gttttgggca ccgtttgtgt ggggttgggg cagaggggct gcgttttcgg
42001 cctcgggaag agcttctcga ctcacggttt cgctttcgcg gtccacgggc cgccctgcca
42061 gccggatctg tctcgctgac gtccgcggcg gttgtcgggc tccatctggc ggccgctttg
42121 agatcgtgct ctcggcttcc ggagctgcgg tggcagctgc cgagggaggg gaccgtcccc
42181 gctgtgagct aggcagagct ccggaaagcc cgcggtcgtc agcccggctg gcccggtggc
42241 gccagagctg tggccggtcg cttgtgagtc acagctctgg cgtgcaggtt tatgtggggg
42301 agaggctgtc gctgcgcttc tgggcccgcg gcgggcgtgg ggctgcccgg gccggtcgac
42361 cagcgcgccg tagctcccga ggcccgagcc gcgacccggc ggacccgccg cgcgtggcgg
42421 aggctgggga cgcccttccc ggcccggtcg cggtccgctc atcctggccg tctgaggcgg
42481 cggccgaatt cgtttccgag atccccgtgg ggagccgggg accgtcccgc ccccgtcccc
42541 cgggtgccgg ggagcggtcc ccgggccggg ccgcggtccc tctgccgcga tcctttctgg
42601 cgagtccccg tggccagtcg gagagcgctc cctgagccgg tgcggcccga gaggtcgcgc
42661 tggccggcct tcggtccctc gtgtgtcccg gtcgtaggag gggccggccg aaaatgcttc
42721 cggctcccgc tctggagaca cgggccggcc cctgcgtgtg gccagggcgg ccgggagggc
42781 tccccggccc ggcgctgtcc ccgcgtgtgt ccttgggttg accagaggga ccccgggcgc
42841 tccgtgtgtg gctgcgatgg tggcgttttt ggggacaggt gtccgtgtcc gtgtcgcgcg
42901 tcgcctgggc cggcggcgtg gtcggtgacg cgacctcccg gacccggggg aggtatatct
42961 ttcgctccga gtcggcaatt ttgggccgcc gggttatat
```

Also included herein are (a) complementary DNA sequences, (b) subsequences of the forgoing, (c) rRNA nucleotide sequences and subsequences complementary to SEQ ID NO: 1 and (c) RNA sequences and subsequences complementary to (c).

### EXAMPLES

The examples set forth below illustrate but do not limit the invention.

### Example I

### Identification of Quadruplex Motif Ribosomal Nucleotide Sequences

Human ribosomal DNA having the sequence of SEQ ID NO: 1 and its transcribed complementary RNA sequence were searched for nucleotide sequences conforming to a quadruplex sequence motif. The rDNA sequence of SEQ ID NO: 1 was not notated in databases that included other genomic DNA sequences. For example, the rDNA sequence of SEQ ID NO: 1 is not part of build 34 or build 35 in the NCBI human genomic DNA sequence.

To find the quadruplex motifs PERL program scanned SEQ ID NO: 1 ((>EMBLRELEASE|U133691HSU13369 HUMAN RIBOSOMAL DNA COMPLETE REPEATING UNIT) and identified any nucleotide bases that appeared within the regular expression GGG(.{1,7})GGG(.{1,7})GGG(.{1,7})GGG. 42999 bases were processed in the rDNA sequence. There were 18 separated potential quadruplex sequence (PQS) regions identified comprising 544 total bases. A second search was carried out on the same sequence using the regular expression CCC(.{1,7})CCC(.{1,7})CCC(.{1,7})CCC. Again, 42999 bases were processed, but there were 30 separated PQS regions found comprising 995 bases. The first set of search parameters were used to search for G-quadruplex forming sequences in coding strand of rDNA and the second search parameters were used to search for G-quadruplex forming sequences in the complementary rRNA and in the non-coding strand of rDNA.

The following rDNA quadruplex motif sequences were identified. The DNA sequences are on the coding strand of rDNA, the nucleotide ranges refer to positions on the 43 kb human ribosomal DNA repeat unit (accession no. U13369). No exact sequence matches were identified within the NCBI build 35 of the human genome on the coding strand (the non-template strand, the plus (+) strand, or the antisense strand) or its reverse complement for the following nucleotide sequences.

(SEQ ID NO:2)

| | |
|---|---|
| 1197-1221: | GGGTGGACGGGGGGGCCTGGTGGGG; |

(SEQ ID NO:3)

| | |
|---|---|
| 2160-2227: | CCCGGGTGCCCTTGCCCTCGCGGTCCCCGGCCCTCGC |
| | CCGTCTGTGCCCTCTTCCCCGCCCGCCGCCC; |

(SEQ ID NO:4)

| | |
|---|---|
| 2958-2985: | GGGTCGGGGGGTGGGGCCCGGGCCGGGG; |

(SEQ ID NO:5)

| | |
|---|---|
| 3468-3491: | CCCCGCCCCGGCCCCACCGGTCCC; |

(SEQ ID NO:6)

| | |
|---|---|
| 3500-3532: | CCCCCGCGCCCGCTCGCTCCCTCCCGTCCGCCC; |

(SEQ ID NO:7)

| | |
|---|---|
| 6184-6213: | GGGTCGGGGGCGGTGGTGGGCCCGCGGGGG; |

(SEQ ID NO:8)

| | |
|---|---|
| 6915-6944: | CCCGCCCCTTCCCCCTCCCCCCGCGGGCCC; |

(SEQ ID NO:9)

| | |
|---|---|
| 6375-6403: | GGGGGCGGGAACCCCCGGGCGCCTGTGGG; |

(SEQ ID NO:10)

| | |
|---|---|
| 6961-6983: | GGGTGGCGGGGGGGAGAGGGGGG; |

(SEQ ID NO:11)

| | |
|---|---|
| 7254-7298: | GGGTCCGGAAGGGGAAGGGTGCCGGCGGGGAGAGAGG |
| | GTCGGGGG; |

(SEQ ID NO:12)

| | |
|---|---|
| 7370-7399: | CCCCGCGCCCCTCCTCCTCCCCGCCGCCCC; |

(SEQ ID NO:13)

| | |
|---|---|
| 7734-7763: | CCCGTCCCGCCCCCGGCCCGTGCCCCTCCC; |

(SEQ ID NO:14)

| | |
|---|---|
| 8440-8494: | CCCGCCCCCCGTTCCTCCCGACCCCTCCACCCGCCCT |
| | CCCTTCCCCCGCCGCCCC; |

(SEQ ID NO:15)

| | |
|---|---|
| 8512-8573: | GGGGGCGGGCTCCGGCGGGTGCGGGGGTGGGCGGGCG |
| | GGGCCGGGGGTGGGGTCGGCGGGGG; |

(SEQ ID NO:16)

| | |
|---|---|
| 8716-8747: | CCCGTCTCCGCCCCCCGGCCCCGCGTCCTCCC; |

(SEQ ID NO:17)

| | |
|---|---|
| 8750-8770: | GGGAGGGCGCGCGGGTCGGGG; |

(SEQ ID NO:18)

| | |
|---|---|
| 8904-8926: | CCCCCCTCCCGGCGCCCACCCCC; |

(SEQ ID NO:19)

| | |
|---|---|
| 9024-9052: | CCCACCCCTCCTCCCCGCGCCCCCGCCCC; |

(SEQ ID NO:20)

| | |
|---|---|
| 10137-10179: | CCCCTCCTCCCGCCCACGCCCCGCTCCCCGCCCCCGG |
| | AGCCCC; |

(SEQ ID NO:21)

| | |
|---|---|
| 10817-10839: | GGGCTGGGTCGGTCGGGCTGGGG; |

(SEQ ID NO:22)

| | |
|---|---|
| 10885-10934: | CCCCCCCCACGCCCGGGGCACCCCCCTCGCGGCCCTC |
| | CCCCGCCCCACCC; |

(SEQ ID NO:23)

| | |
|---|---|
| 10951-10969: | CCCTCCCCACCCCGCGCCC; |

(SEQ ID NO:24)

| | |
|---|---|
| 10985-11012: | CCCCCGCTCCCCGTCCTCCCCCCTCCCC; |

(SEQ ID NO:25)

| | |
|---|---|
| 11029-11066: | GGGGCGCGGCGGGGGGAGAAGGGTCGGGGCGGCAGGG |
| | G; |

(SEQ ID NO:26)

| | |
|---|---|
| 11345-11389: | CCCCCCGCCCTACCCCCCCGGCCCCGTCCGCCCCCCG |
| | TTCCCCCC; |

(SEQ ID NO:27)

| | |
|---|---|
| 11888-11912: | CCCCCGGCGCCCCCCCGGTGTCCCC; |

(SEQ ID NO:28)

| | |
|---|---|
| 13174-13194: | GGGCCGGGACGGGGTCCGGGG; |

(SEQ ID NO:29)

| | |
|---|---|
| 13236-13261: | CCCCGTGGCCCGCCGGTCCCCGTCCC; |

(SEQ ID NO:30)

| | |
|---|---|
| 14930-14963: | CCCTCCCTCCCTCCCCCTCCCTCCCTCTCTCCCC; |

(SEQ ID NO:31)

| | |
|---|---|
| 17978-18013: | CCCCCACCCCCCCGTCACGTCCCGCTACCCTCCCCC; |

(SEQ ID NO:32)

| | |
|---|---|
| 20511-20567: | GGGGGTGCGGGAATGAGGGTGTGTGTGGGGAGGGGGT |
| | GCGGGGTGGGGACGGAGGGG; |

(SEQ ID NO:33)

| | |
|---|---|
| 23408-23434: | GGGGAGAGAGGGGGGAGAGGGGGGGGG; |

(SEQ ID NO:34)

| | |
|---|---|
| 28214-28250: | CCCCAAACCGCCCCCCCCCCCCCGCCTCCCAACACC |
| | C; |

(SEQ ID NO:35)

| | |
|---|---|
| 31239-31275: | CCCCACCCACGCCCCACGCCCCACGTCCCGGGCACC |
| | C; |

(SEQ ID NO:36)

| | |
|---|---|
| 31415-31452: | GGGAGGGGTGGGGGTGGGGTGGGTTGGGGGTTGTGG |
| | GG; |

(SEQ ID NO:37)

| | |
|---|---|
| 37405-37431: | CCCGGACCCCCCCTTTCCCCTTCCCCC; |

(SEQ ID NO:38)

| | |
|---|---|
| 39261-39290: | CCCGCCCTCCCTGGTTGCCCAGACAACCCC; |
| and | |

(SEQ ID NO:39)

| | |
|---|---|
| 41667-41709: | CCCTCCCTCCCTCCCTCCCTCCTCCCTTCCCTCCCT |
| | CCTTCCC. |

Following are examples of rDNA nucleotide sequences that are identical to non-rDNA sequences in human genomic DNA. All DNA sequences are in the rDNA coding strand, and the nucleotide ranges refer to positions on the 43 kb human ribosomal DNA repeat unit (accession no. U13369).

(SEQ ID NO:40)

| | |
|---|---|
| 1310-1333: | CCCCCTCCCTTCCCCAGGCGTCCC; |

(SEQ ID NO:41)

| | |
|---|---|
| 5701-5718: | GGGAGGGAGACGGGGGGG; |

(SEQ ID NO:42)

| | |
|---|---|
| 6535-6553: | GGGCGGGGGGGGCGGGGGG; |

(SEQ ID NO:43)

| | |
|---|---|
| 7499-7517: | CCCGCCCCGCCGCCCGCCC; |

(SEQ ID NO:44)

| | |
|---|---|
| 10111-10127: | CCCCCGCCCCCCCCCCC; |

(SEQ ID NO:45)

| | |
|---|---|
| 13080-13095: | GGGGTGGGGGGGAGGG; |

(SEQ ID NO:46)

| | |
|---|---|
| 14213-14248: | CCCTCCCTCCCTCCCTCCCTCCCTCCCTCCCTCCCC; |

(SEQ ID NO:47)

| | |
|---|---|
| 16166-16189: | GGGGTGGGGTGGGGTGGGGTGGGG; |

(SEQ ID NO:48)

| | |
|---|---|
| 28148-28177: | CCCCCCGGCTCCCCCCACTACCCACGTCCC; |
| and | |

(SEQ ID NO:49)

| | |
|---|---|
| 41842-41876: | CCCTCCCTCCCTCCCTCCCTCCCTCCCTCCCTCCC. |

The following rRNA quadrupled motif sequences were identified. The RNA sequences are inferred from rDNA sequence and annotations found within accession number U13369. No matches were identified within genes (as identified by Curwen et al. The Ensembl Automatic Gene Annotation System, Genome Res. 2004 May; 14(5):942-950) along the coding strand (CDS) of the human genome for the DNA sequence transcribed to produce the rRNA and pre-rRNA.

RNA sequence from 5′ external transcribed spacer
region in rDNA

(SEQ ID NO:107)

GGGGUGGACGGGGGCGCCUGGUCGGG;

(SEQ ID NO:108)

GGGUCGGGGGGUGGGGCCCGGGCCGGGG;
RNA sequence from internal transcribed spacer 1
region in rDNA

(SEQ ID NO:109)

GGGAGGGAGACGGGGGGG;

(SEQ ID NO:110)

GGGUCGGGGGCGGUGGUGGGCCCGCGGGGG;

(SEQ ID NO:111)

GGGGGCGGGAACCCCCGGGCGCCUGUGGG;
RNA sequences from internal transcribed spacer 2
region in rDNA

(SEQ ID NO:112)

GGGUGGCGGGGCGGAGAGGGGGG;

(SEQ ID NO:113)

GGGUCCGGAAGGGGAAGGGUGCCGGCGGGGAGAGAGGGUCGGGGG;
RNA sequences within 28S rRNA

(SEQ ID NO:114)

GGGGGCGGGCUCCGGCGGGUGCGGGGGUGGGCGGGCGGGGCCGGGGGUGG
GGUCGGCGGGGG;

(SEQ ID NO:115)

GGGAGGGCGCGCGGGUCGGGG;

(SEQ ID NO:116)

GGGCUGGGUCGGUCGGGCUGGGG;

(SEQ ID NO:117)

GGGGCGCGGCGGGGGGAGAAGGGUCGGGGCGGCAGGGG;
RNA sequences from 3′ external transcribed spacer
region in rDNA

(SEQ ID NO:118)

GGGCCGGGACGGGGUCCGGGG.

Following are C-rich rRNA and pre-rRNA sequences in the transcribed region of rDNA, which in certain embodiments may form a quadruplex.

RNA sequence from 5′ external transcribed spacer
region in rDNA

(SEQ ID NO:121)

CCCCCUCCCUUCCCCAGGCGUCCC

(SEQ ID NO:122)

CCCGGGUGCCCUUGCCCUCGCGGUCCCCGGCCCUCGCCCGUCUGUGCCCU
CUUCCCCGCCCGCCGCCC

(SEQ ID NO:123)

CCCCGCCCCGGCCCCACCGGUCCC

(SEQ ID NO:124)

CCCCCGCGCCCGCUCGCUCCCUCCCGUCCGCCC
RNA sequences from internal transcribed spacer 2
region in rDNA

(SEQ ID NO:125)

CCCGCCCCUUCCCCCUCCCCCCGCGGGCCC

(SEQ ID NO:126)

CCCCGCCCCCCUCCUCCUCCCCGCCGCCCC

(SEQ ID NO:127)

CCCGCCCCGCCGCCCGCCC

(SEQ ID NO:128)

CCCGUCCCGCCCCCGGCCCGUGCCCCUCCC
RNA sequences within 28S rRNA

(SEQ ID NO:129)

CCCGCCCCCCGUUCCUCCCGACCCCUCCACCCGCCCUCCCUUCCCCCGCC
GCCCC

(SEQ ID NO:130)

CCCGUCUCCGCCCCCCGGCCCCGCGUCCUCCC

(SEQ ID NO:131)

CCCCCCUCCCGGCGCCCACCCCC

(SEQ ID NO:132)

CCCACCCCUCCUCCCCGCGCCCCCGCCCC

(SEQ ID NO:133)

CCCCCGCCCCCCCCCCC

(SEQ ID NO:134)

CCCCUCCUCCCGCCCACGCCCCGCUCCCCGCCCCCGGAGCCCC

(SEQ ID NO:135)

CCCCCCCCACGCCCGGGGCACCCCCCUCGCGGCCCUCCCCCGCCCCACCC

(SEQ ID NO:136)

CCCUCCCCACCCCGCGCCC

(SEQ ID NO:137)

CCCCCGCUCCCCGUCCUCCCCCCUCCCC

(SEQ ID NO:138)

CCCCCCGCCCUACCCCCCCGGCCCCGUCCGCCCCCCGUUCCCCCC

(SEQ ID NO:139)

CCCCCGGCGCCCCCCCGGUGUCCCC
RNA sequence from 3′ external transcribed spacer
region in rDNA

(SEQ ID NO:140)

CCCCGUGGCCCGCCGGUCCCCGUCCC.

Following are rRNA sequences exactly matching RNA transcribed from non-rDNA and a description of the rDNA regions from which they are transcribed or located.

RNA Sequence from Internal Transcribed Spacer 1 Region in rDNA

### (SEQ ID NO:119) GGGCGGGGGGGGCGGGGGG;

RNA Sequence from 3′ External Transcribed Spacer Region in rDNA

### (SEQ ID NO:120) GGGGUGGGGGGGAGGG.

### Example 2

### Human rRNA Interaction Screening Assay

RNA was isolated from HCT116 cells (RNeasy kit, QIAGEN) and contacted in vitro with each compound from a library of compounds. In representative assays, 1 ug of total RNA from HCT116 cells was incubated with 0.5 ug/mL of propidium iodide (PI), 10 uM compound A-I or another compound in the library in a volume of 10 uL for 15 min at room temperature, followed by agarose gel electrophoresis. Fluorescence of the compounds was visualized on each gel. It was determined that PI did not discriminate in its binding to 1 SS and 28S rRNA, while A-1 bound preferentially to 28S rRNA. Compound A-1, compound C-1 and compound C-2 showed selective binding to 28S over 18S in the electrophoresis mobility shift assay. Compounds C-3 and C-4 showed less selectivity for 28S over 118S compared to compounds A-1, C-1 and C-2. Compound C-5 showed specific binding to 28S over 18S in electrophoresis mobility shift assay. Compounds C-1, C-2, C-3, C-4 and C-5 have the following general formula:

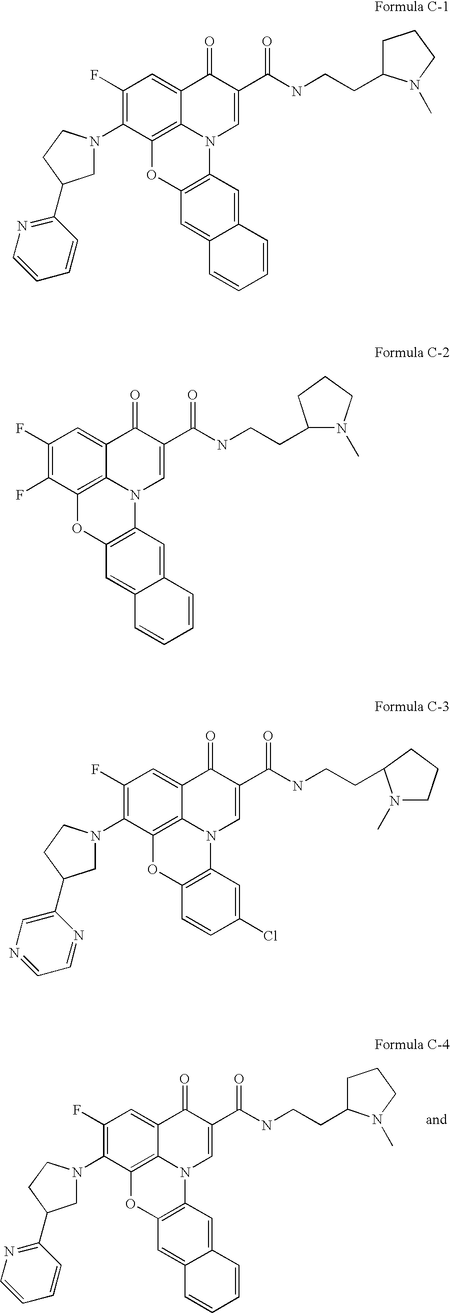

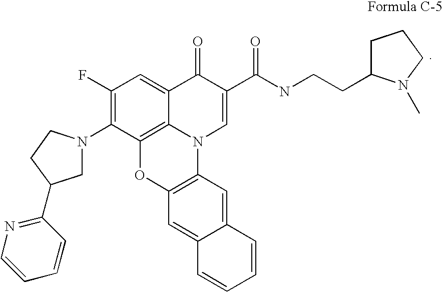

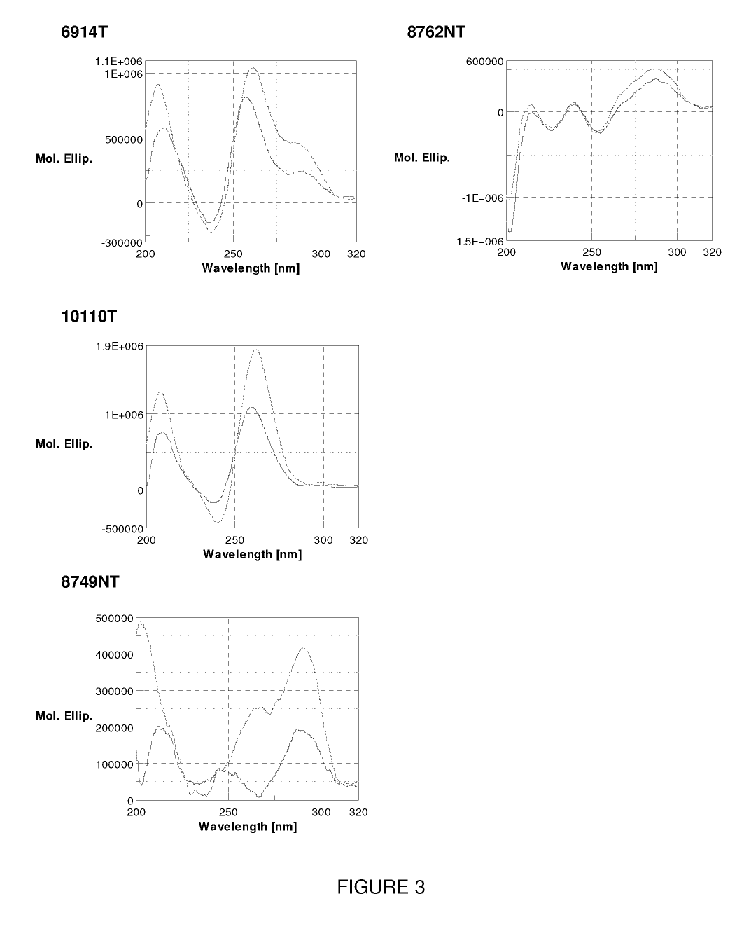

Compounds that bound to the 28S rRNA were subjected to competition for rRNA binding with Actinomycin D, Se₂SAP (FIG. 3A of US20040110820 published on Jun. 10, 2004) and double-stranded DNA (dsDNA) in confirmatory studies. In such assays, 1 ug of total RNA from HCT116 cells was incubated with 10 uM compound A-1 in a volume of 10 uL in the absence/presence of (a) increasing amount of Actinomycin D (10, 100 and 200 uM) for 15 min at room temperature, (b) increasing amount of Se₂SAP (see figure) for 30 min at room temperature, or (c) increasing amount of pUC18 (0.25, 0.5, 1, 2 and 4 ug) for 30 min at room temperature, followed by agarose gel electrophoresis. Based upon the fluorescence intensity of the bands corresponding to 28S and 18S rRNAs, (a) Actinomycin D failed to compete with compound A-1 for rRNA, (b) Se₂SAP competed with compound A-1 for 28S rRNA, and to a lesser extent for 18S rRNA, and (c) dsDNA competed with 28S rRNA for compound A-1.

### Example 3

### Localization of Ribosomal Nucleic Acid Interacting Molecules in Cells

A cell localization assay was utilized to determine cell localization for compounds that interacted with rRNA. In these studies, A549 cells were plated in borosilicate chamber slides. The cells were treated with 2 uM compound A-1 the next day for one hour or two hours, washed with PBS, fixed for 10 min in 4% paraformaldehyde and observed under a fluorescence microscope (Olympus) in the Ex360 nm/Em548 nm channel at 600× magnification. Judging by fluorescence intensity, compound A-1 accumulated in the nucleoli, as well as the cytoplasm/perinuclear space. Accordingly, a compound tested in the assay was localized in cell nucleoli.

### Example 4

### Cellular Target of Ribosomal Nucleic Acid Interacting Molecules

To determine the cellular target of rRNA-interacting compounds, a study was conducted to ascertain whether the compounds could select for cellular DNA or RNA. In these studies A549 cells were plated in borosilicate chamber slides. The cells were treated with 2 uM compound A-1 the next day for one hour or two hours, washed with PBS, fixed for 10 min in 4% paraformaldehyde, permeabilized for 5 min in 1:1 ethanol:acetone mix, treated with 2.5 ug/mL of RNase A or 340 Kunitz units/mL of DNase I and observed under a fluorescence microscope (Olympus) in the Ex360 nm/Em548 nm channel at 600× magnification. Treatment with DNase I had a minimal effect on compound A-I localization, while RNase I significantly reduced nucleolar staining by the drug. Accordingly, a compound tested in the assay interacted with RNA preferentially over DNA in cells.

### Example 5

### Effect of Ribosomal Nucleic Acid Interacting Molecules on Cell Nucleolin Localization

An assay was conducted to determine the effect of ribosomal nucleic acid interacting compounds on cell nucleolin location. In these studies, A549 cells were plated in borosilicate chamber slides. The cells were treated with 10 uM compound A-1 the next day for two hours, washed with phosphate buffered saline, fixed for 10 minutes in 4% paraformaldehyde, permeabilized for 5 minutes in a 1:1 ethanol:acetone mix, incubated with a 1:100 dilution of an anti-nucleolin monoclonal antibody (catalog no. RDI-NUCLEOLabm; Research Diagnostics, Inc.) in 5% donkey serum, followed by incubation with a 1:100 diluted TRITC-labeled secondary anti-mouse antibody and observed under a fluorescence microscope (e.g., Olympus) in the TRITC channel at 600× magnification. In untreated cells nucleolin was localized in nucleoli, while in cells treated with compound A-1 nucleolin was redistributed to the nucleoplasm. Accordingly, nucleolin was redistributed from the nucleolus to the nucleoplasm in cells treated with a compound tested in the assay.

Another assay was conducted to determine the effect of ribosomal nucleic acid interacting compounds on cell fibrillarin location. These studies were conducted using a protocol similar to that described in the preceding paragraph, except an antibody that specifically bound to fibrillarin (catalog no. ab5821; Novus Biologicals, Inc.) was utilized. It was determined that compound A-1 caused redistribution of fibrillarin in a similar time-frame as redistribution of nucleolin.

### Example 6

### Quadruplex Structures of Ribosomal Nucleic Acids

Circular dichroism (CD) was utilized to determine whether subsequences from ribosomal nucleic acids form quadruplex structures. All sequences were HPLC purified DNA oligonucleotides (sequences 5′ to 3′ as represented hereafter). The name of each sample in FIGS. 3A and 3B identifies the approximate location along the rDNA unit as well as the specific strand (NC=non-coding; C=coding). The following procedure was utilized: each oligonucleotide was dissolved at a strand concentration of 5 uM in 200 ul of aqueous buffer containing Tris pH 7.4 (10 mM). The sample was heated to 95° C. for 5 min. then allowed to cool to ambient temperature. CD spectroscopy was performed on a JASCO 810 Spectropolarimeter, using a quartz cell of 1 mm path length. Additional spectra were taken after the addition of 20 ul KCl (1M) to the oligonucleotide solution. Compound A-1 has been shown to interact preferentially with a mixed-parallel quadruplex structure in competition assays (e.g., PCT/US2004/033401 filed on Oct. 7, 2004, entitled “Competition Assay for Identifying Modulators of Quadruplex Nucleic Acids”).

Quadruplex structures for nucleic acids having sequences derived from human ribosomal DNA, template (T) and non-template (NT) strands, were tested by the same methods and spectra are summarized in FIG. 3 and in the following table. The nucleic acid identifier notes (i) whether the nucleotide sequence is from the non-template (NT) strand (e.g., SEQ ID NO: 1) or templates (T) strand (e.g., reverse complement of SEQ ID NO: 1) of human rDNA, and the (ii) the location of the sequence in the NT strand or the location in SEQ ID NO: 1 from which the reverse-complement sequence is derived for the T strand of rDNA. For nucleotide sequences from the NT strand, the number in the identifier delineates the 5′ nucleotide of the oligonucleotide and is the position in SEQ ID NO: 1 less one nucleotide (e.g., the nucleotide sequence of oligonucleotide 13079NT spans sixteen (16) nucleotides in SEQ ID NO: 1 beginning at position 13080 in SEQ ID NO: 1). For nucleotide sequences from the T strand, the number in the identifier defines the 3′ nucleotide of the reverse complement oligonucleotide derived from the position in SEQ ID NO: 1 less one nucleotide (e.g., the nucleotide sequence of 10110T is the reverse complement of a seventeen (17) nucleotide span in SEQ ID NO: 1, with the 3′ terminus of the oligonucleotide defined at position 10111 in SEQ ID NO: 1). Spectra characteristic of parallel, mixed parallel, antiparallel (with mixed parallel characteristics) and complex intramolecular quadruplex structures were observed. Quadruplex conformation determinations are summarized in the following table.

| Nucleic | SEQ | | |
|---|---|---|---|
| acid | ID | | |
| identifier | NO. | Conformation | Nucleotide Sequence |
| 10110T | 158 | Parallel | GGGGGGGGGGGCGGGGG |
| 13079NT | 159 | Parallel | GGGGTGGGGGGGAGGG |
| 6960NT | 160 | Mixed | GGGTGGCGGGGGGGAGAGGGGG |
| | | | G |
| 6534NT | 161 | Mixed | GGGCGGGGGGGGCGGGGGG |
| 1196NT | 162 | Mixed | GGGTGGACGGGGGGGCCTGGTG |
| | | | GGG |
| 2957NT | 163 | Mixed | GGGTCGGGGGGTGGGGCCCGGG |
| | | | CCGGGG |
| 5700NT | 164 | Mixed | GGGAGGGAGACGGGGGGG |
| 8511NT | 165 | Mixed | GGGGGTGGGCGGGCGGGCCCGG |
| | | | GGGTGGG |
| 6163NT | 166 | Mixed | GGGTCGGGGGCGGTGGTGGGCC |
| | | | CGCGGGGG |
| 11028NT | 167 | Mixed | GGGGCGCGGCGGGGGGAGAAGG |
| | | | GTCGGGGCGGCAGGGG |
| 6374NT | 168 | Mixed | GGGGGCGGGAACCCCCGGGCGC |
| | | | CTGTGGG |
| 7733T | 169 | Mixed | GGGAGGGGCACGGGCCGGGGGC |
| | | | GGGACGGG |
| 7253NT | 170 | Mixed | GGGTCCGGAAGGGGAAGGGTGC |
| | | | CGGCGGGGAGAGAGGGTCGGGG |
| | | | G |
| 13173NT | 171 | Mixed | GGGCCGGGACGGGGTCCGGGG |
| 6914T | 172 | Mixed | GGGCCCGCGGGGGGAGGGGGA |
| | | | AGGGGCGGG |
| 8749NT | 173 | Antiparallel | GGGAGGGCGCGCGGGTCGGGG |
| 1OB16NT | 174 | Antiparallel | GGGCTGGGTCGGTCGGGCTGG |
| | | | GG |
| 8762NT | 175 | Complex | CGGAGGGCGCGCGGGTCGGGG |
| | | | CGGCGGCGGCGGCGGCGGTGG |
| | | | CGGCGGCGGCGGGGGCGGCGG |
| | | | G |

### Example 7

### Effects of Ribosomal Nucleic Acid Interacting Molecules on Nucleolin/Nucleic Acid Interactions

The following assays assessed effects of compounds on interactions between nucleolin and nucleic acid ligands capable of forming quadruplex (QP) and hairpin (HP) secondary structures. Nucleic acid ligands tested were a cMyc QP DNA having nucleotide sequence 5′-TGGGGAGGGTGGGGAGGGTGGGGAAGG-3′(SEQ ID NO: 176) and a HP pre-rRNA region to which nucleolin binds, having the sequence 5′-GGCCGAAAUCCCGAAGUAGGCC-3′ (SEQ ID NO: 177). In the assays, recombinant nucleolin (˜250 nM), which was fused to maltose binding protein and had the sequence under accession number NM_{—}005381 without the N-terminal acidic stretches domain, was incubated with each of the two ³²P-labeled nucleic acid ligands (10 or 250 nM). Nucleolin and the nucleic acid ligand were incubated in the presence or absence of a test compound of Formula A-1, B-1, C-6 or C-7 in an incubation buffer (12.5 mM Tris, pH 7.6, 60 mM KCl, 1 mM MgCl₂, 0.1 mM EDTA, 1 mM DTT, 5% glycerol, 0.1 mg/ml BSA) for 30 minutes at room temperature. Structures for A-1 and B-1 are shown above and structures for C-6 and C-7 are shown hereafter:

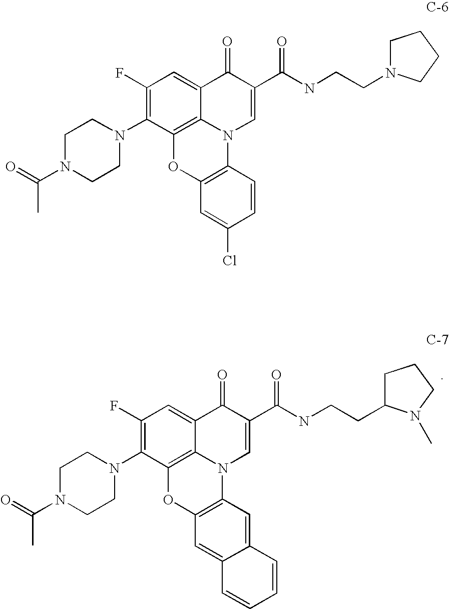

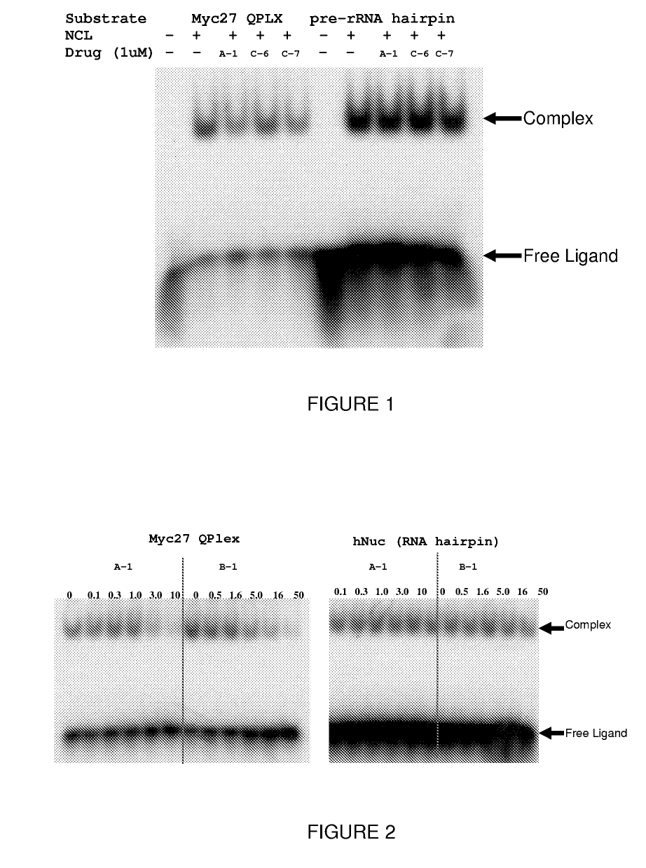

The resulting complexes were separated on a 6% DNA retardation gel using 0.5×TBE with 20 mM KCl as a running buffer (i.e., electrophoresis mobility shift assay (EMSA)). FIG. 1 shows compounds of formulae A-1, C-6 and C-7 interfered with the nucleolin/QP ligand interaction but did not significantly interfere with the nucleolin/HP ligand interaction. FIG. 2 shows each of compounds A-1 and B-1 interfered with the nucleolin/QP ligand interaction in a concentration dependent manner, but did not significantly interfere with the nucleolin/HP interaction.

The assay also was conducted using nucleic acid ligands derived from human ribosomal DNA. Sequences of these nucleic acids are shown in the preceding example. It was determined from these assays that compound A-1, but not Actinomycin D, interfered with nucleolin/nucleic acid ligand interactions. The table directly below shows for each nucleic acid ligand the relative affinity for nucleolin and the relative activity of compound A-I in interfering with the nucleolin/nucleic acid ligand interaction. A “+” represents the weakest nucleolin affinity and least interference by compound A-I and a “++++” represents the strongest nucleolin affinity and greatest interference by compound A-1. The table also shows the conformation of the intramolecular quadruplex structure formed by the nucleic acid ligand determined by circular dichroism, as described above. RND27 is a single-stranded nucleic acid having a random sequence that does not form a quadruplex structure.

| Nucleic acid | | | Activity of |
|---|---|---|---|
| ligand | Conformation | Affinity for Nucleolin | Compound A-1 |
| 1196NT | Mixed | ++ | + |
| 2957NT | Mixed | +++ | +++ |
| 6183NT | Mixed | + | + |
| 6374NT | Mixed | − | NA |
| 6534NT | Parallel | +++ | ++ |
| 6960NT | Parallel | +++ | +++ |
| 7253NT | Mixed | +++ | ++ |
| 7733T | Mixed | + | +++ |
| 8511NT | Mixed | ++++ | − |
| 8749NT | Antiparallel | + | + |
| 8762NT | Complex | ++++ | +/− |
| 10816NT | Antiparallel | − | NA |
| 11028NT | Mixed | + | +++ |
| 13079NT | Parallel | ++ | +++ |
| 13137NT | Mixed | ++ | ++ |
| RND27 | Single-stranded | − | NA |

The assay also was conducted in a filter-binding format. In such forms of the assay, 0.2 nM of ³²P-labeled quadruplexes were incubated in 50 uL of the binding buffer (12.5 mM Tris-HCl, pH 7.6, 60 mM KCl, 1 mM MgCl2, 0.1 mM EDTA, 5% glycerol, 0.1 mg/mL BSA) for 10 min at 85 C and then for 10 min on ice and mixed with another 50 uL of binding buffer containing increasing amounts of recombinant protein Nucleolin. The protein-quadruplex mixtures were incubated for 30 min at ambient temperature and filtered through mixed cellulose ester membrane filters (Millipore) with gentle suction. The filters were washed twice with 300 mL of binding buffer, dried and OptiPhase ‘SuperMix’ scintillation cocktail (Perkin Elmer) was added to the wells. Radioactivity was assayed with MicroBeta scintillation counter (Perkin Elmer). Binding curves were constructed and apparent Kd's and Bmax's for the complexes were calculated using the GraphPad Prizm software program (GraphPad Software). In the following table, the nucleic acid ligand is designated in the first column using the nomenclature described herein; the second column provides the nucleotide sequence of the nucleic acid ligand; the third column is the conformation of the ligand as determined by circular dichroism (M is mixed, P is parallel, A is antiparallel, C is complex, SS is single-stranded and ND is not determined); the fourth column is the dissociation constant determined by the filter binding assay of nucleolin protein and the nucleic acid ligand; the fifth column is a Bmax constant determined by the filter binding assay, which is the percent of active nucleic acid ligand in each assay; and the sixth column presents the concentration of Compound A-1 required to dissociate half of the complexed nucleic acid ligand and nucleolin protein, as determined by the EMSA assay described above.

| | | | | | | |
|---|---|---|---|---|---|---|
| SEQ | | | | | | |
| ID | Nucleic | | | Kd | Bma | IC50 |
| NO | Acid | Sequence | CD | X (%) | (uM) | |

| | | | | | | |
|---|---|---|---|---|---|---|
| 162 | 1196NT | GGGTGGACGGGGGGGCCTG | M | 4.2 | 41 | 3 |
| | | GTGGGG | | | | |
| 163 | 2957NT | GGGTCGGGGGGTGGGGCCC | M | 2.7 | 66 | 1 |
| | | GGGCCGGGG | | | | |
| 178 | 5701N1 | AGGGAGGGAGACGGGGGCG | M | 3.2 | 84 | 3 |
| 166 | 6183NT | GGGTCGGGGGCGGTGGTGG | M | 2.2 | 26 | 3 |
| | | GCCCGCGGGGG | | | | |
| 168 | 6374NT | GGGGGCGGGAACCCCCGGG | M | 5.5 | 47 | 3 |
| | | CGCCTGTGGG | | | | |
| 161 | 6534NT | GGGCGGGGGGGGCGGGGGG | P | 1.1 | 51 | 10 |
| 160 | 6960NT | GGGTGGCGGGGGGGAGAGG | P | 0.5 | 68 | 3 |
| | | GGGG | | | | |
| 170 | 7253NT | GGGTCCGGAAGGGGAAGGG | M | 0.4 | 60 | 10 |
| | | TGCCGGCGGGGAGAGAGGG | | | | |
| | | TCGGGGG | | | | |
| 165 | 8511NT | GGGGGCGGGCTCCGGCGGG | M | 0.6 | 100 | 10 |
| | | TGCGGGGGTGGGCGGGCGG | | | | |
| | | GGCCGGGGGTGGGGTCGGC | | | | |
| | | GGGGG | | | | |
| 173 | 8749NT | GGGAGGGCGCGCGGGTCGG | A | 1.9 | 13 | 10 |
| | | GG | | | | |
| | 8762NT | | C | 0.3 | 100 | 10 |
| 174 | 10816NT | GGGCTGGGTCGGTCGGGCT | A | >30 | | ND |
| | | GGGG | | | | |
| 167 | 11028NT | GGGGCGCGGCGGGGGGAGA | M | 2.7 | 32 | ND |
| | | AGGGTCGGGGCGGCAGGGG | | | | |
| 159 | 13079NT | GGGGTGGGGGGGAGGG | P | 2.6 | 37 | ND |
| 171 | 13173NT | GGGCCGGGACGGGGTCCGG | M | >30 | | ND |
| | | GG | | | | |
| 179 | 13101 | AGGGACGCCTGGGGAAGGG | ND | 4.4 | 50 | ND |
| | | AGGGGG | | | | |
| 180 | 21601 | AGGGCGGCGGGCGGGGAAG | ND | 0.7 | 55 | ND |
| | | AGGGCACAGACGGGCGAGG | | | | |
| | | GCCGGGGACCGCGAGGGCA | | | | |
| | | AGGGCACCCGGG | | | | |
| 181 | 34681 | AGGGACCGGTGGGGCCGGG | ND | 2.4 | 21 | ND |
| | | GCGGGG | | | | |
| 182 | 35001 | AGGGCGGACGGGAGGGAGC | ND | 1.1 | 20 | ND |
| | | GAGCGGGCGCGGGGG | | | | |
| 172 | 69141 | GGGCCCGCGGGGGGAGGGG | M | 0.8 | 50 | ND |
| | | GAAGGGGCGGG | | | | |
| 183 | 73701 | AGGGGCGGCGGGGAGGAGG | ND | 1.1 | 16 | ND |
| | | AGGGGCGCGGGG | | | | |
| 184 | 74991 | AGGGCGGGCGGCGGGGCGG | ND | 1.1 | 16 | ND |
| | | G | | | | |
| 169 | 7733T | GGGAGGGGCACGGGCCGGG | M | 0.8 | 53 | ND |
| | | GGCGGGACGGG | | | | |
| 185 | 8440T | AGGGGCGGCGGGGGAAGGG | ND | 0.3 | 35 | ND |
| | | AGGGCGGGTGGAGGGGTCG | | | | |
| | | GGAGGAACGGGGGGCGGG | | | | |
| 186 | 8716T | AGGGAGGACGCGGGGCCGG | ND | 3.1 | 49 | ND |
| | | GGGGCGGAGACGGG | | | | |
| 187 | 8904T | AGGGGGTGGGCGCCGGGAG | ND | 0.4 | 31 | ND |
| | | GGGGG | | | | |
| 188 | 9024T | AGGGGCGGGGGCGCGGGGA | ND | 0.3 | 43 | ND |
| | | GGAGGGGTGGG | | | | |
| 189 | 10110T | GGGGGGGGGGGCGGGGG | P | ND | | ND |
| 190 | 10137T | AGGGGCTCCGGGGGCGGGG | ND | 0.4 | 65 | ND |
| | | AGCGGGGCGTGGGCGGGAG | | | | |
| | | GAGGGG | | | | |
| 191 | 10885T | AGGGTGGGGCGGGGGAGGG | ND | 0.2 | 82 | ND |
| | | CCGCGAGGGGGGTGCCCCG | | | | |
| | | GGCGTGGGGGGGG | | | | |
| 192 | 10951T | AGGGCGCGGGGTGGGGAGG | ND | 0.2 | 52 | ND |
| | | G | | | | |
| 193 | 10985T | AGGGGAGGGGGGAGGACGG | ND | 0.2 | 35 | ND |
| | | GGAGCGGGGG | | | | |
| 194 | 11345T | AGGGGGGAACGGGGGGCGG | ND | 0.3 | 32 | ND |
| | | ACGGGGCCGGGGGGGTACG | | | | |
| | | GCGGGGGG | | | | |
| 195 | 11888T | AGGGGACACCGGGGGGGCG | ND | 0.2 | 47 | ND |
| | | CCGGGGG | | | | |
| 196 | 132361 | AGGGACGGGGACCGGCGGG | ND | >30 | | ND |
| | | CCACGGGG | | | | |
| 197 | hTeI | AGGGTTAGGGTTAGGGTTA | A | >30 | | ND |
| | | GGG | | | | |
| 198 | Myc27 | TGGGGAGGGTGGGGAGGGT | P | 4.4 | 33 | ND |
| | | GGGGAATT | | | | |
| 199 | RND27 | GTCGTAACGTCGATCACTT | SS | >30 | | ND |
| | | TACGACAT | | | | |
| 200 | GGA4 | GGAGGAGGAGGA | P | >30 | | ND |

### Example 8

### Effects of Compounds on Cell Cycle Progression and Cell Apoptosis

Assays were conducted to determine whether compounds described herein had an effect on cell cycle progression and could induce cell apoptosis. In assays for determining cell cycle progression effects, cells were harvested and single cell suspensions were prepared in buffer (e.g. PBS+2% FBS; PBS+0.1% BSA). Cells were washed twice and resuspend at 1-2×10⁶ cells/ml. One ml cells was aliquotted in a 15 ml polypropylene, V-bottomed tube and 3 ml cold absolute ethanol was added. Cells were fixed for at least one hour at 4° C. Fixed cells were washed twice in PBS and one ml of propidium iodide staining solution (3.8 mM sodium citrate, 50 ug/ml propidium iodide in PBS) was added to each cell pellet and mixed well. Fifty microliters of an RNase A stock solution (10 ug/ml RNase A boiled for 5 minutes and aliquoted and stored frozen at −20° C.) was added and the resulting mixture was incubated for 3 hours at 4° C. Samples were stored at 4° C. until analyzed by flow cytometry.

Apoptosis was assessed by Annexin V binding in flow cytometry fluorescence activated cell sorting (FACS) assays. In such assays, cells were harvested and washed twice in PBS (4° C.) and resuspended at a concentration of 1×10⁶ cells/ml in Binding Buffer (10× solution contains 0.1M HEPES/NaOH, pH7.4; 140 mM NaCl; 25 mM CaCl₂; PharMingen, 66121A). Cells were aliquotted (100 ul) into FACS tubes with Annexin V and/or viability dye. The tube contents were mixed gently and incubated for 15 minutes at room temperature in the dark. Binding Buffer (400 ul) was added to each tube and analyzed immediately by flow cytometry.

Annexin V is available in biotin, FITC (Annexin-V-FITC; PharMingen, 65874×) and PE (Annexin-PE; PharMingen, 65875×) formats. When using Annexin-V-FITC, Propidium Iodide (PI; Sigma, P 4170) was used as the viability marker (5 ul of a 50 ug/ml stock solution). When using Annexin-V-PE, 7-AminoActinomycin D (7-AAD; Sigma, A 9400) was the preferred viability marker (1 ug/ml final concentration) as there is less spectral overlap of PE and 7-AAD than PE and PI. While 7-AAD is not as bright as PI, FITC, PE and PI can be combined effectively. Tubes contained (i) cells alone, (ii) cells+Annexin, (iii) cells+PI (or 7-AAD) or (iv) cells+Annexin+PI (or 7-AAD) in some assays.

In these assays, compound A-1 induced apoptosis with little or no affect on the cell cycle. Compound A-1 was added at various concentrations for varying amounts of time with little to no effect on the cell cycle profile. Cell death induced by compound A-1 matched a classical apoptosis profile as DNA laddering and extracellular phosphatidyl serine (detected by annexin staining) were induced.

Compound B-1 in the assays induced apoptosis following an efficient arrest of cell cycle progression. HCT-116 colon carcinoma cells (p53+) arrested in G1 and G2 phases of the cell cycle. MiaPaCa pancreatic cells and DAOY medulloblastoma cells (p53−) arrested primarily in the S phase with some G2 arrest as well.

### Example 9

### Methods for Determining Quadruplex Formation and Conformation

Known assays can be utilized to determine whether a nucleic acid is capable of adopting a quadruplex structure. These assays include mobility shift assays, DMS methylation protection assays, polymerase arrest assays, transcription reporter assays, circular dichroism assays, and fluorescence assays.

Gel Electrophoretic Mobility Shift Assay (EMSA)

An EMSA is useful for determining whether a nucleic acid forms a quadruplex and whether a nucleotide sequence is quadruplex-altering. EMSA is conducted as described previously (Jin & Pike, Mol. Endocrinol. 10: 196-205 (1996)) with minor modifications. Synthetic single-stranded oligonucleotides are labeled in the 5′-terminus with T4-kinase in the presence of [α-³²P] ATP (1,000 mCi/mmol, Amersham Life Science) and purified through a sephadex column. ³²P-labeled oligonucleotides (˜30,000 cpm) then are incubated with or without various concentrations of a testing compound in 20 μl of a buffer containing 10 mM Tris pH 7.5, 100 mM KCl, 5 mM dithiothreitol, 0.1 mM EDTA, 5 mM MgCl₂, 10% glycerol, 0.05% Nonedit P-40, and 0.1 mg/ml of poly(dI-dC) (Pharmacia). After incubation for 20 minutes at room temperature, binding reactions are loaded on a 5% polyacrylamide gel in 0.25×Tris borate-EDTA buffer (0.25×TBE, 1×TBE is 89 mM Tris-borate, pH 8.0, 1 mM EDTA). The gel is dried and each band is quantified using a phosphorimager.

DMS Methylation Protection Assay

Chemical footprinting assays are useful for assessing quadruplex structure. Quadruplex structure is assessed by determining which nucleotides in a nucleic acid are protected or unprotected from chemical modification as a result of being inaccessible or accessible, respectively, to the modifying reagent. A DMS methylation assay is an example of a chemical footprinting assay. In such an assay, bands from EMSA are isolated and subjected to DMS-induced strand cleavage. Each band of interest is excised from an electrophoretic mobility shift gel and soaked in 100 mM KCl solution (300 μl) for 6 hours at 4° C. The solutions are filtered (microcentrifuge) and 30,000 cpm (per reaction) of DNA solution is diluted further with 100 mM KCl in 0.1×TE to a total volume of 70 μl (per reaction). Following the addition of 1 μl salmon sperm DNA (0.1 μg/μl), the reaction mixture is incubated with 1 μl DMS solution (DMS:ethanol; 4:1; v:v) for a period of time. Each reaction is quenched with 18 μl of stop buffer (b-mercaptoathanol:water:NaOAc (3 M); 1:6:7; v:v:v). Following ethanol precipitation (twice) and piperidine cleavage, the reactions are separated on a preparative gel (16%) and visualized on a phosphorimager.

Polymerase Arrest Assay

An example of the Taq polymerase stop assay is described in Han et al., Nucl. Acids Res. 27: 537-542 (1999), which is a modification of that used by Weitzmann et at, J. Biol. Chem. 271, 20958-20964 (1996). Briefly, a reaction mixture of template DNA (50 nM), Tris.HCl (50 mM), MgCl₂ (10 mM), DTT (0.5 mM), EDTA (0.1 mM), BSA (60 ng), and 5′-end-labeled quadruplex nucleic acid (˜18 nM) is heated to 90° C. for 5 minutes and allowed to cool to ambient temperature over 30 minutes. Taq Polymerase (1 μl) is added to the reaction mixture, and the reaction is maintained at a constant temperature for 30 minutes. Following the addition of 10 μl stop buffer (formamide (20 ml), 1 M NaOH (200 μl), 0.5 M EDTA (400 μl), and 10 mg bromophenol blue), the reactions are separated on a preparative gel (12%) and visualized on a phosphorimager. Adenine sequencing (indicated by “A” at the top of the gel) is performed using double-stranded DNA Cycle Sequencing System from Life Technologies. The general sequence for the template strands is TCCAACTATOTATAC (SEQ ID NO:201)-INSERT-TTAGCGACACGCAATTGCTATAGTGAGTCGTATTA (SEQ ID NO: 202). Bands on the gel that exhibit slower mobility are indicative of quadruplex formation.

Transcription Reporter Assay

A luciferase promoter assay described in He et al., Science 281: 1509-1512 (1998) often is utilized for the study of quadruplex formation. Specifically, a vector utilized for the assay is set forth in reference 11 of the He et al. document. In this assay, HeLa cells are transfected using the lipofectamin 2000-based system (Invitrogen) according to the manufacturer's protocol, using 0.1 μg of pRL-TK (Renilla luciferase reporter plasmid) and 0.9 μg of the quadruplex-forming plasmid. Firefly and Renilla luciferase activities are assayed using the Dual Luciferase Reporter Assay System (Promega) in a 96-well plate format according to the manufacturer's protocol.

Circular Dichroism Assay

Circular dichroism (CD) is utilized to determine whether another molecule interacts with a quadruplex nucleic acid. CD is particularly useful for determining whether a PNA or PNA-peptide conjugate hybridizes with a quadruplex nucleic acid in vitro. PNA probes are added to quadruplex DNA (5 μM each) in a buffer containing 10 mM potassium phosphate (pH 7.2) and 10 or 250 mM KCl at 37° C. and then allowed to stand for 5 min at the same temperature before recording spectra. CD spectra are recorded on a Jasco J-715 spectropolarimeter equipped with a thermoelectrically controlled single cell holder. CD intensity normally is detected between 220 nm and 320 nm and comparative spectra for quadruplex DNA alone, PNA alone, and quadruplex DNA with PNA are generated to determine the presence or absence of an interaction (see, e.g., Datta et al., JACS 123:9612-9619 (2001)). Spectra are arranged to represent the average of eight scans recorded at 100 nm/min.

Fluorescence Binding Assay

50 μl of quadruplex nucleic acid or a nucleic acid not capable of forming a quadruplex is added in 96-well plate. A test molecule or quadruplex-targeted nucleic acid also is added in varying concentrations. A typical assay is carried out in 100 μl of 20 mM HEPES buffer, pH 7.0, 140 mM NaCl, and 100 mM KCl. 50 μl of the signal molecule N-methylmesoporphyrin IX (NMM) then is added for a final concentration of 3 μM. NMM is obtained from Frontier Scientific Inc, Logan, Utah. Fluorescence is measured at an excitation wavelength of 420 nm and an emission wavelength of 660 nm using a FluoroStar 2000 fluorometer (BMG Labtechnologies, Durham, N.C.). Fluorescence often is plotted as a function of concentration of the test molecule or quadruplex-targeted nucleic acid and maximum fluorescent signals for NMM are assessed in the absence of these molecules.

### Example 10

### Inhibition of rRNA Synthesis

Effects of compound A-1 and compound B-1 on DNA synthesis, RNA synthesis and protein synthesis were determined in HCT116 cells. HCT 116 cells were plated overnight at 100,000 cells per mL. Next day cells were treated with increasing amounts of either compound A-1 or compound B-1 followed by one hour incubation with BrdU label (from a BrdU Cell proliferation Assay Kit, Calbiochem) to monitor DNA synthesis; 5 mCi of ³H-uridine to monitor total RNA synthesis; 5 mCi of ³H-methionine to monitor protein synthesis or plain media to monitor RNA Polymerase II-dependent RNA synthesis. DNA synthesis was assessed using a BrdU-ELISA (BrdU Cell proliferation Assay Kit, Calbiochem). To measure total RNA synthesis, total RNA from treated cells was isolated with a RNease kit (QIAGEN), levels of total RNA were assessed with Ribogreen reagent (Invitrogen) and the newly synthesized tritiated RNA was measured in a scintillation Counter (Perkin Elmer). To measure effects on protein synthesis, cells were lysed in a RIPA buffer, and total protein was precipitated with 10% TCA on a glass-filters. Newly synthesized tritiated protein was measured in a scintillation Counter (Perkin Elmer). Effects of drugs on Pol II-dependent RNA synthesis were assessed by monitoring levels of a c-myc mRNA, which has a relatively short half-life of approximately 30 minutes, by Taqman qRT-PCR (ABI).

Compound A-1 had no measureable effect on protein synthesis and c-myc mRNA levels at the tested concentrations. The compound significantly reduced nucleolar RNA synthesis at a 1 mM concentration. At a 10 mM concentration, a concentration at which many of the cells were dead, compound A-1 significantly reduced DNA synthesis. Compound B-1 had no measureable effect on protein synthesis and c-myc mRNA levels at the tested concentrations. Compound B-1 significantly reduced nucleolar RNA synthesis at 10 mM and DNA synthesis at 30 mM.

In a time course study, ³H-uridine incorporation was substantially inhibited by treatment of cells with 3 mM compound A-1 for 15 min, and 10 mM compound B-1 for 30 min. Accordingly, the compounds tested inhibited nucleolar RNA synthesis.

### Example 11

### Inhibition of Protein Kinases

Certain compounds were tested for activity in protein kinase inhibition assays. All substrates were dissolved and diluted to working stocks in de-ionized water, apart from histone H1 (10× working stock in 20 mM MOPS pH 7.0), PDKtide (10× working stock in 50 mM Tris pH 7.0) ATF2 (which is typically stored at a 20× working stock in 50 mM Tris pH 7.5, 150 mM NaCl, 0.1 mM EGTA, 0.03% Brij-35, 50% glycerol, 1 mM benzamidine, 0.2 mM PMSF and 0.1% R-mercaptoethanol), KKLNRTLSFAEPG (SEQ ID NO:203) and RRRLSFAEPG (SEQ ID NO:204) (50 mM HEPES pH 7.4) and GGEEEEYFELVKKKK (SEQ ID NO:205) (20 mM MOPS pH 7.0). All kinases were pre-diluted to a 10× working concentration prior to addition into the assay. The composition of the dilution buffer for each kinase is detailed below.

1. Blk, c-RAF, CSK, IGF-1R, IR, Lyn, MAPK1, MAPK2, MKK4, MKK6, MKK70, SAPK2a, SAPK2b, SAPK3, SAPK4, Syk, ZAP-70: 50 mM Tris pH 7.5, 0.1 mM EGTA, 0.1 mM Na3VO4, 0.1% beta-mercaptoethanol, 1 mg/ml BSA.

2. JNK1α1, JNK2α2, JNK3, PRK2, ROCK-II: 50 mM Tris pH 7.5, 0.1 mM EGTA, 0.1% beta-mercaptoethanol, 1 mg/ml BSA.

3. PDK1: 50 mM Tris pH 7.5, 0.05% Beta-mercaptoethanol, 1 mg/ml BSA.

4. MEK-1: 25 mM Tris pH 7.5, 0.1 mM EGTA, 0.1% beta-mercaptoethanol, 1 mg/ml BSA.

5. Abl, Abl(T315I), ALK, ALK4, Arg, Ask1, Aurora-A, Axl, Bmx, BRK, BTK, CDK1/cyclinB, CDK2/cyclinA, CDK2/cyclinE, CDK3/cyclinE, CDK5/p25, CDK5/p35, CDK6/cyclinD3, CDK7/cyclinH/MAT1, CHK1, CHK2, CK1, CKIδ, cKit, cKit (D816V), cSRC, DDR2, EGFR, EGFR (L858R), EGFR (L861Q), EphA2, EphA3, EphA4, EphA5, EphB2, EphB3, EphB4, ErbB4, Fer, Fes, FGFR1, FGFR2, FGFR3, FGFR4, Fgr, Flt1, Flt3, Flt3 (D835Y), Fms, Fyn, GSK3α, GSK3 β, Hck, HIPK2, IKKα, IKKβ, IRAK4, IRR, JAK2, JAK3, KDR, Lck, MAPKAP-K2, MAPKAP-K3, Met, MINK, MLCK, MRCKβ, MSK1, MSK2, MST1, MST2, MuSK, NEK2, NEK6, Nek7, p70S6K, PAK2, PAK4, PAK6, PAR-1Bα, PDGFRα, PDGFRβ, Pim-1, PKA, PKBα, PKBβ, PKBγ, PKC6, PKCQ, PKG1β, Plk3, Pyk2, Ret, RIPK2, Rse, ROCK-1, Ron, Ros, Rsk1, Rsk2, Rsk3, SGK, SGK2, SGK3, Snk, TAK1, TBK1, Tie2, TrkA, TrkB, TSSK2, Yes, ZIPK: 20 mM MOPS pH 7.0, 1 mM EDTA, 0.1% Beta-mercaptoethanol, 0.01% Brij-35, 5% glycerol, 1 mg/ml BSA.

6. CK2: 20 mM HEPES pH 7.6, 0.15 M NaCl, 0.1 mM EGTA, 5 mM DTT, 0.1% Triton X-100, 50% glycerol.

7. CaMKII, CaMKIV: 40 mM HEPES pH 7.4, 1 mg/ml BSA.

8. PKCα, PKCβ1, PKCβII, PKCγ, PKCδ, PKCε, PKCηI, PKCt, PKCμ, PKD2: 20 mM HEPES pH 7.4, 0.03% Triton X-100.

9. PRAK: Beta-mercaptoethanol, 0.1 mM EGTA, 1 mg/ml BSA.

10. AMPK: 50 mM Na R-glycerophosphate pH 7.0, 0.1%.

Protein kinase assays were conducted as follows:

Abl (h)

In a final reaction volume of 25 μl, Abl (h) (5-10 mU) is incubated with 8 mM MOPS pH 7.0, 0.2 mM EDTA, 50 μM EAIYAAPFAKKK (SEQ ID NO:206), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Abl (T3151) (h)

In a final reaction volume of 25 μl, Abl (T3151) (h) (5-10 mU) is incubated with 8 mM MOPS pH 7.0, 0.2 mM EDTA, 50 μM EAIYAAPFAKKK (SEQ ID NO:206), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Abl (m)

In a final reaction volume of 25 μl, Abl (m) (5-10 mU) is incubated with 8 mM MOPS pH 7.0, 0.2 mM EDTA, 50 μM EAIYAAPFAKKK (SEQ ID NO:206), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

ALK (h)

In a final reaction volume of 25 μl, ALK (h) (5-10 mU) is incubated with 8 mM MOPS pH 7.0, 0.2 mM EDTA, 250 μM KKKSPGEYVNIEFG (SEQ ID NO:207), 10 mM MgAcetate and [y-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

ALK4 (h)

In a final reaction volume of 25 μl, ALK4 (h) (5-10 mU) is incubated with 8 mM MOPS pH 7.0, 0.2 mM EDTA, 2 mg/ml casein, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

AMPK (r)

In a final reaction volume of 25 μl, AMPK (r) (5-10 mU) is incubated with 32 mM HEPES pH 7.4, 0.65 mM DTT, 0.012% Brij-35,200 μM AMP, 200 μM AMARAASAAALARRR (SEQ ID NO:208), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Arg (h)

In a final reaction volume of 25 μl, Arg (h) (5-10 mU) is incubated with 8 mM MOPS pH 7.0, 0.2 mM EDTA, 50 μM EAIYAAPFAKKK (SEQ ID NO:206), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Arg (m)

In a final reaction volume of 25 μl, Arg (m) (5-10 mU) is incubated with 8 mM MOPS pH 7.0, 0.2 mM EDTA, 50 μM EAIYAAPFAKKK (SEQ ID NO:206), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

ASK1 (h)

In a final reaction volume of 25 μl, ASK I (h) (5-10 mU) is incubated with 8 mM MOPS pH 7.0, 0.2 mM EDTA, 0.33 mg/ml myelin basic protein, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Aurora-A (h)

In a final reaction volume of 25 μl, Aurora-A (h) (5-10 mU) is incubated with 8 mM MOPS pH 7.0, 0.2 mM EDTA, 200 μM LRRASLG (SEQ ID NO:209) (Kemptide), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 50 mM phosphoric acid and once in methanol prior to drying and scintillation counting.

Axl (h)

In a final reaction volume of 25 μl, Axl (h) (5-10 mU) is incubated with 8 mM MOPS pH 7.0, 0.2 mM EDTA, 250 μM KKSRGDYMTMQIG (SEQ ID NO:210), 10 mM MgAcetate and [y-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Blk (m)

In a final reaction volume of 25 μl, Blk (m) (5-10 mU) is incubated with 50 mM Tris pH 7.5, 0.1 mM EGTA, 0.1 mM Na₃VO4, 0.1% R-mercaptoethanol, 0.1 mg/ml poly(Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

Bmx (h)

In a final reaction volume of 25 μl, Bmx (h) (5-10 mU) is incubated with 8 mM MOPS pH 7.0, 0.2 mM EDTA, 0.1 mg/ml poly(Glu, Tyr) 4: 1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

BRK (h)

In a final reaction volume of 25 μl, BRK (h) (5-10 mU) is incubated with 8 mM MOPS pH 7.0, 0.2 mM EDTA, 5 mM MnCl2, 0.1 mg/ml poly (Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

BTK (h)

In a final reaction volume of 25 μl, BTK (h) (5-10 mU) is incubated with 8 mM MOPS pH 7.0, 0.2 mM EDTA, 250 μM KVEKIGEGTYGVVYK (SEQ ID NO:211) (Cdc2 peptide), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

CaMKII (r)

In a final reaction volume of 25 μl, CaMKII (r) (5-10 mU) is incubated with 40 mM HEPES pH 7.4, 5 mM CaCl2, 30 μg/ml calmodulin, 30 μM KKLNRTLSVA (SEQ ID NO:212), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

CaMKIV (h)

In a final reaction volume of 25 μl, CaMKIV (h) (5-10 mU) is incubated with 40 mM HEPES pH 7.4, 5 mM CaCl2, 30 μg/ml calmodulin, 30 μM KKLNRTLSVA (SEQ ID NO:212), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

CDK1/cyclinB (h)

In a final reaction volume of 25 μl, CDK1/cyclinB (h) (5-10 mU) is incubated with 8 mM MOPS pH 7.0, 0.2 mM EDTA, 0.1 mg/ml histone Hi, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

CDK2/cyclinA (h)

In a final reaction volume of 251l, CDK2/cyclinA (h) (5-10 mU) is incubated with 8 mM MOPS pH 7.0, 0.2 mM EDTA, 0.1 mg/ml histone Hi, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

CDK2/cyclinE (h)

In a final reaction volume of 25 μl, CDK2/cyclinE (h) (5-10 mU) is incubated with 8 mM MOPS pH 7.0, 0.2 mM EDTA, 0.1 mg/ml histone H1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

CDK3/cyclinE (h)

In a final reaction volume of 25 μl, CDK3/cyclinE (h) (5-10 mU) is incubated with 8 mM MOPS pH 7.0, 0.2 mM EDTA, 0.1 mg/ml histone H1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

CDK5/p25 (h)

In a final reaction volume of 25 μl, CDK5/p25 (h) (5-10 mU) is incubated with 8 mM MOPS pH 7.0, 0.2 mM EDTA, 0.1 mg/ml histone H1, 10 mM MgAcetate and [y-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

CDK5/p35 (h)

In a final reaction volume of 25 μl, CDK5/p35 (h) (5-10 mU) is incubated with 8 mM MOPS pH 7.0, 0.2 mM EDTA, 0.1 mg/ml histone Hi, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

CDK6/cyclinD3 (h)

In a final reaction volume of 25 μl, CDK6/cyclinD3 (h) (5-10 mU) is incubated with 8 mM MOPS pH 7.0, 0.2 mM EDTA, 0.1 mg/ml histone Hi, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

CDK7/cyclinH/MAT1 (h)

In a final reaction volume of 25 μl, CDK7/cyclinH/MAT1 (h) (5-10 mU) is incubated with 8 mM MOPS pH 7.0, 0.2 mM EDTA, 500 μM peptide, 10 mM MgAcetate and [y-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

CHK1 (h)

In a final reaction volume of 25 μl, CHK1 (h) (5-10 mU) is incubated with 8 mM MOPS pH 7.0, 0.2 mM EDTA, 200 μM KKKVSRSSGLYRSPSMPENLNRPR (SEQ ID NO:213), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

CHK2 (h)

In a final reaction volume of 25 μl, CHK2 (h) (5-10 mU) is incubated with 8 mM MOPS pH 7.0, 0.2 mM EDTA, 200 μM KKKVSRSGLYRSPSMPENLNRPR (SEQ ID NO:213), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

CK1 (y)

In a final reaction volume of 25 μl, CK1 (y) (5-10 mU) is incubated with 8 mM MOPS pH 7.0, 0.2 mM EDTA, 200 μM KRRRALS(p)VASLPGL (SEQ ID NO:214), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

CK1δ (h)

In a final reaction volume of 25 μl, CK1 (h) (5-10 mU) is incubated with 8 mM MOPS pH 7.0, 0.2 mM EDTA, 200 μM KRRRALS(p)VASLPGL (SEQ ID NO:214), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

CK2 (h)

In a final reaction volume of 25 μl, CK2 (h) (5-10 mU) is incubated with 20 mM HEPES pH 7.6, 0.15 M NaCl, 0.1 mM EDTA, 5 mM DTT, 0.1% Triton X-100, 165 μM RRRDDDSDDD (SEQ ID NO:215), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

cKit (h)

In a final reaction volume of 25 μl, cKit (h) (5-10 mU) is incubated with 8 mM MOPS pH 7.0, 0.2 mM EDTA, 10 mM MnCl2, 0.1 mg/ml poly(Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

cKit (D816V) (h)

In a final reaction volume of 25 μl, cKit (D816V) (h) (5-10 mU) is incubated with 8 mM MOPS pH 7.0, 0.2 mM EDTA, 10 mM MnCl2, 0.1 mg/ml poly(Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

c-RAF (h)

In a final reaction volume of 25 μl, c-RAF (h) (5-10 mU) is incubated with 25 mM Tris pH 7.5, 0.02 mM EGTA, 0.66 mg/ml myelin basic protein, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

CSK (h)

In a final reaction volume of 25 μl, CSK (h) (5-10 mU) is incubated with 50 mM Tris pH 7.5, 0.1 mM EGTA, 0.1 mM Na₃VO4, 0.1% R-mercaptoethanol, 0.1 mg/ml poly(Glu, Tyr) 4: 1, 10 mM MnCl2, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

cSRC (h)

In a final reaction volume of 25 μl, cSRC (h) (5-10 mU) is incubated with 8 mM MOPS pH 7.0, 0.2 mM EDTA, 250 μM KVEKIGEGTYGVVYK (SEQ ID NO:211) (Cdc2 peptide), 10 mM MgAcetate and [y-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

DDR2 (h)

In a final reaction volume of 25 μl, DDR2 (h) (5-10 mU) is incubated with 8 mM MOPS pH 7.0, 0.2 mM EDTA, 250 μM KKSRGDYMTMQIG (SEQ ID NO:210), 10 mM MnCl2, 10 mM MgAcetate and [y-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

EGFR (h)

In a final reaction volume of 25 μl, EGFR (h) (5-10 mU) is incubated with 8 mM MOPS pH 7.0, 0.2 mM EDTA, 10 mM MnCl2, 0.1 mg/ml poly(Glu, Tyr) 4: 1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

EGFR (L858R) (h)

In a final reaction volume of 25 μl, EGFR (L858R) (h) (5-10 mU) is incubated with 8 mM MOPS pH 7.0, 0.2 mM EDTA, 0.1 mg/ml poly(Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

EGFR (L861O) (h)

In a final reaction volume of 25 μl, EGFR (L861Q) (h) (5-10 mU) is incubated with 8 mM MOPS pH 7.0, 0.2 mM EDTA, 0.1 mg/ml poly(Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

EphA2 (h)

In a final reaction volume of 25 μl, EphA2 (h) (5-10 mU) is incubated with 8 mM MOPS pH 7.0, 0.2 mM EDTA, 0.1 mg/ml poly(Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

EphA3 (h)

In a final reaction volume of 25 μl, EphA3 (h) (5-10 mU) is incubated with 8 mM MOPS pH 7.0, 0.2 mM EDTA, 0.1 mg/ml poly(Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

EphA4 (h)

In a final reaction volume of 25 μl, EphA4 (h) (5-10 mU) is incubated with 8 mM MOPS pH 7.0, 0.2 mM EDTA, 10 mM MnCl2, 0.1 mg/ml poly(Glu, Tyr) 4:1, 10 mM MgAcetate and [y-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

EphA5 (h)

In a final reaction volume of 251p1, EphA5 (h) (5-10 mU) is incubated with 8 mM MOPS pH 7.0, 0.2 mM EDTA, 2.5 mM MnCl2, 0.1 mg/ml poly (Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

EphB2 (h)

In a final reaction volume of 25 μl, EphB2 (h) (5-10 mU) is incubated with 8 mM MOPS pH 7.0, 0.2 mM EDTA, 10 mM MnCl2, 0.1 mg/ml poly(Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

EphB3 (h)

In a final reaction volume of 25 μl, EphB3 (h) (5-10 mU) is incubated with 8 mM MOPS pH 7.0, 0.2 mM EDTA, 10 mM MnCl2, 0.1 mg/ml poly (Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

EphB4 (h)

In a final reaction volume of 25 μl, EphB4 (h) (5-10 mU) is incubated with 8 mM MOPS pH 7.0, 0.2 mM EDTA, 10 mM MnCl2, 0.1 mg/ml poly(Glu, Tyr) 4: 1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

ErbB4 (h)

In a final reaction volume of 25 μl, ErbB4 (h) (5-10 mU) is incubated with 8 mM MOPS pH 7.0, 0.2 mM EDTA, 2.5 mM MnCl2, 0.1 mg/ml poly(Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

Fer (h)

In a final reaction volume of 25 μl, Fer (h) (5-10 mU) is incubated with 8 mM MOPS pH 7.0, 0.2 mM EDTA, 1 mM MnCl2, 250 μM KKKSPGEYVNIEFG (SEQ ID NO:207), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Fes (h)

In a final reaction volume of 25 μl, Fes (h) (5-10 mU) is incubated with 8 mM MOPS pH 7.0, 0.2 mM EDTA, 0.1 mg/ml poly(Glu, Tyr) 4: 1, 10 mM MgAcetate and [y-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

FGFR1 (h)

In a final reaction volume of 25 μl, FGFR1 (h) (5-10 mU) is incubated with 8 mM MOPS pH 7.0, 0.2 mM EDTA, 250 μM KKKSPGEYVNIEFG (SEQ ID NO:207), 10 mM MgAcetate and [y-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

FGFR2 (h)

In a final reaction volume of 25 μl, FGFR2 (h) (5-10 mU) is incubated with 8 mM MOPS pH 7.0, 0.2 mM EDTA, 2.5 mM MnCl2, 0.1 mg/ml poly(Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

FGFR3 (h)

In a final reaction volume of 25 μl, FGFR3 (h) (5-10 mU) is incubated with 8 mM MOPS pH 7.0, 0.2 mM EDTA, 0.1 mg/ml poly(Glu, Tyr) 4:1, 10 mM MnCl2, 10 mM MgAcetate and [y-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

FGFR4 (h)

In a final reaction volume of 25 μl, FGFR4 (h) (5-10 mU) is incubated with 8 mM MOPS pH 7.0, 0.2 mM EDTA, 10 mM MnCl2, 0.1 mg/ml poly(Glu, Tyr) 4:1, 10 mM MgAcetate and [gamma-3 3P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

Fgr (h)

In a final reaction volume of 25 μl, Fgr (h) (5-10 mU) is incubated with 8 mM MOPS pH 7.0, 0.2 mM EDTA, 0.1 mg/ml poly(Glu, Tyr) 4: 1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

Flt1 (h)

In a final reaction volume of 25 μl, Flt1 (h) (5-10 mU) is incubated with 8 mM MOPS pH 7.0, 0.2 mM EDTA, 250 μM KKKSPGEYVNIEFG (SEQ ID NO:207), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Flt3 (h)

In a final reaction volume of 25 μl, Flt3 (h) (5-10 mU) is incubated with 8 mM MOPS pH 7.0, 0.2 mM EDTA, 50 μM EAIYAAPFAKKK (SEQ ID NO:206), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Flt3 (D835Y) (h)

In a final reaction volume of 25 i-1, Flt3 (D835Y) (h) (5-10 mU) is incubated with 8 mM MOPS pH 7.0, 0.2 mM EDTA, 50 μM EAIYAAPFAKKK (SEQ ID NO:206), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Fms (h)

In a final reaction volume of 25 PI, Fms (h) (5-10 mU) is incubated with 8 mM MOPS pH 7.0, 0.2 mM EDTA, 250 μM KKKSPGEYVNIEFG (SEQ ID NO:207), 10 mM MgAcetate and [y-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Fyn (h)

In a final reaction volume of 25 μl, Fyn (h) (5-10 mU) is incubated with 50 mM Tris pH 7.5, 0.1 mM EGTA, 0.1 mM Na₃VO4, 250 μM KVEKIGEGTYGVVYK (SEQ ID NO:220) (Cdc2 peptide), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

GSK3α (h)

In a final reaction volume of 25 μl, GSK3α (h) (5-10 mU) is incubated with 8 mM MOPS pH 7.0, 0.2 mM EDTA, 20 μM YRRAAVPPSPSLSRHSSPHQS(p)EDEEE (SEQ ID NO:216) (phospho GS2 peptide), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 50 mM phosphoric acid and once in methanol prior to drying and scintillation counting.

GSK3β (h)

In a final reaction volume of 25 μl, GSK3β (h) (5-10 mU) is incubated with 8 mM MOPS pH 7.0, 0.2 mM EDTA, 20 μM YRRAAVPPSPSLSRHSSPHQS(p)EDEEE (SEQ ID NO:221) (phospho GS2 peptide), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 50 mM phosphoric acid and once in methanol prior to drying and scintillation counting.

Hck (h)

In a final reaction volume of 25 μl, Hck (h) (5-10 mU) is incubated with 8 mM MOPS pH 7.0, 0.2 mM EDTA, 250 μM KVEKIGEGTYGVVYK (Cd c2 peptide) (SEQ ID NO:211), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

HIPK2 (h)

In a final reaction volume of 25 μl, HIPK2 (h) (5-10 mU) is incubated with 8 mM MOPS pH 7.0, 0.2 mM EDTA, 0.33 mg/ml myelin basic protein, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

IGF-1R (h)

In a final reaction volume of 25 μl, IGF-IR (h) (5-10 mU) is incubated with 50 mM Tris pH 7.5, 0.1 mM EGTA, 0.1 mM Na₃VO4, 0.1% R-mercaptoethanol, 250 μM KKKSPGEYVNIEFG (SEQ ID NO:207), 10 mM MnCl2, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

IKKa (h)

In a final reaction volume of 25 μl, IKKa (h) (5-10 mU) is incubated with 8 mM MOPS pH 7.0, 0.2 mM EDTA, 200 μM peptide, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

IKKβ (h)

In a final reaction volume of 25 μl, IKKβ (h) (5-10 mU) is incubated with 8 mM MOPS pH 7.0, 0.2 mM EDTA, 100 μM peptide, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

IR (h)

In a final reaction volume of 25 μl, IR (h) (5-10 mU) is incubated with 50 mM Tris pH 7.5, 0.1 mM EGTA, 0.1 mM Na₃VO4, 0.1% R-mercaptoethanol, 250 μM KKSRGDYMTMQIG (SEQ ID NO:210), 10 mM MnCl2, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

IRAK4 (h)

In a final reaction volume of 25 μl, IRAK4 (h) (5-10 mU) is incubated with 8 mM MOPS pH 7.0, 0.2 mM EDTA, 0.33 mg/ml myelin basic protein, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

IRR (h)

In a final reaction volume of 25 μl, IRR (h) (5-10 mU) is incubated with 8 mM MOPS pH 7.0, 0.2 mM EDTA, 0.33 mg/ml myelin basic protein, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

JAK2 (h)

In a final reaction volume of 25 μl, JAK2 (h) (5-10 mU) is incubated with 8 mM MOPS pH 7.0, 0.2 mM EDTA, 100 μM KTFCGTPEYLAPEVRREPRILSEEEQEMFRDFDYIADWC (SEQ ID NO:217), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

JAK3 (h)

In a final reaction volume of 25 μl, JAK3 (h) (5-10 mU) is incubated with 8 mM MOPS pH 7.0, 0.2 mM EDTA, 500 μM GGEEEEYFELVKKKK (SEQ ID NO:218), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

JNK1α (h)

In a final reaction volume of 25 μl, JNK1a (h) (5-10 mU) is incubated with 50 mM Tris pH 7.5, 0.1 mM EGTA, 0.1% R-mercaptoethanol, 3 μM ATF2, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

JNK2α2 (h)

In a final reaction volume of 25 μl, JNK2α2 (h) (5-10 mU) is incubated with 50 mM Tris pH 7.5, 0.1 mM EGTA, 0.1% R-mercaptoethanol, 3 μM ATF2, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

JNK3 (h)

In a final reaction volume of 25 μl, JNK3 (h) (5-10 mU) is incubated with 50 mM Tris pH 7.5, 0.1 mM EGTA, 0.1% R-mercaptoethanol, 250 μM peptide, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

KDR (h)

In a final reaction volume of 25 μl, KDR (h) (5-10 mU) is incubated with 8 mM MOPS pH 7.0, 0.2 mM EDTA, 0.33 mg/ml myelin basic protein, 10 mM MgAcetate and [y-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Lek (h)

In a final reaction volume of 25 μl, Lck (h) (5-10 mU) is incubated with 50 mM Tris pH 7.5, 0.1 mM EGTA, 0.1 mM Na₃VO4,250 μM KVEKIGEGTYGVVYK (SEQ ID NO:211) (Cdc2 peptide), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Lyn (h)

In a final reaction volume of 25 μl, Lyn (h) (5-10 mU) is incubated with 50 mM Tris pH 7.5, 0.1 mM EGTA, 0.1 mM Na₃VO4, 0.1% R-mercaptoethanol, 0.1 mg/ml poly(Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

Lyn (m)

In a final reaction volume of 25 μl, Lyn (m) (5-10 mU) is incubated with 50 mM Tris pH 7.5, 0.1 mM EGTA, 0.1 mM Na₃VO4, 0.1% R-mercaptoethanol, 0.1 mg/ml poly(Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

MAPK1 (h)

In a final reaction volume of 25 μl, MAPK1 (h) (5-10 mU) is incubated with 25 mM Tris pH 7.5, 0.02 mM EGTA, 250 μM peptide, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

MAPK2 (h)

In a final reaction volume of 25 μl, MAPK2 (h) (5-10 mU) is incubated with 25 mM Tris pH 7.5, 0.02 mM EGTA, 0.33 mg/ml myelin basic protein, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

MAPK2 (m)

In a final reaction volume of 25 μl, MAPK2 (m) (5-10 mU) is incubated with 25 mM Tris pH 7.5, 0.02 mM EGTA, 0.33 mg/ml myelin basic protein, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

MAPKAP-K2 (h)

In a final reaction volume of 25 μl, MAPKAP-K2 (h) (5-10 mU) is incubated with 50 mM Na R-glycerophosphate pH 7.5, 0.1 mM EGTA, 30 μM KKLNRTLSVA (SEQ ID NO:212), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

MAPKAP-K3 (h)

In a final reaction volume of 25 μl, MAPKAP-K3 (h) (5-10 mU) is incubated with 50 mM Na R-glycerophosphate pH 7.5, 0.1 mM EGTA, 30 μM KKLNRTLSVA (SEQ ID NO:212), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

MEK1 (h)

In a final reaction volume of 25 μl, MEK1 (h) (1-5 mU) is incubated with 50 mM Tris pH 7.5, 0.2 mM EGTA, 0.1% R-mercaptoethanol, 0.01% Brij-35, 1 μM inactive MAPK2 (m), 10 mM MgAcetate and cold ATP (concentration as required). The reaction is initiated by the addition of the MgATP. After incubation for 40 minutes at room temperature, 5 μl of this incubation mix is used to initiate a MAPK2 (m) assay, which is described on page 12 of this book.

Met (h)

In a final reaction volume of 25 μl, Met (h) (5-10 mU) is incubated with 8 mM MOPS pH 7.0, 0.2 mM EDTA, 250 μM KKKSPGEYVNIEFG (SEQ ID NO:207), 10 mM MgAcetate and [y-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

MINK (h)

In a final reaction volume of 25 μl, MINK (h) (5-10 mU) is incubated with 8 mM MOPS pH 7.0, 0.2 mM EDTA, 0.33 mg/ml myelin basic protein, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

MKK4 (m)

In a final reaction volume of 25 μl, MKK4 (m) (1-5 mU) is incubated with 50 mM Tris pH 7.5, 0.1 mM EGTA, 0.1% R-mercaptoethanol, 0.1 mM Na₃VO4, 2 μM inactive JNK1a1 (h), 10 mM MgAcetate and cold ATP (concentration as required). The reaction is initiated by the addition of the MgATP. After incubation for 40 minutes at room temperature, 5 μl of this incubation mix is used to initiate a JNK1a1 (h) assay, which is exactly as described on page 11 of this book except that ATF2 is replaced with 250 μM peptide.

MKK6 (h)

In a final reaction volume of 25 μl, MKK6 (h) (1-5 mU) is incubated with 50 mM Tris pH 7.5, 0.1 mM EGTA, 0.1% R-mercaptoethanol, 0.1 mM Na3VO4, 1 mg/ml BSA, 1 μM inactive SAPK2a (h), 10 mM MgAcetate and cold ATP (concentration as required). The reaction is initiated by the addition of the MgATP. After incubation for 40 minutes at room temperature, 5 μl of this incubation mix is used to initiate a SAPK2a (h) assay, which is described on page 18 of this book.

MKK7β (h)

In a final reaction volume of 25 μl, MKK7β (h) (1-5 mU) is incubated with 50 mM Tris pH 7.5, 0.1 mM EGTA, 0.1% R-mercaptoethanol, 0.1 mM Na₃VO4, 2 μM inactive JNK1 al (h), 10 mM MgAcetate and cold ATP (concentration as required). The reaction is initiated by the addition of the MgATP. After incubation for 40 minutes at room temperature, 5 μl of this incubation mix is used to initiate a JNK1a1 (h) assay, which is exactly as described on page 11 of this book except that ATF2 is replaced with 250 μM peptide.

MLCK (h)

In a final reaction volume of 25 μl, MLCK (h) (5-10 mU) is incubated with 8 mM MOPS pH 7.0, 0.2 mM EDTA, 0.5 mM CaCl₂, 16 μg/ml calmodulin, 250 μM KKLNRTLSFAEPG (SEQ ID NO:203), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

MRCKβ (h)

In a final reaction volume of 25 μl, MRCKβ (h) (5-10 mU) is incubated with 8 mM MOPS pH 7.0, 0.2 mM EDTA, 100 μM KKRNRTLTV (SEQ ID NO:219), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

MSK1 (h)

In a final reaction volume of 25 μl, MSK1 (h) (5-10 mU) is incubated with 8 mM MOPS pH 7.0, 0.2 mM EDTA, 30 μM GRPRTSSFAEGKK (SEQ ID NO:220), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

MSK2 (h)

In a final reaction volume of 25 μl, MSK2 (h) (5-10 mU) is incubated with 8 mM MOPS pH 7.0, 0.2 mM EDTA, 30 μM GRPRTSSFAEGKK (SEQ ID NO:220), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

MST1 (h)

In a final reaction volume of 25 μl, MST1 (h) (5-10 mU) is incubated with 8 mM MOPS pH 7.0, 0.2 mM EDTA, 250 μM KKSRGDYMTMQIG (SEQ ID NO:210), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

MST2 (h)

In a final reaction volume of 25 PI, MST2 (h) (5-10 mU) is incubated with 8 mM MOPS pH 7.0, 0.2 mM EDTA, 0.33 mg/ml myelin basic protein, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

MuSK (h)

In a final reaction volume of 25 μl, MuSK (h) (5-10 mU) is incubated with 8 mM MOPS pH 7.0, 0.2 mM EDTA, 5 mM MnCl2, 0.33 mg/ml myelin basic protein, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

NEK2 (h)

In a final reaction volume of 25 μl, NEK2 (h) (5-10 mU) is incubated with 8 mM MOPS pH 7.0, 0.2 mM EDTA, 0.33 mg/ml myelin basic protein, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

NEK6 (h)

In a final reaction volume of 25 μl, NEK6 (h) (5-10 mU) is incubated with 8 mM MOPS pH 7.0, 0.2 mM EDTA, 300 μM FLAKSFGSPNRAYKK (SEQ ID NO:221), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

NEK7 (h)

In a final reaction volume of 25 μl, NEK7 (h) (5-10 mU) is incubated with 8 mM MOPS pH 7.0, 0.2 mM EDTA, 300 μM FLAKSFGSPNRAYKK (SEQ ID NO:221), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PAK2 (h)

In a final reaction volume of 25 μl, PAK2 (h) (5-10 mU) is incubated with 8 mM MOPS pH 7.0, 0.2 mM EDTA, 30 μM KEAKEKRQEQIAKRRRLSSLRASTSKSGGSQK (SEQ ID NO:222), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PAK4 (h)

In a final reaction volume of 25 μl, PAK-4 (h) (5-10 mU) is incubated with 8 mM MOPS pH 7.0, 0.2 mM EDTA, 0.8 mg/ml myelin basic protein, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PAK6 (h)

In a final reaction volume of 25 μl, PAK6 (h) (5-10 mU) is incubated with 8 mM MOPS pH 7.0, 0.2 mM EDTA, 200 μM RRRLSFAEPG (SEQ ID NO:223), 10 mM MgAcetate and [y-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PAR-1Bα (h)

In a final reaction volume of 25 μl, PAR-1Bα (h) (5-10 mU) is incubated with 8 mM MOPS pH 7.0, 0.2 mM EDTA, 100 μM KKKVSRSGLYRSPSMPENLNRPR (SEQ ID NO:213), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PDGFRα (h)

In a final reaction volume of 25 μl, PDGFRα (h) (5-10 mU) is incubated with 8 mM MOPS pH 7.0, 0.2 mM EDTA, 0.1 mg/ml poly(Glu, Tyr) 4: 1, 10 mM MnCl2, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

PDGFRβ (h)

In a final reaction volume of 25 μl, PDGFRβ (h) (5-10 mU) is incubated with 8 mM MOPS pH 7.0, 0.2 mM EDTA, 0.1 mg/ml poly(Glu, Tyr) 4:1, 10 mM MnCl2, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

PDK1 (h)

In a final reaction volume of 25 μl, PDK1 (h) (5-10 mU) is incubated with 50 mM Tris pH 7.5, 100 μM KTFCGTPEYLAPEVRREPRILSEEEQEMFRDFDYIADWC (SEQ ID NO:217) (PDKtide), 0.1% R-mercaptoethanol, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PI3Kγ (h) [Non-Radioactive Assay]

In a final reaction volume of 20 μl, PI3Kγ (h) is incubated in assay buffer containing 10 μM phosphatidylinositol-4,5-bisphosphate and MgATP (concentration as required). The reaction is initiated by the addition of the MgATP mix. After incubation for 30 minutes at room temperature, the reaction is stopped by the addition of 5 μl of stop solution containing EDTA and biotinylated phosphatidylinositol-3,4,5-trisphosphate. Finally, 5 μl of detection buffer is added, which contains europium-labelled anti-GST monoclonal antibody, GST-tagged GRP1 PH domain and streptavidin-allophycocyanin. The plate is then read in time-resolved fluorescence mode and the homogenous time-resolved fluorescence (HTRF®)* signal is determined according to the formula HTRF®=10000×(Em665 nm/Em620 nm).

Pim-1 (h)

In a final reaction volume of 25 μl, Pim-1 (h) (5-10 mU) is incubated with 8 mM MOPS pH 7.0, 0.2 mM EDTA, 100 μM KKRNRTLTV (SEQ ID NO:219), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PKA (h)

In a final reaction volume of 25 μl, PKA (h) (5-10 mU) is incubated with 8 mM MOPS pH 7.0, 0.2 mM EDTA, 30 μM LRRASLG (SEQ ID NO:209) (Kemptide), 10 mM MgAcetate and [y-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 50 mM phosphoric acid and once in methanol prior to drying and scintillation counting.

PKA (b)

In a final reaction volume of 25 μl, PKA (b) (5-10 mU) is incubated with 8 mM MOPS pH 7.0, 0.2 mM EDTA, 30 μM LRRASLG (SEQ ID NO:209) (Kemptide), 10 mM MgAcetate and [y-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 50 mM phosphoric acid and once in methanol prior to drying and scintillation counting.

PKBα (h)

In a final reaction volume of 25 μl, PKBα (h) (5-10 mU) is incubated with 8 mM MOPS pH 7.0, 0.2 mM EDTA, 30 μM GRPRTSSFAEGKK (SEQ ID NO:232), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PKBβ (h)

In a final reaction volume of 25 μl, PKBβ (h) (5-10 mU) is incubated with 8 mM MOPS pH 7.0, 0.2 mM EDTA, 30 μM GRPRTSSFAEGKK (SEQ ID NO:232), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PKBγ (h)

In a final reaction volume of 25 μl, PKBγ (h) (5-10 mU) is incubated with 8 mM MOPS pH 7.0, 0.2 mM EDTA, 30 μM GRPRTSSFAEGKK (SEQ ID NO:232), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PKCα (h)

In a final reaction volume of 25 μl, PKCα (h) (5-10 mU) is incubated with 20 mM HEPES pH 7.4, 0.03% Triton X-100, 0.1 mM, 0.1 mg/ml phosphatidylserine, 10 μg/ml diacylglycerol, 0.1 mg/ml histone Hi, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PKCβI (h)

In a final reaction volume of 25 μl, PKCβI (h) (5-10 mU) is incubated with 20 mM HEPES pH 7.4, 0.03% Triton X-100, 0.1 mM CaCl₂, 0.1 mg/ml phosphatidylserine, 10 μg/ml diacylglycerol, 0.1 mg/ml histone H1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PKCβII (h)

In a final reaction volume of 25 μl, PKCβII (h) (5-10 mU) is incubated with 20 mM HEPES pH 7.4, 0.03% Triton X-100, 0.1 mM, 0.1 mg/ml phosphatidylserine, 10 μg/ml diacylglycerol, 0.1 mg/ml histone H1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PKCγ (h)

In a final reaction volume of 25 μl, PKCγ (h) (5-10 mU) is incubated with 20 mM HEPES pH 7.4, 0.03% Triton X-100, 0.1 mM, 0.1 mg/ml phosphatidylserine, 10 μg/ml diacylglycerol, 0.1 mg/ml histone H1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PKCδ (h)

In a final reaction volume of 25 μl, PKCδ (h) (5-10 mU) is incubated with 20 mM HEPES pH 7.4, 0.03% Triton X-100, 0.1 mg/ml phosphatidylserine, 10 μg/ml diacylglycerol, 50 μM ERMRPRKRQGSVRRRV (SEQ ID NO:224), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PKCε (h)

In a final reaction volume of 25 μl, PKCε (h) (5-10 mU) is incubated with 20 mM HEPES pH 7.4, 0.03% Triton X-100, 0.1 mg/ml phosphatidylserine, 10 μg/ml diacylglycerol, 50 μM ERMRPRKRQGSVRRRV (SEQ ID NO:224), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PKCη (h)

In a final reaction volume of 25 μl, PKCη (h) (5-10 mU) is incubated with 20 mM HEPES pH 7.4, 0.03% Triton X-100, 0.1 mM CaCl₂, 0.1 mg/ml phosphatidylserine, 10 μg/ml diacylglycerol, 50 μM ERMRPRKRQGSVRRRV (SEQ ID NO:234), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PKCt (h)

In a final reaction volume of 25 μl, PKCt (h) (5-10 mU) is incubated with 20 mM HEPES pH 7.4, 0.03% Triton X-100, 50 μM ERMRPRKRQGSVRRRV (SEQ ID NO:224), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PKCμ (h)

In a final reaction volume of 25 μl, PKCμ (h) (5-10 mU) is incubated with 20 mM HEPES pH 7.4, 0.03% Triton X-100, 30 μM KKLNRTLSVA (SEQ ID NO:212), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PKCθ (h)

In a final reaction volume of 25 μl, PKCθ (h) (5-10 mU) is incubated with 8 mM MOPS pH 7.0, 0.2 mM EDTA, 0.1 mg/ml histone H1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PKCξ (h)

In a final reaction volume of 25 μl, PKCξ (h) (5-10 mU) is incubated with 8 mM MOPS pH 7.0, 0.2 mM EDTA, 50 μM ERMRPRKRQGSVRRRV (SEQ ID NO:224), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PKD2 (h)

In a final reaction volume of 25 μl, PKD2 (h) (5-10 mU) is incubated with 20 mM HEPES pH 7.4, 0.03% Triton X-100, 30 μM KKLNRTLSVA (SEQ ID NO:212) 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PKG1β (h)

In a final reaction volume of 25 μl, PKG1β (h) (5-10 mU) is incubated with 8 mM MOPS pH 7.0, 0.2 mM EDTA, 10 μM cGMP, 200 μM RRRLSFAEPG (SEQ ID NO:223), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Plk3 (h)

In a final reaction volume of 25 μl, Plk3 (h) (5-10 mU) is incubated with 8 mM MOPS pH 7.0, 0.2 mM EDTA, 2 mg/ml casein, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

PRAK (h)

In a final reaction volume of 25 μl, PRAK (h) (5-10 mU) is incubated with 50 mM Na R-glycerophosphate pH 7.5, 0.1 mM EGTA, 30 μM KKLRRTLSVA (SEQ ID NO:212), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 50 mM phosphoric acid and once in methanol prior to drying and scintillation counting.

PRK2 (h)

In a final reaction volume of 25 μl, PRK2 (h) (5-10 mU) is incubated with 50 mM Tris pH 7.5, 0.1 mM EGTA, 0.1% R-mercaptoethanol, 30 μM AKRRRLSSLRA (SEQ ID NO:226), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Pyk2 (h)

In a final reaction volume of 25 p-1, Pyk2 (h) (5-10 mU) is incubated with 8 mM MOPS pH 7.0, 0.2 mM EDTA, 0.1 mg/ml poly(Glu, Tyr) 4: 1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

p70S6K (h)

In a final reaction volume of 25 μl, p70S6K (h) (5-10 mU) is incubated with 8 mM MOPS pH 7.0, 0.2 mM EDTA, 100 μM KKRNRTLTV (SEQ ID NO:212), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Ret (h)

In a final reaction volume of 25 μl, Ret (h) (5-10 mU) is incubated with 8 mM MOPS pH 7.0, 0.2 mM EDTA, 250 μM KKKSPGEYVNIEFG (SEQ ID NO:207), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

RIPK2(h)

In a final reaction volume of 25 μl, RIPK2 (h) (5-10 mU) is incubated with 8 mM MOPS pH 7.0, 0.2 mM EDTA, 0.33 mg/ml myelin basic protein, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

ROCK-I (h)

In a final reaction volume of 25 μl, ROCK-I (h) (5-10 mU) is incubated with 8 mM MOPS pH 7.0, 0.2 mM EDTA, 30 μM KEAKEKRQEQIAKRRRLSSLRASTSKSGGSQK (SEQ ID NO:222), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

ROCK-II (h)

In a final reaction volume of 25 μl, ROCK-II (h) (5-10 mU) is incubated with 50 mM Tris pH 7.5, 0.1 mM EGTA, 30 μM KEAKEKRQEQIAKRRRLSSLRASTSKSGGSQK (SEQ ID NO:222), 10 mM MgAcetate and [y-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

ROCK-II (r)

In a final reaction volume of 25 μl, ROCK-II (r) (5-10 mU) is incubated with 50 mM Tris pH 7.5, 0.1 mM EGTA, 30 μM KEAKEKRQEQIAKRRRLSSLRASTSKSGGSQK (SEQ ID NO:222), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Ron (h)

In a final reaction volume of 25 μl, Ron (h) (5-10 mU) is incubated with 8 mM MOPS pH 7.0, 0.2 mM EDTA, 250 μM KKSRGDYMTMQIG (SEQ ID NO:210), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Ros (h)

In a final reaction volume of 25 μl, Ros (h) (5-10 mU) is incubated with 8 mM MOPS pH 7.0, 0.2 mM EDTA, 10 mM MnCl2, 250 μM KKKSPGEYVNIEFG (SEQ ID NO:207), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Rse (h)

In a final reaction volume of 25 μl, Rse (h) (5-10 mU) is incubated with 8 mM MOPS pH 7.0, 0.2 mM EDTA, 250 μM KVEKIGEGTYGVVYK (SEQ ID NO:211), 1 mM MnCl2, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Rsk1 (h)

In a final reaction volume of 25 μl, Rsk1 (h) (5-10 mU) is incubated with 8 mM MOPS pH 7.0, 0.2 mM EDTA, 30 μM KKKNRTLSVA (SEQ ID NO:212), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Rsk1 (r)

In a final reaction volume of 25 μl, Rsk1 (r) (5-10 mU) is incubated with 8 mM MOPS pH 7.0, 0.2 mM EDTA, 30 μM KKKNRTLSVA (SEQ ID NO:212), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Rsk2 (h)

In a final reaction volume of 25 μl, Rsk2 (h) (5-10 mU) is incubated with 8 mM MOPS pH 7.0, 0.2 mM EDTA, 30 μM KKKNRTLSVA (SEQ ID NO:212), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Rsk3 (h)

In a final reaction volume of 25 μl, Rsk3 (h) (5-10 mU) is incubated with 8 mM MOPS pH 7.0, 0.2 mM EDTA, 30 μM KKKNRTLSVA (SEQ ID NO:212), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

SAPK2a (h)

In a final reaction volume of 25 μl, SAPK2a (h) (5-10 mU) is incubated with 25 mM Tris pH 7.5, 0.02 mM EGTA, 0.33 mg/ml myelin basic protein, 10 mM MgAcetate and [gamma-S 3P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

SAPK2b (h)

In a final reaction volume of 25 μl, SAPK2b (h) (5-10 mU) is incubated with 25 mM Tris pH 7.5, 0.02 mM EGTA, 0.33 mg/ml myelin basic protein, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

SAPK3 (h)

In a final reaction volume of 25 μl, SAPK3 (h) (5-10 mU) is incubated with 25 mM Tris pH 7.5, 0.02 mM EGTA, 0.33 mg/ml myelin basic protein, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

SAPK4 (h)

In a final reaction volume of 25 μl, SAPK4 (h) (5-10 mU) is incubated with 25 mM Tris pH 7.5, 0.02 mM EGTA, 0.33 mg/ml myelin basic protein, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

SGK (h)

In a final reaction volume of 25 μl, SGK (h) (5-10 mU) is incubated with 8 mM MOPS pH 7.0, 0.2 mM EDTA, 30 μM GRPRTSSFAEGKK (SEQ ID NO:226), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

SGK2 (h)

In a final reaction volume of 25 μl, SGK2 (h) (5-10 mU) is incubated with 8 mM MOPS pH 7.0, 0.2 mM EDTA, 30 μM GRPRTSSFAEGKK (SEQ ID NO:226), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

SGK3 (h)

In a final reaction volume of 25 μl, SGK3 (h) (5-10 mU) is incubated with 8 mM MOPS pH 7.0, 0.2 mM EDTA, 250 μM GRPRTSSFAEGKK (SEQ ID NO:226), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Snk (h)

In a final reaction volume of 25 μl, Snk (h) (5-10 mU) is incubated with 8 mM MOPS pH 7.0, 0.2 mM EDTA, 2 mg/ml casein, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Syk (h)

In a final reaction volume of 25 μl, Syk (h) (5-10 mU) is incubated with 50 mM Tris pH 7.5, 0.1 mM EGTA, 0.1 mM Na3VO4, 0.1% R-mercaptoethanol, 0.1 mg/ml poly(Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

TAK1 (h)

In a final reaction volume of 25 μl, TAK1 (h) (5-10 mU) is incubated with 8 mM MOPS pH 7.0, 0.2 mM EDTA, 2 mg/ml casein, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

TBK1 (h)

In a final reaction volume of 25 μl, TBK1 (h) (5-10 mU) is incubated with 8 mM MOPS pH 7.0, 0.2 mM EDTA, 200 μM KRRRALS(p)VASLPGL (SEQ ID NO:214), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Tie2 (h)

In a final reaction volume of 25 μl, Tie2 (h) (5-10 mU) is incubated with 8 mM MOPS pH 7.0, 0.2 mM EDTA, 0.5 mM MnCl2, 0.1 mg/ml poly(Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

TrkA (h)

In a final reaction volume of 25 μl, TrkA (h) (5-10 mU) is incubated with 8 mM MOPS pH 7.0, 0.2 mM EDTA, 250 μM KKKSPGEYVNIEFG (SEQ ID NO:207), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

TrkB (h)

In a final reaction volume of 25 μl, TrkB (h) (5-10 mU) is incubated with 8 mM MOPS pH 7.0, 0.2 mM EDTA, 0.1 mg/ml poly(Glu, Tyr) 4: 1,10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

TSSK2 (h)

In a final reaction volume of 25 μl, TSSK2 (h) (5-10 mU) is incubated with 8 nM MOPS pH 7.0, 0.2 mM EDTA, 100 μM KKKVSRSGLYRSPSMPENLNRPR (SEQ ID NO:213), 10 mM MgAcetate and [y-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

Yes (h)

In a final reaction volume of 25 μl, Yes (h) (5-10 mU) is incubated with 8 mM MOPS pH 7.0, 0.2 mM EDTA, 0.1 mg/ml poly(Glu, Tyr) 4:1, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

ZAP-70 (h)

In a final reaction volume of 25 μl, ZAP-70 (h) (5-10 mU) is incubated with 50 mM Tris pH 7.5, 0.1 mM EGTA, 0.1 mM Na₃VO4, 0.1% R-mercaptoethanol, 0.1 mg/ml poly(Glu, Tyr) 4: 1, 10 mM MnCl2, 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a Filtermat A and washed three times for 5 minutes in 75 mM phosphoric acid and once in methanol prior to drying and scintillation counting.

ZIPK (h)

In a final reaction volume of 25 μl, ZIPK (h) (5-10 mU) is incubated with 8 mM MOPS pH 7.0, 0.2 mM EDTA, 250 μM KKLNRTLSFAEPG (SEQ ID NO:203), 10 mM MgAcetate and [gamma-33P-ATP] (specific activity approx. 500 cpm/pmol, concentration as required). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

The following table denotes percent residual activity of each protein kinase when incubated with compound A-1 or B-1.

| | A-1 @ 2.5 μM | B-1 @ 2.5 μM |
|---|---|---|
| Abl(h) | 26 | 100 |
| Abl(m) | 9 | 104 |
| Abl(T315I)(h) | 52 | 106 |
| ALK(h) | 69 | 97 |
| ALK4(h) | 108 | 103 |
| Arg(h) | 46 | 94 |
| AMPK(r) | 82 | 95 |
| Arg(m) | 43 | 102 |
| ARK5(h) | 79 | 104 |
| ASK1(h) | 98 | 110 |
| Aurora-A(h) | 43 | 104 |
| Axl(h) | 75 | 102 |
| Blk(m) | 117 | 106 |
| Bmx(h) | 68 | 105 |
| BRK(h) | 131 | 98 |
| BrSK1(h) | 99 | 107 |
| BrSK2(h) | 96 | 96 |
| BTK(h) | 60 | 101 |
| CaMKI(h) | 95 | 106 |
| CaMKII(r) | 107 | 113 |
| CaMKIIβ(h) | 101 | 100 |
| CaMKIIγ(h) | 110 | 104 |
| CaMKIIδ(h) | 88 | 96 |
| CaMKIV(h) | 124 | 122 |
| CDK1/cyclinB(h) | 84 | 93 |
| CDK2/cyclinA(h) | 86 | 94 |
| CDK2/cyclinE(h) | 102 | 117 |
| CDK3/cyclinE(h) | 93 | 103 |
| CDK5/p25(h) | 77 | 99 |
| CDK5/p35(h) | 83 | 104 |
| CDK6/cyclinD3(h) | 85 | 100 |
| CDK7/cyclinH/MAT1(h) | 72 | 95 |
| CDK9/cyclin T1(h) | 116 | 100 |
| CHK1(h) | 95 | 106 |
| CHK2(h) | 62 | 99 |
| CK1γ1(h) | 90 | 100 |
| CK1γ2(h) | 96 | 103 |
| CK1γ3(h) | 103 | 114 |
| CK1δ(h) | 87 | 92 |
| CK1(y) | 78 | 77 |
| CK2(h) | 104 | 102 |
| CK2α2(h) | 115 | 116 |
| CLK3(h) | 84 | 96 |
| cKit(D816V)(h) | 104 | 103 |
| cKit(D816H)(h) | 73 | 92 |
| cKit(h) | 111 | 114 |
| c-RAF(h) | 100 | 96 |
| CSK(h) | 116 | 92 |
| cSRC(h) | 33 | 137 |
| DAPK1(h) | 40 | 89 |
| DAPK2(h) | 109 | 102 |
| DCAMKL2(h) | 48 | 111 |
| DDR2(h) | 77 | 95 |
| DMPK(h) | 92 | 105 |
| DRAK1(h) | 11 | 117 |
| DYRK2(h) | 70 | 75 |
| eEF-2K(h) | 96 | 98 |
| EGFR(h) | 122 | 117 |
| EGFR(L858R)(h) | 113 | 109 |
| EGFR(L861Q)(h) | 94 | 109 |
| EGFR(T790M)(h) | 91 | 114 |
| EGFR(T790M, L858R)(h) | 93 | 99 |
| EphA1(h) | 69 | 96 |
| EphA2(h) | 101 | 102 |
| EphA3(h) | 91 | 118 |
| EphA4(h) | 107 | 134 |
| EphA5(h) | 111 | 101 |
| EphA7(h) | 109 | 100 |
| EphA8(h) | 100 | 100 |
| EphB1(h) | 125 | 75 |
| EphB2(h) | 96 | 106 |
| EphB3(h) | 111 | 115 |
| EphB4(h) | 96 | 108 |
| ErbB4(h) | 92 | 107 |
| FAK(h) | 94 | 102 |
| Fer(h) | 84 | 94 |
| Fes(h) | 82 | 101 |
| FGFR1(h) | 84 | 118 |
| FGFR2(h) | 76 | 89 |
| FGFR3(h) | 101 | 116 |
| FGFR4(h) | 112 | 104 |
| Fgr(h) | 78 | 98 |
| Flt1(h) | 78 | 106 |
| Flt3(D835Y)(h) | 36 | 78 |
| Flt3(h) | 32 | 100 |
| Flt4(h) | 70 | 103 |
| Fms(h) | 131 | 107 |
| Fyn(h) | 97 | 85 |
| GRK5(h) | 95 | 99 |
| GRK6(h) | 90 | 83 |
| GSK3α(h) | 101 | 89 |
| GSK3β(h) | 135 | 76 |
| Hck(h) | 84 | 89 |
| HIPK1(h) | 95 | 101 |
| HIPK2(h) | 106 | 111 |
| HIPK3(h) | 102 | 97 |
| IGF-1R(h) | 103 | 113 |
| IKKα(h) | 68 | 118 |
| IKKβ(h) | 56 | 105 |
| IR(h) | 96 | 110 |
| IRR(h) | 97 | 103 |
| IRAK1(h) | 86 | 102 |
| IRAK4(h) | 98 | 107 |
| Itk(h) | 117 | 102 |
| JAK2(h) | 105 | 116 |
| JAK3(h) | 112 | 109 |
| JNK1α1(h) | 94 | 100 |
| JNK2α2(h) | 101 | 106 |
| JNK3(h) | 97 | 98 |
| KDR(h) | 90 | 95 |
| Lck(h) | 170 | 115 |
| LIMK1(h) | 97 | 100 |
| LKB1(h) | 98 | 101 |
| LOK(h) | 88 | 106 |
| Lyn(h) | 110 | 122 |
| Lyn(m) | 89 | 106 |
| MAPK1(h) | 92 | 94 |
| MAPK2(h) | 101 | 108 |
| MAPK2(m) | 95 | 102 |
| MAPKAP-K2(h) | 79 | 99 |
| MAPKAP-K3(h) | 94 | 109 |
| MARK1(h) | 83 | 95 |
| MEK1(h) | 87 | 90 |
| MELK(h) | 40 | 94 |
| Mer(h) | 111 | 125 |
| Met(h) | 132 | 115 |
| MINK(h) | 72 | 97 |
| MKK4(m) | 108 | 99 |
| MKK6(h) | 100 | 90 |
| MKK7β(h) | 111 | 1 |
| MLCK(h) | 77 | 94 |
| MLK1(h) | 85 | 86 |
| Mnk2(h) | 98 | 115 |
| MRCKα(h) | 85 | 98 |
| MRCKβ(h) | 84 | 102 |
| MSK1(h) | 61 | 99 |
| MSK2(h) | 50 | 94 |
| MSSK1(h) | 41 | 107 |
| MST1(h) | 76 | 96 |
| MST2(h) | 76 | 106 |
| MST3(h) | 45 | 110 |
| MuSK(h) | 110 | 108 |
| NEK2(h) | 65 | 106 |
| NEK3(h) | 94 | 118 |
| NEK6(h) | 78 | 111 |
| NEK7(h) | 102 | 101 |
| NEK11(h) | 70 | 107 |
| NLK(h) | 90 | 109 |
| p70S6K(h) | 18 | 97 |
| PAK2(h) | 73 | 95 |
| PAK3(h) | 88 | 91 |
| PAK4(h) | 99 | 96 |
| PAK5(h) | 103 | 101 |
| PAK6(h) | 123 | 112 |
| PAR-1Bα(h) | 88 | 100 |
| PASK(h) | 10 | 100 |
| PDGFRα(h) | 108 | 106 |
| PDGFRβ(h) | 103 | 103 |
| PDK1(h) | 121 | 109 |
| PhKγ2(h) | 46 | 118 |
| Pim-1(h) | 30 | 104 |
| Pim-2(h) | 77 | 110 |
| PKA(b) | 99 | 96 |
| PKA(h) | 105 | 97 |
| PKBα(h) | 91 | 103 |
| PKBβ(h) | 48 | 106 |
| PKBγ(h) | 64 | 95 |
| PKCα(h) | 90 | 101 |
| PKCβI(h) | 94 | 97 |
| PKCβII(h) | 94 | 99 |
| PKCγ(h) | 110 | 102 |
| PKCδ(h) | 105 | 100 |
| PKCε(h) | 99 | 109 |
| PKCη(h) | 80 | 80 |
| PKCι(h) | 72 | 95 |
| PKCμ(h) | 49 | 94 |
| PKCθ(h) | 91 | 108 |
| PKCζ(h) | 56 | 113 |
| PKD2(h) | 79 | 98 |
| PKG1α(h) | 57 | 101 |
| PKG1β(h) | 48 | 107 |
| Plk3(h) | 92 | 86 |
| PRAK(h) | 105 | 111 |
| PRK2(h) | 36 | 110 |
| PrKX(h) | 75 | 96 |
| PTK5(h) | 116 | 107 |
| Pyk2(h) | 95 | 105 |
| Ret(h) | 123 | 92 |
| RIPK2(h) | 95 | 99 |
| ROCK-I(h) | 91 | 101 |
| ROCK-II(h) | 64 | 115 |
| ROCK-II(r) | 48 | 103 |
| Ron(h) | 71 | 95 |
| Ros(h) | 115 | 113 |
| Rse(h) | 92 | 92 |
| Rsk1(h) | 85 | 103 |
| Rsk1(r) | 85 | 110 |
| Rsk2(h) | 60 | 106 |
| Rsk3(h) | 89 | 107 |
| Rsk4(h) | 69 | 94 |
| SAPK2a(h) | 97 | 103 |
| SAPK2a(T106M)(h) | 94 | 99 |
| SAPK2b(h) | 70 | 94 |
| SAPK3(h) | 85 | 111 |
| SAPK4(h) | 31 | 105 |
| SGK(h) | 50 | 103 |
| SGK2(h) | 38 | 104 |
| SGK3(h) | 44 | 128 |
| SIK(h) | 79 | 94 |
| Snk(h) | 125 | 125 |
| SRPK1(h) | 91 | 99 |
| SRPK2(h) | 88 | 88 |
| STK33(h) | 90 | 102 |
| Syk(h) | 67 | 111 |
| TAK1(h) | 98 | 95 |
| TBK1(h) | 71 | 114 |
| Tie2(h) | 13 | 115 |
| TrkA(h) | 25 | 80 |
| TrkB(h) | 44 | 118 |
| TSSK1(h) | 98 | 107 |
| TSSK2(h) | 105 | 110 |
| WNK2(h) | 97 | 108 |
| WNK3(h) | 98 | 120 |
| Yes(h) | 81 | 96 |
| ZAP-70(h) | 130 | 105 |
| ZIPK(h) | 27 | 116 |

Inhibition of Abl(h) was characterized further in the presence of 45 micromolar ATP, as reflected in the following table.

| | | Mean | | | |
|---|---|---|---|---|---|
| | | (Counts − | Activity (% | | |
| Sample | Counts | Blanks) | Control) | Mean | SD* |
| A-1 @ 2.5 μM | 15236 | 12522 | 27 | 26 | 1 |
| | 14630 | | 25 | | |
| B-1 @ 2.5 μM | 51051 | 47944 | 101 | 100 | 2 |
| | 49659 | | 98 | | |
| CONTROL | 49343 | 48060 | 98 | 100 | 2 |
| | 52034 | | 103 | | |
| | 49823 | | 99 | | |
| | 50684 | | 100 | | |
| BLANK | 2423 | / | / | / | / |
| | 2399 | | / | | |

*NB. Where n = 2, the value reported here is actually range/√2

The data above show compound B-1 inhibits MKK7B. Compound A-I inhibits Abl, DRAK1, p70S6K, PASK and Tie2 with greater than 80% inhibition, and other kinases with between 60-80% inhibition.

### Example 12

### Effects of Compounds on Ribosomal RNA Synthesis

Assays were conducted to determine the effects of compounds on rRNA synthesis from 45S rDNA. In particular, compound A-1 at various concentrations was incubated with cells and tested for an effect on rRNA synthesis after a two hour or four hour incubation with the compound. Synthesized rRNA was quantified by a polymerase chain reaction (PCR) assay. A primer/probe set was designed using Primer Express software and synthesized by Applied Biosystems. The 5′ ETS Probe utilized had the following sequence (@ its 3′ end): 6FAM-TTG ATC CTG CCA GTA GC-MGBNFQ (SEQ ID NO:227). The primer sequences were as follows:

(SEQ ID NO:228)

Forward Primer: CCG CGC TCT ACC TTA CCT ACC T

(SEQ ID NO:229)

Reverse Primer: GCA TGG CTT AAT CTT TGA GAC AAG.

A control assay that detected effects of the compounds on C-myc transcription also was conducted using a primer/probe set purchased from ABI (TaqMan Gene Expression Assay with assay ID: Hs99999003_m1). The following assay protocol was utilized:

Step 1. Reverse transcription of RNA to DNA

Mix

1 ug RNA

2.5 ul 10×Taq Man buffer

5.5 ul 25 mM MgCl₂

5 ul of a mix of dNTP (500 uM each)

1.2 ul random hexamer primer (2.5 uM stock)

0.5 ul RNase inhibitor (0.4 units/ul)

0.6 ul Reverse Transcriptase (1.2 units/ul)

bring to 25 ul total volume with water

Incubate at 48 degrees C. for 30 minutes

Inactivate Reverse Transcriptase by incubating at 95 for 5 minutes

Step 2. PCR

Mix

5 ul Reverse Transcriptase reaction product

12.5 ul 2×PCR mix

1 uM forward primer

1 uM reverse primer

0.5 uM Taq Man probe

500 nM Rox

Adjust to 25 ul final volume with water

PCR Cycles

95 degrees C. 1 minute

40 cycles of

95 degrees C. 15 seconds

60 degrees C. 1 minute.

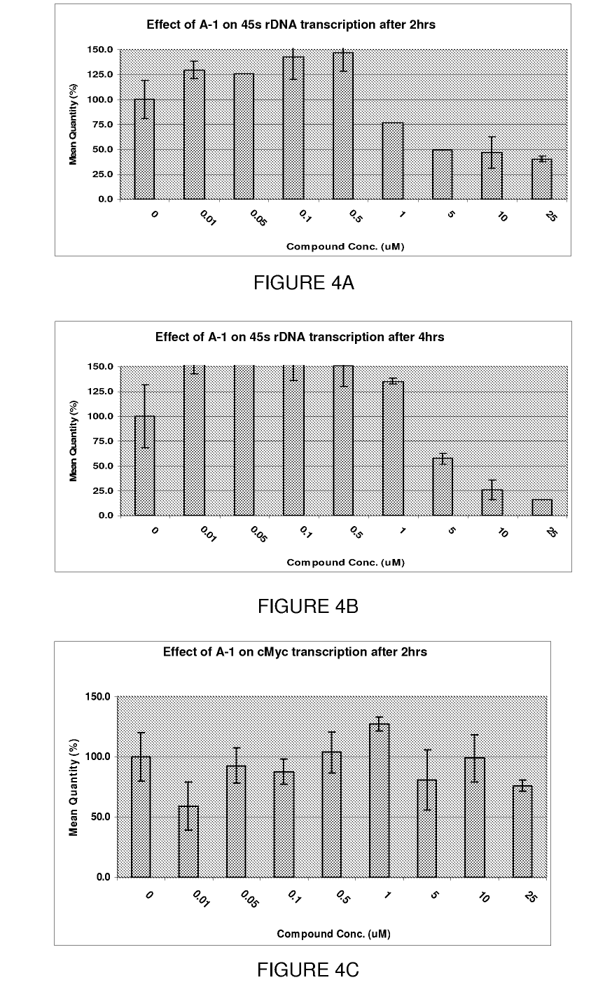

Fluorescence of digested label was detected and quantified. As shown in FIGS. 4A and 4B, compound A-1 inhibited rRNA synthesis at two hours and four hours. As a comparison, the effect of compound A-1 on c-Myc transcription is shown in FIG. 4C. The effect of other compounds on rRNA synthesis also were assessed in the assay, and provided in the table hereafter are IC50 values of selected compounds pertaining to rRNA synthesis and cMYC RNA synthesis.

| | | QPCRrDNA | QPCRMYC |
|---|---|---|---|
| Cmpd. | | IC50_HCT-116 | IC50_HCT-116 |
| Number | Structure | (μM) | (μM) |
| 1 | 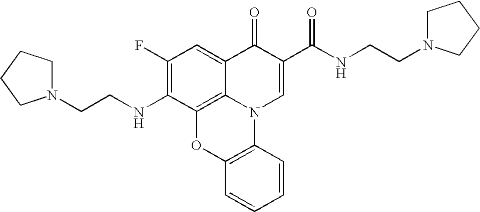 | 0.05 | 0.01 |
| 2 | 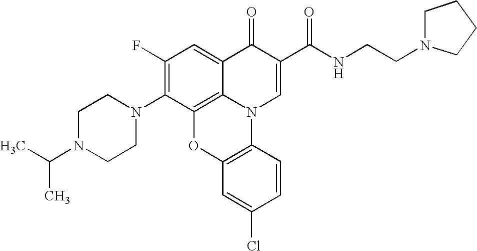 | 0.05 | 0.1 |
| 3 | 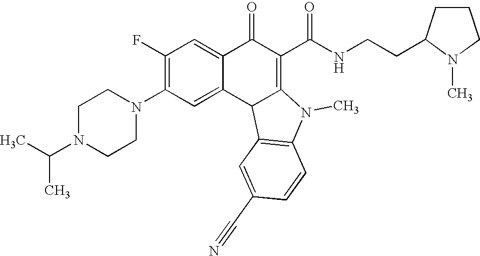 | 0.05 | INACTIVE |
| 4 | 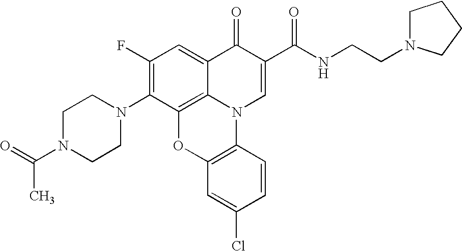 | 0.05 | 0.3 |
| 5 | 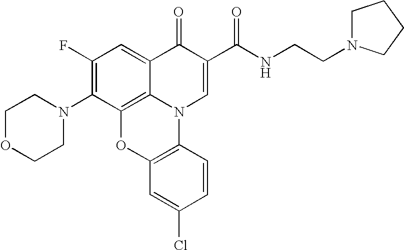 | 0.05 | 0.3 |
| 6 | 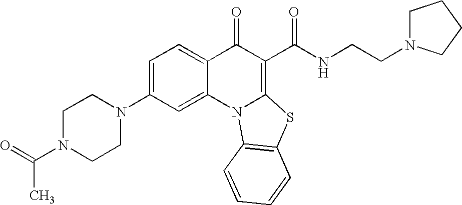 | 0.08 | 0.3 |
| 7 | 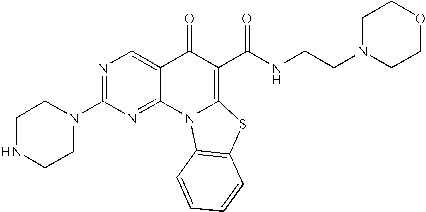 | 0.08 | 0.3 |
| 8 | 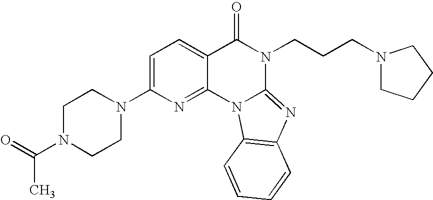 | 0.05 | 0.3 |
| 9 | 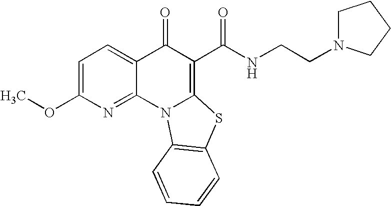 | 0.05 | 0.3 |

IC50 values determined by the assay for multiple compounds were plotted against IC50 values for the same compounds in cell viability assays. Specifically, the log of the IC50 for rDNA suppression as measured by PCR was plotted against the log IC50 for cell viability as measured by Alamar Blue (4 days; HCT-116 Cells). A clear, positive correlation between cell viability and the suppression of rDNA transcription was evident from the plot, which underscores the biological relevance of the PCR assay.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following aspects.

What is claimed is:

1. An isolated nucleic acid which comprises a nucleotide sequence ((G₃₊)N₁₋₇)₃G₃₊ (SEQ ID NO: 230) or ((C₃₊)N₁₋₇)₃C₃₊ (SEQ ID NO:231) in a human rRNA or rDNA nucleotide sequence, wherein:
G is guanine, C is cytosine, 3+ is three or more nucleotides and N is any nucleotide;
the nucleic acid is a circular or linear nucleic acid; and
the nucleotide sequence is 100 or fewer nucleotides in length.

2. The isolated nucleic acid of claim 1, wherein the nucleotide sequence is 50 or fewer nucleotides in length.

3. The isolated nucleic acid of claim 1, wherein the isolated nucleic acid is a linear nucleic acid.

4. The isolated nucleic acid of claim 1, wherein the nucleic acid is 100 or fewer nucleotides in length.

5. The isolated nucleic acid of claim 1, wherein the nucleic acid is DNA.

6. The isolated nucleic acid of claim 5, wherein the nucleotide sequence is a subsequence of SEQ ID NO: 1.

7. The isolated nucleic acid of claim 6, wherein the nucleotide sequence encodes a human 28S ribosomal RNA.

8. The isolated nucleic acid of claim 6, wherein the nucleotide sequence comprises one or more nucleotide sequences selected from the group consisting of
(SEQ ID NO:2)

| | |
|---|---|
| 1197-1221: | GGGTGGACGGGGGGGCCTGGTGGGG; |

(SEQ ID NO:3)

| | |
|---|---|
| 2160-2227: | CCCGGGTGCCCTTGCCCTCGCGGTCCCCGGCCCTCGC |
| | CCGTCTGTGCCCTCTTCCCCGCCCGCCGCCC; |

(SEQ ID NO:4)

| | |
|---|---|
| 2958-2985: | GGGTCGGGGGGTGGGGCCCGGGCCGGGG; |

(SEQ ID NO:5)

| | |
|---|---|
| 3468-3491: | CCCCGCCCCGGCCCCACCGGTCCC; |

(SEQ ID NO:6)

| | |
|---|---|
| 3500-3532: | CCCCCGCGCCCGCTCGCTCCCTCCCGTCCGCCC; |

(SEQ ID NO:7)

| | |
|---|---|
| 6184-6213: | GGGTCGGGGGCGGTGGTGGGCCCGCGGGGG; |

(SEQ ID NO:8)

| | |
|---|---|
| 6915-6944: | CCCGCCCCTTCCCCCTCCCCCCGCGGGCCC; |

(SEQ ID NO:9)

| | |
|---|---|
| 6375-6403: | GGGGGCGGGAACCCCCGGGCGCCTGTGGG; |

(SEQ ID NO:10)

| | |
|---|---|
| 6961-6983: | GGGTGGCGGGGGGGAGAGGGGGG; |

(SEQ ID NO:11)

| | |
|---|---|
| 7254-7298: | GGGTCCGGAAGGGGAAGGGTGCCGGCGGGGAGAGAGG |
| | GTCGGGGG; |

(SEQ ID NO:12)

| | |
|---|---|
| 7370-7399: | CCCCGCGCCCCTCCTCCTCCCCGCCGCCCC; |

(SEQ ID NO:13)

| | |
|---|---|
| 7734-7763: | CCCGTCCCGCCCCCGGCCCGTGCCCCTCCC; |

(SEQ ID NO:14)

| | |
|---|---|
| 8440-8494: | CCCGCCCCCCGTTCCTCCCGACCCCTCCACCCGCCCT |
| | CCCTTCCCCCGCCGCCCC; |

(SEQ ID NO:15)

| | |
|---|---|
| 8512-8573: | GGGGGCGGGCTCCGGCGGGTGCGGGGGTGGGCGGGCG |
| | GGGCCGGGGGTGGGGTCGGCGGGGG; |

(SEQ ID NO:16)

| | |
|---|---|
| 8716-8747: | CCCGTCTCCGCCCCCCGGCCCCGCGTCCTCCC; |

(SEQ ID NO:17)

| | |
|---|---|
| 8750-8770: | GGGAGGGCGCGCGGGTCGGGG; |

(SEQ ID NO:18)

| | |
|---|---|
| 8904-8926: | CCCCCCTCCCGGCGCCCACCCCC; |

(SEQ ID NO:19)

| | |
|---|---|
| 9024-9052: | CCCACCCCTCCTCCCCGCGCCCCCGCCCC; |

(SEQ ID NO:20)

| | |
|---|---|
| 10137-10179: | CCCCTCCTCCCGCCCACGCCCCGCTCCCCGCCCCCGG |
| | AGCCCC; |

(SEQ ID NO:21)

| | |
|---|---|
| 10817-10839: | GGGCTGGGTCGGTCGGGCTGGGG; |

(SEQ ID NO:22)

| | |
|---|---|
| 10885-10934: | CCCCCCCCACGCCCGGGGCACCCCCCTCGCGGCCCTC |
| | CCCCGCCCCACCC; |

(SEQ ID NO:23)

| | |
|---|---|
| 10951-10969: | CCCTCCCCACCCCGCGCCC; |

(SEQ ID NO:24)

| | |
|---|---|
| 10985-11012: | CCCCCGCTCCCCGTCCTCCCCCCTCCCC; |

(SEQ ID NO:25)

| | |
|---|---|
| 11029-11066: | GGGGCGCGGCGGGGGGAGAAGGGTCGGGGCGGCAGGG |
| | G; |

(SEQ ID NO:26)

| | |
|---|---|
| 11345-11389: | CCCCCCGCCCTACCCCCCCGGCCCCGTCCGCCCCCCG |
| | TTCCCCCC; |

(SEQ ID NO:27)

| | |
|---|---|
| 11888-11912: | CCCCCGGCGCCCCCCCGGTGTCCCC; |

(SEQ ID NO:28)

| | |
|---|---|
| 13174-13194: | GGGCCGGGACGGGGTCCGGGG; |

(SEQ ID NO:29)

| | |
|---|---|
| 13236-13261: | CCCCGTGGCCCGCCGGTCCCCGTCCC; |

(SEQ ID NO:30)

| | |
|---|---|
| 14930-14963: | CCCTCCCTCCCTCCCCCTCCCTCCCTCTCTCCCC; |

(SEQ ID NO:31)

| | |
|---|---|
| 17978-18013: | CCCCCACCCCCCCGTCACGTCCCGCTACCCTCCCCC; |

(SEQ ID NO:32)

| | |
|---|---|
| 20511-20567: | GGGGGTGCGGGAATGAGGGTGTGTGTGGGGAGGGGGT |
| | GCGGGGTGGGGACGGAGGGG; |

(SEQ ID NO:33)

| | |
|---|---|
| 23408-23434: | GGGGAGAGAGGGGGGAGAGGGGGGGGG; |

(SEQ ID NO:34)

| | |
|---|---|
| 28214-28250: | CCCCAAACCGCCCCCCCCCCCCCGCCTCCCAACACC |
| | C; |

(SEQ ID NO:35)

| | |
|---|---|
| 31239-31275: | CCCCACCCACGCCCCACGCCCCACGTCCCGGGCACC |
| | C; |

(SEQ ID NO:36)

| | |
|---|---|
| 31415-31452: | GGGAGGGGTGGGGGTGGGGTGGGTTGGGGGTTGTGG |
| | GG; |

(SEQ ID NO:37)

| | |
|---|---|
| 37405-37431: | CCCGGACCCCCCCTTTCCCCTTCCCCC; |

(SEQ ID NO:38)

| | |
|---|---|
| 39261-39290: | CCCGCCCTCCCTGGTTGCCCAGACAACCCC; |
| and | |

(SEQ ID NO:39)

| | |
|---|---|
| 41667-41709: | CCCTCCCTCCCTCCCTCCCTCCTCCCTTCCCTCCCT |
| | CCTTCCC. |

9. The isolated nucleic acid of claim 6, wherein the nucleotide sequence comprises one or more nucleotide sequences selected from the group consisting of
(SEQ ID NO:40)

| | |
|---|---|
| 1310-1333: | CCCCCTCCCTTCCCCAGGCGTCCC; |

(SEQ ID NO:41)

| | |
|---|---|
| 5701-5718: | GGGAGGGAGACGGGGGGG; |

(SEQ ID NO:42)

| | |
|---|---|
| 6535-6553: | GGGCGGGGGGGGCGGGGGG; |

(SEQ ID NO:43)

| | |
|---|---|
| 7499-7517: | CCCGCCCCGCCGCCCGCCC; |

(SEQ ID NO:44)

| | |
|---|---|
| 10111-10127: | CCCCCGCCCCCCCCCCC; |

(SEQ ID NO:45)

| | |
|---|---|
| 13080-13095: | GGGGTGGGGGGGAGGG; |

(SEQ ID NO:46)

| | |
|---|---|
| 14213-14248: | CCCTCCCTCCCTCCCTCCCTCCCTCCCTCCCTCCCC; |

(SEQ ID NO:47)

| | |
|---|---|
| 16166-16189: | GGGGTGGGGTGGGGTGGGGTGGGG; |

(SEQ ID NO:48)

| | |
|---|---|
| 28148-28177: | CCCCCCGGCTCCCCCCACTACCCACGTCCC; |
| and | |

(SEQ ID NO:49)

| | |
|---|---|
| 41842-41876: | CCCTCCCTCCCTCCCTCCCTCCCTCCCTCCCTCCC. |

10. The isolated nucleic acid of claim 1, wherein the nucleic acid is RNA.

11. The isolated nucleic acid of claim 10, wherein the nucleotide sequence is encoded by SEQ ID NO: 1.

12. The isolated nucleic acid of claim 11, wherein the nucleotide sequence is from a human 28S ribosomal RNA.

13. The isolated nucleic acid of claim 11, wherein the nucleotide sequence comprises one or more nucleotide sequences selected from the group consisting of
(SEQ ID NO:107)
GGGGUGGACGGGGGGGCCUGGUGGGG;
(SEQ ID NO:108)
GGGUCGGGGGGUGGGGCCCGGGCCGGGG;
(SEQ ID NO:109)
GGGAGGGAGACGGGGGGG;
(SEQ ID NO:110)
GGGUCGGGGGCGGUGGUGGGCCCGCGGGGG;
(SEQ ID NO:111)
GGGGGCGGGAACCCCCGGGCGCCUGUGGG;
(SEQ ID NO:112)
GGGUGGCGGGGGGGAGAGGGGGG;
(SEQ ID NO:113)
GGGUCCGGAAGGGGAAGGGUGCCGGCGGGGAGAGAGGGUCGGGGG;
(SEQ ID NO:114)
GGGGGCGGGCUCCGGCGGGUGCGGGGGUGGGCGGGCGGGGCCGGGGGUGG
GGUCGGCGGGGG;
(SEQ ID NO:115)
GGGAGGGCGCGCGGGUCGGGG;
(SEQ ID NO:116)
GGGCUGGGUCGGUCGGGCUGGGG;
(SEQ ID NO:117)
GGGGCGCGGCGGGGGGAGAAGGGUCGGGGCGGCAGGGG;
and
(SEQ ID NO:118)
GGGCCGGGACGGGGUCCGGGG.

14. The isolated nucleic acid of claim 11, wherein the nucleotide sequence comprises one or more nucleotide sequences selected from the group consisting of
(SEQ ID NO:121)
CCCCCUCCCUUCCCCAGGCGUCCC;
(SEQ ID NO:122)
CCCGGGUGCCCUUGCCCUCGCGGUCCCCGGCCCUCGCCCGUCUGUGCCCU
CUUCCCCGCCCGCCGCCC;
(SEQ ID NO:123)
CCCCGCCCCGGCCCCACCGGUCCC;
(SEQ ID NO:124)
CCCCCGCGCCCGCUCGCUCCCUCCCGUCCGCCC;
(SEQ ID NO:125)
CCCGCCCCUUCCCCCUCCCCCCGCGGGCCC;
(SEQ ID NO:126)
CCCCGCGCCCCUCCUCCUCCCCGCCGCCCC;
(SEQ ID NO:127)
CCCGCCCCGCCGCCCGCCC;
(SEQ ID NO:128)
CCCGUCCCGCCCCCGGCCCGUGCCCCUCCC;
(SEQ ID NO:129)
CCCGCCCCCCGUUCCUCCCGACCCCUCCACCCGCCCUCCCUUCCCCCGCC
GCCCC;
(SEQ ID NO:130)
CCCGUCUCCGCCCCCCGGCCCCGCGUCCUCCC;
(SEQ ID NO:131)
CCCCCCUCCCGGCGCCCACCCCC;
(SEQ ID NO:132)
CCCACCCCUCCUCCCCGCGCCCCCGCCCC;
(SEQ ID NO:133)
CCCCCGCCCCCCCCCCC;
(SEQ ID NO:134)
CCCCUCCUCCCGCCCACGCCCCGCUCCCCGCCCCCGGAGCCCC;
(SEQ ID NO:135)
CCCCCCCCACGCCCGGGGCACCCCCCUCGCGGCCCUCCCCCGCCCCACC
C;
(SEQ ID NO:136)
CCCUCCCCACCCCGCGCCC;
(SEQ ID NO:137)
CCCCCGCUCCCCGUCCUCCCCCCUCCCC;
(SEQ ID NO:138)
CCCCCCGCCCUACCCCCCCGGCCCCGUCCGCCCCCCGUUCCCCCC;
(SEQ ID NO:139)
CCCCCCGCGCCCCCCCGGUCUCCCC;
and
(SEQ ID NO:140)
CCCCGUGGCCCGCCGGUCCCCGUCCC.

15. The isolated nucleic acid of claim 11, wherein the nucleotide sequence comprises one or more of the nucleotide sequences GGGCGGGGGGGGCGGGGGG (SEQ ID NO:42) or GGGGUGGGGGGGAGGG (SEQ ID NO:120).

16. The isolated nucleic acid of claim 1, which comprises one or more nucleotide analogs or derivatives.

17. The isolated nucleic acid of claim 1, which includes one or more nucleotide substitutions.

18. The isolated nucleic acid of claim 1, wherein the nucleotide sequence forms a quadruplex structure.

19. The isolated nucleic acid of claim 18, wherein the quadruplex structure is an intramolecular quadruplex structure.

20. The isolated nucleic acid of claim 18, wherein the quadruplex is a G-quadruplex.

21. The isolated nucleic acid of claim 19, wherein the intramolecular quadruplex is a parallel quadruplex.

22. The isolated nucleic acid of claim 19, wherein the intramolecular quadruplex is a mixed parallel quadruplex.

23. A composition comprising a nucleic acid of claim 1 in combination with a small molecule.

24. The composition of claim 23, wherein the small molecule is a quinolone analog.

25. A composition comprising a nucleic acid of claim 1 in combination with a protein that binds to the nucleic acid.

26. The composition of claim 25, wherein the protein is selected from the group consisting of Nucleolin, Fibrillarin, RecQ, QPNI and functional fragments of the foregoing.


SEQUENCE LISTING (231 sequences)

SEQ ID NO: 1 - DNA, 42999 bp, Homo sapiens
  misc_feature (0)...(42999): n = a, g, c, or t
gctgacacgctgtcctctggcgacctgtcgtcggagaggttgggcctccggatgcgcgcggggctctggcctcacggtgaccggctagccggccgcgctcctgccttgagccgcctgccgcggcccgcgggcctgctgttctctcgcgcgtccgagcgtcccgactcccggtgccggcccgggtccgggtctctgacccacccgggggcggcggggaaggcggcgagggccaccgtgccccgtgcgctctccgctgcgggcgcccggggcgccgcacaaccccacccgctggctccgtgccgtgcgtgtcaggcgttctcgtctccgcggggttgtccgccgccccttccccggagtggggggtggccggagccgatcggctcgctggccggccggcctccgctcccggggggctcttcgatcgatgtggtgacgtcgtgctctcccgggccgggtccgagccgcgacgggcgaggggcggacgttcgtggcgaacgggaccgtccttctcgctccgcccgcgcggtcccctcgtctgctcctctccccgcccgccggccggcgtgtgggaaggcgtggggtgcggaccccggcccgacctcgccgtcccgcccgccgccttcgcttcgcgggtgcgggccggcggggtcctctgacgcggcagacagccctgcctgtcgcctccagtggttgtcgacttgcgggcggcccccctccgcggcggtgggggtgccgtcccgccggcccgtcgtgctgccctctcggggggggtttgcgcgagcgtcggctccgcctgggcccttgcggtgctcctggagcgctccgggttgtccctcaggtgcccgaggccgaacggtggtgtgtcgttcccgcccccggcgccccctcctccggtcgccgccgcggtgtccgcgcgtgggtcctgagggagctcgtcggtgtggggttcgaggcggtttgagtgagacgagacgagacgcgcccctcccacgcggggaagggcgcccgcctgctctcggtgagcgcacgtcccgtgctcccctctggcgggtgcgcgcgggccgtgtgagcgatcgcggtgggttcgggccggtgtgacgcgtgcgccggccggccgccgaggggctgccgttctgcctccgaccggtcgtgtgtgggttgacttcggaggcgctctgcctcggaaggaaggaggtgggtggacgggggggcctggtggggttgcgcgcacgcgcgcaccggccgggcccccgccctgaacgcgaacgctcgaggtggccgcgcgcaggtgtttcctcgtaccgcagggccccctcccttccccaggcgtccctcggcgcctctgcgggcccgaggaggagcggctggcgggtggggggagtgtgacccaccctcggtgagaaaagccttctctagcgatctgagaggcgtgccttgggggtaccggatcccccgggccgccgcctctgtctctgcctccgttatggtagcgctgccgtagcgacccgctcgcagaggaccctcctccgcttccccctcgacggggttgggggggagaagcgagggttccgccggccaccgcggtggtggccgagtgcggctcgtcgcctactgtggcccgcgcctcccccttccgagtcgggggaggatcccgccgggccgggcccggcgctcccacccagcgggttgggacgcggcggccggcgggcggtgggtgtgcgcgcccggcgctctgtccggcgcgtgaccccctccgtccgcgagtcggctctccgcccgctcccgtgccgagtcgtgaccggtgccgacgaccgcgtttgcgtggcacggggtcgggcccgcctggccctgggaaagcgtcccacggtgggggcgcgccggtctcccggagcgggaccgggtcggaggatggacgagaatcacgagcgacggtggtggtggcgtgtcgggttcgtggctgcggtcgctccggggcccccggtggcggggccccggggctcgcgaggcggttctcggtgggggccgagggccgtccggcgtcccaggcggggcgccgcgggaccgccctcgtgtctgtggcggtgggatcccgcggccgtgttttcctggtggcccggccgtgcctgaggtttctccccgagccgccgcctctgcgggctcccgggtgcccttgccctcgcggtccccggccctcgcccgtctgtgccctcttccccgcccgccgcccgccgatcctcttcttccccccgagcggctcaccggcttcacgtccgttggtggccccgcctgggaccgaacccggcaccgcctcgtggggcgccgccgccggccactgatcggcccggcgtccgcgtcccccggcgcgcgccttggggaccgggtcggtggcgcgccgcgtggggcccggtgggcttcccggagggttccgggggtcggcctgcggcgcgtgcgggggaggagacggttccgggggaccggccgcggctgcggcggcggcggtggtggggggagccgcggggatcgccgagggccggtcggccgccccgggtgccccgcggtgccgccggcggcggtgaggccccgcgcgtgtgtcccggctgcggtcggccgcgctcgaggggtccccgtggcgtccccttccccgccggccgcctttctcgcgccttccccgtcgccccggcctcgcccgtggtctctcgtcttctcccggcccgctcttccgaaccgggtcggcgcgtcccccgggtgcgcctcgcttcccgggcctgccgcggcccttccccgaggcgtccgtcccgggcgtcggcgtcggggagagcccgtcctccccgcgtggcgtcgccccgttcggcgcgcgcgtgcgcccgagcgcggcccggtggtccctcccggacaggcgttcgtgcgacgtgtggcgtgggtcgacctccgccttgccggtcgctcgccctctccccgggtcggggggtggggcccgggccggggcctcggccccggtcgctgcctcccgtcccgggcgggggcgggcgcgccggccggcctcggtcgccctcccttggccgtcgtgtggcgtgtgccacccctgcgccggcgcccgccggcggggctcggagccgggcttcggccgggccccgggccctcgaccggaccggctgcgcgggcgctgcggccgcacggcgcgactgtccccgggccgggcaccgcggtccgcctctcgctcgccgcccggacgtcggggccgccccgcggggcgggcggagcgccgtccccgcctcgccgccgcccgcgggcgccggccgcgcgcgcgcgcgcgtggccgccggtccctcccggccgccgggcgcgggtcgggccgtccgcctcctcgcgggcgggcgcgacgaagaagcgtcgcgggtctgtggcgcggggcccccggtggtcgtgtcgcgtggggggcgggtggttggggcgtccggttcgccgcgccccgccccggccccaccggtcccggccgccgcccccgcgcccgctcgctccctcccgtccgcccgtccgcggcccgtccgtccgtccgtccgtcgtcctcctcgcttgcggggcgccgggcccgtcctcgcgaggccccccggccggccgtccggccgcgtcgggggctcgccgcgctctaccttacctacctggttgatcctgccagtagcatatgcttgtctcaaagattaagccatgcatgtctaagtacgcacggccggtacagtgaaactgcgaatggctcattaaatcagttatggttcctttggtcgctcgctcctctcctacttggataactgtggtaattctagagctaatacatgccgacgggcgctgacccccttcgcgggggggatgcgtgcatttatcagatcaaaaccaacccggtcagcccctctccggccccggccggggggcgggcgccggcggctttggtgactctagataacctcgggccgatcgcacgccccccgtggcggcgacgacccattcgaacgtctgccctatcaactttcgatggtagtcgccgtgcctaccatggtgaccacgggtgacggggaatcagggttcgattccggagagggagcctgagaaacggctaccacatccaaggaaggcagcaggcgcgcaaattacccactcccgacccggggaggtagtgacgaaaaataacaatacaggactctttcgaggccctgtaattggaatgagtccactttaaatcctttaacgaggatccattggagggcaagtctggtgccagcagccgcggtaattccagctccaatagcgtatattaaagttgctgcagttaaaaagctcgtagttggatcttgggagcgggcgggcggtccgccgcgaggcgagccaccgcccgtccccgccccttgcctctcggcgccccctcgatgctcttagctgagtgtcccgcggggcccgaagcgtttactttgaaaaaattagagtgttcaaagcaggcccgagccgcctggataccgcagctaggaataatggaataggaccgcggttctattttgttggttttcggaactgaggccatgattaagagggacggccgggggcattcgtattgcgccgctagaggtgaaattcttggaccggcgcaagacggaccagagcgaaagcatttgccaagaatgttttcattaatcaagaacgaaagtcggaggttcgaagacgatcagataccgtcgtagttccgaccataaacgatgccgaccggcgatgcggcggcgttattcccatgacccgccgggcagcttccgggaaaccaaagtctttgggttccggggggagtatggttgcaaagctgaaacttaaaggaattgacggaagggcaccaccaggagtggagcctgcggcttaatttgactcaacacgggaaacctcacccggcccggacacggacaggattgacagattgatagctctttctcgattccgtgggtggtggtgcatggccgttcttagttggtggagcgatttgtctggttaattccgataacgaacgagactctggcatgctaactagttacgcgacccccgagcggtcggcgtcccccaacttcttagagggacaagtggcgttcagccacccgagattgagcaataacaggtctgtgatgcccttagatgtccggggctgcacgcgcgctacactgactggctcagcgtgtgcctaccctacgccggcaggcgcgggtaacccgttgaaccccattcgtgatggggatcggggattgcaattattccccatgaacgagggaattcccgagtaagtgcgggtcataagcttgcgttgattaagtccctgccctttgtacacaccgcccgtcgctactaccgattggatggtttagtgaggccctcggatcggccccgccggggtcggcccacggccctggcggagcgctgagaagacggtcgaacttgactatctagaggaagtaaaagtcgtaacaaggtttccgtaggtgaacctgcggaaggatcattaacggagcccggagggcgaggcccgcggcggcgccgccgccgccgcgcgcttccctccgcacacccacccccccaccgcgacgcggcgcgtgcgcgggcggggcccgcgtgcccgttcgttcgctcgctcgttcgttcgccgcccggccccgccgccgcgagagccgagaactcgggagggagacgggggggagagagagagagagagagagagagagagagagagagagagaaagaagggcgtgtcgttggtgtgcgcgtgtcgtggggccggcgggcggcggggagcggtccccggccgcggccccgacgacgtgggtgtcggcgggcgcgggggcggttctcggcggcgtcgcggcgggtctgggggggtctcggtgccctcctccccgccggggcccgtcgtccggccccgccgcgccggctccccgtcttcggggccggccggattcccgtcgcctccgccgcgccgctccgcgccgccgggcacggccccgctcgctctccccggccttcccgctagggcgtctcgagggtcgggggccggacgccggtcccctcccccgcctcctcgtccgcccccccgccgtccaggtacctagcgcgttccggcgcggaggtttaaagaccccttggggggatcgcccgtccgcccgtgggtcgggggcggtggtgggcccgcgggggagtcccgtcgggaggggcccggcccctcccgcgcctccaccgcggactccgctccccggccggggccgcgccgccgccgccgccgcggcggccgtcgggtgggggctttacccggcggccgtcgcgcgcctgccgcgcgtgtggcgtgcgccccgcgccgtgggggcgggaacccccgggcgcctgtggggtggtgtccgcgctcgcccccgcgtgggcggcgcgcgcctccccgtggtgtgaaaccttccgacccctctccggagtccggtcccgtttgctgtctcgtctggccggcctgaggcaaccccctctcctcttgggcggggggggcggggggacgtgccgcgccaggaagggcctcctcccggtgcgtcgtcgggagcgccctcgccaaatcgacctcgtacgactcttagcggtggatcactcggctcgtgcgtcgatgaagaacgcagctagctgcgagaattaatgtgaattgcaggacacattgatcatcgacacttcgaacgcacttgcggccccgggttcctcccggggctacgcctgtctgagcgtcgcttgccgatcaatcgccccgggggtgcctccgggctcctcggggtgcgcggctgggggttccctcgcagggcccgccgggggccctccgtccccctaagcgcagacccggcggcgtccgccctcctcttgccgccgcgcccgccccttccccctccccccgcgggccctgcgtggtcacgcgtcgggtggcgggggggagaggggggcgcgcccggctgagagagacggggagggcggcgccgccgccggaagacggagagggaaagagagagccggctcgggccgagttcccgtggccgccgcctgcggtccgggttcctccctcggggggctccctcgcgccgcgcgcggctcggggttcggggttcgtcggccccggccgggtggaaggtcccgtgcccgtcgtcgtcgtcgtcgcgcgtcgtcggcggtgggggcgtgttgcgtgcggtgtggtggtgggggaggaggaaggcgggtccggaaggggaagggtgccggcggggagagagggtcgggggagcgcgtcccggtcgccgcggttccgccgcccgcccccggtggcggcccggcgtccggccgaccggccgctccccgcgcccctcctcctccccgccgcccctcctccgaggccccgcccgtcctcctcgccctccccgcgcgtacgcgcgcgcgcccgcccgcccggctcgcctcgcggcgcgtcggccggggccgggagcccgccccgccgcccgcccgtggccgcggcgccggggttcgcgtgtccccggcggcgacccgcgggacgccgcggtgtcgtccgccgtcgcgcgcccgcctccggctcgcggccgcgccgcgccgcgccggggccccgtcccgagcttccgcgtcggggcggcgcggctccgccgccgcgtcctcggacccgtccccccgacctccgcgggggagacgcgccggggcgtgcggcgcccgtcccgcccccggcccgtgcccctccctccggtcgtcccgctccggcggggcggcgcgggggcgccgtcggccgcgcgctctctctcccgtcgcctctccccctcgccgggcccgtctcccgacggagcgtcgggcgggcggtcgggccggcgcgattccgtccgtccgtccgccgagcggcccgtccccctccgagacgcgacctcagatcagacgtggcgacccgctgaatttaagcatattagtcagcggaggaaaagaaactaaccaggattccctcagtaacggcgagtgaacagggaagagcccagcgccgaatccccgccccgcggggcgcgggacatgtggcgtacggaagacccgctccccggcgccgctcgtggggggcccaagtccttctgatcgaggcccagcccgtggacggtgtgaggccggtagcggccggcgcgcgcccgggtcttcccggagtcgggttgcttgggaatgcagcccaaagcgggtggtaaactccatctaaggctaaataccggcacgagaccgatagtcaacaagtaccgtaagggaaagttgaaaagaactttgaagagagagttcaagagggcgtgaaaccgttaagaggtaaacgggtggggtccgcgcagtccgcccggaggattcaacccggcggcgggtccggccgtgtcggcggcccggcggatctttcccgccccccgttcctcccgacccctccacccgccctcccttcccccgccgcccctcctcctcctccccggagggggcgggctccggcgggtgcgggggtgggcgggcggggccgggggtggggtcggcgggggaccgtcccccgaccggcgaccggccgccgccgggcgcatttccaccgcggcggtgcgccgcgaccggctccgggacggctgggaaggcccggcggggaaggtggctcggggggccccgtccgtccgtccgtcctcctcctcccccgtctccgccccccggccccgcgtcctccctcgggagggcgcgcgggtcggggcggcggcggcggcggcggtggcggcggcggcgggggcggcgggaccgaaaccccccccgagtgttacagcccccccggcagcagcactcgccgaatcccggggccgagggagcgagacccgtcgccgcgctctcccccctcccggcgcccacccccgcggggaatcccccgcgaggggggtctcccccgcgggggcgcgccggcgtctcctcgtgggggggccgggccacccctcccacggcgcgaccgctctcccacccctcctccccgcgcccccgccccggcgacggggggggtgccgcgcgcgggtcggggggcggggcggactgtccccagtgcgccccgggcgggtcgcgccgtcgggcccgggggaggttctctcggggccacgcgcgcgtcccccgaagagggggacggcggagcgagcgcacggggtcggcggcgacgtcggctacccacccgacccgtcttgaaacacggaccaaggagtctaacacgtgcgcgagtcgggggctcgcacgaaagccgccgtggcgcaatgaaggtgaaggccggcgcgctcgccggccgaggtgggatcccgaggcctctccagtccgccgagggcgcaccaccggcccgtctcgcccgccgcgccggggaggtggagcacgagcgcacgtgttaggacccgaaagatggtgaactatgcctgggcagggcgaagccagaggaaactctggtggaggtccgtagcggtcctgacgtgcaaatcggtcgtccgacctgggtataggggcgaaagactaatcgaaccatctagtagctggttccctccgaagtttccctcaggatagctggcgctctcgcagacccgacgcacccccgccacgcagttttatccggtaaagcgaatgattagaggtcttggggccgaaacgatctcaacctattctcaaactttaaatgggtaagaagcccggctcgctggcgtggagccgggcgtggaatgcgagtgcctagtgggccacttttggtaagcagaactggcgctgcgggatgaaccgaacgccgggttaaggcgcccgatgccgacgctcatcagaccccagaaaaggtgttggttgatatagacagcaggacggtggccatggaagtcggaatccgctaaggagtgtgtaacaactcacctgccgaatcaactagccctgaaaatggatggcgctggagcgtcgggcccatacccggccgtcgccggcagtcgagagtggacgggagcggcgggggcggcgcgcgcgcgcgcgcgtgtggtgtgcgtcggagggcggcggcggcggcggcggcgggggtgtggggtccttcccccgcccccccccccacgcctcctcccctcctcccgcccacgccccgctccccgcccccggagccccgcggacgctacgccgcgacgagtaggagggccgctgcggtgagccttgaagcctagggcgcgggcccgggtggagccgccgcaggtgcagatcttggtggtagtagcaaatattcaaacgagaactttgaaggccgaagtggagaagggttccatgtgaacagcagttgaacatgggtcagtcggtcctgagagatgggcgagcgccgttccgaagggacgggcgatggcctccgttgccctcggccgatcgaaagggagtcgggttcagatccccgaatccggagtggcggagatgggcgccgcgaggcgtccagtgcggtaacgcgaccgatcccggagaagccggcgggagccccggggagagttctcttttctttgtgaagggcagggcgccctggaatgggttcgccccgagagaggggcccgtgccttggaaagcgtcgcggttccggcggcgtccggtgagctctcgctggcccttgaaaatccgggggagagggtgtaaatctcgcgccgggccgtacccatatccgcagcaggtctccaaggtgaacagcctctggcatgttggaacaatgtaggtaagggaagtcggcaagccggatccgtaacttcgggataaggattggctctaagggctgggtcggtcgggctggggcgcgaagcggggctgggcgcgcgccgcggctggacgaggcgcgcgccccccccacgcccggggcacccccctcgcggccctcccccgccccacccgcgcgcgccgctcgctccctccccaccccgcgccctctctctctctctctcccccgctccccgtcctcccccctccccgggggagcgccgcgtgggggcgcggcggggggagaagggtcggggcggcaggggccgcgcggcggccgccggggcggccggcgggggcaggtccccgcgaggggggccccggggacccggggggccggcggcggcgcggactctggacgcgagccgggcccttcccgtggatcgccccagctgcggcgggcgtcgcggccgcccccggggagcccggcggcggcgcggcgcgccccccacccccaccccacgtctcggtcgcgcgcgcgtccgctgggggcgggagcggtcgggcggcggcggtcggcgggcggcggggcggggcggttcgtccccccgccctacccccccggccccgtccgccccccgttcccccctcctcctcggcgcgcggcggcggcggcggcaggcggcggaggggccgcgggccggtcccccccgccgggtccgcccccggggccgcggttccgcgcgcgcctcgcctcggccggcgcctagcagccgacttagaactggtgcggaccaggggaatccgactgtttaattaaaacaaagcatcgcgaaggcccgcggcgggtgttgacgcgatgtgatttctgcccagtgctctgaatgtcaaagtgaagaaattcaatgaagcgcgggtaaacggcgggagtaactatgactctcttaaggtagccaaatgcctcgtcatctaattagtgacgcgcatgaatggatgaacgagattcccactgtccctacctactatccagcgaaaccacagccaagggaacgggcttggcggaatcagcggggaaagaagaccctgttgagcttgactctagtctggcacggtgaagagacatgagaggtgtagaataagtgggaggcccccggcgcccccccggtgtccccgcgaggggcccggggcggggtccgcggccctgcgggccgccggtgaaataccactactctgatcgttttttcactgacccggtgaggcgggggggcgagcccgaggggctctcgcttctggcgccaagcgcccgcccggccgggcgcgacccgctccggggacagtgccaggtggggagtttgactggggcggtacacctgtcaaacggtaacgcaggtgtcctaaggcgagctcagggaggacagaaacctcccgtggagcagaagggcaaaagctcgcttgatcttgattttcagtacgaatacagaccgtgaaagcggggcctcacgatccttctgaccttttgggttttaagcaggaggtgtcagaaaagttaccacagggataactggcttgtggcggccaagcgttcatagcgacgtcgctttttgatccttcgatgtcggctcttcctatcattgtgaagcagaattcgccaagcgttggattgttcacccactaatagggaacgtgagctgggtttagaccgtcgtgagacaggttagttttaccctactgatgatgtgttgttgccatggtaatcctgctcagtacgagaggaaccgcaggttcagacatttggtgtatgtgcttggctgaggagccaatggggcgaagctaccatctgtgggattatgactgaacgcctctaagtcagaatcccgcccaggcgaacgatacggcagcgccgcggagcctcggttggcctcggatagccggtcccccgcctgtccccgccggcgggccgcccccccctccacgcgccccgccgcgggagggcgcgtgccccgccgcgcgccgggaccggggtccggtgcggagtgcccttcgtcctgggaaacggggcgcggccggaaaggcggccgccccctcgcccgtcacgcaccgcacgttcgtggggaacctggcgctaaaccattcgtagacgacctgcttctgggtcggggtttcgtacgtagcagagcagctccctcgctgcgatctattgaaagtcagccctcgacacaagggtttgtccgcgcgcgcgtgcgtgcggggggcccggcgggcgtgcgcgttcggcgccgtccgtccttccgttcgtcttcctccctcccggcctctcccgccgaccgcggcgtggtggtggggtgggggggagggcgcgcgaccccggtcggccgccccgcttcttcggttcccgcctcctccccgttcacgccggggcggctcgtccgctccgggccgggacggggtccggggagcgtggtttgggagccgcggaggcgccgcgccgagccgggccccgtggcccgccggtccccgtcccgggggttggccgcgcggcgcggtggggggccacccggggtcccggccctcgcgcgtccttcctcctcgctcctccgcacgggtcgaccgacgaaccgcgggtggcgggcggcgggcggcgagccccacgggcgtccccgcacccggccgacctccgctcgcgacctctcctcggtcgggcctccggggtcgaccgcctgcgcccgcgggcgtgagactcagcggcgtctcgccgtgtcccgggtcgaccgcggccttctccaccgagcggcggtgtaggagtgcccgtcgggacgaaccgcaaccggagcgtccccgtctcggtcggcacctccggggtcgaccagctgccgcccgcgagctccggacttagccggcgtctgcacgtgtcccgggtcgaccagcaggcggccgccggacgcagcggcgcacgcacgcgagggcgtcgattccccttcgcgcgcccgcgcctccaccggcctcggcccgcggtggagctgggaccacgcggaactccctctcccacatttttttcagccccaccgcgagtttgcgtccgcgggacctttaagagggagtcactgctgccgtcagccagtactgcctcctcctttttcgcttttaggttttgcttgcctttttttttttttttttttttttttttttctttctttctttctttctttctttctttctttctttctttctttcgcttgtcttcttcttgtgttctcttcttgctcttcctctgtctgtctctctctctctctctctctctgtctctcgctctcgccctctctctcttctctctctctctctctctctctctgtctctcgctctcgccctctctctctctcttctctctgtctctctctctctctctctctctctctctctctgtcgctctcgccctctcgctctctctctgtctctgtctgtgtctctctctctccctccctccctccctccctccctccctccctccccttccttggcgccttctcggctcttgagacttagccgctgtctcgccgtaccccgggtcgaccggcgggccttctccaccgagcggcgtgccacagtgcccgtcgggacgagccggacccgccgcgtccccgtctcggtcggcacctccggggtcgaccagctgccgcccgcgagctccggacttagccggcgtctgcacgtgtcccgggtcgaccagcaggcggccgccggacgcagcggcgcaccgacggagggcgctgattcccgttcacgcgcccgcgcctccaccggcctcggcccgccgtggagctgggaccacgcggaactccctctcctacatttttttcagccccaccgcgagtttgcgtccgcgggacctttaagagggagtcactgctgccgtcagccagtactgcctcctcctttttcgcttttaggttttgcttgcctttttttttttttttttttttttttttttctttctttctttctttctttctttctttctttctttctttctttctttcgctctcgctctctcgctctctccctcgctcgtttctttctttctctttctctctctctctctctctctctctctctctgtctctcgctctcgccctctctctctctttctctctctctctgtctctctctctctctctctctctctctctctctccctccctccctccccctccctccctctctccccttccttggcgccttctcggctcttgagacttagccgctgtctcgccgtgtcccgggtcgaccggcgggccttctccaccgagcggcgtgccacagtgcccgtcgggacgagccggacccgccgcgtccccgtctcggtcggcacctccggggtcgaccagctgccgcccgcgagctccggacttagccggcgtctgcacgtgtcccgggtcgaccagcaggcggccgccggacgctgcggcgcaccgacgcgagggcgtcgattccggttcacgcgccggcgacctccaccggcctcggcccgcggtggagctgggaccacgcggaactccctctcccacatttttttcagccccaccgcgagtttgcgtccgcgggacttttaagagggagtcactgctgccgtcagccagtaatgcttcctccttttttgctttttggttttgccttgcgttttctttctttctttctttctttctttctttctttctttctttctctctctctctctctctctctctctgtctctctctctctgtctctctcccctccctccctccttggtgccttctcggctcgctgctgctgctgcctctgcctccacggttcaagcaaacagcaagttttctatttcgagtaaagacgtaatttcaccattttggccgggctggtctcgaactcccgacctagtgatccgcccgcctcggcctcccaaagactgctgggagtacagatgtgagccaccatgcccggccgattccttccttttttcaatcttattttctgaacgctgccgtgtatgaacatacatctacacacacacacacacacacacacacacacacacacacacacacacacacaccccgtagtgataaaactatgtaaatgatatttccataattaatacgtttatattatgttacttttaatggatgaatatgtatcgaagccccatttcatttacatacacgtgtatgtatatccttcctcccttccttcattcattatttattaataattttcgtttatttattttcttttcttttggggccggcccgcctggtcttctgtctctgcgctctggtgacctcagcctcccaaatagctgggactacagggatctcttaagcccgggaggagaggttaacgtgggctgtgatcgcacacttccactccagcttacgtgggctgcggtgcggtggggtggggtggggtggggtggggtgcagagaaaacgattgattgcgatctcaattgccttttagcttcattcataccctgttatttgctcgtttattctcatgggttcttctgtgtcattgtcacgttcatcgtttgcttgcctgcttgcctgtttatttccttccttccttccttccttccttccttccttccttccttccttccctcccttactggcagggtcttcctctgtctctgccgcccaggatcaccccaacctcaacgctttggaccgaccaaacggtcgttctgcctctgatccctcccatccccattacctgagactacaggcgcgcaccaccacaccggctgacttttatgttgtttctcatgttttccgtaggtaggtatgtgtgtgtgtgtgtgtgtgtgtgtgtgtgtgtgtgtgtgtgtgtgtgtgtgtgtgtgtatctatgtatgtacgtatgtatgtatgtatgtgagtgagatgggtttcggggttctatcatgttgcccacgctggtctcgaactcctgtcctcaagcaatccgcctgcctgcctcggccgcccacactgctgctattacaggcgtgagacgctgcgcctggctccttctacatttgcctgcctgcctgcctgcctgcctgcctatcaatcgtcttctttttagtacggatgtcgtctcgctttattgtccatgctctgggcacacgtggtctcttttcaaacttctatgattattattattgtaggcgtcatctcacgtgtcgaggtgatctcgaacttttaggctccagagatcctcccgcatcggcctcccggagtgctgtgatgacacgcgtgggcacggtacgctctggtcgtgtttgtcgtgggtcggttctttccgtttttaatacggggactgcgaacgaagaaaattttcagacgcatctcaccgatccgccttttcgttctttctttttattctctttagacggagtttcactcttgtcgcccagggtggagtacgatggcggctctcggctcaccgcaccctccgcctcccaggttcaagtgattctcctgcctcagccttcccgagtagctggaatgacagagatgagccatcgtgcccggctaatttttctatttttagtacagatggggtttctccatcttggtcaggctggtcttcaacttccgaccgttggagaatcttaactttcttggtggtggttgttttcctttttctttttttttcttttcttttctttccttctcctcccccccccaccccccttgtcgtcgtcctcctcctcctcctcctcctcctcctcctcctcctcctcctcctcctcctctttcatttctttcagctgggctctcctacttgtgttgctctgttgctcacgctggtctcaaactcctggccttgactcttctcccgtcacatccgccgtctggttgttgaaatgagcatctctcgtaaaatggaaaagatgaaagaaataaacacgaagacggaaagcacggtgtgaacgtttctcttgccgtctcccggggtgtaccttggacccggaaacacggagggagcttggctgagtgggttttcggtgccgaaacctcccgagggcctccttccctctcccccttgtccccgcttctccgccagccgaggctcccaccgccgcccctggcattttccataggagaggtatgggagaggactgacacgccttccagatctatatcctgccggacgtctctggctcggcgtgccccaccggctacctgccaccttccagggagctctgaggcggatgcgacccccacccccccgtcacgtcccgctaccctcccccggctggcctttgccgggcgaccccaggggaaccgcgttgatgctgcttcggatcctccggcgaagacttccaccggatgccccgggtgggccggttgggatcagactggaccaccccggaccgtgctgttcttgggggtgggttgacgtacagggtggactggcagccccagcattgtaaagggtgcgtgggtatggaaatgtcacctaggatgccctccttcccttcggtctgccttcagctgcctcaggcgtgaagacaacttcccatcggaacctcttctcttccctttctccagcacacagatgagacgcacgagagggagaaacagctcaatagataccgctgaccttcatttgtggaatcctcagtcatcgacacacaagacaggtgactaggcagggacacagatcaaacactatttccgggtcctcgtggtgggattggtctctctctctctctctctctctctctctctctctctctctctcgcacgcgcacgcgcgcacacacacacacaatttccatatctagttcacagagcacactcacttccccttttcacagtacgcaggctgagtaaaacgcgccccaccctccacccgttggctgacgaaaccccttctctacaattgatgaaaaagatgatctgggccgggcacgctagctcacgcctgtcactccggcactttgggaggccgaggcgggtggatcgcttggggccgggagttcgagaccaggctggccgacgtggcgaaaccccgtctctctgaaaaatagaacgattagccgggcctggtggcgtgggcttggaatcacgaccgctcgggagactggggcgggcgacttgttccaaccggggaggccgaggccgcgatgagctgagatcgtgccgtggcgatgcggcctggatgacggagcgagaccccgtctcgagagaatcatgatgttattataagatgagttgtgcgcggtgatggccgcctgtagtcgcggctactcgggaggctgagacgaggagaagatcacttgaggccccacaggtcgaggcttcggtcggccgtgacccactgtatcctgggcagtcaccggtcaaggagatatgccccttccccgtttgcttttcttttcttcccttctcttttcttctttttgcttctcttttctttctttctttctttctttctttctttctttctttctttctttttctttttctctcttcccctctttctttcctgccttcctgcctttcttcttttcttctttcctcccttcctcccttccttctttcctcccgcctcagcctcccaaagtgctgggatgactggcgggaggcaccatgcctgcttggcccaaagagaccctcttggaaagtgagacgcagagagcgccttccagtgatctcattgactgatttagagacggcatctcgctccgtcaccccggcagtggtgccgtcgtaactcactccctgcagcgtggacgctcctggactcgagcgatccttccacctcagcctccagagtacagagcctgggaccgcgggcacgcgccactgtgcccacaccgtttttaattgtttttttttcccccgagacagagtttcactctcgtggcctagactgcagtgcggtggcgcgatcttggctcaccgcaacctctgcctcccggtttcaagcgattctcctgcatcggcctcctgagtagccgggattgcgggcatgcgctgccacgtctggctgatttcgtatttttagtggagacggggcttctccatgtcgatcgggctggtttcgaactcccgacctcaggtgatccgccctccccggcctccggaagtgctgggatgacaggcgtgagccaccgcgcccggccttcatttttaaatgttttcccacagacggggtctcatcatttctttgcaaccctcctgcccggcgtctcaaagtgctggcgtgacgggcgtgagccactgcgcctggactccggggaatgactcacgaccaccatcgctctactgatcctttctttctttctttctttctttctttctttctttctttctttctttctttctttcttgatgaattatcttatgatttatttgtgtacttattttcagacggagtctcgctctgggcggggcgaggcgaggcgaggcacagcgcatcgctttggaagccgcggcaacgcctttcaaagccccattcgtatgcacagagccttattcccttcctggagttggagctgatgccttccgtagccttgggcttctctccattcggaagcttgacaggcgcagggccacccagaggctggctgcggctgaggattagggggtgtgttggggctgaaaactgggtcccctatttttgatacctcagccgacacatcccccgaccgccatcgcttgctcgccctctgagatcccccgcctccaccgccttgcaggctcacctcttactttcatttcttcctttcttgcgtttgaggagggggtgcgggaatgagggtgtgtgtggggagggggtgcggggtggggacggaggggagcgtcctaagggtcgatttagtgtcatgcctctttcaccaccaccaccaccaccgaagatgacagcaaggatcggctaaataccgcgtgttctcatctagaagtgggaacttacagatgacagttcttgcatgggcagaacgagggggaccggggacgcggaagtctgcttgagggaggaggggtggaaggagagacagcttcaggaagaaaacaaaacacgaatactgtcggacacagcactgactacccgggtgatgaaatcatctgcacactgaacacccccgtcacaagtttacctatgtcacaatcttgcacatgtatcgcttgaacgacaaataaaagttaggggggagaagagaggagagagagagagagagagagacagagagagacagagagagagagagaggagggagagaggaaaacgaaacaccacctccttgacctgagtcagggggtttctggccttttgggagaacgttcagcgacaatgcagtatttgggcccgttctttttttttcttcttcttttctttctttttttttggactgagtctctctcgctctgtcacccaggctgcggtcgcggtggcgctctctcggctcactgaaacctctgcttcccgggttccagtgattcttcttcggtagctgggattacaggcgcacaccatgacggcgggctcatattcctattttcagtagagacggggtttctccacgttggccacgctggtctcgaactcctgacctcaaatgatccgccttcctgggcctcccaaagtgctggaaacgacaggcctgagccgccgggatttcagcctttaaaagcgcggccctgccacctttcgctgtggcccttacgctcagaatgacgtgtcctctctgccgtaggttgactccttgagtcccctaggccattgcactgtagcctgggcagcaagagccaaactccgnncccccacctcctcgcgcacataataactaactaacaaactaactaactaactaaactaactaactaactaaaatctctacacgtcacccataagtgtgtgttcccgtgagagtgatttctaagaaatggtactgtacactgaacgcagtggctcacgtctgtcatcccgaggtcaggagttcgagaccagcccggccaacgtggtgaaaccccgtctctactgaaaatacgaaatggagtcaggcgccgtggggcaggcacctgtaaccccagctactcgggaggctggggtggaagaattgcttgaacctggcaggcggaggctgcagtgacccaagatcgcaccactgcactacagcctgggcgacagagtgagacccggtctccagataaatacgtacataaataaatacacacatacatacatacatacatacatacatacatacatacatccatgcatacagatatacaagaaagaaaaaaagaaaagaaaagaaagagaaaatgaaagaaaaggcactgtattgctactgggctagggccttctctctgtctgtttctctctgttcgtctctgtctttctctctgtgtctctttctctgtctgtctgtctctttctttctctctgtctctgtctctgtctttgtctctctctctccctctctgcctgtctcactgtgtctgtcttctgtcttactctctttctctccccgtctgtctctctctctctctctccctccctgtttgtttctctctctccctccctgtctgtttctctctctctctttctgtctgtttctgtctctctctgtctgtctatgtctttctctgtctgtctctttctctgtctgtctgcctctctctttctttttctgtgtctctctgtcggtctctctctctctgtctgtctgtctgtctctctctctctctctctgtgcctatcttctgtcttactctctttctctgcctgtctgtctgtctctccctccctttctgtttctctctctctctctctctctctccccctctccctgtctgtttctctccgtctctctctctttctgtctgtttctcactgtctctctctgtccatctctctctctctctgtctgtctctttcgttctctctgtctgtctgtctctctctctctctctctctctctctctctctccctgtctgtctgtttctctctatctctcgctgtccatctctgtctttctatgtctgtctctttctctgtcagtctgtcagacacccccgtgccgggtagggccctgccccttccacgaaagtgagaagcgcgtgcttcggtgcttagagaggccgagaggaatctagacaggcgggccttgctgggcttccccactcggtgtatgatttcgggaggtcgaggccgggtccccgcttggatgcgaggggcattttcagacttttctctcggtcacgtgtggcgtccgtacttctcctatttccccgataagctcctcgacttcaacataaacggcgtcctaagggtcgatttagtgtcatgcctctttcaccgccaccaccgaagatgaaagcaaagatcggctaaataccgcgtgttctcatctagaagtgggaacttacagatgacagttcttgcatgggcagaacgagggggaccgggnacgcggaagcctgcttgagggrggaggggyggaaggagagacagcttcaggaagaaaacaaaacacgaatactgtcggacacagcactgactacccgggtgatgaaatcatctgcacactgaacacccccgtcacaagtttacctatgtcacagtcttgctcatgtatgcttgaacgacaaataaaagttcgggggggagaagagaggagagagagagagagacggggagagaggggggagagggggggggagagagagagagagagagagagagagagagagagagagaaagagaagtaaaaccaaccaccacctccttgacctgagtcagggggtttctggccttttgggagaacgttcagcgacaatgcagtatttgggcccgttctttttttcttcttcttcttttctttctttttttttggactgagtctctctcgctctgtcacccaggctgcggtgcggtggcgctctctcggctcactgaaacctctgcttcccgggttccagtgattcttcttcggtagctgggattacaggtgcgcaccatgacggccggctcatcgttctatttttagtagagacggggtttctccacgttggccacgctggtctcgaactcctgaccacaaatgatccaccttcctgggcctcccaaagtgctggaaacgacaggcctgagccgccgggatttcagcctttaaaagcgcgcggccctgccacctttcgctgcggcccttacgctcagaatgacgtgtcctctctgccataggttgactccttgagtcccctaggccattgcactgtagcctgggcagcaagagccaaactccgtccccccacctccccgcgcacataataactaactaactaactaactaactaaaatctctacacgtcacccataagtgtgtgttcccgtgaggagtgatttctaagaaatggtactgtacactgaacgcaggcttcacgtctgtcatcccgaggtcaggagttcgagaccagcccggcccacgtggtgaaacccccgtctctactgaaaatacgaaatggagtcaggcgccgtggggcaggcacctgtaaccccagctactcgggaggctggggtggaagaattgcttgaacctggcaggcggaggctgcagtgacccaagatcgcaccactgcactacagcctgggcgacagagtgagacccggtctccagataaatacgtacataaataaatacacacatacatacatacatacatacaacatacatacatacagatatacaagaaagaaaaaaagaaaagaaaagaaagagaaaatgaaagaaaaggcactgtattgctactgggctagggccttctctctgtctgtttctctctgttcgtctctgtctttctctctgtgtctctttctctgtctgtctgtctgtctgtctgtctgtctctttctttctttctgtctctgtctttgtccctctctctccctctctgccctgtctcactgtgtctgtcttctatcttactctctttctctccccgtctgtctctctctcactccctccctgtctgtttctctctctctctctttctgtctgtttctgtctctctctgtctgcctctctctttctctatctgtctctttctctgtctgtctgcccctctctttctttttctgtgtctctctgtctgtctctctctctctctgtgcctatcttctgtcttactctctttctctgcctgtctgtctgtctctctctgtctctccctccctttctgcttctctctctctctctctctctnnnccctccctgtctgtttctctctgtctccctctctttctgtctgtttctcactgtctctctctgtctgtctgtttcattctctctgtctctgtctctgtctctctctctctctgtctctccctctctgtgtgtatcttttgtcttactctccttctctgcctgtccgtctgtctgtctgtctctctctctccctgtccctctctctttctgtctgtttctctctctctctctctctctctctctctgtctctgtctttctctgtctgtccctttctctgtctgtctgcctctctctttctctttctgtgtctctctgtctctctctctgtgcctatcttctgtcttactctctttctctgcctgtctatctgtctgtctctctctgtctctctccctgcctttctgtttctctctctctccctctctcgctctctctgtctttctctctttctctctgtttctctgtctctctctgtccgtctctgtctttttctgtctgtctgtctctctctttctttctgtcgtctgtctctgtctctgtctctgtctctctctctctctctctccttgtctctctcactgtgtctgtcttctgtcttactctccttctctgcctgtccatctgtctgtctgtctctctctctctctccctacctttctgtttctctctcgctagctctctctctctctgcctgtttctctctttctctctctgtctttctctgtctgtctctttctctgtctgtctgtctctttctctctgtctctgtctctgtctctctctctctctctctctctctctctgcctctctcactgtgtctgtcttctgtcttattctctttctctctctgtctctctctctctctcctttactgtctgtttctctctctctctctctctttctgcctgtttctctctgtctgtctctgtctttctctgtctgtctgcctctctctttctttttctgcgtctctctgtctctctctctctctctctgttcctatcttctgtcttactctgtttccttgcctgcctgcctgtctgtgtgtctgtctctctctctctctctctctctctctccctccctttctctttctctgtctctctctctctttctgggtgtttctctctgtctctctgtccatctctgtctttctatgtctgtctctctctttctctctgtctctgtctctgcctctctctctctctctctctctctctctctgtctgtctctctcactgtgtgtgtctgtcttctgtcttactctccttctctgcctgtccgtctgtctgtctgtctctccctctctctccctccctttctgtttctctctctctctctttctgtctgtttctctctttctctctctgtctgtctctttctctgtctgtctgtctctctctttctttttctctgtctctctgtctctctctgtgtctgtctctctgtctgtgcctatcttctgtcttactctctttctctggctgtctgcctgtctctctctctctctctgtctgtctccgtccctctctccctgtctgtctgtttctctctctgcctctctctctctctgtctgtctctttctctgtctgtctgtctctctctttctttttctctgtctctctgtctctctctgtgtctgtctctctttctgtgcctatcttctgtcttactctctttctctggctgtctgcctgtctctctctctctgcctgtctccgtccctccctccctgtctgtctgtttctctctctgtctctgtctctctgtccatctctgtctgtctctttctctttctctctctctgtctctgtctctctctctctctgcctgtctctctcactgtgtctgtcttctgtcttactctctttctcttgcctgcctctctgtctgtctgtctctctccctccatgtctctctctctctctcactcactctctctccgtctctctctctttctgtctgtttctctctctgtctgtctctctccctccatgtctctctctctctctctcactcactctctctccgtctctctctctctttctgtctgtttctctctctgtctgtctctctccctccatgtctctctctctccctctcactcactctctctccgtctctctctctctttctgtctgtttctttgtctgtctgtctgtctgtctgtctgtctctctctctctctctctctctctctctctctgtttgtctttctccctccctgtctgtctgtctgtctctctctctctgtctctgtctctgtctctctctctttctctttctgtctgtttctctctatctctcgctgtccatctctgtctttctatgtctgtctctttctctgtcagtctgtcagacacacccgtgccggtagggccctgcccttccacgagagtgagaagcgcgtgcttcggtgcttagagaggccgagaggaatctagacaggcgggccttgctgggcttccccactcggtgtacgatttcgggaggtcgaggccgggtccccgcttggatgcgaggggcattttcagacttttctctcggtcacgtgtggcgtccgtacttctcctatttccccgataagtctcctcgacttcaacataaactgttaaggccggacgccaacacggcgaaaccccgtctctactaaaaatacaaagctgagtcgggagcggtggggcaggccctgtaatgccagctcctcgggaggctgaggcgggagaatcgcttgaaccagggaagcggaggctgcagggagccgagatcgcgccactgcactacggcccaggctgtagagtgagtgagactcggtctctaaataaatacggaaattaattaattcattaattcttttccctgctgacggacatttgcaggcaggcatcggttgtcttcgggcatcacctagcggccactgttattgaaagtcgacgttgacacggagggaggtctcgccgacttcaccgagcctggggcaacgggtttctctctctcccttctggaggcccctccctctctccctcgttgcctagggaacctcgcctagggaacctccgccctgggggccctattgttctttgatcggcgctttacttttctttgtgttttggcgcctagactcttctacttgggctttgggaagggtcagtttaattttcaagttgccccccggctccccccactacccacgtcccttcaccttaatttagtgagncggttaggtgggtttcccccaaaccgcccccccccccccgcctcccaacaccctgcttggaaaccttccagagccaccccggtgtgcctccgtcttctctccccttcccccaccccttgccggcgatctcattcttgccaggctgacatttgcatcggtgggcgtcaggcctcactcgggggccaccgtttttgaagatgggggcggcacggtcccacttccccggaggcagcttgggccgatggcatagccccttgacccgcgtgggcaagcgggcgggtctgcagttgtgaggcttttccccccgctgcttcccgctcaggcctccctccctaggaaagcttcaccctggctgggtctcggtcaccttttatcacgatgttttagtttctccgccctccggccagcagagtttcacaatgcgaagggcgccacggctctagtctgggccttctcagtacttgcccaaaatagaaacgctttctgaaaactaataactttnctcacttaagatttccagggacggcgccttggcccgtgtttgttggcttgttttgtttcgttctgttttgttttgttcgtgtttttcctttctcgtatgtctttcttttcaggtgaagtagaaatccccagttttcaggaagacgtctattttccccaagacacgttagctgccgttttttcctgttgtgaactagcgcttttgtgactctctcaacgctgcagtgagagccggttgatgtttacnatccttcatcatgacatcttattttctagaaatccgtaggcgaatgctgctgctgctcttgttgctgttgttgttgttgttgttgtcgtcgttgctgttgtcgttgtcgttgttgttgtcgttgtcgttgttttcaaagtataccccggccaccgtttatgggatcaaaagcattataaaatatgtgtgattatttcttgagcacgcccttcctccccctctctctgtctctctgtctgtctctgtctctctctttctctgtctgtcttctctctctctctctctctgtgtctctctctctctgcctgtctgtttctctctctctgcctctctctctctctctctctctgcctgtctctctcactgtgtctgtcttctgtcttactccctttctctgtctgtctgtcggtctctctctctctctctccctgtctgtatgtttctctctgtctctgtctctctctctctttctgtttctctctctccgtctctgtctttctctgactgtctctctctttccttctctctgtctctctctgcctgtctctctcactctgtcttctgtcttatctctctctctgcctgcctgtctctctcactctctctctctgtgtgtctctctctctctttctgtttctctctgtctctctgtccgtctctgtctttctctgtctgtctctttgtctgtctgtctttgtctttccttctctctgtctctgtctctctcactgtgtctgtcttctgtcttagtctctctctctctctctccctgtctgtctgtctctctctctctctccccctgtctgtttctctctctctctctctctctctctctctctgtctttgtctttctttctgtctctgtctctctctctctctctgtgtgtctgtcttctgtcttactgtctttctctgcctgtctgtctgtctgtctctctctgtctgtctctctctctctctccccctgtcggctgtttctctgtctctgtctgtgtctctctttctgtctgtttctctctgtctgtctttctctctctgtctctttctctctgtctctctgtctgtctctgtctctctctctgtctctctctctctgtgggggtgtgtgtgtgtgtgtgtatgtgtgtgtgtgtgtgtgtgtgtgtctgccttctgtcttactctctttctctgcctgtctgtctgcctgtctgtttgtctctctctctctgcctgtctctctcccttcctgtctgtttctctctctttctgtttctctctgtctctgtccatctctgtctttctccgtctgtctctttatctgtctctctccgtctgtctctttatctgtctctctctctctttctgtctttctctctctgtgtatcgttgtctctctctgtctgtctctgtctctgtctctctgtctctctctctctctctctctctctgtctgtctgtccgtctgtctgtctcggtctctgcgtctcgctatctcccgccctctctttttttgcaaaagaagctcaagtacatctaatctaatcccttaccaaggcctgaattcttcacttctgacatcccagatttgatctccctacagaatgctgtacagaactggcgagttgatttctggacttggatacctcatagaaactacatatgaataaagatccaatcctaaaatctggggtggcttctccctcgactgtctcgaaaaatcgtacctctgttcccctaggatgccggaagagttttctcaatgtgcatctgcccgtgtcctaagtgatctgtgaccgagccctgtccgtcctgtctcaaatatgtacgtgcaaacacttctctccatttccacaactacccacggccccttgtggaaccactggctctttgaaaaaaatcccagaagtggttttggctttttggctaggaggcctaagcctgctgagaactttcctgcccaggatcctcgggaccatgcttgctagcgctggatgagtctctggaaggacgcacgggactccgcaaagctgacctgtcccaccgaggtcaaatggatacctctgcattggcccgaggcctccgaagtacatcaccgtcaccaaccgtcaccgtcagcatccttgtgagcctgcccaaggccccgcctccggggagactcttgggagcccggccttcgtcggctaaagtccaaagggatggtgacttccacccacaaggtcccactgaacggcgaagatgtggagcgtaggtcagagaggggaccaggaggggagacgtcccgacaggcgacgagttcccaaggctctggccaccccacccacgccccacgccccacgtcccgggcacccgcgggacaccgccgctttatcccctcctctgtccacagccggccccaccccaccacgcaacccacgcacacacgctggaggttccaaaaccacacggtgtgactagagcctgacggagcgagagcccatttcacgaggtgggaggggtgggggtggggtgggttgggggttgtggggtctgtggcgagcccgattctccctcttgggtggctacaggctagaaatgaatatcgcttcttggggggaggggcttccttaggccatcaccgcttgcgggactacctctcaaaccctcccttgaggccacaaaatagattccaccccacccatcgacgtttcccccgggtgctggatgtatcctgtcaagagacctgagcctgacaccgtcgaattaaacaccttgactggctttgtgtgtttgtttgtttctgagatggagtcttgctctgtcccccaggctggagtgcagtggcgtgatctcagctcactggaacctctgcctcctgggttcaagtgattctcctgtctcagcgccaccatggccggctcattttttttttttttttttttggtagacacggggtttcaccctctttcattggttttcactggagattctagattcgagccacacctcattccgtgccacagagagacttcttttttttttttttttttttaagcgcaacgcaacatgtctgccttatttgagtggcttcctatatcattataattgtgttatagatgaagaaacggtattaaacactgtgctaatgatagtgaaagtgaagacaaaagaaaggctatctattttgtggttagaataaagttgctcagtatttagaagctacctaaatacgtcagcatttacactcttcctagtaaaagctggccgatctgaataatcctcctttaaacaaacacaatttttgatagggttaagatttttttaagaatgcgactcctgcaaaatagctgaacagacgatacacatttaaaaaaataacaacacaaggatcaaccagacttgggaaaaaatcgaaaaccacacaagtcttatgaagaactgagttcttaaaataggacggagaacgtagctatcggaagagaaggcagtattggcaagttgattgttacgttggtcagcagtagctggcactatctttttggccatctttcgggcaatgtaactactacagcaaaatgagatatgatccattaaacaacatattcgcaaatcaaaaagtgtttcagtaatataatgcttcagatttagaagcaaatcaaatgatagaactccactgctgtaataagtcaccccaaagatcaccgtatctgacaaaataactaccacagggttatgacttcagaatcatactttcttcttgatatttacttatgtatttattttttttaatttatttctcttgagacgcgtctcgctctgtcgcccaggctggagtgcgatggtgtgatctcggctcactgcaaccgccacctccctgggttcaagcgattctcctgcctcagcctcccgagtagctgggactacaggtgcccgccaccacgcccagctaatctttatacttttaatagagacggggtttcaccgtgtcggcccggatggtctcgatctcttgacctcgtgacccgcccgcctcggcctcccaaagtgctgggatgacaggcgtgagccactgagcccggccttctcttgacgtttaaactatgaagtcagtccagagaaacgcaataaatgtcaacggtgaggatggtgttgaggcagaagtaggaccacactttttcctatcttattcagttgataacaatatgacctaggtagtaatttcctatgtgcctacttatacacgagtacaaaagagtaaaacagagagactgctaaattaaagggtacgtgaagttcttcatagtaactccgtaaactggaacactgtcaaaaagcagcagctagtgaattgtttccatgtatttttctattatccaataagtgaactatgctattcctttccagtctcccaagcacttcttgtccccatcaccacttcggtgctcgaagaaaaagtaagcaaatcaaggaacacaagctaaagaaacacacacacaaaccaaagacaactacagcgtctgcaaaagtttgctagaagactgaaactgttgagtataaggatctggtattctacgatcatgagttcacttcagagtttgttcaagacatacgtttcgtaaggaaacatcttagttagaagttattcagcagtaggtaccatccctaagtatttttcaccaaatccgtgacaataaagagctatctaaccagaaaaattagcgagtacgggcaccatccatagggctttgtctttacgcttcattagcacttaccatgccttacaatgtctaggattgaccctgatagcatttcgaaaacaagctaatgctttgtccagttcttcagtgaagacaactcacgccctaatgcgctataggcataagcatcatttggatccacttcgagagttctctggaagaattgaatcgcaatatcgtgttcccgtttgcagaccgaaacagtttccctgcagcacaccaggcctctggctggcgaatttttatccatgtctgtgaagtctttggacagaactgaaagagcaacctctttcggaggatgccaaagtgttgtagagtagatctccatgccttcgactctgtaattctcaatcctcctaacctctgagaattgtctttcagcttgcgtggactctgaaagtttacaataggccntttccgatttggcacagtacccaaccggtattgcagtggtgagaagctagatggctcaagatgctgatagcttctttgccgtggtaagaacacaaagctaaataacctttccccctttcacgaagaaggctcatcaagccttccgctgctgctttttgtagattaaaagcctgaatctgaggcgcgattgcggctattttcccttctgaaatgacggaagagtccaattttgtcacttccaggctatcacttatgttcggtggagttattgctcctttattagttttacttttggttcttctgtttgggattttaggtggaaacttcatttttaattttctcctaattctcctcggttgtggagctgtcactagtcaagagtcgtgaatttcttcgaggncggtgcatttgggggagatgccatagtggggctcaatacctgaggtgttgcccttgtcggcggaccagaactttgtgtttttgcaaggactggagttacctttcggctctttcccctctgcgagaagacagacggtgttccggtttggccgattctggcaacaggcttttctgaaggggctccggtggatggcacgtcagtgacagacggtgtctcataccagtgcagttttgtcaatagggtccgtctccgggacttggggtttctaatggcaaaatgccaacacttggggttaatggactaacagctgctggtcctcctaataaacttcgaccagtttttggtttatgttgaacctgtttagatcatatggaagttcctgttcccagtgggacagtatcaggtgaaaggacagctgaatcgatagaagacactggggagtctgtattcaaggagtactttgaattggaagattctaaattccatccgtttcattcgacggtgtcctggggtgtttccgtaagaacggtctcgggctgtctgtgacataaactaggacgaggtccaagtgttgtggcgcaacacttggacaggcagttgctaaagctctctagagaggtgaatcaaaatgtttggtcaggatctggcttttcccccctatttcacatcatgattcaaagggacaccagaggaaaggatttcaacgaaggctcttttggtcacattctgatcctttggtaagccgatctgtcttgcaatatacatgtcccgacgatggaaggggaaagcgagctgaatcaccaaactcaggaacgataatatcatcgtggcttttctgcttatgaaacactccacccgataagatttgatccccttctgcaagcttgctgagatcaacacaacatttcgcaagcaggcatttgcattgcggggtagtacaactgtgtcctttcaagagtctatatgttttataggcctttcctgagcggtaagaacaggtcgccagtaagaacaaggcttcttctgagtgtacttctgcataaaggcgttctgcgggggaaaccgcatctcggtaggcatagtggtttagtgcttgccatatagcagcctggacgggtccctgcagcaccgccatcctcgaggctcaggcccactttctgcagtgccacaggcacccccccccccccatagcggctccggcccggccagccccggctcatttaaaggcaccagccgccgttaccgggggatgggggagtccgagacagaatgacttctttatcctgctgactctggaaagcccggcgccttgtgatccattgcaaaccgagagtcacctcgtgtttagaacacggatccactcccaagttcagtggggggatgtgaggggtgtggcaggtaggacgaaggactctcttccttctgattcggtctgcacagtggggcctagggctggagctctctccgtgcggaccgctgactccctctaccttgggttccctcggccccaccctggaacgccgggccttggcagattctggccctttctggcccttcagtcgctgtcagaaaccccatctcatgctcggatgccccgagtgactgtggctcgcacctctccggaaacattggaaatctctcctctacgcgcggccacctgaaaccacaggagctcgggacacacgtgctttcgggagagaatgctgagagtctctcgccgactctctcttgacttgagttcttcgtgggtgcgtggttaagacgtagtgagaccagatgtattaactcaggccgggtgctggtggctcacgcctgtaaccccaacactttgggaggccgaggccgtaggatccctcgaggaatcgcctaaccctggggaggttgaggttgcagtgagtgagccatagttgtgtcactgtgctccagtctgggcgaaagacagaatgaggccctgccacaggcaggcaggcaggcaggcaggcagaaagacaacagctgtattatgttcttctcagggtaggaagcaaaaataacagaatacagcacttaattaattttttttttttccttcggacggagtttcactcttggtgcccacgctggagtgcagtggcaccatctcggctcaccgcaacctccacctcccgcgttcaagcgattctcctgcctcagcctcctgagtagctgggattacagggaggagccaccacacccagctgattttgtattgttagtagagacggcatttctccatgtgggtcaggctggtctcgaactggcgaccccagtggatctgcccgccccggcctcccaaagtgctggggtgacaggcgtgagccatcgtgactggccggctacgtttatttatttatttttttaattattttacttttttttagttttccattttaatctatttatttatttacatttatttatttatttatttatttacttatttatttattttcgagacagactctcgctctgctgcccaggctggagtgcagcggcgtgatctcggctcactgcaacgtccgcctcccgggttcacgccattctcctgcctcagcctcccaagtagctgggactacaggcgcccgccaccgtgcccggctaactttttgtattttgagtagagatggggtttcactgtggtagccaggatggtctcgatctcctgaccccgtgatccgtccacctcggcctcccaaagtgctgggatgacaggcgtgagccaccggccccggcctatttatctatttattaactttgagtccaggttatgaaaccagttagtttttgtaattttttttttttttttttttttttgagacgaggtttcaccgtgttgccaaggcttggaccgagggatccaccggccctcggcctcccaaaagtgcggggatgacaggcgcgagcctaccgcgcccggaccccccctttccccttcccccgcttgtcttcccgacagacagtttcacggcagagcgtttggctggcgtgcttaaactcattctaaatagaaatttgggacgtcagcttctggcctcacggactctgagccgaggagtcccctggtctgtctatcacaggaccgtacacgtaaggaggagaaaaatcgtaacgttcaaagtcagtcattttgtgatacagaaatacacggattcacccaaaacacagaaaccagtcttttagaaatggccttagccctggtgtccgtgccagtgattcttttcggtttggaccttgactgagaggattcccagtcggtctctcgtctctggacggaagttccagatgatccgatgggtgggggacttaggctgcgtccccccaggagccctggtcgattagttgtggggatcgccttggagggcgcggtgacccactgtgctgtgggagcctccatccttccccccaccccctccccagggggatcccaattcattccgggctgacacgctcactggcaggcgtcgggcatcacctagcggtcactgttactctgaaaacggaggcctcacagaggaagggagcaccaggccgcctgcgcacagcctggggcaactgtgtcttctccaccgcccccgcccccacctccaagttcctccctcccttgttgcctaggaaatcgccactttgacgaccgggtctgattgacctttgatcaggcaaaaacgaacaaacagataaataaataaaataacacaaaagtaactaactaaataaaataagtcaatacaacccattacaatacaataagatacgatacgataggatgcgataggatacgataggatacaatacaataggatacgatacaatacaatacaatacaatacaatacaatacaatacaatacaatacaatacaatacaatacgccgggcgcggtggctcatgcctgtcatcccgtcactttgggatgccgaggtggacgcatcacctgaagtcgggagttggagacaagcccgaccaacatggagaaatcccgtctcaattgaaaatacaaaactagccgggcgcggtggcacatgcctataatcccagctgctaggaaggctgaggcaggagaatcgcttgaacctgggaagcggaggttgcagtgagccgagattgcgccatcgcactccagtctgagcaacaagagcgaaactccgtctcaaaaataaatacataaataaatacatacatacatacatacatacatacatacatacatacataaattaaaataaataaataaaataaaataaataaatgggccctgcgcggtggctcaagcctgtcatcccctcactttgggaggccaaggccggtggatcaagaggcggtcagaccaacagggccagtatggtgaaaccccgtctctactcacaatacacaacattagccgggcgctgtgctgtgctgtactgtctgtaatcccagctactcgggaggccgagctgaggcaggagaatcgcttgaacctgggaggcggaggttgcagtgagccgagatcgcgccactgcaacccagcctgggcgacagagcgagactccgtctccaaaaaatgaaaatgaaaatgaaacgcaacaaaataattaaaaagtgagtttctggggaaaaagaagaaaagaaaaaagaaaaaaacaacaaaacagaacaaccccaccgtgacatacacgtacgcttctcgcctttcgaggcctcaaacacgttaggaattatgcgtgatttctttttttaacttcattttatgttattatcatgattgatgtttcgagacggagtctcggaggcccgccctccctggttgcccagacaaccccgggagacagaccctggctgggcccgattgttcttctccttggtcaggggtttccttgtctttcttcgtgtctttaacccgcgtggactcttccgcctcgggtttgacagatggcagctccactttaggccttgttgttgttggggactttcctgattctccccagatgtagtgaaagcaggtagattgccttgcctggccttgcctggccttgccttttctttctttctttctttctttattactttctctttttcttcttcttcttcttcttttttttgagacagagtttcactcttgttgcccaggctagagggcaatggcgcgatctcggctcaccgcaccctccgcctcccaggttcaagcgattctcctgcctcagcctcctgattagctgggattacaggcatgggccaccgtgctggctgatgtttgtacttttagtagagacggtgtttttccatgttggtcaggctggtctcccactcccaacctcaggtggtccgcctgccttagcctcccaaagtgctgggatgacaggcgtgcaaccgcgcccagcctctctctctctctctctctctctcgctcgcttgcttgcttgctttcgtgctttcttgctttcccgttttcttgctttctttctttctttcgtttctttcatgcttgctttcttgcttgcttgcttgctttcgtgctttcttgctttcctgttttctttctttctttctttctttctttcttttgtttctttcttgcttgctttcttgcttgcttgcttgctttcgtgctttcttgctttcctgttttctttctttctttctttcttttctttctttcttgcttgctttcctgcttgcttgctttcgtgctttcttgttttctcgatttctttctttcttttgtttctttcctgcttgctttcttgcttgcttgctttcgtgcttcttgctttcctgttttctttctttctttctttcttttgtttctttcttgcttgctttcttgcttgcttgctttcgtgctgtcttgtttctcgatttctttctttcttttgtttctttcctgcttgctttcttgcttgattgctttcgtgctttcttgctttcttgttttctttctttcttttgtttctttctttcttgcttccttgttttcttgctttcttgcttgcttgctttcgtgctttcttgttttcttgctttctttcttttgtttctttcttgcttgctttcttgcttccttgttttcttgctttcttgcttgcttgctttcgtgctttctttcttgctttcttttctttctttcttttctttttctttctttcttgctttcttttctttcatcatcatctttctttctttcctttctttctttctttctttctatctttctttctttctttctttctttctttctttctttctttctgtttcgtccttttgagacagagtttcactcttgtttccacggctagagtgcaatggcgcgatcttggctcaccgcaccttccgcctcccgggttcgagcgcttctcctgcctccagcctcccgattagcggggattgacagggaggcacccccacgcctggcttggctgatgtttgtgtttttagtaggcacgccgtgtctctccatgttgctcaggctggtctccaactcccgacctcctgtgatgcgcccacctcggcctctcgaagtgctgggatgacgggcgtgacgaccgtgcccggcctgttgactcatttcgcttttttatttctttcgtttccacgcgtttacttatatgtattaatgtaaacgtttctgtacgcttatatgcaaacaacgacaacgtgtatctctgcattgaatactcttgcgtatggtaaatacgtatcggttgtatggaaatagacttctgtatgatagatgtaggtgtctgtgttatacaaataaatacacatcgctctataaagaagggatcgtcgataaagacgtttattttacgtatgaaaagcgtcgtatttatgtgtgtaaatgaaccgagcgtacgtagttatctctgttttctttcttcctctccttcgtgtttttcttccttcctttcttcctttctctccttctttaggtttttcttcctctcttcctttccttctttctctctttctgtccttttttccttcgtgctttatttctctttcgttccctgtgtttccttcttttttctttcctctctgtttctttttcccttctttccttcgtttctttcctcattctttctctctttttcgttgtttctttccttcccgtctgtcttttaaaaaattggagtgtttcagaagtttactttgtgtatctacgttttctaaattgtctctcttttctccattttcttcctccctccctccctccctccctgctcccttccctccctccttccctttcgccatctgtctcttttccccactcccctccccccgtctgtctctgcgtggattccggaagagcctaccgattctgcctctccgtgtgtctgcagcgaccccgcgaccgagtccttgtgtgttctttctccctccctccctccctccctccctccctccctccctgcttccgagaggcatctccagagaccgcgccgtgggttgtcttctgactctgtcgcggtcgaggcagagacgcgttttgggcaccgtttgtgtggggttggggcagaggggctgcgttttcggcctcgggaagagcttctcgactcacggtttcgctttcgcggtccacgggccgccctgccagccggatctgtctcgctgacgtccgcggcggttgtcgggctccatctggcggccgctttgagatcgtgctctcggcttccggagctgcggtggcagctgccgagggaggggaccgtccccgctgtgagctaggcagagctccggaaagcccgcggtcgtcagcccggctggcccggtggcgccagagctgtggccggtcgcttgtgagtcacagctctggcgtgcaggtttatgtgggggagaggctgtcgctgcgcttctgggcccgcggcgggcgtggggctgcccgggccggtcgaccagcgcgccgtagctcccgaggcccgagccgcgacccggcggacccgccgcgcgtggcggaggctggggacgcccttcccggcccggtcgcggtccgctcatcctggccgtctgaggcggcggccgaattcgtttccgagatccccgtggggagccggggaccgtcccgcccccgtcccccgggtgccggggagcggtccccgggccgggccgcggtccctctgccgcgatcctttctggcgagtccccgtggccagtcggagagcgctccctgagccggtgcggcccgagaggtcgcgctggccggccttcggtccctcgtgtgtcccggtcgtaggaggggccggccgaaaatgcttccggctcccgctctggagacacgggccggcccctgcgtgtggccagggcggccgggagggctccccggcccggcgctgtccccgcgtgtgtccttgggttgaccagagggaccccgggcgctccgtgtgtggctgcgatggtggcgtttttggggacaggtgtccgtgtccgtgtcgcgcgtcgcctgggccggcggcgtggtcggtgacgcgacctcccggccccgggggaggtatatctttcgctccgagtcggcaattttgggccgccgggttatat

SEQ ID NO: 2 - DNA, 25 bp, Homo sapiens
gggtggacgggggggcctggtgggg

SEQ ID NO: 3 - DNA, 68 bp, Homo sapiens
cccgggtgcccttgccctcgcggtccccggccctcgcccgtctgtgccctcttccccgcccgccgccc

SEQ ID NO: 4 - DNA, 28 bp, Homo sapiens
gggtcggggggtggggcccgggccgggg

SEQ ID NO: 5 - DNA, 24 bp, Homo sapiens
ccccgccccggccccaccggtccc

SEQ ID NO: 6 - DNA, 33 bp, Homo sapiens
cccccgcgcccgctcgctccctcccgtccgccc

SEQ ID NO: 7 - DNA, 30 bp, Homo sapiens
gggtcgggggcggtggtgggcccgcggggg

SEQ ID NO: 8 - DNA, 30 bp, Homo sapiens
cccgccccttccccctccccccgcgggccc

SEQ ID NO: 9 - DNA, 29 bp, Homo sapiens
gggggcgggaacccccgggcgcctgtggg

SEQ ID NO: 10 - DNA, 23 bp, Homo sapiens
gggtggcgggggggagagggggg

SEQ ID NO: 11 - DNA, 45 bp, Homo sapiens
gggtccggaaggggaagggtgccggcggggagagagggtcggggg

SEQ ID NO: 12 - DNA, 30 bp, Homo sapiens
ccccgcgcccctcctcctccccgccgcccc

SEQ ID NO: 13 - DNA, 30 bp, Homo sapiens
cccgtcccgcccccggcccgtgcccctccc

SEQ ID NO: 14 - DNA, 55 bp, Homo sapiens
cccgccccccgttcctcccgacccctccacccgccctcccttcccccgccgcccc

SEQ ID NO: 15 - DNA, 62 bp, Homo sapiens
gggggcgggctccggcgggtgcgggggtgggcgggcggggccgggggtggggtcggcggggg

SEQ ID NO: 16 - DNA, 32 bp, Homo sapiens
cccgtctccgccccccggccccgcgtcctccc

SEQ ID NO: 17 - DNA, 21 bp, Homo sapiens
gggagggcgcgcgggtcgggg

SEQ ID NO: 18 - DNA, 23 bp, Homo sapiens
cccccctcccggcgcccaccccc

SEQ ID NO: 19 - DNA, 29 bp, Homo sapiens
cccacccctcctccccgcgcccccgcccc

SEQ ID NO: 20 - DNA, 43 bp, Homo sapiens
cccctcctcccgcccacgccccgctccccgcccccggagcccc

SEQ ID NO: 21 - DNA, 23 bp, Homo sapiens
gggctgggtcggtcgggctgggg

SEQ ID NO: 22 - DNA, 50 bp, Homo sapiens
ccccccccacgcccggggcacccccctcgcggccctcccccgccccaccc

SEQ ID NO: 23 - DNA, 19 bp, Homo sapiens
ccctccccaccccgcgccc

SEQ ID NO: 24 - DNA, 28 bp, Homo sapiens
cccccgctccccgtcctcccccctcccc

SEQ ID NO: 25 - DNA, 38 bp, Homo sapiens
ggggcgcggcggggggagaagggtcggggcggcagggg

SEQ ID NO: 26 - DNA, 45 bp, Homo sapiens
ccccccgccctacccccccggccccgtccgccccccgttcccccc

SEQ ID NO: 27 - DNA, 25 bp, Homo sapiens
cccccggcgcccccccggtgtcccc

SEQ ID NO: 28 - DNA, 21 bp, Homo sapiens
gggccgggacggggtccgggg

SEQ ID NO: 29 - DNA, 26 bp, Homo sapiens
ccccgtggcccgccggtccccgtccc

SEQ ID NO: 30 - DNA, 34 bp, Homo sapiens
ccctccctccctccccctccctccctctctcccc

SEQ ID NO: 31 - DNA, 36 bp, Homo sapiens
cccccacccccccgtcacgtcccgctaccctccccc

SEQ ID NO: 32 - DNA, 57 bp, Homo sapiens
gggggtgcgggaatgagggtgtgtgtggggagggggtgcggggtggggacggagggg

SEQ ID NO: 33 - DNA, 27 bp, Homo sapiens
ggggagagaggggggagaggggggggg

SEQ ID NO: 34 - DNA, 37 bp, Homo sapiens
ccccaaaccgcccccccccccccgcctcccaacaccc

SEQ ID NO: 35 - DNA, 37 bp, Homo sapiens
ccccacccacgccccacgccccacgtcccgggcaccc

SEQ ID NO: 36 - DNA, 38 bp, Homo sapiens
gggaggggtgggggtggggtgggttgggggttgtgggg

SEQ ID NO: 37 - DNA, 27 bp, Homo sapiens
cccggaccccccctttccccttccccc

SEQ ID NO: 38 - DNA, 30 bp, Homo sapiens
cccgccctccctggttgcccagacaacccc

SEQ ID NO: 39 - DNA, 43 bp, Homo sapiens
ccctccctccctccctccctgctcccttccctccctccttccc

SEQ ID NO: 40 - DNA, 24 bp, Homo sapiens
ccccctcccttccccaggcgtccc

SEQ ID NO: 41 - DNA, 18 bp, Homo sapiens
gggagggagacggggggg

SEQ ID NO: 42 - DNA, 19 bp, Homo sapiens
gggcggggggggcgggggg

SEQ ID NO: 43 - DNA, 19 bp, Homo sapiens
cccgccccgccgcccgccc

SEQ ID NO: 44 - DNA, 17 bp, Homo sapiens
cccccgccccccccccc

SEQ ID NO: 45 - DNA, 16 bp, Homo sapiens
ggggtgggggggaggg

SEQ ID NO: 46 - DNA, 36 bp, Homo sapiens
ccctccctccctccctccctccctccctccctcccc

SEQ ID NO: 47 - DNA, 24 bp, Homo sapiens
ggggtggggtggggtggggtgggg

SEQ ID NO: 48 - DNA, 30 bp, Homo sapiens
ccccccggctccccccactacccacgtccc

SEQ ID NO: 49 - DNA, 35 bp, Homo sapiens
ccctccctccctccctccctccctccctccctccc

SEQ ID NO: 50 - DNA, 30 bp, Homo sapiens; identical to SEQ ID NO: 7

SEQ ID NO: 51 - DNA, 55 bp, Homo sapiens; identical to SEQ ID NO: 14

SEQ ID NO: 52 - DNA, 62 bp, Homo sapiens; identical to SEQ ID NO: 15

SEQ ID NO: 53 - DNA, 32 bp, Homo sapiens; identical to SEQ ID NO: 16

SEQ ID NO: 54 - DNA, 21 bp, Homo sapiens
gggagggcgcgcgggtcgggg

SEQ ID NO: 55 - DNA, 17 bp, Homo sapiens
cccccgccccccccccc

SEQ ID NO: 56 - DNA, 43 bp, Homo sapiens; identical to SEQ ID NO: 20

SEQ ID NO: 57 - DNA, 23 bp, Homo sapiens
gggctgggtcggtcgggctgggg

SEQ ID NO: 58 - DNA, 25 bp, Homo sapiens
cccccggcgcccccccggtgtcccc

SEQ ID NO: 59 - DNA, 25 bp, Homo sapiens
ccccaccaggcccccccgtccaccc

SEQ ID NO: 60 - DNA, 24 bp, Homo sapiens
gggacgcctggggaagggaggggg

SEQ ID NO: 61 - DNA, 68 bp, Homo sapiens
gggcggcgggcggggaagagggcacagacgggcgagggccggggaccgcgagggcaagggcacccggg

SEQ ID NO: 62 - DNA, 28 bp, Homo sapiens
ccccggcccgggccccaccccccgaccc

SEQ ID NO: 63 - DNA, 24 bp, Homo sapiens
gggaccggtggggccggggcgggg

SEQ ID NO: 64 - DNA, 33 bp, Homo sapiens
gggcggacgggagggagcgagcgggcgcggggg

SEQ ID NO: 65 - DNA, 18 bp, Homo sapiens
cccccccgtctccctccc

SEQ ID NO: 66 - DNA, 30 bp, Homo sapiens
cccccgcgggcccaccaccgcccccgaccc

SEQ ID NO: 67 - DNA, 30 bp, Homo sapiens
gggcccgcggggggagggggaaggggcggg

SEQ ID NO: 68 - DNA, 29 bp, Homo sapiens
cccacaggcgcccgggggttcccgccccc

SEQ ID NO: 69 - DNA, 19 bp, Homo sapiens
ccccccgccccccccgccc

SEQ ID NO: 70 - DNA, 23 bp, Homo sapiens
cccccctctcccccccgccaccc

SEQ ID NO: 71 - DNA, 45 bp, Homo sapiens
cccccgaccctctctccccgccggcacccttccccttccggaccc

SEQ ID NO: 72 - DNA, 30 bp, Homo sapiens
ggggcggcggggaggaggaggggcgcgggg

SEQ ID NO: 73 - DNA, 19 bp, Homo sapiens
gggcgggcggcggggcggg

SEQ ID NO: 74 - DNA, 30 bp, Homo sapiens
gggaggggcacgggccgggggcgggacggg

SEQ ID NO: 75 - DNA, 55 bp, Homo sapiens
ggggcggcgggggaagggagggcgggtggaggggtcgggaggaacggggggcggg

SEQ ID NO: 76 - DNA, 62 bp, Homo sapiens
cccccgccgaccccacccccggccccgcccgcccacccccgcacccgccggagcccgccccc

SEQ ID NO: 77 - DNA, 32 bp, Homo sapiens
gggaggacgcggggccggggggcggagacggg

SEQ ID NO: 78 - DNA, 21 bp, Homo sapiens
ccccgacccgcgcgccctccc

SEQ ID NO: 79 - DNA, 23 bp, Homo sapiens
gggggtgggcgccgggagggggg

SEQ ID NO: 80 - DNA, 29 bp, Homo sapiens
ggggcgggggcgcggggaggaggggtggg

SEQ ID NO: 81 - DNA, 17 bp, Homo sapiens
gggggggggggcggggg

SEQ ID NO: 82 - DNA, 43 bp, Homo sapiens
ggggctccgggggcggggagcggggcgtgggcgggaggagggg

SEQ ID NO: 83 - DNA, 23 bp, Homo sapiens
ccccagcccgaccgacccagccc

SEQ ID NO: 84 - DNA, 50 bp, Homo sapiens
gggtggggcgggggagggccgcgaggggggtgccccgggcgtgggggggg

SEQ ID NO: 85 - DNA, 19 bp, Homo sapiens
gggcgcggggtggggaggg

SEQ ID NO: 86 - DNA, 28 bp, Homo sapiens
ggggaggggggaggacggggagcggggg

SEQ ID NO: 87 - DNA, 38 bp, Homo sapiens
cccctgccgccccgacccttctccccccgccgcgcccc

SEQ ID NO: 88 - DNA, 45 bp, Homo sapiens
ggggggaacggggggcggacggggccgggggggtagggcgggggg

SEQ ID NO: 89 - DNA, 25 bp, Homo sapiens
ggggacaccgggggggcgccggggg

SEQ ID NO: 90 - DNA, 16 bp, Homo sapiens
ccctcccccccacccc

SEQ ID NO: 91 - DNA, 21 bp, Homo sapiens
ccccggaccccgtcccggccc

SEQ ID NO: 92 - DNA, 26 bp, Homo sapiens
gggacggggaccggcgggccacgggg

SEQ ID NO: 93 - DNA, 36 bp, Homo sapiens
ggggagggagggagggagggagggagggagggaggg

SEQ ID NO: 94 - DNA, 34 bp, Homo sapiens
ggggagagagggagggagggggagggagggaggg

SEQ ID NO: 95 - DNA, 24 bp, Homo sapiens
ccccaccccaccccaccccacccc

SEQ ID NO: 96 - DNA, 36 bp, Homo sapiens
gggggagggtagcgggacgtgacgggggggtggggg

SEQ ID NO: 97 - DNA, 57 bp, Homo sapiens
cccctccgtccccaccccgcaccccctccccacacacaccctcattcccgcaccccc

SEQ ID NO: 98 - DNA, 27 bp, Homo sapiens
ccccccccctctcccccctctctcccc

SEQ ID NO: 99 - DNA, 30 bp, Homo sapiens
gggacgtgggtagtggggggagccgggggg

SEQ ID NO: 100 - DNA, 37 bp, Homo sapiens
gggtgttgggaggcgggggggggggggcggtttgggg

SEQ ID NO: 101 - DNA, 37 bp, Homo sapiens
gggtgcccgggacgtggggcgtggggcgtgggtgggg

SEQ ID NO: 102 - DNA, 38 bp, Homo sapiens
ccccacaacccccaacccaccccacccccacccctccc

SEQ ID NO: 103 - DNA, 27 bp, Homo sapiens
gggggaaggggaaagggggggtccggg

SEQ ID NO: 104 - DNA, 30 bp, Homo sapiens
ggggttgtctgggcaaccagggagggcggg

SEQ ID NO: 105 - DNA, 43 bp, Homo sapiens
gggaaggagggagggaagggagcagggagggagggagggaggg

SEQ ID NO: 106 - DNA, 35 bp, Homo sapiens
gggagggagggagggagggagggagggagggaggg

SEQ ID NO: 107 - RNA, 26 nt, Homo sapiens
gggguggacgggggggccuggugggg

SEQ ID NO: 108 - RNA, 28 nt, Homo sapiens
gggucgggggguggggcccgggccgggg

SEQ ID NO: 109 - RNA, 18 nt, Homo sapiens
gggagggagacggggggg

SEQ ID NO: 110 - RNA, 30 nt, Homo sapiens
gggucgggggcgguggugggcccgcggggg

SEQ ID NO: 111 - RNA, 29 nt, Homo sapiens
gggggcgggaacccccgggcgccuguggg

SEQ ID NO: 112 - RNA, 23 nt, Homo sapiens
ggguggcgggggggagagggggg

SEQ ID NO: 113 - RNA, 45 nt, Homo sapiens
ggguccggaaggggaagggugccggcggggagagagggucggggg

SEQ ID NO: 114 - RNA, 62 nt, Homo sapiens
gggggcgggcuccggcgggugcgggggugggcgggcggggccggggguggggucggcggggg

SEQ ID NO: 115 - RNA, 21 nt, Homo sapiens
gggagggcgcgcgggucgggg

SEQ ID NO: 116 - RNA, 23 nt, Homo sapiens
gggcugggucggucgggcugggg

SEQ ID NO: 117 - RNA, 38 nt, Homo sapiens
ggggcgcggcggggggagaagggucggggcggcagggg

SEQ ID NO: 118 - RNA, 21 nt, Homo sapiens
gggccgggacgggguccgggg

SEQ ID NO: 119 - RNA, 19 nt, Homo sapiens
gggcggggggggcgggggg

SEQ ID NO: 120 - RNA, 16 nt, Homo sapiens
ggggugggggggaggg

SEQ ID NO: 121 - RNA, 24 nt, Homo sapiens
cccccucccuuccccaggcguccc

SEQ ID NO: 122 - RNA, 68 nt, Homo sapiens
cccgggugcccuugcccucgcgguccccggcccucgcccgucugugcccucuuccccgcccgccgccc

SEQ ID NO: 123 - RNA, 24 nt, Homo sapiens
ccccgccccggccccaccgguccc

SEQ ID NO: 124 - RNA, 33 nt, Homo sapiens
cccccgcgcccgcucgcucccucccguccgccc

SEQ ID NO: 125 - RNA, 30 nt, Homo sapiens
cccgccccuucccccuccccccgcgggccc

SEQ ID NO: 126 - RNA, 30 nt, Homo sapiens
ccccgcgccccuccuccuccccgccgcccc

SEQ ID NO: 127 - RNA, 19 nt, Homo sapiens
cccgccccgccgcccgccc

SEQ ID NO: 128 - RNA, 30 nt, Homo sapiens
cccgucccgcccccggcccgugccccuccc

SEQ ID NO: 129 - RNA, 55 nt, Homo sapiens
cccgccccccguuccucccgaccccuccacccgcccucccuucccccgccgcccc

SEQ ID NO: 130 - RNA, 32 nt, Homo sapiens
cccgucuccgccccccggccccgcguccuccc

SEQ ID NO: 131 - RNA, 23 nt, Homo sapiens
ccccccucccggcgcccaccccc

SEQ ID NO: 132 - RNA, 29 nt, Homo sapiens
cccaccccuccuccccgcgcccccgcccc

SEQ ID NO: 133 - RNA, 17 nt, Homo sapiens
cccccgccccccccccc

SEQ ID NO: 134 - RNA, 43 nt, Homo sapiens
ccccuccucccgcccacgccccgcuccccgcccccggagcccc

SEQ ID NO: 135 - RNA, 50 nt, Homo sapiens
ccccccccacgcccggggcaccccccucgcggcccucccccgccccaccc

SEQ ID NO: 136 - RNA, 19 nt, Homo sapiens
cccuccccaccccgcgccc

SEQ ID NO: 137 - RNA, 28 nt, Homo sapiens
cccccgcuccccguccuccccccucccc

SEQ ID NO: 138 - RNA, 45 nt, Homo sapiens
ccccccgcccuacccccccggccccguccgccccccguucccccc

SEQ ID NO: 139 - RNA, 25 nt, Homo sapiens
cccccggcgcccccccggugucccc

SEQ ID NO: 140 - RNA, 26 nt, Homo sapiens
ccccguggcccgccgguccccguccc

SEQ ID NO: 141 - RNA, 30 nt, Homo sapiens
auucauaaggaguacucgaucacgcgaagu

SEQ ID NO: 142 - RNA, 32 nt, Homo sapiens
acauucgaaccgacaccugugccuuaccgcgu

SEQ ID NO: 143 - RNA, 30 nt, Homo sapiens
auugucagagacucgagcguaccaacuggu

SEQ ID NO: 144 - RNA, 32 nt, Homo sapiens
acauuaucaaucuagcuaggguguacacaagu

SEQ ID NO: 145 - RNA, 32 nt, Homo sapiens
acauucgaaccaaccugacacccuaucccagu

SEQ ID NO: 146 - RNA, 32 nt, Homo sapiens
auugcgaccgguucugccaauacucgagguug

SEQ ID NO: 147 - RNA, 30 nt, Homo sapiens
auuagggugugaaugugcugaucaacgcgu

SEQ ID NO: 148 - RNA, 32 nt, Homo sapiens
acauucgaaugucaaugcgcaaguagaccggu

SEQ ID NO: 149 - RNA, 30 nt, Homo sapiens
auugaucaauauucgaccacccugcagcgu

SEQ ID NO: 150 - RNA, 30 nt, Homo sapiens
auugcgcaugucacgcuucgaagccgcugu

SEQ ID NO: 151 - RNA, 9 nt, Homo sapiens
auucgaccg

SEQ ID NO: 152 - RNA, 11 nt, Homo sapiens
gaucgaugugg

SEQ ID NO: 153 - RNA, 11 nt, Homo sapiens
gaucgaucugg

SEQ ID NO: 154 - DNA, 41 bp, Homo sapiens
tctctcggtggccggggctcgtcggggttttgggtccgtcc

SEQ ID NO: 155 - DNA, 32 bp, Homo sapiens
actgtcgtacttgatattttggggttttgggg

SEQ ID NO: 156 - DNA, 48 bp, Homo sapiens
tggaccagacctagcagctatgggggagctggggaaggtgggatgtga

SEQ ID NO: 157 - DNA, 32 bp, Homo sapiens
agacctagcagctatgggggagctggggtata

SEQ ID NO: 158 - DNA, 17 bp, Homo sapiens
gggggggggggcggggg

SEQ ID NO: 159 - DNA, 16 bp, Homo sapiens
ggggtgggggggaggg

SEQ ID NO: 160 - DNA, 23 bp, Homo sapiens
gggtggcgggggggagagggggg

SEQ ID NO: 161 - DNA, 19 bp, Homo sapiens
gggcggggggggcgggggg

SEQ ID NO: 162 - DNA, 25 bp, Homo sapiens
gggtggacgggggggcctggtgggg

SEQ ID NO: 163 - DNA, 28 bp, Homo sapiens
gggtcggggggtggggcccgggccgggg

SEQ ID NO: 164 - DNA, 18 bp, Homo sapiens
gggagggagacggggggg

SEQ ID NO: 165 - DNA, 29 bp, Homo sapiens
gggggtgggcgggcggggccgggggtggg

SEQ ID NO: 166 - DNA, 30 bp, Homo sapiens; identical to SEQ ID NO: 7

SEQ ID NO: 167 - DNA, 38 bp, Homo sapiens; identical to SEQ ID NO: 25

SEQ ID NO: 168 - DNA, 29 bp, Homo sapiens
gggggcgggaacccccgggcgcctgtggg

SEQ ID NO: 169 - DNA, 30 bp, Homo sapiens; identical to SEQ ID NO: 74

SEQ ID NO: 170 - DNA, 45 bp, Homo sapiens; identical to SEQ ID NO: 11

SEQ ID NO: 171 - DNA, 21 bp, Homo sapiens
gggccgggacggggtccgggg

SEQ ID NO: 172 - DNA, 30 bp, Homo sapiens; identical to SEQ ID NO: 67

SEQ ID NO: 173 - DNA, 21 bp, Homo sapiens
gggagggcgcgcgggtcgggg

SEQ ID NO: 174 - DNA, 23 bp, Homo sapiens
gggctgggtcggtcgggctgggg

SEQ ID NO: 175 - DNA, 64 bp, Homo sapiens
cggagggcgcgcgggtcggggcggcggcggcggcggcggtggcggcggcggcgggggcggcggg

SEQ ID NO: 176 - DNA, 27 bp, Homo sapiens
tggggagggtggggagggtggggaagg

SEQ ID NO: 177 - RNA, 21 nt, Homo sapiens
gccgaaaucccgaaguaggcc

SEQ ID NO: 178 - DNA, 19 bp, Homo sapiens
agggagggagacggggggg

SEQ ID NO: 179 - DNA, 25 bp, Homo sapiens
agggacgcctggggaagggaggggg

SEQ ID NO: 180 - DNA, 69 bp, Homo sapiens
agggcggcgggcggggaagagggcacagacgggcgagggccggggaccgcgagggcaagggcacccggg

SEQ ID NO: 181 - DNA, 25 bp, Homo sapiens
agggaccggtggggccggggcgggg

SEQ ID NO: 182 - DNA, 34 bp, Homo sapiens
agggcggacgggagggagcgagcgggcgcggggg

SEQ ID NO: 183 - DNA, 31 bp, Homo sapiens
aggggcggcggggaggaggaggggcgcgggg

SEQ ID NO: 184 - DNA, 20 bp, Homo sapiens
agggcgggcggcggggcggg

SEQ ID NO: 185 - DNA, 56 bp, Homo sapiens
aggggcggcgggggaagggagggcgggtggaggggtcgggaggaacggggggcggg

SEQ ID NO: 186 - DNA, 33 bp, Homo sapiens
agggaggacgcggggccggggggcggagacggg

SEQ ID NO: 187 - DNA, 24 bp, Homo sapiens
agggggtgggcgccgggagggggg

SEQ ID NO: 188 - DNA, 30 bp, Homo sapiens
aggggcgggggcgcggggaggaggggtggg

SEQ ID NO: 189 - DNA, 17 bp, Homo sapiens
gggggggggggcggggg

SEQ ID NO: 190 - DNA, 44 bp, Homo sapiens
aggggctccgggggcggggagcggggcgtgggcgggaggagggg

SEQ ID NO: 191 - DNA, 51 bp, Homo sapiens
agggtggggcgggggagggccgcgaggggggtgccccgggcgtgggggggg

SEQ ID NO: 192 - DNA, 20 bp, Homo sapiens
agggcgcggggtggggaggg

SEQ ID NO: 193 - DNA, 29 bp, Homo sapiens
aggggaggggggaggacggggagcggggg

SEQ ID NO: 194 - DNA, 46 bp, Homo sapiens
aggggggaacggggggcggacggggccgggggggtagggcgggggg

SEQ ID NO: 195 - DNA, 26 bp, Homo sapiens
aggggacaccgggggggcgccggggg

SEQ ID NO: 196 - DNA, 27 bp, Homo sapiens
agggacggggaccggcgggccacgggg

SEQ ID NO: 197 - DNA, 22 bp, Homo sapiens
agggttagggttagggttaggg

SEQ ID NO: 198 - DNA, 27 bp, Homo sapiens
tggggagggtggggagggtggggaatt

SEQ ID NO: 199 - DNA, 27 bp, Homo sapiens
gtcgtaacgtcgatcagtttacgacat

SEQ ID NO: 200 - DNA, 12 bp, Homo sapiens
ggaggaggagga

SEQ ID NO: 201 - DNA, 15 bp, Homo sapiens
tccaactatgtatac

SEQ ID NO: 202 - DNA, 35 bp, Homo sapiens
ttagcgacacgcaattgctatagtgagtcgtatta

SEQ ID NO: 203 - protein, 13 aa, Artificial Sequence
  Substrate peptide
KKLNRTLSFAEPG

SEQ ID NO: 204 - protein, 10 aa, Artificial Sequence
  Substrate peptide
RRRLSFAEPG

SEQ ID NO: 205 - protein, 15 aa, Artificial Sequence
  Substrate peptide
GGEEEEYFELVKKKK

SEQ ID NO: 206 - protein, 12 aa, Artificial Sequence
  Substrate peptide
EAIYAAPFAKKK

SEQ ID NO: 207 - protein, 14 aa, Artificial Sequence
  Substrate peptide
KKKSPGEYVNIEFG

SEQ ID NO: 208 - protein, 15 aa, Artificial Sequence
  Substrate peptide
AMARAASAAALARRR

SEQ ID NO: 209 - protein, 7 aa, Artificial Sequence
  Substrate peptide
LRRASLG

SEQ ID NO: 210 - protein, 13 aa, Artificial Sequence
  Substrate peptide
KKSRGDYMTMQIG

SEQ ID NO: 211 - protein, 15 aa, Artificial Sequence
  Substrate peptide
KVEKIGEGTYGVVYK

SEQ ID NO: 212 - protein, 10 aa, Artificial Sequence
  Substrate peptide
KKLNRTLSVA

SEQ ID NO: 213 - protein, 23 aa, Artificial Sequence
  Substrate peptide
KKKVSRSGLYRSPSMPENLNRPR

SEQ ID NO: 214 - protein, 14 aa, Artificial Sequence
  Substrate peptide
KRRRALXVASLPGL

SEQ ID NO: 215 - protein, 10 aa, Artificial Sequence
  Substrate peptide
RRRDDDSDDD

SEQ ID NO: 216 - protein, 26 aa, Artificial Sequence
  Substrate peptide
YRRAAVPPSPSLSRHSSPHQXEDEEE

SEQ ID NO: 217 - protein, 39 aa, Artificial Sequence
  Substrate peptide
KTFCGTPEYLAPEVRREPRILSEEEQEMFRDFDYIADWC

SEQ ID NO: 218 - protein, 15 aa, Artificial Sequence
  Substrate peptide
GGEEEEYFELVKKKK

SEQ ID NO: 219 - protein, 9 aa, Artificial Sequence
  Substrate peptide
KKRNRTLTV

SEQ ID NO: 220 - protein, 13 aa, Artificial Sequence
  Substrate peptide
GRPRTSSFAEGKK

SEQ ID NO: 221 - protein, 15 aa, Artificial Sequence
  Substrate peptide
FLAKSFGSPNRAYKK

SEQ ID NO: 222 - protein, 32 aa, Artificial Sequence
  Substrate peptide
KEAKEKRQEQIAKRRRLSSLRASTSKSGGSQK

SEQ ID NO: 223 - protein, 10 aa, Artificial Sequence
  Substrate peptide
RRRLSFAEPG

SEQ ID NO: 224 - protein, 16 aa, Artificial Sequence
  Substrate peptide
ERMRPRKRQGSVRRRV

SEQ ID NO: 225 - protein, 10 aa, Artificial Sequence
  Substrate peptide
KKLRRTLSVA

SEQ ID NO: 226 - protein, 11 aa, Artificial Sequence
  Substrate peptide
AKRRRLSSLRA

SEQ ID NO: 227 - DNA, 17 bp, Artificial Sequence
  Probe
ttgatcctgccagtagc

SEQ ID NO: 228 - DNA, 22 bp, Artificial Sequence
  Forward primer
ccgcgctctaccttacctacct

SEQ ID NO: 229 - DNA, 24 bp, Artificial Sequence
  Reverse primer
gcatggcttaatctttgagacaag

SEQ ID NO: 230 - DNA, 7 bp, Artificial Sequence
  Quadruplex forming subsequence
gngngng

SEQ ID NO: 231 - DNA, 7 bp, Artificial Sequence
  Quadruplex forming subsequence
cncncnc